(19) United States
(12) Patent Application Publication
(10) Pub. No.: US 2024/0358829 A1
(43) Pub. Date: Oct. 31, 2024

(54) Lymphocyte Population and Methods for Producing Same

(71) Applicant: AVM Biotechnology, LLC, Seattle, WA (US)

(72) Inventors: Theresa Deisher, Seattle, WA (US); Scot Wayne McKay, US, WA (US); Vaishnavi Parthasarathy, Vancouver BC (CA); Yumna Zahid, Seattle, WA (US)

(73) Assignee: AVM Biotechnology, LLC, Seattle, WA (US)

(21) Appl. No.: 18/683,814

(22) PCT Filed: Aug. 31, 2022
(86) PCT No.: PCT/US2022/042147

§ 371 Date: Feb. 15, 2024

Related U.S. Application Data
(60) Provisional application No. 63/239,566, filed on Sep. 1, 2021.

(51) Int. Cl. A61K 39/00; A61K 31/573; A61P 35/00; A61P 35/02; A61P 37/04; C12N 5/0783

(52) U.S. Cl. CPC A61K 39/4613; A61K 31/573; A61P 35/00; A61P 35/02; A61P 37/04; C12N 5/0646; A61K 2239/38

(57) ABSTRACT

This invention pertains to a novel population of lymphocytes, methods for producing these, and their use in the treatment of diseases.

### FIELD

This disclosure pertains to novel populations of lymphocytes, methods for producing these, and their use in the treatment of diseases. More particularly, the disclosure relates to methods for producing novel populations of a natural killer T cell-like cell (NKT-like cells) using high dose glucocorticoids, glucocorticoid receptor agonists, and ICAM3 modulating agents.

### BACKGROUND

The present authors have previously found that high concentrations of glucocorticoids could be used to condition patients to enhance the efficacy of cellular immunotherapies such as adoptive T cell therapy; described in International patent application PCT/US2018/025517 (published as WO2018/183927). In that application, the authors noted the toxicities associated with chemotherapy and radiation mediated preconditioning, which is believed to non-selectively destroy the cellularity of the spleen. The authors provided glucocorticoids (a subclass of steroids) and other non-toxic lymphodepleting agents, at acute doses, to benefit cancer patients who receive cellular immunotherapies.

In international patent application PCT/US2019/054395 (published as WO2020/072713) the present authors have also described the use of high concentrations of glucocorticoids to cause lymphodepletion of peripheral blood lymphocytes without substantially affecting the cell count of other cells. In that application, the authors reported that high concentrations of glucocorticoids can deplete peripheral blood lymphocytes including, for example, islet-specific autoreactive T-cells responsible for diabetes autoimmunity, but spares neutrophils, platelets, RBCs and stem cells (both HSCs and MSCs). The authors provided glucocorticoids as a non-myeloablative regimen that can perform a safe immunologic reset with efficacy comparable to chemotherapy.

Reducing cytotoxic chemotherapy use is a top priority goal of the National Cancer Institute. Carcinomas, often called solid tumors, represent 80-90% of total cancers, but have proven difficult to target with newer cancer therapy developments. Chimeric antigen receptor (CAR) T-cell therapy has shown remarkable success in the treatment of CD-19-expressing B-cell acute lymphocytic leukemia. However, there are a number of obstacles that limit CAR T-cell therapy for solid tumors: ineffective trafficking to the tumor as well immunosuppressive microenvironments in solid tumors limit T-cell efficacy. In addition, CAR T therapies have been associated with serious adverse effects, including cytokine release syndrome (CRS), neuroedema, and graft versus host disease (GvHD). Furthermore, CAR T therapies are not curative, with up to 50% or subjects relapsing within 12 months even with negative minimal residual disease (Nie et al, 2020). Pre-treatment or “preconditioning” before the CAR T infusion is associated with prolonged survival, and while preconditioning with higher dose chemotherapy is associated with best outcomes, it also has the most severe toxicities. Bi-specific CAR T products, designed to reduce relapse, thought to be due to either tumor escape by lost expression of the CAR T targeted antigen or by heterogeneic expression of the antigen in the tumor, do not appear to be more effective than first-generation CAR T (Gill et al, 2021). To address these limitations of CAR T, the field is moving towards Natural Killer (NK), NK/NKT cell CAR, and gamma delta (γδ) T cell products.

Natural Killer T Cells (NKTs) are a heterogeneous group of T cells that share properties of both T cells and natural killer (NK) cells. In contrast to conventional T cells, NKTs are functionally mature when they exit the thymus, primed for rapid cytokine production. NKTs can directly kill CD1d expressing cancer cells and tumor microenvironment macrophages, rapidly produce and release immune activating cytokines such as IFNgamma and IL-4, and activate other immune cells such as dendritic cells (DCs), NK cells, and B and T lymphocytes. Clinically, invariant NKTs (iNKTs) have been used against a variety of different cancers, either by injection of ‘autologous culture activated iNKTs’, by administering alpha Gal Cer (an NKT activator) loaded dendritic cells or monocytes to activate endogenous NKTs, or by administering NKT activator antibodies or ligands such as KRN7000, a synthetic analogue of alpha Gal Cer.

However, none of these methods used to induce iNKT production have been demonstrated to be effective in cancer patients; iNKT levels are reduced in cancer patients and clinical trials have been disappointing. INKT levels are similarly low in the elderly (Tarazona et al, 2003, which is hereby incorporated by reference in its entirety). Use of ‘autologous culture activated NKTs’ in melanoma was effective in 3 of 9 patients, with outcome directly associated with the number of tumor infiltrating NKTs (Wolf et al, 2018 and Nair et al, 2017, which is hereby incorporated by reference in its entirety). This approach, however, was also limited by the low numbers of NKTs in cancer patients, and by the plasticity of iNKT to move between IFN gamma type 1 and tumor promoting IL-4 type 2.

In cancer treatment, kinase inhibitors (KIs) are well tolerated compared to conventional cytotoxic chemotherapy. However, significant toxicities are still associated with the kinase inhibitors including fatigue, hypertension, rash, impaired wound healing, myelosuppression, and diarrhea, and abnormalities in thyroid function, bone metabolism, linear growth, gonadal function, fetal development, adrenal function, and glucose metabolism. Many patients require dose-reduction because of the toxicities of the KIs, which must be taken chronically (Lodisch et al, 2013, which is hereby incorporated by reference in its entirety). Additionally, resistance to the KIs is common and time-dependent with treatment (Bhullar 2018, which is hereby incorporated by reference in its entirety).

Despite efforts to reduce the toxicities associated with cancer treatments, the physical toll and medical costs to manage these toxicities remain a significant concern. For example, up to 41% of blood cancer patients choose to stop taking the new kinase/proteasome inhibitors or biologics due to the physical and financial toxicities associated with these drugs (Mato 2018, Kadri 2017, Mato 2016 and Barrett 2010, each of which is hereby incorporated by reference in its entirety).

T cells are a type of lymphocyte that play a key role in the immune response. T cells are distinguished from other types of lymphocytes by the presence of T-cell receptors on their cell surface. T-cell receptors (TCRs) are responsible for recognizing fragments of antigen bound to major histocompatibility complex (MHC) molecules, and are heterodimers of two different protein chains. In humans, in 95% of T cells the TCR consists of an alpha (α) chain and a beta (β) chain (encoded by TRA and TRB, respectively), whereas in 5% of T cells the TCR consists of gamma and delta (γ/δ) chains (encoded by TRG and TRD, respectively). This ratio changes in diseased states (such as leukemia).

In contrast to MHC-restricted alpha beta T cells, gamma delta T cells do not require antigen processing and major-histocompatibility-complex (MHC) presentation of peptide epitopes for activation, although some recognize MHC class Ib molecules. Some gamma delta T cells recognise markers of cellular stress resulting from infection or tumorigenesis. Gamma delta T cells are also believed to have a role in recognition of lipid antigens.

Gamma delta T cells display broad functional plasticity following recognition of infected/transformed cells by production of cytokines (IFN-γ, TNF-α, IL-17) and chemokines (RANTES, IP-10, lymphotactin), cytolysis of infected or transformed target cells (perforin, granzymes, TRAIL), and interaction with other cells. Gamma delta T cells have been shown to be capable of recognising and lysing diverse cancers in an MHC-unrestricted manner, to have a protective function in infectious disease, and to be associated with progression and prognosis in various infectious diseases (Gogoi et al, 2013; Pauza et al, 2018; Zheng et al, 2012; Dong et al, 2018; Zhao et al 2018; all hereby incorporated by reference in their entirety). Some gamma delta T cells can also behave as antigen presenting cells in some circumstances (Himoudi et al, 2012). Gamma delta T cells are thus of considerable interest in immunotherapy development.

A need exists for further treatments for cancer, autoimmune disorders, and infectious (also called microbial) diseases that are safer and associated with fewer toxicities and/or greater efficacy than currently available therapies. Treatments that are simpler, less toxic, and less costly are desired.

### SUMMARY

The present invention is based on the surprising finding that while high doses of glucocorticoids act to cause lymphodepletion of many types of peripheral blood lymphocytes in naïve subjects, they also induce production/activation/mobilization of a novel population of Natural Killer T cell-like cells (NKT-like cells). In addition to presenting with the properties of known NKT cells, this novel population of NKT-like cells is able to directly engulf cancer cells, thus expanding the potential of high concentrations of glucocorticoids as a therapeutic treatment for solid cancers. These cells are CD3 high CD49b+, originate from a CD3 high CD49b-population, and may be characterized by the pattern of surface proteins which they express, as described more fully elsewhere herein.

In non-naïve subjects, such as those having a cancer, autoimmune disease, or infectious disease, high dose glucocorticoids deplete diseased/cancerous lymphocytes but spare normal lymphocytes. Without being bound by theory, the present authors hypothesize that lymphodepletion in naïve subjects but not cancer subjects occurs because the NKT-like cells that are induced/mobilized by high dose glucocorticoids express gamma delta TCR, which recognizes phosphoantigens that are either expressed 100-1000 fold higher or selectively expressed on stressed cells that include cancer, autoreactive lymphocytes, and infected cells. In naïve subjects that are pathogen/disease free environments, there are no stressed cells for the NKT-like cells to recognize, and in the absence of stressed cells the NKT-like cells may recognize and deplete normal lymphocytes.

The present authors have also discovered that, following high dose administration, glucocorticoid molecules can bind and block intercellular adhesion molecules such as ICAM3. The binding is cooperative and up to 26 molecules can bind the first Ig domain of ICAM3. ICAM3 is expressed at substantial levels on cells such as lymphocytes, monocytes and neutrophils, as well as on cancer cell types such as melanoma and osteosarcoma. Molecular modelling of the interaction between dexamethasone and ICAM3 confirms these interact via low affinity hydrogen bonding. Without being bound by theory, the present authors hypothesize that the induction and/or mobilization of the novel NKT-like cells of the invention may occur via these low affinity hydrogen bonding interactions between ICAM3 and glucocorticoids, glucocorticoid receptor agonists, and ICAM3 modulating agents such as those described more fully elsewhere herein.

Accordingly, in a first aspect, the invention provides a method of producing a population of natural killer T cell-like cells (NKT-like cells), the method comprising administering to a subject a glucocorticoid-receptor (GR) modulating agent or ICAM3 modulating agent (which may be a glucocorticoid, such as dexamethasone) at a dose equivalent to about at least 6 mg/kg human equivalent dose (HED) of dexamethasone base, wherein the glucocorticoid receptor (GR) modulating agent or ICAM3 modulating agent induces and/or mobilizes the population of NKT-like cells in the subject.

The NKT-like cells of the invention exhibit a novel pattern of marker expression. In particular, the NKT-like cells produced/mobilized by the methods described herein express CD56 and TCR gamma/delta (γδTCR) as well as the invariant TCR (iTCR). Thus, in some embodiments, the population of NKT-like cells are characterized in that at least 60, 70, 80, 90, 95, 96, 97, 98, or 99% of the cells express CD56, TCR gamma/delta, and iTCR. The NKT-like cells produced/mobilized by the methods described herein also express CD16 and NKp44, which are markers of activated cells. Thus, in some embodiments, the population of NKT-like cells are characterized in that at least 60, 70, 80, 90, 95, 96, 97, 98, or 99% of the cells express CD16 and NKp44. In some embodiments, the population of NKT-like cells are characterized in that at least 60, 70, 80, 90, 95, 96, 97, 98, or 99% of the cells express CD56, TCR gamma/delta, iTCR, CD16, and NKp44. In some embodiments, the population of NKT-like cells are characterized in that at least 60, 70, 80, 90, 95, 96, 97, 98, or 99% of the cells express CD56, TCR gamma/delta, iTCR, CD16, NKp44, CD3, CD8, CD14, CD19, CD45, and/or TCR alpha/beta; and/or do not express: CD4, In some embodiments, the population of NKT-like cells are characterized in that at least 60, 70, 80, 90, 95, 96, 97, 98, or 99% of the cells express CD56, TCR gamma/delta, iTCR, CD16, NKp44, CD3, CD8, CD14, CD19, CD45, TCR alpha/beta, CD34, and/or ICAM3; and/or do not express: CD4.

In some embodiments, the NKT-like cells express CD56, TCR gamma/delta, and iTCR. In some embodiments, the NKT-like cells express CD16 and NKp44. In some embodiments, the NKT-like cells express CD56, TCR gamma/delta, iTCR, CD16, and NKp44. In some embodiments, the NKT-like cells express CD56, TCR gamma/delta, iTCR, and TCR alpha/beta. In some embodiments, the NKT-like cells express CD56, TCR gamma/delta, iTCR, CD16, NKp44, and TCR alpha/beta. In some embodiments, the NKT-like cells express CD56, TCR gamma/delta, iTCR, CD16, NKp44, CD8, CD14, and CD19. In some embodiments, the NKT-like cells express CD56, TCR gamma/delta, iTCR, CD16, NKp44, CD8, CD14, CD19, and TCR alpha/beta. In some embodiments, the NKT-like cells express CD56, TCR gamma/delta, iTCR, CD16, NKp44, CD3, CD8, CD19, and CD45. In some embodiments, the NKT-like cells express CD56, TCR gamma/delta, iTCR, CD16, NKp44, CD3, CD8, CD19, CD45, and TCR alpha/beta.

In some embodiments, the population of NKT-like cells are characterized in that at least 60, 70, 80, 90, 95, 96, 97, 98, or 99% of the cells express CD56, TCR gamma/delta, and iTCR. In some embodiments, the population of NKT-like cells are characterized in that at least 60, 70, 80, 90, 95, 96, 97, 98, or 99% of the cells express CD16 and NKp44. In some embodiments, the population of NKT-like cells are characterized in that at least 60, 70, 80, 90, 95, 96, 97, 98, or 99% of the cells express CD56, TCR gamma/delta, iTCR, CD16, and NKp44. In some embodiments, the population of NKT-like cells are characterized in that at least 60, 70, 80, 90, 95, 96, 97, 98, or 99% of the cells express CD56, TCR gamma/delta, iTCR, and TCR alpha/beta. In some embodiments, the population of NKT-like cells are characterized in that at least 60, 70, 80, 90, 95, 96, 97, 98, or 99% of the cells express CD56, TCR gamma/delta, iTCR, CD16, NKp44, and TCR alpha/beta. In some embodiments, the population of NKT-like cells are characterized in that at least 60, 70, 80, 90, 95, 96, 97, 98, or 99% of the cells express CD56, TCR gamma/delta, iTCR, CD16, NKp44, CD8, CD14, and CD19. In some embodiments, the population of NKT-like cells are characterized in that at least 60, 70, 80, 90, 95, 96, 97, 98, or 99% of the cells express CD56, TCR gamma/delta, iTCR, CD16, NKp44, CD8, CD14, CD19, and TCR alpha/beta. In some embodiments, the population of NKT-like cells are characterized in that at least 60, 70, 80, 90, 95, 96, 97, 98, or 99% of the cells express CD56, TCR gamma/delta, iTCR, CD16, NKp44, CD3, CD8, CD19, and CD45. In some embodiments, the population of NKT-like cells are characterized in that at least 60, 70, 80, 90, 95, 96, 97, 98, or 99% of the cells express CD56, TCR gamma/delta, iTCR, CD16, NKp44, CD3, CD8, CD19, CD45, and TCR alpha/beta. In some embodiments, the population of NKT-like cells are characterized in that at least 60, 70, 80, 90, 95, 96, 97, 98, or 99% of the cells express CD56, TCR gamma/delta, iTCR, CD16, NKp44, CD3, CD8, CD14, CD19, CD45, and TCR alpha/beta.

In some embodiments, the NKT-like cells do not express CD4. In some embodiments, the population of NKT-like cells are characterized in that at least 60, 70, 80, 90, 95, 96, 97, 98, or 99% of the cells do not express CD4.

In some embodiments the NKT-like cells of the disclosure may express CD3. In some such embodiments, the NKT-like cells of the disclosure may be CD3+/dim. In some embodiments, the NKT-like cells of the disclosure may express CD8. In some such embodiments, the NKT-like cells of the disclosure may be CD8+/dim. The NKT-like cells may be described as: CD3+/dim and/or CD8+/dim.

The NKT-like cells may be described as having these properties in naïve subjects. The NKT-like cells may be described as having these properties in a tumour/cancerous or autoimmune state. The expression levels of the cell markers can be determined relative to the average expression level in a population of reference NKT-like cells, derived from a common source, which have not been contacted with the glucocorticoid-receptor (GR) modulating agent or ICAM3 modulating agent. Expression of the markers can be measured by flow cytometry, e.g. performed using the equipment, reagents, and/or conditions described herein (taken in isolation or in combination).

The glucocorticoid-receptor (GR) modulating agent or ICAM3 modulating agent may be a glucocorticoid. In some embodiments, the glucocorticoid is selected from the group consisting of: dexamethasone, hydrocortisone, methylprednisolone, prednisone, prednisolone, prednylidene, cortisone, budesonide, betamethasone, flumethasone and beclomethasone.

In preferred embodiments, the glucocorticoid is selected from the group consisting of: dexamethasone, betamethasone, and methylprednisone (preferably dexamethasone or betamethasone).

In some embodiments, the glucocorticoid is selected from the group consisting of dexamethasone base, dexamethasone sodium phosphate, dexamethasone hemisuccinate, dexamethasone sodium succinate, dexamethasone succinate, dexamethasone isonicotinate, dexamethasone-21-acetate, dexamethasone phosphate, dexamethasone-21-phosphate, dexamethasone tebutate, dexamethasone-17-valerate, dexamethasone acetate monohydrate, dexamethasone pivalate, dexamethasone palmitate, dexamethasone-21-palmitate, dexamethasone dipropionate, dexamethasone propionate, dexamethasone acetate anhydrous, dexamethasone-21-phenylpropionate, dexamethasone-21-sulfobenzoate, dexamethasone hemo-sulfate, dexamethasone sulfate, dexamethasone beloxil, dexamethasone acid, dexamethasone acefurate, dexamethasone carboximide, dexamethasone cipecilate, dexamethasone 21-phosphate disodium salt, dexamethasone mesylate, dexamethasone linoleate, dexamethasone glucoside, dexamethasone glucuronide, dexamethasone iodoacetate, dexamethasone oxetanone, carboxymethylthio-dexamethasone, dexamethasonebisethoximes, dexamethasone epoxide, dexamethasonelinolelaidate, dexamethasone methylorthovalerate, dexamethasone spermine, 6-hydroxy dexamethasone, dexamethasone tributylacetate, dexamethasone aspartic acid, dexamethasone galactopyranose, dexamethasone hydrochloride, hydroxy dexamethasone, carboxy dexamethasone, desoxy dexamethasone, dexamethasone butazone, dexamethasone cyclodextrin, dihydro dexamethasone, oxo dexamethasone, propionyloxy dexamethasone, dexamethasone galactodie, dexamethasone isonicotinate, dexamethasone sodium hydrogen phosphate, dexamethasone aldehyde, dexamethasone pivlate, dexamethasone tridecylate, dexamethasone crotonate, dexamethasone methanesulfonate, dexamethasone butylacetate, dehydro dexamethasone, dexamethasone isothiocyanatoethyl thioether, dexamethasone bromoacetate, dexamethasone hemiglutarate, deoxy dexamethasone, dexamethasone chlorambucilate, dexamethasone melphalanate, formyloxy dexamethasone, dexamethasone butyrate, dexamethasone laurate, dexamethasone acetate, and any combination treatment that contains a form of dexamethasone.

In some embodiments, the glucocorticoid is dexamethasone, which may be dexamethasone sodium phosphate.

The methods of the invention can involve the administration of a particular glucocorticoid dose. In some embodiments, the glucocorticoid is administered at a dose equivalent to about:
6-12 mg/kg human equivalent dose (HED) of dexamethasone base;
at least 6 mg/kg human equivalent dose (HED) of dexamethasone base;
at least 12 mg/kg human equivalent dose (HED) of dexamethasone base;
at least 15 mg/kg human equivalent dose (HED) of dexamethasone base;
at least 18 mg/kg human equivalent dose (HED) of dexamethasone base;
at least 21 mg/kg human equivalent dose (HED) of dexamethasone base;
at least 24 mg/kg human equivalent dose (HED) of dexamethasone base;
up to 45 mg/kg human equivalent dose (HED) of dexamethasone base.
   In some preferred embodiments, the glucocorticoid is administered at a dose equivalent to about at least 18 mg/kg human equivalent dose (HED) of dexamethasone base. In some other preferred embodiments, the glucocorticoid is administered at a dose equivalent to about at least 6-18 mg/kg human equivalent dose (HED) of dexamethasone base. In some other preferred embodiments, the glucocorticoid is administered at a dose equivalent to about at least 15-18 mg/kg human equivalent dose (HED) of dexamethasone base.

In some embodiments, the glucocorticoid is administered at a dose equivalent to about:
6-12 mg/kg human equivalent dose (HED) of dexamethasone phosphate;
at least 6 mg/kg human equivalent dose (HED) of dexamethasone phosphate;
at least 12 mg/kg human equivalent dose (HED) of dexamethasone phosphate;
at least 15 mg/kg human equivalent dose (HED) of dexamethasone phosphate;
at least 18 mg/kg human equivalent dose (HED) of dexamethasone phosphate;
at least 21 mg/kg human equivalent dose (HED) of dexamethasone phosphate;
at least 24 mg/kg human equivalent dose (HED) of dexamethasone phosphate;
up to 45 mg/kg human equivalent dose (HED) of dexamethasone phosphate.
   In some preferred embodiments, the glucocorticoid is administered at a dose equivalent to about at least 18 mg/kg human equivalent dose (HED) of dexamethasone phosphate. In some other preferred embodiments, the glucocorticoid is administered at a dose equivalent to about at least 6-18 mg/kg human equivalent dose (HED) of dexamethasone phosphate. In some other preferred embodiments, the glucocorticoid is administered at a dose equivalent to about at least 15-18 mg/kg human equivalent dose (HED) of dexamethasone phosphate.

The glucocorticoid dose can be defined as a human equivalent dose (HED) of dexamethasone having a mg/kg value from a range of mg/kg values, wherein said range is bound by two of the mg/kg values set forth above. For instance, the glucocorticoid dose can be defined as a dexamethasone HED of 6-45 mg/kg. In another example, the glucocorticoid dose can be defined as a dexamethasone HED of 12-24 mg/kg.

The glucocorticoid may be administered as a single acute dose, or as a total dose given over about a 72 hour period. Moreover, the method may comprise administering one or more further doses of a glucocorticoid. In some embodiments, one or more further doses are administered: between 24 hours and 120 hours after a preceding glucocorticoid administration; between 24 hours and 48 hours after a preceding glucocorticoid administration; between 72 hours and 120 hours after a preceding glucocorticoid administration; every 24, 48, 72, 96, 120, 144, or 168 hours after a first glucocorticoid administration; once every week after a first glucocorticoid administration; once every two weeks after a first glucocorticoid administration; once monthly after a first glucocorticoid administration; or twice weekly after a first glucocorticoid administration.

The disclosed methods may include steps for activation of the NKT-like cells of the disclosure. Thus, in some embodiments, the methods may further comprise a step of administering an NKT cell activator, T cell activator, and/or NK cell activator to the subject. The NKT cell activator may be selected from the group consisting of: alpha GalCer, Sulfatide, or an NKT-activating antibody. The NKT cell activator may be alpha GalCer loaded dendritic cells or monocytes. The T cell activator may be selected from the group consisting of: zoledronate, mevastatin, or a T cell-activating antibody. The NK cell activator may be selected from the group consisting of: IL-2, IL-12, IL-15, IL-18, IL-21, or an NK cell-activating antibody. The NKT cell activator, T cell activator, and/or NK cell activator may be administered within or around 1-48 hours after administration of glucocorticoid.

In some embodiments, the subject is a human being. In some embodiments, the subject is a mammalian subject with a humanised immune system, such as a human immune system (HIS) mouse. Preferably the subject is a human.

The subject may have, or be suspected of having (or have been diagnosed with) cancer, an autoimmune disease, or infectious disease (also called microbial disease). The cancer may be a solid tumour. Alternatively, the cancer may be a lymphoma, preferably a B cell lymphoma or a T cell lymphoma. In some preferred embodiments, the cancer may be non-Hodgkin lymphoma.

The cancer may be selected from the group consisting of: squamous cell cancer (such as epithelial squamous cell cancer); lung cancer, including small-cell lung cancer, non-small cell lung cancer, adenocarcinoma of the lung and squamous carcinoma of the lung; cancer of the peritoneum; hepatocellular cancer; gastric or stomach cancer, including gastrointestinal cancer; pancreatic cancer; glioblastoma; cervical cancer; ovarian cancer; liver cancer; bladder cancer; hepatoma; breast cancer; colon cancer; rectal cancer; colorectal cancer; endometrial or uterine carcinoma; salivary gland carcinoma; kidney or renal cancer; prostate cancer; vulval cancer; thyroid cancer; hepatic carcinoma; anal carcinoma; penile carcinoma; and head and neck cancer.

The NKT-like cells of the invention may treat cancer via tumour infiltration. The NKT-like cells of the invention may treat cancer via release of immune activating cytokines. The NKT-like cells of the invention may treat cancer by engulfing and killing cancer cells. The NKT-like cells of the invention may treat cancer by promoting infiltration of other immune cells into the tumour. The NKT-like cells of the invention may treat cancer via CD1d-directed apoptosis. The NKT-like cells of the invention may treat cancer via tumour necrosis. The NKT-like cells of the invention may treat cancer by recognizing high levels of phosphoantigens made by tumor cells via expression of the gamma-delta T cell receptor on the NKT-like cells of the present invention. Thus, in some embodiments, the invention provides methods of causing tumour necrosis by inducing, mobilizing, or administering the NKT-like cells of the invention. In some embodiments, the invention provides methods of causing CD1d-directed apoptosis of cancer cells by inducing, mobilizing, or administering the NKT-like cells of the invention. In some embodiments, the invention provides methods of engulfing and/or killing cancer cells using the NKT-like cells of the invention. In some embodiments, the invention provides methods of activation of the gamma-delta expressing NKT-like cells by cancer cell phosphoantigens which then recognize and kill cancer cells via the NK receptor(s) on the NKT-like cells.

In embodiments where the subject has, is suspected of having (or has been diagnosed with) an autoimmune disease, the autoimmune disease may be multiple sclerosis, systemic sclerosis, amyotrophic lateral sclerosis, type 1 diabetes mellitus (TID), scleroderma, pemphigus or lupus. In embodiments where the subject has, is suspected of having (or has been diagnosed with) an infectious disease, the infectious disease may be HIV, herpes, hepatitis or human papilloma virus. In some embodiments, the infectious disease is HIV. In some preferred embodiments, the infectious disease may be COVID-19 (coronavirus 2019; the disease caused by severe acute respiratory syndrome coronavirus 2, SARS-CoV-2).

The methods of the invention may include isolation and/or expansion steps. For instance, the method may comprise a step of isolating a population of NKT-like cells from the subject or from a sample derived from the subject. Optionally, the step of isolating may be performed at least 48 hours after glucocorticoid administration; between 48 hours and 13 days after glucocorticoid administration; or between 6 and 48 hours after glucocorticoid administration. In some embodiments (such as embodiments in which the subject has cancer, an infectious disease or microbial disease, or autoimmune disease), the step of isolating the NKT-like cells may be performed within 3 hours after glucocorticoid administration, and preferably within 1 hour after glucocorticoid administration. In some embodiments, the step of isolating may be performed between 30 and 60 minutes after glucocorticoid administration.

The sample may be selected from the group consisting of: blood, plasma, a tumor biopsy or surgically removed tumor, bone marrow, liver, spleen biopsy, and fat or adipose tissue. In some embodiments, the methods further comprise a step of expanding the isolated NKT-like cells. In some embodiments, the method comprises a step of activating the isolated NKT-like cells with an NKT cell activator, T cell activator, and/or NK cell activator. The NKT cell activator, T cell activator, and/or NK cell activator may be as described elsewhere herein.

The isolated NKT-like cells of the invention can be further engineered e.g. by transfecting the cells with a nucleic acid. Accordingly, in some embodiments, the method further comprises a step of introducing a nucleic acid encoding a protein into the isolated NKT-like cells, and culturing the cells under conditions that facilitate expression of said protein. The protein may be one or more of: a T-cell receptor (TCR), a chimeric antigen receptor (CAR), a split, universal and programmable CAR (SUPRA-CAR). The CAR and/or TCR comprises an antigen-binding domain which binds to an antigen selected from the group consisting of: CD19, CD20, CD22, GD2, CD133, EGFR, GPC3, CEA, MUC1, Mesothelin, IL-13R, PSMA, ROR1, CAIX, Her2.

The NKT-like cells of the invention find uses in medicine. For instance, isolated NKT cells of the invention can be used medically, e.g. in the treatment of cancer, autoimmune disease, or infectious disease (also called microbial disease) in a subject. In these embodiments, the method may comprise administering a therapeutically effective dose of NKT-like cells isolated via methods disclosed herein, to a subject who suffers one of the aforementioned diseases. In some embodiments, the subject to whom the isolated NKT-like cells are administered is the same subject from whom the NKT-like cells were isolated. Alternatively, the subject to whom the isolated NKT-like cells are administered is different to the subject from whom the NKT-like cells were isolated.

The NKT-like cells are administered to the subject by a method selected from the group consisting of: intravenous injection, intraperitoneal injection, intra-lymphatic injection, intrathecal injection, injection into the cerebrospinal fluid (CSF), direct injection into a tumour, and as a gel placed on or near a solid tumour.

This invention also extends to the use of a glucocorticoid in the manufacture of a medicament for use in a method of treatment disclosed herein.

This invention further extends to the use of dexamethasone or other glucocorticoid to induce a population of NKT-like cells, wherein the population of NKT cells is induced by a method according to any one of statements 101-148.

This invention further extends to the use of dexamethasone or other glucocorticoid to mobilize a population of NKT-like cells, where in the population of NKT cells are mobilized by a method according to any one of statements 101-148.

Also provided are induced pluripotent stem cells that are derived from the NKT-like cells of the invention. Thus, in one aspect, the invention provides a method of producing induced pluripotent stem cells (iPSCs), the method comprising reprogramming NKT-like cells isolated by a method disclosed herein to produce iPSCs. The reprogramming may involve introducing one or more nucleic acids encoding Oct3/4, Klf4, Sox2, and C-myc into the NKT-like cells. The nucleic acid may be a DNA (e.g. a DNA expression cassette) or an RNA molecule. The reprogramming may further comprise introducing one or more expression cassettes encoding one or more of: Sox1, Sox3, Sox15, Klf1, Klf2, Klf5, L-myc, N-myc, Nanog, and/or LIN28 into the NKT-like cells. The reprogramming may further comprise introducing one or more of: Sox1, Sox3, Sox15, Klf1, Klf2, Klf5, L-myc, N-myc, Nanog, and/or LIN28 encoding mRNA into the NKT-like cells. The iPSCs may then be induced to differentiate, e.g. into NKT-like cells or into an NKT cell lineage.

This invention also provides an isolated natural killer T cell-like cell (NKT-like cell) or a population of NKT-like cells produced by a method disclosed herein. Relatedly, the NKT-like cells of the invention may be defined by their expression profile(s), which may be as described elsewhere herein. For instance, the invention provides an isolated natural killer T cell-like cell (NKT-like cell), characterized in that the cell expresses CD56, TCR gamma/delta, and iTCR and optionally expresses one or more of CD16, NKp44, CD3, CD8, CD14, CD19, CD45, and/or TCR alpha/beta; and/or does not express: CD4. The isolated NKT-like cell may be from a non-diseased subject.

The invention also provides an isolated population of natural killer T cell-like cells (NKT-like cells). The isolated population of NKT-like cells may be defined by their expression profile(s), which may be as described elsewhere herein. For instance, an isolated population of NKT-like cells may be characterized in that at least 60, 70, 80, 90, 95, 96, 97, 98, or 99% of the cells express CD56, TCR gamma/delta, and iTCR, and/or express one or more of CD16, NKp44, CD3, CD8, CD14, CD19, CD45, and/or TCR alpha/beta; and/or do not express: CD4.

The invention provides a glucocorticoid for use in a method of treatment of cancer, autoimmune disease, or infectious disease (also called microbial disease) in a subject, the method comprising administering a glucocorticoid to the subject at a dose equivalent to about 6-45 mg/kg human equivalent dose (HED) of dexamethasone, wherein the glucocorticoid induces/activates/mobilizes a population of NKT-like cells of the invention, as defined herein. For instance, the invention provides a glucocorticoid for use in a method of inducing tumor necrosis, causing tumour infiltration, releasing immune activating cytokines, engulfing and killing tumour cells, promoting infiltration of other immune cells into the tumour, and/or causing CD1d-directed apoptosis in a cancer patient, the method comprising administering a glucocorticoid to the subject at a dose equivalent to about 6-45 mg/kg human equivalent dose (HED) of dexamethasone, to induce a population of NKT-like cells of the invention, as defined herein. For instance, the invention provides a glucocorticoid for use in a method of inducing tumour necrosis, causing tumour infiltration, releasing immune activating cytokines, engulfing and killing tumour cells, promoting infiltration of other immune cells into a tumour, and/or causing CD1d-directed apoptosis in a cancer patient, the method comprising administering a glucocorticoid to the patient at a dose equivalent to about 6-45 mg/kg human equivalent dose (HED) dexamethasone, to mobilize a population of NKT-like cells of the invention, as defined herein. For instance, the invention provides a glucocorticoid for use in a method of inducing virus death, releasing immune activating cytokines, engulfing and killing virus-infected cells, promoting infiltration of other immune cells into the virus infected organs, the method comprising administering a glucocorticoid to the subject at a dose equivalent to about 6-45 mg/kg human equivalent dose (HED) of dexamethasone, to induce a population of NKT-like cells of the invention, as defined herein. The HED of dexamethasone may take any value from the range of values disclosed herein.

### SUMMARY OF THE FIGURES

Embodiments and experiments illustrating the principles of the disclosure will now be discussed with reference to the accompanying figures in which:

FIG. 1. Acute high dose dexamethasone reduces mouse lymphocyte number in naïve mice. Absolute lymphocyte numbers (ALC minus NK and NKT cells) measured by complete blood count (cells/ul=absolute numbers obtained from CBC) 6 hours, 24 hours, 48 hours, 7 days, 13 days, and 21 days after high-dose dexamethasone (18 mg/kg HED Dexamethasone Phosphate (DP)) are significantly reduced as compared to Placebo. At 6 and 48 hours after administration almost complete lymphoablation is observed, with the effect comparable to that achieved with standard Cy/Flu chemotherapy (13 mg/kg HED cyclophosphamide and 0.8 mg/kg HED fludarabine).

FIG. 2. Acute high dose dexamethasone reduces mouse B lymphocyte numbers in naïve mice. B lymphocyte numbers measured by complete blood count (cells/ul=absolute numbers obtained from CBC) 6 hours, 24 hours, 48 hours, 7 days, 13 days, and 21 days after high-dose dexamethasone (18 mg/kg HED DP) are significantly reduced as compared to Placebo. The lymphoablative effect on B lymphocytes is comparable to that achieved with standard Cy/Flu chemotherapy (13 mg/kg HED cyclophosphamide and 0.8 mg/kg HED fludarabine).

FIG. 3. Acute high dose dexamethasone reduces mouse monocyte numbers in naïve mice. Monocyte numbers measured by complete blood count (cells/ul=absolute numbers obtained from CBC) 6 hours, 24 hours, and 48 hours after high-dose dexamethasone (18 mg/kg HED DP) are significantly reduced as compared to Placebo. The ablative effect on monocytes is superior to that achieved by standard Cy/Flu chemotherapy (13 mg/kg HED cyclophosphamide and 0.8 mg/kg HED fludarabine).

Acute high dose dexamethasone reduces mouse neutrophil numbers in naïve mice. Neutrophil numbers measured by complete blood count (cells/ul=absolute numbers obtained from CBC) 6 hours, 24 hours, and 48 hours after high-dose dexamethasone (18 mg/kg HED DP) are significantly reduced as compared to Placebo.

FIG. 5. Acute high dose dexamethasone spares mouse platelets. Acute high dose dexamethasone (18 mg/kg HED DP) does not affect platelet numbers measured by complete blood count (cells/ul=absolute numbers obtained from CBC). Acute high dose dexamethasone therefore eliminates the need for transfusion, and provides a safer, non-toxic alternative to chemotherapeutic regimens. Platelets express glucocorticoid receptors (GRs), and thus absence of effect on platelets suggests a glucocorticoid receptor independent mechanism of action.

FIG. 6. Acute high dose dexamethasone spares hematopoietic stem cells. Shown are the number of live hematopoietic stem cells measured at time points between 6 hours and 35 days after treatment of naïve mice with placebo or acute high dose dexamethasone. The acute high dose dexamethasone (18 mg/kg HED DP) does not significantly alter the number of live hematopoietic stem cells. The non-myeloablative regimen represented by acute high dose dexamethasone could, therefore, eliminate the need for transfusions of stem cells for hematopoietic recovery after immune-reset.

FIG. 7. Acute high dose dexamethasone induces NKT upregulation (FIG. 7) and production of a novel population of NKT cells (AVM-NKT). Total NKT cell numbers measured by complete blood count (cells/ul=absolute numbers obtained from CBC) at 6 hours, and 24 hours after high-dose dexamethasone (18× mg/kg HED DP) are reduced as compared to Placebo. Surprisingly, by 48 hours after high-dose dexamethasone the total NKT cell numbers measured by complete blood count has increased, then reduces gradually until around 13 days after high-dose dexamethasone treatment. With administration of standard Cy/Flu chemotherapy (13 mg/kg HED cyclophosphamide and 0.8 mg/kg HED fludarabine) no such increase in NKT cell numbers is observed at 48 hours after treatment.

FIG. 8. After treatment with high dose dexamethasone, two NKT populations can be identified in peripheral blood. Examination of peripheral blood by flow cytometry after acute high dose dexamethasone identified two NKT cell populations: NKT cells defined as CD3medCD49b+ (CD56 in humans), corresponding to previously described NKT cells (central rectangular gate); and, a novel population of NKT cells defined as CD3highCD49b+ (CD56 in humans; AVM-NKT cells; center-right rectangular gate). AVM-NKT cells are CD49b+ (CD56 in humans) and CD3 very bright, as compared to the known NKT cells which express CD3 with Mean Fluorescent Intensity (MFI) one-half to one log lower than the AVM-NKT.

FIG. 9. Time course of AVM-NKT upregulation. Quantification of the AVM-NKT cells per microliter blood using CBC and flow cytometry results. The AVM-NKT cells are evident in blood of naïve mice between 48 hours-13 days after treatment with one high-dose dexamethasone (HED 18.1 mg/kg DP PO); *=statistically significant.

FIG. 10A. Changes in the A20 Tumor Environment induced by treatment with high-dose dexamethasone (HED 18 mg/kg DP). After 48 hours, increased necrosis is evident in tumors of mice treated with high-dose dexamethasone as compared to placebo.

FIG. 10B. AVM-NKT cells maximally ablate A20 lymphoma implanted in the flanks of mice within 3 hours after 18 mg/kg HED dexamethasone phosphate (left side figure), while A20 metastasis to blood and thymus is maximally eradicated 24 hours after dosing (middle figure) and A20 metastasis to bone marrow is maximally eradicated 48 hours after dosing (right side figure). Mice were inoculated with A20 lymphoma 2×10⁶ cells in 100 μL of PBS mixed with 100 μL of chilled Matrigel into their flank. Three, 24 and 48 hours later mice were euthanized and tumors, blood, bone marrow and thymus were taken and made into single cell suspensions for flow cytometry detection of live A20 cells.

FIG. 11. Acute high dose dexamethasone (AVM0703; HED 18.1 mg/kg PO) significantly delays growth of the A20 B cell lymphoma as compared to placebo. Days of high dose dexamethasone or placebo dosing are indicated by arrows.

FIG. 12. CD45/CD56 scattergrams from an osteoarthritis patient treated with 3-6 mg/kg DSP. AVM-NKT cells (indicated by a rectangular box) were identified and like in mice are CD45 dim and CD56 very bright (CD49b in mice).

FIG. 13. Flow cytometry data from a healthy blood donor and prostate cancer patient 1 hour and 3 hours after administration of 6 mg/kg AVM0703. In the prostate cancer patient a novel CD45dim CD56bright cell population (circled) is evident 1 hour after infusion. These data indicate that human patients mobilize cells corresponding to the AVM-NKT cells identified in mice.

FIG. 14. Lysed whole blood flow cytometry results for AVM0703 treated patient demonstrating double positive γδTCR and iTCR cells from the live cell CD56+ gate. CD56+ cells are shown on a scattergram for γδTCR and iTCR (left): 51% of the CD56+ cells are positive for both γδTCR and iTCR. The γδTCR+iTCR+ quadrant (AVM_NKT) was then evaluated for CD16 and NKp44 expression (right). CD16 and NKp46 were expressed on almost 100% of the AVM_NKT cells indicating an activated state.

FIG. 15. 101-001 size (FSC) and complexity (SSC) scattergram of AVM_NKT cells. One hour after 6 mg/kg AVM0703 (DP) infusion, infused IV over one hour, whole blood was drawn and shipped ambient to AVM Biotechnology. Blood was stained with panel antibodies based for 15 minutes in the dark at Room Temperature. RBC lysis was performed by addition of 1 mL of 1×BD FACS Lysing Solution (BD Bioscience), diluted with MilliQ water, for 10 minutes in the dark at Room Temperature. Samples were washed with 2 mL 1×DPBS CMF (Gibco) and resuspended in 300 μL of 1×DPBS CMF before 5 μL of 7AAD (Biolegend) was added to each sample for live/dead cell determination and incubated for 5 minutes in the dark at Room Temperature. 250 μL of sample were taken up for acquisition on Macsquant 16 Flow Cytometer (Miltenyi, Serial #40150). Live white blood cells were gated for CD56+ cells co-expressing gamma delta TCR and the invariant TCR and labelled red in the forward versus side scattergram. Red-labelled cells fall within the forward versus side scattergrams where large granular cells such as neutrophils and large granular lymphocytes are known to fall.

FIG. 16. Flow cytometry data for patient 101-001 CD56+γδTCR+invTCR+ cells. Upper left scattergram: pre-infusion; upper right scattergram: 1 hour post-infusion; lower left scattergram: day 3 post-infusion; lower right scattergram: day 14 post-infusion. Whole blood was processed as described for FIG. 15 and samples acquired on Macsquant 16 Flow Cytometer (Miltenyi, Serial #40150). Live white blood cells were gated on CD56+ cells and these CD56+ cells were then broadcast onto a scattergram showing gamma delta TCR versus invariant TCR staining. Cells expressing CD56 and gamma delta TCR and invariant TCR are found in the upper right quadrant of each scattergram. This patient showed evidence of significant numbers of CD56+gdTCR+invTCR+ cells (79 cells per microliter blood) 14 days after AVM0703 6 mg/kg infusion.

FIG. 17. Flow cytometry data for patient 103-002 CD56+γδTCR+invTCR+ cells. Upper left scattergram: pre-infusion; upper right scattergram: 1 hour post-infusion; lower left scattergram: day 3 post-infusion; lower right scattergram: day 14 post-infusion. Whole blood was processed as described for FIG. 15 and samples acquired on Macsquant 16 Flow Cytometer (Miltenyi, Serial #40150). Live white blood cells were gated on CD56+ cells and these CD56+ cells were then broadcast onto a scattergram showing gamma delta TCR versus invariant TCR staining. Cells expressing CD56 and gamma delta TCR and invariant TCR are found in the upper right quadrant of each scattergram. This patient had baseline evidence of circulating CD56+gdTCR+invTCR+ cells (89 cells per microliter) consistent with observations in tumor bearing mice that a tumor environment can induce and mobilize these NKT-like cells. Three days after 6 mg/kg dosing these CD56+gdTCR+invTCR+ cells reached circulating levels of 366 cells per microliter.

FIG. 18. Flow cytometry data for patient 103-005 CD56+γδTCR+invTCR+ cells. Upper left scattergram: pre-infusion; upper right scattergram: 1 hour post-infusion; bottom: day 3 post-infusion. Whole blood was processed as described for FIG. 15 and samples acquired on Macsquant 16 Flow Cytometer (Miltenyi, Serial #40150). Live white blood cells were gated on CD56+ cells and these CD56+ cells were then broadcast onto a scattergram showing gamma delta TCR versus invariant TCR staining. Cells expressing CD56 and gamma delta TCR and invariant TCR are found in the upper right quadrant of each scattergram. One hour after 9 mg/kg infusion baseline CD56+gdTCR+invTCR+ cells (347 cells per microliter) were significantly reduced to 22 cells per microliter, suggesting activation and tumor homing of the circulating cells in response to 9 mg/kg DP, consistent with clinical observations of tumor flare in this patient and consistent with observations in A20 lymphoma bearing mice that a tumor environment can induce the production of these NKT-like cells but that dexamethasone phosphate or another glucocorticoid are required to optimally activate the cells and trigger them to home to and eradicate tumor cells.

FIG. 19. Flow cytometry data for patient 108-001 1st infusion CD56+γδTCR+invTCR+ cells. Upper left scattergram: pre-infusion; upper right scattergram: 1 hour post-infusion; bottom: day 3 post-infusion. Whole blood was processed as described for FIG. 15 and samples acquired on Macsquant 16 Flow Cytometer (Miltenyi, Serial #40150). Live white blood cells were gated on CD56+ cells and these CD56+ cells were then broadcast onto a scattergram showing gamma delta TCR versus invariant TCR staining. Cells expressing CD56 and gamma delta TCR and invariant TCR are found in the upper right quadrant of each scattergram. One hour after 9 mg/kg AVM0703 infusion CD56+gdTCR+iTCR+ cells in blood increased ˜20 fold from baseline (5.8 to 112 cells/uL), and remained elevated on day 3 (36 cells/uL). This patient had a significant clinical response with restoration of vision on day 3 after AVM0703 infusion.

FIG. 20. Flow cytometry data for patient 108-003 CD56+γδTCR+invTCR+ cells. Upper left scattergram: pre-infusion; upper right scattergram: 1 hour post-infusion; lower left scattergram: day 3 post-infusion; lower right scattergram: day 14 post-infusion. Whole blood was processed as described for FIG. 15 and samples acquired on Macsquant 16 Flow Cytometer (Miltenyi, Serial #40150). Live white blood cells were gated on CD56+ cells and these CD56+ cells were then broadcast onto a scattergram showing gamma delta TCR versus invariant TCR staining. Cells expressing CD56 and gamma delta TCR and invariant TCR are found in the upper right quadrant of each scattergram. CD56+γδTCR+invTCR+ cells were increased over time from 24 cells per microliter blood at baseline to 94 cells per microliter 14 days after 12 mg/kg infusion. This patient had a tumor flare response and dramatic immune homing to neck lymph nodes which were the site of disease and symptoms of pharyngitis that required hospitalization on day 14. This patients clinical response and flow cytometry detection of CD56+γδTCR+invTCR+ cells are consistent with preferential homing of these cells to tumor sites.

FIG. 21. Flow cytometry data for patient 108-004 CD56+γδTCR+invTCR+ cells. Upper left scattergram: pre-infusion; upper right scattergram: 1 hour post-infusion; bottom: day 3 post-infusion. Whole blood was processed as described for FIG. 15 and samples acquired on Macsquant 16 Flow Cytometer (Miltenyi, Serial #40150). Live white blood cells were gated on CD56+ cells and these CD56+ cells were then broadcast onto a scattergram showing gamma delta TCR versus invariant TCR staining. Cells expressing CD56 and gamma delta TCR and invariant TCR are found in the upper right quadrant of each scattergram. Patient 108-004 showed no evidence of CD56+γδTCR+invTCR+ cells at any time point and is the only patient treated with acute high dose DP at 6 mg/kg or greater who did not have a clinical response to treatment, consistent with anti-tumor activity being mediated by the induction and mobilization of these NKT-like cells.

FIG. 22. Flow cytometry data for patient 108-002 CD56+γδTCR+invTCR+ cells. Upper left scattergram: pre-infusion; upper right scattergram: 1 hour post-infusion; lower left scattergram: day 3 post-infusion; lower right scattergram: day 14 post-infusion. Whole blood was processed as described for FIG. 15 and samples acquired on Macsquant 16 Flow Cytometer (Miltenyi, Serial #40150). Live white blood cells were gated on CD56+ cells and these CD56+ cells were then broadcast onto a scattergram showing gamma delta TCR versus invariant TCR staining. Cells expressing CD56 and gamma delta TCR and invariant TCR are found in the upper right quadrant of each scattergram.

FIG. 23A-23C. γδTCR+invTCR+bi-specific CD56+ cell scattergrams from apparently healthy blood donors. Shown are scatter plots of γδTCR+iTCR+ cells from total CD56+ WBCs for 12 healthy blood donors. Whole blood was processed as described for FIG. 15 and samples acquired on Macsquant 16 Flow Cytometer (Miltenyi, Serial #40150). Live white blood cells were gated on CD56+ cells and these CD56+ cells were then broadcast onto a scattergram showing gamma delta TCR versus invariant TCR staining. Cells expressing CD56 and gamma delta TCR and invariant TCR are found in the upper right quadrant of each scattergram. Some ‘healthy’ blood donors have circulating levels of CD56+γδTCR+invTCR+ cells, in contrast to naïve mice that never have these cells. Naïve mice are maintained in essentially pathogen free conditions and are free of infections, while ‘healthy’ blood donors are not and may have an asymptomatic or undiagnosed infection, or autoimmune disease or cancer that has induced the expression of these NKT-like cells.

FIG. 24. AVM0703 induces mobilization of γδTCR+invTCR+bi-specific CD56+ cells into blood of humanized mice. AVM0703 induces CD56+ TCRγδ+ (12% of hCD45+ cells) that are CD16+, suggesting an activated state (mouse 10 Taconic NOG-EXL). Mouse white blood cells are gated on the LIVE population and then human CD45+ cells. Human CD45+ dim cells (upper left figure, labelled blue and circled), identified from other experiments to be the population containing the novel AVM-NKT cells, are broadcast into a scattergram of CD56 and gdTCR (upper right figure). CD56+gdTCR+ cells are in the upper right quadrant. Almost all of the human CD45+ dim cells, colored in blue, are found in the CD56+gdTCR+ upper right quadrant. The human CD45+ dim, CD56+, gdTCR+ cells are then identified by blue color on histograms of mean fluorescent intensity (MFI) for CD8 (lower left figure) and CD16 (lower right figure) expression. More than 80% of the cells are positive for CD8, indicating a cytotoxic cell type, and for CD16, indicating an activated cell type.

FIG. 25. >18 mg/kg AVM0703 HED induces bi-specific immune cell mobilization between 2-12% of hCD45+ cells (upper figures: mouse M5 5.14%; lower figures mouse M7 2.37%; CRL-NCG humanized mice). White blood cells are gated for LIVE cells and then human CD45+ cells and then broadcast into a scattergram for CD56 and gdTCR expression (left side figures). hCD45+, CD56+, gdTCR+ cells are found in the upper right quadrant and labelled red. The human CD45+CD56+gdTCR+ cells are then broadcast onto a histogram showing MFI for invariant TCR (right side figures). More than 97% of these cells also express invariant TCR.

FIG. 26. >18 mg/kg AVM0703 HED induces bi-specific immune cell mobilization between 2-12% of hCD45+ cells (upper figures: mouse M1 3.35%; lower figures mouse M3 4.13%; CRL-NCG humanized mice). White blood cells are gated for LIVE cells and then human CD45+ cells and then broadcast into a scattergram for CD56 and gdTCR expression (left side figures). hCD45+, CD56+, gdTCR+ cells are found in the upper right quadrant and labelled red. The human CD45+CD56+gdTCR+ cells are then broadcast onto a histogram showing MFI for invariant TCR (right side figures). More than 97% of these cells also express invariant TCR.

FIG. 27. AVM0703 induces γδTCR+invTCR+ bispecific activated CD56+ bone marrow cells in humanized mice. >18 mg/kg AVM0703 HED treated humanized mice have bi-specific immune cells in bone marrow between 0.3-8.5% of hCD45+ cells (upper figures: mouse M90; lower figures mouse M88; Taconic-NOG-EXL humanized mice). Around 90% of the bispecific γδTCR+invTCR+ cells are CD16+, indicating an activated state. In the upper left figure iTCD is iTCR. Bone marrow cells are gated for LIVE cells and then human CD45+ cells expressing CD56 are broadcast into a scattergram for gdTCR and inv TCR expression (left side figures). The human CD45+CD56+gdTCR+invTCR+ cells are then broadcast onto a histogram showing MFI for CD16 (right side figures). Around 90% of these cells express CD16, indicating an activated state.

FIG. 28. AVM0703 induces γδTCR+invTCR+ bispecific activated CD56+ bone marrow cells in humanized mice. >18 mg/kg AVM0703 HED treated humanized mice have bi-specific immune cells in bone marrow between 0.3-8.5% of hCD45+ cells (upper figures: mouse M5; lower figures mouse M7; CRL-NCG humanized mice). Around 90% of the bispecific γδTCR+invTCR+ cells are CD16+, indicating an activated state. Bone marrow cells are gated for LIVE cells and then human CD45+ cells expressing CD56 are broadcast into a scattergram for gdTCR and inv TCR expression (left side figures). The human CD45+CD56+gdTCR+invTCR+ cells are then broadcast onto a histogram showing MFI for CD16 (right side figures). Around 90% of these cells express CD16, indicating an activated state.

FIG. 29. AVM0703 induces myeloid cell production in humanized mice. Shown are data from two placebo mice (upper plots) and one AVM0703 treated mouse (lower plot) after a first AVM0703 or Placebo dose-upper left: M12 placebo mouse; upper right: M90 placebo mouse; lower: M88 32 mg/kg AVM0703 treated mouse. Forward and side scattergrams are shown, with lymphocytes circled on the scattergram. Placebo treated humanized mice (upper plots) have random appearance of non-lymphocytes without any distinct population evident. AVM0703 dosed mouse (lower plot) has evidence of a distinct non-lymphocyte population of cells with higher side scatter than the lymphocyte population after the first dose. Repeat dosing, as shown in FIG. 30 further induces this non-lymphocyte population, which has forward versus side scatter signal similar to signals expected for myeloid cells and large granular lymphocytes.

FIG. 30. AVM0703 induces myeloid cell production in humanized mice. Shown are data from two placebo mice (upper plots) and one AVM0703 treated mouse (lower plot) after a second AVM0703 or Placebo dose-upper left: M12 placebo mouse; upper right: M90 placebo mouse; lower: M88 32 mg/kg AVM0703 treated mouse. Repeat dosing further induces a non-lymphocyte population in the AVM0703 dosed mouse, which has forward versus side scatter signal similar to signals expected for myeloid cells and large granular lymphocytes

FIG. 31. Humanized mice have largely human Lymphoid cells. In M12 Placebo mouse after a first dose lymphocytes are mostly human CD45+ (upper plot) and the few myeloid cells are mostly mCD45+ (lower plot). Forward versus side scattergrams are shown for a placebo treated mouse and demonstrate that in the humanized mice lymphocytes are mostly human CD45+ (labelled green in upper plot) and the few myeloid cells are mostly mCD45+ (labelled red in lower plot).

FIG. 32. AVM0703 dosing induces myeloid cell production in humanized mice. M12 Placebo: mouse lymphocytes are 13% of total mouse WBCs (upper left); human lymphocytes are 60% of total human WBCs (upper right); total lymphocytes are 45% of total WBCs.

FIG. 33. AVM0703 dosing induces myeloid cell production in humanized mice. M90 Placebo: mouse lymphocytes are only 12.5% of total WBCs; human lymphocytes are 31.5% of total human WBCs; total lymphocytes are 30% of total WBCs.

FIG. 34. AVM0703 dosing induces myeloid cell production in humanized mice. M88 AVM0703: mouse lymphocytes are only 5.7% of total WBCs; human lymphocytes are 58% of total human WBCs; total lymphocytes are 32% of total WBCs. The data in this figure illustrates that after AVM0703 dosing, compared to Placebo treated humanized mice the total lymphocyte population is reduced from about 45% of all WBCs to about 32% of all WBCs. Induction of myeloid cell production reduces the percentage of WBCs that are lymphoid.

FIG. 35. AVM0703 dosing induces myeloid cell production in humanized mice. M01 AVM0703: mouse lymphocytes are only 6.7% of total WBCs; human lymphocytes are 67% of total human WBCs; total lymphocytes are 35% of total WBCs. The data in this figure illustrates that after AVM0703 dosing, compared to Placebo treated humanized mice the total lymphocyte population is reduced from about 45% of all WBCs to about 35% of all WBCs. Induction of myeloid cell production reduces the percentage of WBCs that are lymphoid.

FIG. 36. AVM0703 dosing induces myeloid cell production in humanized mice. M03 AVM0703: mouse lymphocytes are only 23.7% of total WBCs; human lymphocytes are 47% of total human WBCs; total lymphocytes are 40% of total WBCs. The data in this figure illustrates that after AVM0703 dosing, compared to Placebo treated humanized mice the total lymphocyte population is reduced from about 45% of all WBCs to about 40% of all WBCs. Induction of myeloid cell production reduces the percentage of WBCs that are lymphoid.

FIG. 37. AVM0703 dosing induces myeloid cell production in humanized mice. M05 AVM0703: mouse lymphocytes are only 2.0% of total WBCs; human lymphocytes are 50.1% of total human WBCs; total lymphocytes are 20.9% of total WBCs. The data in this figure illustrates that after AVM0703 dosing, compared to Placebo treated humanized mice the total lymphocyte population is reduced from about 45% of all WBCs to about 21% of all WBCs. Induction of myeloid cell production reduces the percentage of WBCs that are lymphoid.

FIG. 38. AVM0703 dosing induces myeloid cell production in humanized mice. M07 AVM0703: mouse lymphocytes are only 20.4% of total WBCs; human lymphocytes are 58.2% of total human WBCs; total lymphocytes are 41.9% of total WBCs.

FIG. 39. AVM0703 dosing induces myeloid cell production in humanized mice. M10 AVM0703: mouse lymphocytes are only 5.2% of total WBCs; human lymphocytes are 37.5% of total human WBCs; total lymphocytes are 28.1% of total WBCs. The data in this figure illustrates that after AVM0703 dosing, compared to Placebo treated humanized mice the total lymphocyte population is reduced from about 45% of all WBCs to about 30% of all WBCs. Induction of myeloid cell production reduces the percentage of WBCs that are lymphoid.

FIG. 40. ACT AVM-NKT cells from AVM0703 treated mice significantly reduced the total number of live MOPC315 cells in tumors (upper left) and spleens (upper right) of mice preconditioned with AVM0703. AVM0703 preconditioning followed by ACT also showed trends of reduced live MOPC315 in blood (lower left) and bone marrow (lower right). Live MOPC315 cell distribution of the different groups in the subcutaneous (upper left) tumor, (upper right) spleen, (lower left) bone marrow and (lower right) blood of balb/c mice analyzed after single cell processing and flow cytometry (CD138+CD4+) are shown. The cell recipient groups (n=8) were preconditioned with 18 mg/kg HED AVM0703 (oral gavage) 48 h prior to adoptive cell transfer (ACT). 49 naïve donor balb/c mice were dosed P.O. with 45 mg/kg of AVM0703 to induce the bi-specific γδTCR+invTCR+ NKT-like cells for the ACT, while 8 naïve balb/c mice were dosed P.O. with Placebo, 96 h prior to ACT (3.3 million splenocytes I.V. injected per mouse; splenocytes from AVM0703 or Placebo dosed mice were pooled, respectively). The first part of the group name designates the preconditioned (PC) acceptor group, while the second part designates whether the donor cells were sourced from AVM0703 mice or placebo (for example, AVM18 PC-AVM ACT is the group preconditioned with 18 mg/kg AVM0703 48 h before ACT and I.V. injected with splenocytes from mice dosed with 1×45 mg/kg 96 h prior to harvest). All mice were sacrificed about 18 h after ACT. Average Tumor Volume at preconditioning was ˜130 mm³. (*) P<0.05 (Kruskal-Wallis test-groups compared to the ‘Placebo PC-Placebo ACT’-group). ACT cells from AVM0703 treated mice significantly reduced the total number of live MOPC315 cells in tumors and spleens of mice preconditioned with AVM0703. Additionally, while the reductions were not statistically significant, preconditioning with AVM0703 followed by ACT of cells from placebo treated mice showed trends towards reduced live MOPC315 cells as expected based on the ability of AVM0703 preconditioning to induce/mobilize endogenous bi-specific NKT-like cells in MOPC315 inoculated mice. While results were not statistically significant, AVM0703 preconditioning followed by ACT showed trends of reduced live MOPC315 in blood and bone marrow also.

FIG. 41. Patient 103-007 (mantle cell lymphoma). Absolute lymphocyte counts (ALC) demonstrating AVM0703 reduced lymphocytes only in one patient who had baseline lymphocytosis.

FIG. 42. In patient 103-007, monocytes, platelets, hematocrit and RBCs were not reduced after AVM0703 dosing.

FIG. 43. Ex Vivo/In Vitro Dexamaethasone CRC Demonstrating Absence of GCR activation at concentrations equivalent to suprapharmacologic doses. Mouse whole blood (WB) and splenocytes (spl) were incubated for 6 hours with increasing concentrations of dexamethasone base and then cell counts (WB) or apoptosis (spl) was determined by CBC analysis (WB) or flow cytometry (spl) after co-staining for live/dead cells using Viobility™ (Miltenyi Biotec) and eBioscience™ Calcein AM Viability Dye (Invitrogen, ThermoFisher Scientific).

### DETAILED DESCRIPTION

The present disclosure pertains to: methods of producing/activating/mobilizing a population of natural killer T cell-like cells (NKT-like cells), isolated NKT-like cells or isolated populations of NKT-like cells produced by such methods, and methods of treatment in which NKT-like cells are induced in a subject, or are administered to a subject. The disclosure is based on the authors' finding that high doses of a glucocorticoid receptor modulating agent, such as the glucocorticoid dexamethasone, are able to induce the production and mobilization of a γδ Natural Killer T-like cell (CD56+γδTCR+), which also expresses the invariant TCR (iTCR+). These newly discovered cells, and populations of these cells, are referred to herein as natural killer T cell-like cells (NKT-like cells), but can also be referred to as, e.g. natural killer T cells (NKT cells), CD56+γδTCR+ iTCR+ NKT cells, or AVM-NKT cells. As used herein, the term “population of cells” may refer to collection or group of cells which share similar properties—for example, a collection or group of multiple cells which share a characteristic pattern of expression of surface proteins. By way of example, a population of cells may refer to a group or collection of cells which all express CD56. TCR gamma/delta, and iTCR.

As used herein, to “mobilize” such cells can mean to promote movement of these out of lymphoid organs/tissues (for example, the thymus and spleen) and into the systemic circulation (where they may then move to other sites, e.g. tumour sites). The disclosed methods may include multiple of the above aspects. For example, a method of the disclosure may induce production of a population of NKT-like cells as described herein in the thymus and/or spleen and/or bone marrow, and mobilize a population of NKT-like cells as described herein from the thymus and/or spleen and/or bone marrow.

As disclosed herein, the methods of producing a population of NKT-like cells comprise administering to a subject a glucocorticoid-receptor (GR) modulating agent or ICAM3 modulating agent. The ICAM3 modulating agent could activate ICAM3 signaling or cause ICAM3 shedding to cause the NKT-like cells to be induced in the subject. The glucocorticoid-receptor (GR) modulating agent or ICAM3 modulating agent induces the population of NKT-like cells in the subject. The glucocorticoid-receptor (GR) modulating agent or ICAM3 modulating agent may mobilize the population of NKT-like cells in the subject.

Also disclosed are isolated populations of NKT-like cells and isolated NKT-like cells which may be produced by the disclosed methods.

The disclosed NKT-like cells may be characterized by the pattern of surface proteins which they express. In some embodiments, the disclosed NKT-like cells may express CD56, TCR gamma/delta, iTCR, CD16, NKp44, CD3, CD8, CD14, CD19, CD45, TCR alpha/beta, CD34 and/or ICAM3. In some embodiments, the disclosed NKT-like cells may express CD56, TCR gamma/delta, iTCR, CD16, NKp44, CD3, CD8, CD14, CD19, CD45, and/or TCR alpha/beta. In some embodiments, the disclosed NKT cells may not express CD4.

In some embodiments, the NKT-like cells express TCR gamma/delta and iTCR. In some embodiments, the NKT-like cells express CD56, TCR gamma/delta, and iTCR. In some embodiments, the NKT-like cells express CD45, TCR gamma/delta, and iTCR. In some embodiments, the NKT-like cells express CD45, CD56, TCR gamma/delta, and iTCR.

In some embodiments, the NKT-like cells express CD16. In some embodiments, the NKT-like cells express TCR gamma/delta, iTCR, and CD16. In some embodiments, the NKT-like cells express CD56, TCR gamma/delta, iTCR, and CD16. In some embodiments, the NKT-like cells express CD45, TCR gamma/delta, iTCR, and CD16. In some embodiments, the NKT-like cells express CD45, CD56, TCR gamma/delta, iTCR, and CD16.

In some embodiments, the NKT-like cells express CD16 and NKp44. In some embodiments, the NKT-like cells express TCR gamma/delta, iTCR, and CD16. In some embodiments, the NKT-like cells express CD56, TCR gamma/delta, iTCR, CD16, and NKp44. In some embodiments, the NKT-like cells express CD45, TCR gamma/delta, iTCR, CD16, and NKp44. In some embodiments, the NKT-like cells express CD45, CD56, TCR gamma/delta, iTCR, CD16, and NKp44.

In some embodiments, the NKT-like cells express TCR alpha/beta. In some embodiments, the NKT-like cells express TCR gamma/delta, iTCR, and TCR alpha/beta. In some embodiments, the NKT-like cells express CD56, TCR gamma/delta, iTCR, and TCR alpha/beta. In some embodiments, the NKT-like cells express CD45, TCR gamma/delta, iTCR, and TCR alpha/beta. In some embodiments, the NKT-like cells express CD45, CD56, TCR gamma/delta, iTCR, and TCR alpha/beta. In some embodiments, the NKT-like cells express CD45, CD56, TCR gamma/delta, iTCR, TCR alpha/beta, and CD16. In some embodiments, the NKT-like cells express CD45, CD56, TCR gamma/delta, iTCR, TCR alpha/beta, CD16, and NKp44.

In some embodiments, the NKT-like cells express CD56, TCR gamma/delta, iTCR, CD16, NKp44, and TCR alpha/beta. In some embodiments, the NKT-like cells express CD56, TCR gamma/delta, iTCR, CD16, NKp44, CD3, CD8, CD19, and CD45. In some embodiments, the NKT-like cells express CD56, TCR gamma/delta, iTCR, CD16, NKp44, CD3, CD8, CD19, CD45, and TCR alpha/beta. In some embodiments, the NKT-like cells express CD56, TCR gamma/delta, iTCR, CD16, NKp44, CD8, CD14, and CD19. In some embodiments, the NKT-like cells express CD56, TCR gamma/delta, iTCR, CD16, NKp44, CD8, CD14, CD19, and TCR alpha/beta. In some embodiments, the NKT-like cells express CD45, CD56, TCR gamma/delta, and iTCR. In some embodiments, the NKT-like cells express CD45, CD56, TCR gamma/delta, iTCR, and TCR alpha/beta. In some embodiments, the NKT-like cells express CD45, CD56, TCR gamma/delta, iTCR, TCR alpha/beta, and CD8. In some embodiments, the NKT-like cells express CD56, TCR gamma/delta, iTCR, and CD8. In some embodiments, the NKT-like cells express CD56, TCR gamma/delta, iTCR, CD8, and CD3. In some embodiments, the NKT-like cells express CD56, TCR gamma/delta, iTCR, CD16, CD34, and ICAM3. In some embodiments, the NKT-like cells express CD56, TCR gamma/delta, iTCR, CD16, CD34, ICAM3, and NKp44.

In embodiments relating to populations of the disclosed NKT-like cells, the population of NKT-like cells may be characterized in that at least 60, 70, 80, 90, 95, 96, 97, 98, or 99% of the cells express the markers or marker combinations outlined above.

Thus, in embodiments relating to populations of the disclosed NKT-like cells, the population of NKT-like cells may be characterized in that at least 60, 70, 80, 90, 95, 96, 97, 98, or 99% of the cells express CD56, TCR gamma/delta, iTCR, CD16, NKp44, CD3, CD8, CD14, CD19, CD45, TCR alpha/beta, CD34 and/or ICAM3. In embodiments relating to populations of the disclosed NKT-like cells, the population of NKT-like cells may be characterized in that at least 60, 70, 80, 90, 95, 96, 97, 98, or 99% of the cells express CD56, TCR gamma/delta, iTCR, CD16, NKp44, CD3, CD8, CD14, CD19, CD45, and/or TCR alpha/beta. In some embodiments, the population of NKT-like cells may be characterized in that at least 60, 70, 80, 90, 95, 96, 97, 98, or 99% of the cells do not express CD4.

In some embodiments, the population of NKT-like cells are characterized in that at least 60, 70, 80, 90, 95, 96, 97, 98, or 99% of the cells express TCR gamma/delta and iTCR. In some embodiments, the population of NKT-like cells are characterized in that at least 60, 70, 80, 90, 95, 96, 97, 98, or 99% of the cells express CD56, TCR gamma/delta, and iTCR. In some embodiments, the population of NKT-like cells are characterized in that at least 60, 70, 80, 90, 95, 96, 97, 98, or 99% of the cells express CD45, TCR gamma/delta, and iTCR. In some embodiments, the population of NKT-like cells are characterized in that at least 60, 70, 80, 90, 95, 96, 97, 98, or 99% of the cells express CD45, CD56, TCR gamma/delta, and iTCR.

In some embodiments, the population of NKT-like cells are characterized in that at least 60, 70, 80, 90, 95, 96, 97, 98, or 99% of the cells express CD16. In some embodiments, the population of NKT-like cells are characterized in that at least 60, 70, 80, 90, 95, 96, 97, 98, or 99% of the cells express TCR gamma/delta, iTCR, and CD16. In some embodiments, the population of NKT-like cells are characterized in that at least 60, 70, 80, 90, 95, 96, 97, 98, or 99% of the cells express CD56, TCR gamma/delta, iTCR, and CD16. In some embodiments, the population of NKT-like cells are characterized in that at least 60, 70, 80, 90, 95, 96, 97, 98, or 99% of the cells express CD45, TCR gamma/delta, iTCR, and CD16. In some embodiments, the population of NKT-like cells are characterized in that at least 60, 70, 80, 90, 95, 96, 97, 98, or 99% of the cells express CD45, CD56, TCR gamma/delta, iTCR, and CD16.

In some embodiments, the population of NKT-like cells are characterized in that at least 60, 70, 80, 90, 95, 96, 97, 98, or 99% of the cells express CD16 and NKp44. In some embodiments, the population of NKT-like cells are characterized in that at least 60, 70, 80, 90, 95, 96, 97, 98, or 99% of the cells express TCR gamma/delta, iTCR, and CD16. In some embodiments, the population of NKT-like cells are characterized in that at least 60, 70, 80, 90, 95, 96, 97, 98, or 99% of the cells express CD56, TCR gamma/delta, iTCR, CD16, and NKp44. In some embodiments, the population of NKT-like cells are characterized in that at least 60, 70, 80, 90, 95, 96, 97, 98, or 99% of the cells express CD45, TCR gamma/delta, iTCR, CD16, and NKp44. In some embodiments, the population of NKT-like cells are characterized in that at least 60, 70, 80, 90, 95, 96, 97, 98, or 99% of the cells express CD45, CD56, TCR gamma/delta, iTCR, CD16, and NKp44.

In some embodiments, the population of NKT-like cells are characterized in that at least 60, 70, 80, 90, 95, 96, 97, 98, or 99% of the cells express TCR alpha/beta. In some embodiments, the population of NKT-like cells are characterized in that at least 60, 70, 80, 90, 95, 96, 97, 98, or 99% of the cells express TCR gamma/delta, iTCR, and TCR alpha/beta. In some embodiments, the population of NKT-like cells are characterized in that at least 60, 70, 80, 90, 95, 96, 97, 98, or 99% of the cells express CD56, TCR gamma/delta, iTCR, and TCR alpha/beta. In some embodiments, the population of NKT-like cells are characterized in that at least 60, 70, 80, 90, 95, 96, 97, 98, or 99% of the cells express CD45, TCR gamma/delta, iTCR, and TCR alpha/beta. In some embodiments, the population of NKT-like cells are characterized in that at least 60, 70, 80, 90, 95, 96, 97, 98, or 99% of the cells express CD45, CD56, TCR gamma/delta, iTCR, and TCR alpha/beta. In some embodiments, the population of NKT-like cells are characterized in that at least 60, 70, 80, 90, 95, 96, 97, 98, or 99% of the cells express CD45, CD56, TCR gamma/delta, iTCR, TCR alpha/beta, and CD16. In some embodiments, the population of NKT-like cells are characterized in that at least 60, 70, 80, 90, 95, 96, 97, 98, or 99% of the cells express CD45, CD56, TCR gamma/delta, iTCR, TCR alpha/beta, CD16, and NKp44.

In some embodiments, the population of NKT-like cells are characterized in that at least 60, 70, 80, 90, 95, 96, 97, 98, or 99% of the cells express CD56, TCR gamma/delta, iTCR, CD16, NKp44, and TCR alpha/beta. In some embodiments, the population of NKT-like cells are characterized in that at least 60, 70, 80, 90, 95, 96, 97, 98, or 99% of the cells express CD56, TCR gamma/delta, iTCR, CD16, NKp44, CD3, CD8, CD19, and CD45. In some embodiments, the population of NKT-like cells are characterized in that at least 60, 70, 80, 90, 95, 96, 97, 98, or 99% of the cells express CD56, TCR gamma/delta, iTCR, CD16, NKp44, CD3, CD8, CD19, CD45, and TCR alpha/beta. In some embodiments, the population of NKT-like cells are characterized in that at least 60, 70, 80, 90, 95, 96, 97, 98, or 99% of the cells express CD56, TCR gamma/delta, iTCR, CD16, NKp44, CD8, CD14, and CD19. In some embodiments, the population of NKT-like cells are characterized in that at least 60, 70, 80, 90, 95, 96, 97, 98, or 99% of the cells express CD56, TCR gamma/delta, iTCR, CD16, NKp44, CD8, CD14, CD19, and TCR alpha/beta. In some embodiments, the population of NKT-like cells are characterized in that at least 60, 70, 80, 90, 95, 96, 97, 98, or 99% of the cells express CD45, CD56, TCR gamma/delta, and iTCR. In some embodiments, the population of NKT-like cells are characterized in that at least 60, 70, 80, 90, 95, 96, 97, 98, or 99% of the cells express CD45, CD56, TCR gamma/delta, iTCR, and TCR alpha/beta. In some embodiments, the population of NKT-like cells are characterized in that at least 60, 70, 80, 90, 95, 96, 97, 98, or 99% of the cells express CD45, CD56, TCR gamma/delta, iTCR, TCR alpha/beta, and CD8. In some embodiments, the population of NKT-like cells are characterized in that at least 60, 70, 80, 90, 95, 96, 97, 98, or 99% of the cells express CD56, TCR gamma/delta, iTCR, and CD8. In some embodiments, the population of NKT-like cells are characterized in that at least 60, 70, 80, 90, 95, 96, 97, 98, or 99% of the cells express CD56, TCR gamma/delta, iTCR, CD8, and CD3. In some embodiments, the population of NKT-like cells are characterized in that at least 60, 70, 80, 90, 95, 96, 97, 98, or 99% of the cells express CD56, TCR gamma/delta, iTCR, CD16, CD34, and ICAM3. In some embodiments, the population of NKT-like cells are characterized in that at least 60, 70, 80, 90, 95, 96, 97, 98, or 99% of the cells express CD56, TCR gamma/delta, iTCR, CD16, CD34, ICAM3, and NKp44.

Expression of surface proteins on cells can be readily determined using techniques well-known to the skilled person—for example, enzyme-linked immunosorbent assays (ELISA), magnetic-activated cell sorting (MACS), or flow cytometry techniques. Flow cytometry uses the light properties scattered from cells bound by fluorescently-tagged antibodies to identify cells expressing surface proteins of interest. Flow cytometry can determine not only whether a cell is expressing a protein of interest but can also indicate the amount of protein expressed by cells on the basis of intensity of fluorescence. In flow cytometric readouts, and as used herein: “+” (or “positive”) indicates expression of a given surface protein; “−” (or “negative”) indicates no expression of a given surface protein; and “+/−” indicates bimodal expression of a given surface protein. Expressions such as “bright” (sometimes “high” or “++”), “dim” (sometimes “low”), and “moderate” are used to indicate the relative amount of a particular cell surface protein.

### CD3

CD3 (cluster of differentiation 3) is a T-cell co-receptor, which helps to activate cytotoxic T cells (CD8+ naive T cells) and T helper cells (CD4+ naive T cells). Because CD3 is required for T cell activation, drugs (e.g. monoclonal antibodies) that target it are being investigated as immunosuppressant therapies (e.g. otelixizumab) for type 1 diabetes and other autoimmune diseases. The NKT-like cells of the invention lose CD3 expression following activation (a known phenomenon of T cell activation-see, for example, Valle et al, J Immunol. 2015 Mar. 1; 194 (5): 2117-27); therefore, CD3 fluorescence intensity (e.g. whether the cells are CD3+/dim or CD3+/bright) may depend on whether the cells are activated or not.

In some embodiments, the NKT-like cells of the disclosure express CD3. In some embodiments, the NKT cells of the disclosure are CD3+/dim. In embodiments relating to populations of the NKT-like cells of the disclosure, at least 5, 10, 20, 30, 40, 50, 60, 70, 80, 90, 95, 96, 97, 98, or 99% of the NKT-like cells may express CD3. In some embodiments at least 5, 10, 20, 30, 40, 50, 60, 70, 80, 90, 95, 96, 97, 98, or 99% of the NKT-like cells may be CD3+/dim. In some embodiments, the NKT cells of the disclosure are CD3+/bright. In some embodiments at least 5, 10, 20, 30, 40, 50, 60, 70, 80, 90, 95, 96, 97, 98, or 99% of the NKT-like cells may be CD3+/bright.

### CD4

CD4 (cluster of differentiation 4) is a glycoprotein found on the surface of immune cells including T-helper cells and monocytes. CD4 is a co-receptor of the T cell receptor (TCR), which it assists in communicating with antigen presenting cells for antigen-induced T cell activation. Cross-linking of CD4 can induce T cell apoptosis via the Fas Ligand pathway.

In some embodiments, the NKT-like cells of the disclosure do not express CD4. In embodiments relating to populations of the NKT-like cells of the disclosure, at least 5, 10, 20, 30, 40, 50, 60, 70, 80, 90, 95, 96, 97, 98, or 99% of the NKT-like cells may not express CD4.

### Cd8

CD8 (cluster of differentiation 8) is a transmembrane glycoprotein that serves as a co-receptor for the T cell receptor (TCR). It is predominantly expressed on the surface of cytotoxic T cells, but is also expressed on natural killer cells. On T cells it plays roles in T cell-antigen interaction and T cell signalling.

In some embodiments, the NKT-like cells of the disclosure express CD8. In some embodiments, the NKT-like cells of the disclosure are CD8+/dim. In some embodiments, the NKT-like cells of the disclosure are CD8+/moderate. In some embodiments, the NKT-like cells of the disclosure are CD8+/bright. In embodiments relating to populations of the NKT-like cells of the disclosure, at least 5, 10, 20, 30, 40, 50, 60, 70, 80, 90, 95, 96, 97, 98, or 99% of the NKT-like cells may express CD8. In some embodiments at least 5, 10, 20, 30, 40, 50, 60, 70, 80, 90, 95, 96, 97, 98, or 99% of the NKT-like cells may be CD8+/dim. In some embodiments at least 5, 10, 20, 30, 40, 50, 60, 70, 80, 90, 95, 96, 97, 98, or 99% of the NKT-like cells may be CD8+/moderate. In some embodiments at least 5, 10, 20, 30, 40, 50, 60, 70, 80, 90, 95, 96, 97, 98, or 99% of the NKT-like cells may be CD8+/bright.

### CD14

CD14 (cluster of differentiation 14) is a protein expressed mostly by macrophages as part of the innate immune system. It helps to detect bacteria in the body by binding lipopolysaccharide, and was the first described patter recognition receptor.

In some embodiments, the NKT-like cells of the disclosure express CD14. In embodiments relating to populations of the NKT-like cells of the disclosure, at least 5, 10, 20, 30, 40, 50, 60, 70, 80, 90, 95, 96, 97, 98, or 99% of the NKT-like cells may express CD14.

### CD19

CD19 (cluster of differentiation 19; also known as B-lymphocyte antigen CD19, B-Lymphocyte Surface Antigen B4, T-Cell Surface Antigen Leu-12, and CVID3) is a transmembrane protein expressed in all B lineage cells. In human B cells it acts as an adaptor protein to recruit cytoplasmic signaling proteins to the cell membrane, and also works within the CD19/CD21 complex to decrease the threshold for B cell receptor signaling pathways.

In some embodiments, the NKT-like cells of the disclosure express CD19. In embodiments relating to populations of the NKT-like cells of the disclosure, at least 5, 10, 20, 30, 40, 50, 60, 70, 80, 90, 95, 96, 97, 98, or 99% of the NKT-like cells may express CD19.

### CD34

CD34 (cluster of differentiation 34) is a cell surface glycoprotein which functions as a cell-cell adhesion factor and is required for T cells to enter lymph nodes. Cells expressing CD34 are normally found in the umbilical cord and bone marrow as haematopoietic cells, or in endothelial progenitor cells, endothelial cells of blood vessels but not lymphatics (except pleural lymphatics), mast cells, a sub-population of dendritic cells (which are factor XIIIa-negative) in the interstitium and around the adnexa of dermis of skin, as well as cells in soft tissue tumours.

In some embodiments, the NKT-like cells of the disclosure express CD34. In embodiments relating to populations of the NKT-like cells of the disclosure, at least 5, 10, 20, 30, 40, 50, 60, 70, 80, 90, 95, 96, 97, 98, or 99% of the NKT-like cells may express CD34.

### CD45

CD45 (cluster of differentiation 45; also known as Protein tyrosine phosphatase, receptor type; PTPRC) is an essential regulator of T- and B-cell antigen receptor signalling, and a marker for all white blood cells. CD45 expression is essential for T cell activation by the TCR. CD45 may be a receptor for CD26.

In some embodiments, the NKT-like cells of the disclosure express CD45. In embodiments relating to populations of the NKT-like cells of the disclosure, at least 5, 10, 20, 30, 40, 50, 60, 70, 80, 90, 95, 96, 97, 98, or 99% of the NKT-like cells may express CD45.

The CD45 may be any isoform of CD45, such as CD45RA, CD45RO and/or CD45RABC (also known as CD45R; also known as B220).

### CD56

CD56 (cluster of differentiation 56; also known as neural cell adhesion molecule, NCAM) is a homophilic binding glycoprotein expressed on the surface of neurons, glia and skeletal muscle. CD56 expression is associated with natural killer cells.

In some embodiments, the NKT-like cells of the disclosure express CD56. In embodiments relating to populations of the NKT-like cells of the disclosure, at least 5, 10, 20, 30, 40, 50, 60, 70, 80, 90, 95, 96, 97, 98, or 99% of the NKT-like cells may express CD56.

### ICAM3

ICAM-3 (Intercellular Adhesion Molecule 3; also known as CD50) is expressed by lymphocytes, monocytes, eosinophils and neutrophils (as well as on bronchioles, and by lymphoma cells and some melanoma, sarcoma, and other cancer cells).

In some embodiments, the NKT-like cells of the disclosure express ICAM3. In embodiments relating to populations of the NKT-like cells of the disclosure, at least 5, 10, 20, 30, 40, 50, 60, 70, 80, 90, 95, 96, 97, 98, or 99% of the NKT-like cells may express ICAM3.

### Major Histocompatibility Complex; MHC

The MHC was discovered by Gorer and Snell et al in 1936. Their skin transplantation experiments with mice revealed that self- and non-self recognition depended on the genetic background. Snell et al named the group of mouse genes that determine self/non-self as histocompatibility-2 (H-2). The genomic loci of the MHC encode polymorphic cell-membrane-bound glycoproteins known as MHC classical class I and class II molecules (antigens), which regulate the immune response by presenting peptides of fragmented proteins to circulating cytotoxic and helper T lymphocytes, respectively. Classical MHC class 1 proteins have been subdivided as HLA-A, HLA-B and HLA-C(Nakamura et al, 2019, which is hereby incorporated by reference in its entirety). On the other hand, HLA-E, HLA-F, HLA-G, MHC class I polypeptide-related sequence A (MICA) and FcRn etc. are classified as non-classical MHC class I.

MHC classical class I molecules are expressed in most tissues and they associate non-covalently with b2-microglobulin to present intracellularly processed peptide antigens (which are 8-11 amino acids in length) to T-cell receptors of specific CD8+ T cells in order to induce their activation and/or cytotoxicity (Shiina et al 2016, which is hereby incorporated by reference in its entirety). The processed peptides may arise from the cell's own proteome or from foreign intracellular pathogens. Mature dendritic cells use the MHC class I system to present peptides deriving from antigens captured by endocytosis. This process, called cross-presentation, plays a crucial role in the initiation of responses of specific T CD8+ lymphocytes in peripheral lymphoid organs (Shiina et al). In addition, the MHC classical class I proteins may act as ligands for killer-cell immunoglobulin-like receptors that regulate the cytotoxic activity of cytotoxic T cells and natural killer cell and leucocyte immunoglobulin-like receptors expressed on myelomonocytes and other leucocyte lineages. In contrast to the classical class I antigens, the classical class II antigens form heterodimeric structures specialized in the presentation of exogenous peptides (15-25 amino acids in length) on the surface of lymphoid cells to the CD4+ helper T lymphocytes of the immune system. The class II gene expression is predominantly restricted to the lymphoid cells, such as B cells, monocytes, macrophages, endothelial cells, dendritic cells and activated T cells. MHC class II proteins are identified as HLA-DR, HLA-DP and HLA-DQ. The MHC class II genes include HLA-DRA1, HLA-DQA1, HLA-DPA1 encoding a chain, HLA-DRB1, HLA-DRB3, HLA-DRB4, HLA-DRB5 (HLA-DRB3/4/5), HLA-DQB1, and HLA-DPB1 encoding β chain. HLA-DRA1 forms a heterodimer with HLA-DRB1 or HLA-DRB3/4/5 (Nakamura et al). Similarly, HLA-DQA1 and HLA-DPA1 are also associated with HLA-DQB1 and HLA-DPB1, respectively. The HLA-DR is divided into 5 groups consisting of DR1, DR51, DR52, DR53 and DR8 depending on the antigen group. The DR1 and DR8 groups both consist only of DRB1 as an expressed gene. On the other hand, The DR51, DR52, and DR53 groups contain DRB1 in common and furthermore consist of DRB5, DRB3, and DRB4, which is considered to be generated from DRB1 gene by gene duplication, as expressed genes, respectively (Nakamura et al).

Both the classical class I and class II genes are often highly polymorphic, presumably to preserve the inter-individual variability of the antigen-presenting ability and help the species to defend against and survive the natural selection pressure from various infectious agents. The non-classical class I and class II antigens, although similar in structure to their classical class I or class II counterparts, are usually far less polymorphic, have variable or limited tissue expression and functions that are often distinctly different to those of the classical class I or class II antigens. Moreover, several non-classical MHC class I genes are located outside the MHC (Shiina et al).

The loci of the HLA complex (such as HLA-A, HLA-B, HLA-C, HLA-DR, HLA-DQ, and HLA-DP) have many polymorphisms, so the combination (haplotype) is exceedingly large. However, the MHC exhibits strong linkage disequilibrium, which is the appearance of non-random association of alleles at multiple loci. This linkage disequilibrium in the MHC region often causes a specific combination for each locus of MHC. When two genetic polymorphisms are present on the same chromosome, the two polymorphisms are classified as linked (Nakamura et al). Given that genetic recombination has occurred in a biologically conventional manner, polymorphisms at separate sites are not able to be determined as in the linked state. However, linkage disequilibrium is a state where certain gene polymorphism can be predicted with extremely high probability based on information of the polymorphism at a distant site. In the MHC, the gene loci are concentrated in a narrow region of chromosome 6, so recombination between each gene is less likely to occur. Therefore, genes such as HLA-A, HLA-B, HLA-C, and HLA-DRB1 are often inherited in a linkage disequilibrium state. As HLA gene polymorphism analysis progresses, haplotypes that are associated with specific diseases that that are frequently found in specific ethnic groups have been elucidated. These ethnic group-specific haplotypes are thought to be involved in the process of forming ethnic groups. Thus, these haplotypes are commonly used to search for ethnic roots.

In humans, the MHC classical class I genes are involved critically in organ transplant rejection and graftversus-host disease following haematopoietic stem cell transplants. Various associations have been evidenced between HLA class I molecules and the numerous autoimmune diseases, as well as infectious diseases and drug adverse reactions. Apart from their essential role in the elaboration of adaptive immune responses, the role of MHC class I genes was demonstrated in various steps of reproduction such as pregnancy maintenance, mate selection and kin recognition. The MHC has also been considered to be a system primarily for sexual selection and avoidance of inbreeding with histocompatibility fulfilling a secondary role. The MHC class I gene products also have impact on central nervous system development and plasticity, neurological cell interactions, synaptic function and behaviour, cerebral hemispheric specialization, and neurological and psychiatric disorders. Hence, the human MHC class I region is one of the most biomedically diverse and important genomic regions (Shiina et al).

### TCR Gamma Delta

T-cell receptor gamma delta (TCR gamma/delta; TCR γδ) is a T-cell receptor that is made up of one γ (gamma) chain and one δ (delta) chain. TCR gamma/delta expressing T-cells (gamma delta T cells) are important recognizers of lipid antigens expressed by cancer cells as well as stressed cells such as cancer cells, microbial and viral infected cells and autoreactive lymphocytes. Gamma delta T cells exhibit several characteristics that place them at the border between the more evolutionarily primitive innate immune system that permits a rapid beneficial response to a variety of foreign agents and the adaptive immune system, where B and T cells coordinate a slower but highly antigen-specific immune response leading to long-lasting memory against subsequent challenges by the same antigen. Gamma delta T cells may be considered a component of adaptive immunity in that they rearrange TCR genes to produce junctional diversity and can develop a memory phenotype.

The most common human gamma delta variant is the Vgamma9/Vdelta2 variant in blood, while Vdelta1 type gamma delta T cells in tumors have been associated with prognosis. A Vdelta3 variant has also been described, as has a Vdelta2 negative variant following CMV infection which reduced cancer risk. In contrast to MHC-restricted alpha beta T cells, gamma delta T cells do not require antigen processing and MHC presentation of peptide epitopes, although some can recognize MHC class Ib. Consequently, tumor cells cannot evade detection by down-regulating MHC and gamma delta T cells thus also have equal potential for killing tumors with low mutational load, and are less likely to be affected by resistance issues. Gamma delta T cell tumor infiltration has also been correlated highest with survival and lower incidence of graft versus host disease. Gamma delta T cells naturally home to various tissues to detect tumors and are preferred for allogeneic therapy over alpha beta T cells.

In some embodiments, the NKT-like cells of the disclosure express TCR gamma/delta. In embodiments relating to populations of the NKT-like cells of the disclosure, at least 5, 10, 20, 30, 40, 50, 60, 70, 80, 90, 95, 96, 97, 98, or 99% of the NKT-like cells may express TCR gamma/delta. The NKT-like cells may express TCR gamma/delta that comprises a delta 1 (81), delta 2 (82), delta 3 (83), or delta 5 (85) delta chain. That is, the NKT-like cells of the disclosure may be delta 1, or delta 2, or delta 3, or delta 5 positive. In embodiments relating to populations of the NKT-like cells of the disclosure, at least 5, 10, 20, 30, 40, 50, 60, 70, 80, 90, 95, 96, 97, 98, or 99% of the NKT-like cells may be delta 1, or delta 2, or delta 3, or delta 5 positive.

invariant TOR (iTCR)

The invariant TCR (iTCR) is a highly conserved invariant receptor that in humans consists of the Vα24-Jα18 chain joined to the Vβ11 chain, and in mouse consists of a Vα14-Jα18 chain that pairs preferentially with Vβ2, Vβ7, or Vβ8.2 chains. The iTCR is expressed by invariant natural killer T cells (iNKTs), a unique innate-type T lymphocyte that has characteristics of both conventional T cells and natural killer cells. These cells directly kill tumor cells and trans-activate the anti-tumor functions of dendritic cells (DC), natural killer (NK) cells, and T and B cells. iNKT cell activation commonly requires engagement of the iTCR by CD1d presenting glycolipid antigens.

In some embodiments, the NKT-like cells of the disclosure express iTCR. In embodiments relating to populations of the NKT-like cells of the disclosure, at least 5, 10, 20, 30, 40, 50, 60, 70, 80, 90, 95, 96, 97, 98, or 99% of the NKT-like cells may express iTCR.

TCR alpha/beta

T-cell receptor alpha beta (TCR alpha/beta; TCR αβ) is the predominant TCR heterodimer that is made up of one α (alpha) chain and one β (beta) chain.

In some embodiments, the NKT-like cells of the disclosure may express TCR alpha/beta. In embodiments relating to populations of the NKT-like cells of the disclosure, at least 5, 10, 20, 30, 40, 50, 60, 70, 80, 90, 95, 96, 97, 98, or 99% of the NKT-like cells may express TCR alpha/beta.

### CD16

CD16 (cluster of differentiation 16; also known as FcγRIII) is a transmembrane protein present on activated natural killer cells, and a marker of cell activation.

In some embodiments, the NKT-like cells of the disclosure may express CD16. In embodiments relating to populations of the NKT-like cells of the disclosure, at least 5, 10, 20, 30, 40, 50, 60, 70, 80, 90, 95, 96, 97, 98, or 99% of the NKT-like cells may express CD16.

### NKp44

NKp44 (also known as cluster of differentiation 336, natural cytotoxicity triggering receptor 2) is a cell-surface receptor selectively expressed on activated NK cells, and a marker of cell activation.

In some embodiments, the NKT-like cells of the disclosure express NKp44. In embodiments relating to populations of the NKT-like cells of the disclosure, at least 5, 10, 20, 30, 40, 50, 60, 70, 80, 90, 95, 96, 97, 98, or 99% of the NKT-like cells may express NKp44.

The NKT-like cells of the disclosure may express CD56, TCR gamma/delta, iTCR, CD16, NKp44, CD3, CD8, CD14, CD19, CD45, and/or TCR alpha/beta. The NKT-like cells of the disclosure may not express CD4. In some preferred embodiments, the NKT-like cells of the disclosure express CD56, TCR gamma/delta, and iTCR. In some preferred embodiments, the NKT-like cells of the disclosure express CD16 and NKp44. In some preferred embodiments, the NKT-like cells of the disclosure express CD56, TCR gamma/delta, iTCR, CD16, and NKp44. In some preferred embodiments, the NKT-like cells of the disclosure express CD56, TCR gamma/delta, iTCR, and TCR alpha/beta. In some preferred embodiments the NKT-like cells of the disclosure express CD56, TCR gamma/delta, iTCR, CD16, NKp44, and TCR alpha/beta. In some embodiments the NKT-like cells of the disclosure express CD56, TCR gamma/delta, iTCR, CD16, and NKp44, and one or more of CD3, CD8, CD14, CD19, CD45, and/or TCR alpha/beta. In some embodiments the NKT-like cells of the disclosure express CD56, TCR gamma/delta, iTCR, CD16, NKp44, and TCR alpha/beta, and one or more of CD3, CD8, CD14, CD19, and/or CD45.

In some particularly preferred embodiments the NKT-like cells of the disclosure express CD56, TCR gamma/delta, and/or iTCR.

In embodiments relating to populations of the NKT-like cells of the disclosure, the population of NKT-like cells may be characterized in that at least 60, 70, 80, 90, 95, 96, 97, 98, or 99% of the NKT cells express CD56, TCR gamma/delta, iTCR, CD16, NKp44, CD3, CD8, CD14, CD19, CD45, and/or TCR alpha/beta. In some such embodiments, the population of NKT-like cells may be characterized in that at least 60, 70, 80, 90, 95, 96, 97, 98, or 99% of the NKT-like cells do not express: CD4. In some embodiments, the population of NKT-like cells may be characterized in that at least 60, 70, 80, 90, 95, 96, 97, 98, or 99% of the NKT-like cells express CD56, TCR gamma/delta, and iTCR. In some embodiments, the population of NKT-like cells may be characterized in that at least 60, 70, 80, 90, 95, 96, 97, 98, or 99% of the NKT-like cells express CD16 and NKp44. In some embodiments, the population of NKT-like cells may be characterized in that at least 60, 70, 80, 90, 95, 96, 97, 98, or 99% of the NKT-like cells express CD56, TCR gamma/delta, iTCR, CD16, and NKp44. In some embodiments, the population of NKT-like cells may be characterized in that at least 60, 70, 80, 90, 95, 96, 97, 98, or 99% of the NKT-like cells express CD56, TCR gamma/delta, iTCR, and TCR alpha/beta. In some embodiments, the population of NKT-like cells may be characterized in that at least 60, 70, 80, 90, 95, 96, 97, 98, or 99% of the NKT-like cells express CD56, TCR gamma/delta, iTCR, CD16, NKp44, and TCR alpha/beta. In some embodiments, the population of NKT-like cells may be characterized in that at least 60, 70, 80, 90, 95, 96, 97, 98, or 99% of the NKT-like cells express CD56, TCR gamma/delta, iTCR, CD16, and NKp44, and one or more of CD3, CD8, CD14, CD19, CD45, and/or TCR alpha/beta. In some embodiments, the population of NKT-like cells may be characterized in that at least 60, 70, 80, 90, 95, 96, 97, 98, or 99% of the NKT-like cells express CD56, TCR gamma/delta, iTCR, CD16, NKp44, and TCR alpha/beta, and one or more of CD3, CD8, CD14, CD19, and/or CD45.

In some particularly preferred embodiments, the population of NKT-like cells may be characterized in that at least 60, 70, 80, 90, 95, 96, 97, 98, or 99% of the NKT-like cells express CD56, TCR gamma/delta, and iTCR.

### Gamma Delta T Cells

Gamma delta T cell surface marker characteristics may include (but are not limited to) CD3, CD4, CD8, CD69, CD56, CD27, CD40, CD40L, CD45RA, CD45, CD83, CD16, CD16a, CD16b, ICOS, CD161, Fas, CLEC7A/Dectin-1, FasL, Ecadherin, IL-18R alpha, IL-23R, NKG2D/CD314, NKG2E, Occludin, TKR2, TRAIL, TCR-Vg9, TCR-Vd2, TCR-Vd1, TCR-Vd3, TCR-pan g/d, NKG2D, monoclonal chemokine receptor antibodies CCR5, CCR6, CCR7, CXCR3, CXCR4, or CXCR5 or combinations thereof. The surface marker characteristics of the NKT-like cells of the invention may include one/more of these. Gamma delta T cells may secrete (including but not limited to) CCL2/JE/MCP-1, CXCL13/BLC/BCA-1, beta-Defensin 2, beta-Defensin 3, alpha-Defensin 1, EGF, KGF/FGF-7, FGF-10, GM-CSF, Granulysin, Granzyme A, Granzyme B, IFN-gamma, IGF-I/IGF-1, IL-2, IL-4, IL-5, IL-6, IL-10, IL-12, IL-12/IL-23 p40, IL-12 p70, IL-13, IL-17/IL-17A, IL-22, IL-6/IL-6R alpha Complex, LAP (TGF-beta 1), TGF-beta, and/or TNF-alpha. The NKT-like cells of the invention may secrete one/more of these.

The NKT-like cells and populations of NKT-like cells of the disclosure may be characterised in that these cells express CD56, TCR gamma/delta, and iTCR. While cells which express both TCR gamma/delta and iTCR can be manufactured (for example by transduction of TCR gamma/delta positive cells with the iTCR), to the best of the authors' knowledge these have not previously been described as occurring naturally (that is, without one or both of these being recombinantly introduced). The NKT-like cells of the disclosure are therefore unique in that they are naturally occurring, produced and/or mobilized in subjects following administration of high dose glucocorticoids. That is, the NKT-like cells of the disclosure are unique in that they express both TCR gamma/delta and iTCR without the need for recombinant expression of one or both of these- and advantageously avoid drawbacks associated with the use of manufactured γδTCR/iTCR cell lines. Thus, isolated NKT-like cells and populations of NKT-like cells of the disclosure may be described as naturally-occurring. The cells and populations of cells of the disclosure have not been transfected, transduced, or otherwise genetically modified to express TCR gamma/delta. The cells and populations of cells of the disclosure have not been modified by introducing a nucleic acid encoding TCR gamma/delta into the cell or cells. The cells and populations of cells of the disclosure have not been transfected, transduced, or otherwise genetically modified to express iTCR. The cells and populations of cells of the disclosure have not been modified by introducing a nucleic acid encoding iTCR into the cell or cells. In some embodiments, the cells and populations of cells of the disclosure may not have been transfected, transduced, or otherwise genetically modified to express TCR alpha/beta. In some embodiments, the cells and populations of cells of the disclosure may not have been modified by introducing a nucleic acid encoding TCR alpha/beta into the cell or cells. In some embodiments, the cells and populations of cells of the disclosure may not have been transfected, transduced, or otherwise genetically modified to express CD56, TCR gamma/delta, iTCR, CD16, NKp44, CD3, CD8, CD14, CD19, CD45, TCR alpha/beta, CD34, and/or ICAM3. In some embodiments, the cells and populations of cells of the disclosure may not have been transfected, transduced, or otherwise genetically modified to express CD56, TCR gamma/delta, iTCR, CD16, NKp44, CD3, CD8, CD14, CD19, CD45, and/or TCR alpha/beta. In some embodiments, the cells and populations of cells of the disclosure may not have been modified by introducing a nucleic acid encoding CD56, TCR gamma/delta, iTCR, CD16, NKp44, CD3, CD8, CD14, CD19, CD45, TCR alpha/beta, CD34, and/or ICAM3 into the cell or cells. In some embodiments, the cells and populations of cells of the disclosure may not have been modified by introducing a nucleic acid encoding CD56, TCR gamma/delta, iTCR, CD16, NKp44, CD3, CD8, CD14, CD19, CD45, and/or TCR alpha/beta into the cell or cells. In some embodiments, the cells and populations of cells of the disclosure may not have been transfected, transduced, or otherwise genetically modified to express CD56, TCR gamma/delta, iTCR, CD16, NKp44, CD3, CD8, CD14, CD19, CD45, TCR alpha/beta, CD34, or ICAM3. In some embodiments, the cells and populations of cells of the disclosure may not have been transfected, transduced, or otherwise genetically modified to express CD56, TCR gamma/delta, iTCR, CD16, NKp44, CD3, CD8, CD14, CD19, CD45, or TCR alpha/beta. In some embodiments, the cells and populations of cells of the disclosure may not have been modified by introducing a nucleic acid encoding CD56, TCR gamma/delta, iTCR, CD16, NKp44, CD3, CD8, CD14, CD19, CD45, TCR alpha/beta, CD34, or ICAM3 into the cell or cells. In some embodiments, the cells and populations of cells of the disclosure may not have been modified by introducing a nucleic acid encoding CD56, TCR gamma/delta, iTCR, CD16, NKp44, CD3, CD8, CD14, CD19, CD45, or TCR alpha/beta into the cell or cells. In some embodiments, the cells and populations of cells of the disclosure may be isolated from a subject. The subject may be a subject as defined elsewhere herein. In some embodiments, the cells and populations of cells of the disclosure may be isolated from a subject in which they were induced/mobilized. In some embodiments, the cells and populations of cells of the disclosure may be isolated from a subject in which they were induced/mobilized following administration of a glucocorticoid to the subject. In some embodiment the cells and populations of cells of the disclosure may be isolated from a subject in which they were induced/mobilized via a method as disclosed elsewhere herein. In some embodiments, the cells and populations of cells of the disclosure may be produced and isolated by a method as disclosed elsewhere herein.

ICAM3 modulating agents in the context of the present disclosure are those which bind ICAM3 and promote the induction and/or mobilization of the NKT-like cells of the invention. ICAM3 modulating agents which bind ICAM3 may alternatively be referred to as ICAM3 binding molecules, ICAM3 binding agents, etc. The ICAM3 modulating agent may be an ICAM3 antagonist/ICAM3 inhibitor, or may be an ICAM3 agonist/activator.

Such ICAM3 modulating agents may include, for example, anti-ICAM3 antibodies raised against ICAM3 or a portion thereof, small molecule modulators of ICAM3 (such as activators or inhibitors of ICAM3), and peptide agents/proteins which bind ICAM3. Suitable means of identifying ICAM3 modulating agents will be well known to those of skill in the art—for example, anti ICAM3 antibodies may be identified by a method which may include bringing into contact a library of antibody molecules and an ICAM3 epitope, and selecting one or more specific antibody molecules of the library able to bind said epitope. Alternatively, these could be identified using competition binding assays employing known anti ICAM3 antibodies, with competition determined, for example, using ELISA or flow cytometry. Similarly, small molecule modulators of ICAM3 may be identified by routine screening experiments such as radioligand binding assays and functional assays.

As already described above, the present authors have discovered the surprising capacity of glucocorticoid receptor modulating agents (such as dexamethasone and other glucocorticoids) to bind ICAM3 and exert modulating actions upon ICAM3. Thus, in some embodiments, the ICAM3 modulating agent may be a glucocorticoid-receptor (GR) modulating agent. In some embodiments, the ICAM3 modulating agent may be a glucocorticoid, for example dexamethasone or betamethasone. The ICAM3 modulating agent may be a molecule that binds to the same region of ICAM3 as glucocorticoids such as dexamethasone. The ICAM3 modulating agent may be a molecule that binds to ICAM3 via interaction with the SER31 and/or MET49 residues in ICAM3. The ICAM3 modulating agent may be a molecule that binds to ICAM3 via interaction with the THR38, LEU40, LEU56, VAL59, and/or ILE65 residues in ICAM3. The ICAM3 modulating agent may be a molecule that binds to ICAM3 via interaction with the PHE21, VAL22, GLU32, LYS33, TRP51, and/or ALA52 residues in ICAM3. The ICAM3 modulating agent may be a molecule that binds to ICAM3 via interaction with the SER25, ASN23, GLU37, PHE54, and/or GLN75 residues in ICAM3. The ICAM3 modulating agent may be a molecule that binds to ICAM3 via interaction with the PHE21, VAL22, ASN23, SER25, SER31, GLU32, LYS33, GLU37, THR38, LEU40, MET49, TRP51, ALA52, PHE54, LEU56, VAL59, ILE65, and/or GLN75 residues in ICAM3. The ICAM3 modulating agent may be any molecule, such as an anti-ICAM3 antibody, small molecule modulator of ICAM3 (including activators and inhibitors of ICAM3), or peptide agent/protein which binds ICAM3, which competes with a glucocorticoid such as dexamethasone for binding to ICAM3. The ICAM3 modulating agent may be an anti-ICAM3 antibody, for example ICR 8.1 or a humanised version thereof. The skilled person is aware of suitable techniques by which binding to the same region of ICAM3 could be determined—for example, by molecular modelling or competition binding assays.

As used herein, the term glucocorticoid-receptor (GR) modulating agent includes glucocorticoids, glucocorticoid receptor agonists, and any compound that binds to the glucocorticoid receptor. Glucocorticoid-receptor (GR) modulating agents such as glucocorticoids exert their effects through both membrane GRs and cytoplasmic GRs which activate or repress gene expression. Some of the desirable lymphodepletive effects of glucocorticoids and GR modulating agents are believed to be mediated via membrane GRs or other non-genomic effects in addition to their genomic effects. Glucocorticoids have been reported to have varied effects on lymphocyte levels, depending on the concentration of the glucocorticoid administered and the duration of treatment. In general, at low doses typically used for chronic therapy, glucocorticoids have been reported to redistribute lymphocytes from the peripheral blood into the bone marrow, at medium doses glucocorticoids have been reported to cause leukocytosis thought to be a redistribution of leukocytes from the bone marrow, spleen and thymus into the peripheral blood, and at high doses glucocorticoids have a lymphotoxic action on lymphocytes by triggering apoptosis and necroptosis. The duration of effect also depends on the dose level; for instance Fauci et al (1976) reports a single oral 0.24 mg/kg dexamethasone dose suppresses peripheral blood T and B lymphocytes 80% with recovery beginning at 12 hours and normal levels by 24 hours. The present authors have previously demonstrated (in international patent application PCT/US2019/054395) that acute oral doses of 3 mg/kg or greater dexamethasone are necessary to reduce peripheral blood T and B cells 24-48 hours after administration, with return to baseline levels occurring around 5 to 14 days after dosing.

Glucocorticoid-receptor (GR) modulating agents which may be used in the disclosed methods include, for example, selective glucocorticoid receptor modulators (SEGRMs) and selective glucocorticoid receptor agonists (SEGRAs). Glucocorticoids, selective glucocorticoid receptor modulators, and selective glucocorticoid receptor agonists (SEGRAs) that may be utilized in the disclosed methods are well known to those skilled in the art.

Some such glucocorticoids include, but are not limited to, dexamethasone, dexamethasone containing agents, hydrocortisone, methylpredisone, prednisone, corticone, budesonide, betamethasone and beclomethasone. Other glucocorticoids include prednisolone, mometasone furoate, Triamcinolone Acetonide, and methylprednisolone.

Accordingly, in some embodiments of the methods of the disclosure, the glucocorticoid-receptor (GR) modulating agent may be a glucocorticoid. In some such embodiments, the glucocorticoid may be selected from the group consisting of: dexamethasone, hydrocortisone, methylprednisolone, prednisone, prednisolone, prednylidene, cortisone, budesonide, betamethasone, flumethasone and beclomethasone. In some preferred embodiments, the glucocorticoid may be selected from the group consisting of: dexamethasone, betamethasone, and methylprednisone. In some particularly preferred embodiments the glucocorticoid may be dexamethasone or betamethasone.

In some embodiments of the methods of the disclosure, the glucocorticoid may be selected from the group consisting of: dexamethasone base, dexamethasone sodium phosphate, dexamethasone hemisuccinate, dexamethasone sodium succinate, dexamethasone succinate, dexamethasone isonicotinate, dexamethasone-21-acetate, dexamethasone phosphate, dexamethasone-21-phosphate, dexamethasone tebutate, dexamethasone-17-valerate, dexamethasone acetate monohydrate, dexamethasone pivalate, dexamethasone palmitate, dexamethasone-21-palmitate, dexamethasone dipropionate, dexamethasone propionate, dexamethasone acetate anhydrous, dexamethasone-21-phenylpropionate, dexamethasone-21-sulfobenzoate, dexamethasone hemo-sulfate, dexamethasone sulfate, dexamethasone beloxil, dexamethasone acid, dexamethasone acefurate, dexamethasone carboximide, dexamethasone cipecilate, dexamethasone 21-phosphate disodium salt, dexamethasone mesylate, dexamethasone linoleate, dexamethasone glucoside, dexamethasone glucuronide, dexamethasone iodoacetate, dexamethasone oxetanone, carboxymethylthio-dexamethasone, dexamethasonebisethoximes, dexamethasone epoxide, dexamethasonelinolelaidate, dexamethasone methylorthovalerate, dexamethasone spermine, 6-hydroxy dexamethasone, dexamethasone tributylacetate, dexamethasone aspartic acid, dexamethasone galactopyranose, dexamethasone hydrochloride, hydroxy dexamethasone, carboxy dexamethasone, desoxy dexamethasone, dexamethasone butazone, dexamethasone cyclodextrin, dihydro dexamethasone, oxo dexamethasone, propionyloxy dexamethasone, dexamethasone galactodie, dexamethasone isonicotinate, dexamethasone sodium hydrogen phosphate, dexamethasone aldehyde, dexamethasone pivlate, dexamethasone tridecylate, dexamethasone crotonate, dexamethasone methanesulfonate, dexamethasone butylacetate, dehydro dexamethasone, dexamethasone Isothiocyanatoethyl)Thioether, dexamethasone bromoacetate, dexamethasone hemiglutarate, deoxy dexamethasone, dexamethasone chlorambucilate, dexamethasone melphalanate, formyloxy dexamethasone, dexamethasone butyrate, dexamethasone laurate, dexamethasone acetate, and any combination treatment that contains a form of dexamethasone. In some preferred embodiments, the glucocorticoid may be dexamethasone base or dexamethasone sodium phosphate.

In some embodiments of the disclosure, the glucocorticoid receptor modulating agent may not be one or more of the above recited agents.

In the methods of the disclosure, the glucocorticoid-receptor (GR) modulating agent or ICAM3 modulating agent is administered at a dose equivalent to about at least 6 mg/kg human equivalent dose (HED) of dexamethasone base, or at a dose equivalent to about at least 6 mg/kg HED of dexamethasone phosphate.

Equivalent doses of another glucocorticoid or glucocorticoid receptor modulating agent can be readily and easily calculated using publicly available corticoid conversion algorithms, preferably http://www.medcalc.com. By way of example, 3 to 12 mg/kg dexamethasone converts to 19 to 75 mg/kg prednisone. Since prednisone's biologic half-life is about 20 hours, while dexamethasone's biologic half-life is about 36 to 54 hours prednisone would be dosed between 19 to 75 mg/kg every 24 hours for equivalent biologic dosing. More specifically, a 12 mg/kg dose of dexamethasone corresponds to a 75 mg/kg dose of prednisolone that would require repeat dosing of about two to about three doses every 24 hours. A 10 mg/kg dose of betamethasone is about 12 mg/kg dexamethasone and has a pharmacodynamic (biologic) half-life similar to dexamethasone.

Dexamethasone doses in the examples in the present application are given as human equivalent doses (HED). Methods for calculating the human equivalent dose (HED) are known in the art. For example the FDA's Centre for Drug Evaluation and Research (CDER) issued a highly-cited guidance document in 2005 (U.S Department of Health CDER, 2005), which sets out the established algorithm for converting animal doses to HED based on body surface area (the generally accepted method for extrapolating doses between species) at Table 1 on page 7 of that document. For reference, Table 1 is reproduced below. The skilled person understands that the animal dose in mg/kg, explained below, the HED is calculated easily using the standard conversion factors in the right hand columns of Table 1:

TABLE 1
Conversion of Animal Doses to Human Equivalent
Doses Based on Body Surface Area

| | | To Convert Animal Dose in mg/ | |
|---|---|---|---|
| | To Convert Animal | kg to HED^{a} in mg/kg, Either: | |
| | Dose in mg/kg to | Divide | Multiply |
| | Dose in mg/m², | Animal | Animal |
| Species | Multiply by kₘ | Dose By | Dose By |
| Human | 37 | — | — |
| Child (20 kg)^{b} | 25 | — | — |
| Mouse | 3 | 12.3 | 0.08 |
| Hamster | 5 | 7.4 | 0.13 |
| Rat | 6 | 6.2 | 0.16 |
| Ferret | 7 | 5.3 | 0.19 |
| Guinea pig | 8 | 4.6 | 0.22 |
| Rabbit | 12 | 3.1 | 0.32 |
| Dog | 20 | 1.8 | 0.54 |

Primates:

| | | | |
|---|---|---|---|
| Monkeys^{c} | 12 | 3.1 | 0.32 |
| Marmoset | 6 | 6.2 | 0.16 |
| Squirrel monkey | 7 | 5.3 | 0.19 |
| Baboon | 20 | 1.8 | 0.54 |
| Micro-pig | 27 | 1.4 | 0.73 |
| Mini-pig | 35 | 1.1 | 0.95 |

^{a}Assumes 60 kg human. For species not listed or for weights outside the standard ranges, HED can be calculated from the following formula: HED = animal dose in mg/kg × (animal weight in kg/human weight in kg)^{0.33}.
^{b}This kₘ value is provided for reference only since healthy children will rarely be volunteers for phase 1 trials.
^{c}For example, cynomolgus, rhesus, and stumptail.

In some embodiments of the methods of the disclosure, the glucocorticoid-receptor (GR) modulating agent or ICAM3 modulating agent is administered at a dose equivalent to about at least 12 mg/kg human equivalent dose (HED) of dexamethasone base or dexamethasone phosphate. In other preferred embodiments, the glucocorticoid-receptor (GR) modulating agent is administered at a dose equivalent to about at least 15 mg/kg or about at least 18 mg/kg human equivalent dose (HED) of dexamethasone base or dexamethasone phosphate. In other preferred embodiments, the glucocorticoid-receptor (GR) modulating agent is administered at a dose equivalent to about at least 21 mg/kg or at least about 24 mg/kg human equivalent dose (HED) of dexamethasone base or dexamethasone phosphate. In some preferred embodiments, the glucocorticoid-receptor (GR) modulating agent is administered at a dose equivalent to about 12 mg/kg human equivalent dose (HED) of dexamethasone base or dexamethasone phosphate, about 15 mg/kg human equivalent dose (HED) of dexamethasone base or dexamethasone phosphate, or about 18 mg/kg human equivalent dose (HED) of dexamethasone base or dexamethasone phosphate, or about 21 mg/kg human equivalent dose (HED) of dexamethasone base or dexamethasone phosphate, or about 24 mg/kg human equivalent dose (HED) of dexamethasone base or dexamethasone phosphate, or about 30 mg/kg human equivalent dose (HED) of dexamethasone base or dexamethasone phosphate, or about 45 mg/kg human equivalent dose (HED) of dexamethasone base or dexamethasone phosphate.

In some embodiments of the methods of the disclosure, the glucocorticoid-receptor (GR) modulating agent or ICAM3 modulating agent is administered at a dose equivalent to about at least 6-45 mg/kg human equivalent dose (HED) of dexamethasone base or dexamethasone phosphate; about at least 15-24 mg/kg human equivalent dose (HED) of dexamethasone base or dexamethasone phosphate; about at least 6-12 mg/kg human equivalent dose (HED) of dexamethasone base or dexamethasone phosphate; about at least 6-18 mg/kg human equivalent dose (HED) of dexamethasone base or dexamethasone phosphate, or about at least 12-15 mg/kg human equivalent dose (HED) of dexamethasone base or dexamethasone phosphate; or about at least 18-30 mg/kg human equivalent dose (HED) of dexamethasone base or dexamethasone phosphate; or about at least 15-18 mg/kg human equivalent dose (HED) of dexamethasone base or dexamethasone phosphate. In embodiments in which the infectious disease is a disease resulting from infection with a coronavirus, for example COVID-19, the glucocorticoid-receptor (GR) modulating agent may preferably be administered at a dose equivalent to between about 18-30 mg/kg human equivalent dose (HED) of dexamethasone base or dexamethasone phosphate.

In the methods of the disclosure, the glucocorticoid-receptor (GR) modulating agent or ICAM3 modulating agent may be administered as a single acute dose, or as a total dose given over about a 24, 48, or 72 hour period. In some preferred embodiments, the glucocorticoid-receptor (GR) modulating agent is administered as a single acute dose. In other preferred embodiments, the glucocorticoid-receptor (GR) modulating agent is administered as a total dose given over about a 72 hour period.

In some embodiments in which the subject has, is suspected of having, or has been diagnosed with an infectious disease, such as a disease resulting from infection with a coronavirus (such as COVID-19), the glucocorticoid receptor modulating agent (which may preferably be dexamethasone or betamethasone) may be administered as a solution in aqueous media. In some such embodiments, the glucocorticoid receptor modulating agent may be provided at a concentration equivalent to about 24 mg/ml dexamethasone phosphate (20 mg/ml dexamethasone base; 26.2 mg/ml dexamethasone sodium phosphate), and administered by intravenous (IV) infusion over a period of about 1 to 2 hours, at an ultimate target dose of between about 18 to 30 mg/kg human equivalent dose (HED) of dexamethasone base. In other embodiments, the glucocorticoid receptor modulating agent may be provided as dexamethasone tablets dissolved in orange juice or citric acid (pH 3.3-4.2) and administered orally or by stomach tube, at an ultimate target dose of between about 18 to 30 mg/kg human equivalent dose (HED) of dexamethasone base.

In some embodiments of the methods of the disclosure, the methods may comprise a step of administering one or more further doses of a glucocorticoid-receptor (GR) modulating agent or ICAM3 modulating agent to the subject.

In this context, the one or more doses are administered further to a first or preceding dose of glucocorticoid-receptor (GR) modulating agent or ICAM3 modulating agent and may therefore be termed subsequent or second, third, fourth, etc. doses. Accordingly, in some embodiments, the one or more further doses may be administered about 24, 48, 72, 96, 120, 144, or 168 hours after a preceding dose (administration). In some embodiments, the one or more further doses may be administered every about 24, 48, 72, 96, 120, 144, or 168 hours after a preceding dose (administration). In some other embodiments, the one or more further doses may be administered once every week, once every two weeks, once every three weeks, or once every month after a preceding dose (administration). In some other embodiments, the one or more further doses may be administered twice every week after a preceding dose (administration).

In some embodiments, the one or more further doses may be administered between about 24 hours and 168 hours after a preceding dose (administration). In other embodiments, the one or more further doses may be administered between about 24 hours and 120 hours, between about 24 hours and 72 hours, or between about 24 hours and 48 hours after a preceding dose (administration). In some other embodiments, the one or more further doses may be administered between about 48 hours and 168 hours, between about 48 hours and 120 hours, or between about 48 hours and 72 hours after a preceding dose (administration). In some other embodiments, the one or more further doses may be administered between about 72 hours and 168 hours, or between about 72 hours and 120 hours after a preceding dose (administration).

In some embodiments, a subsequent dose is given 7 days after the initial dose. In some embodiments, a subsequent dose is given 14 days after the initial dose. In some embodiments, a subsequent dose is given 21 days after the initial dose.

In some embodiments in which the subject has, is suspected of having, or has been diagnosed with a T cell lymphoma, the one or more further doses may be administered every 21 days, or every 14 days or every 5-7 days for a period of time that can be determined by a physician.

In some embodiments in which the subject has, is suspected of having, or has been diagnosed with a B cell lymphoma, the one or more further doses may be administered every 21 days, or every 14 days or every 5-7 days for a period of time that can be determined by a physician.

In some embodiments of the methods of the disclosure, the methods may further comprise a step of administering an NKT cell activator, T cell activator, and/or NK cell activator to the subject.

As used herein, the term NKT cell activator includes any agent or molecule triggering activation of NKT cells. Activation of NKT cells is associated with upregulation of activation markers and Th1 and Th2 cytokines and chemokines. NKT cell activators that may be utilized in the disclosed methods are well known to those skilled in the art.

Some such NKT cell activators include, but are not limited to, Adipokines, Leptin, adiponectin, apelin, chemerin, MCP-1, PAI-1, RBP4, visfatin, omentin, vaspin, progranulin, CTRP-4, Cytokines, IL-1α, IL-1β, IL-1RA. IL-18, IL-33, IL-36α, IL-36β, IL-36γ. IL-36RA, IL-37, IL-38, IL-2, IL-4, IL-7, IL-9, IL-15, IL-21, IFN-α, IFN-β, IFN-8, IFN-ε, IFN-κ, IFN-τ, IFN-ω, IFN-γ, IFN-λ1, IFN-λ2, IFN-λ3, IFN-λ4, IL-6, IL-11, IL-31, CLCF1, CNTF, leptin, LIF, OSM, iL-12, IL-17A, IL-17B, IL-17C, IL-17D, IL-17E, IL-17F, 4-1BBL, BAFF, CD40LG, CD70, CD95L/CD178, EDA-A1, LTA/TNF-β, TNF-α, TNFSF4, TNFS8, TNFSF10, TNFSF11, TNFSF12, TNFSF13, TNFSF15, TGF-β1, TGF-β2, TGF-β3, IL-13, G-CSF, GM-CSF, CSF1. Chemokines, CXCL1-CXCL17, CC, CCL1-CCL28, CX3CL1, XCL1, XCL2, Myokines, BDNF, Decorin, irisin, myostatin, myonectin, osteonectin, Prostaglandins, PGI2, PGD2, PGE2, PGF2α, Prostamides, Prostamide 12, prostamide D2, prostamide E2, prostamide F2a, Virokines, Growth Factors, Adrenomedullin, angiopoietin, autocrine motility factor, bone morphogenic proteins, ciliary neurotrophic factor, leukemia inhibitory factor, M-CSF, EGF, ephrine A1-A5, ephrine B1-B3, erythropoietin, FGF1-FGF23, fetal bovine somatotrophin, GDNF, neurturin, persephin, artemin, growth differentiation factor-9, hepatocyte growth factor, hepatocyte-derived growth factor, insulin, insulin-like growth factor ½, keratinocyte growth factor, migration-stimulating factor, macrophage-stimulating protein, neuregulin 1-4, neurotrophin ¾, nerve growth factor, placental growth factor, platelet-derived growth factor, renalase, T-cell growth factor, TGF-α, TGF-β, VEGF, Wnt signaling pathway, anti-NKG2D antibody or its ligand MICA (MHC class I chain-related sequence A), DNAM-1 engagement, 4-1BB engagement, PD-1 inhibitor, NKT Activators, α-galactosylceramide, α-glucoronosylceramide, α-galcturonsylceramide, α-galactosyldiacylgylocerol, phosphatidylinositol-manosidase, α-glucosyldiacylglycerol, cholesterol α-glucoside, β-glaactocsylceramide, isoglobotrihexosylceramide, diasialoganglioside, phosphatidylethanolamine, phosphatidylinositol, phosphatidylcholine, house dust extract, GSL-1, NKp44L, ULBP, Pathogen-derived molecular structures, PAMP, LPS, pathogen-derived RNA, pathogen-derived DNA, viral ligands, Synthetic α-galacosylceramide, KRN7000, PBS44, PBS57, Anti-inflammatory, IL-10, IL-19, IL-20, IL-22, IL-24, IL-28A, IL-28B, IL-29.

In some embodiments of the disclosure, the NKT cell activator may not be one or more of the above recited agents.

Following activation, NKT cells express NKp44, lower CD3 and CD49b expression and express IL-10, TGF-β, IFNgamma, IL-4 and several Th1 and Th2 cytokines, Human class-I restricted T cell associated molecule (CRTAM), CCL3/MIP1a, CCL4/MIP1h and CCL5/Rantes and XCL1/lymphotactin, granzyme, CD45RO+CD62L+, CD25, IL2Rbeta, GM-CSF, IL-2, IL-13, TNFalpha, IL-17, IL-21, CD44, CD69, and IL-22. Additionally, in a tumour environment, NKT cells become organized in lines moving in towards tumor cells from all sides.

In some preferred embodiments of the methods of the disclosure, the NKT cell activator may be selected from the group consisting of: alpha GalCer (alpha-Galactosylceramide; α-GalCer) sulfatide (3-O-sulfogalactosylceramide; SM4; sulfated galactocerebroside), or an NKT-activating antibody, or may be Perforin, nitric oxide, IL-2, interferons alpha and gamma, TGFbeta, TNFalpha, TNFbeta, G-CSF, VEGF, FGF-18, IL-17, CXCL5, CXCR2, CXCR5, CCR4-CCL17/22, CCR8-CCL1, CCR10-CCL28, and CXCR3-CCL9/10/11, CCL5, CXCR9, CCL2, CCL3, CCL4, CCL5, CXCL9 or CXCL10, interferon (IFN) γ inducible chemokines CXCL9, CXCL10, and CXCL11, CCL5 and CXCL9, CCR5, IL-32, IL-6, IL-7, IL-10, IL-18, G-CSF, M-CSF, MCP-1, MCP-3, IP-10, MIG, or MIP-1α. In some other preferred embodiments of the methods of the disclosure, the NKT cell activator may be alpha GalCer loaded dendritic cells or monocytes.

As used herein, the term T cell activator includes any agent or molecule triggering activation of T cells. T cells can be activated via interaction of TCRs with antigenic peptide and MHC and via non-antigen specific costimulators (such as the cytokine interleukin 1). Activation of T cells is associated with increased cytokine and chemokine production, induction of dendritic cell maturation, recruitment of macrophages, and increased cytolytic activity. Activation of gamma delta T cells may also be associated with increased production of growth factors that maintain epidermal integrity (such as IGF-1, VEGF and FGF-2), as well as antigen presentation for alpha beta T cells. Activation of T cells may also be associated with changes in the pattern of expression of surface markers. For gamma delta T cells, this may include one or more of the following marker phenotypes: CD5−, CD4−/CD8− (double negative), CD3+, CD69, CD56, CD27, CD45RA+, CD45, TCR-Vg9+, TCR-Vd2+, TCR-Vd1+, and/or TCR-Vd3+. T cell activators that may be utilized in the disclosed methods are well known to those skilled in the art.

Some such T cell activators include, but are not limited to, Adipokines, Leptin, adiponectin, apelin, chemerin, MCP-1, PAI-1, RBP4, visfatin, omentin, vaspin, progranulin, CTRP-4, Cytokines, IL-1α, IL-1β, IL-IRA. IL-18, IL-33, IL-36a, IL-36B, IL-36γ. IL-36RA, IL-37, IL-38, IL-2, IL-4, IL-7, IL-9, IL-15, IL-21, IFN-α, IFN-β, IFN-δ, IFN-ε, IFN-κ, IFN-τ, IFN-ω, IFN-γ, IFN-λ1, IFN-λ2, IFN-λ3, IFN-λ4, IL-6, IL-11, IL-31, CLCF1, CNTF, leptin, LIF, OSM, iL-12, IL-17A, IL-17B, IL-17C, IL-17D, IL-17E, IL-17F, 4-1BBL, BAFF, CD40LG, CD70, CD95L/CD178, EDA-A1, LTA/TNF-β, TNF-α, TNFSF4, TNFS8, TNFSF10, TNFSF11, TNFSF12, TNFSF13, TNFSF15, TGF-β1, TGF-β2, TGF-β3, IL-13, G-CSF, GM-CSF, CSF1. Chemokines, CXCL1-CXCL17, CC, CCL1-CCL28, CX3CL1, XCL1, XCL2, Myokines, BDNF, Decorin, irisin, myostatin, myonectin, osteonectin, Prostaglandins, PGI2, PGD2, PGE2, PGF2a, Prostamides, Prostamide 12, prostamide D2, prostamide E2, prostamide F2a, Virokines, Growth Factors, Adrenomedullin, angiopoietin, autocrine motility factor, bone morphogenic proteins, ciliary neurotrophic factor, leukemia inhibitory factor, M-CSF, EGF, ephrine A1-A5, ephrine B1-B3, erythropoietin, FGF1-FGF23, fetal bovine somatotrophin, GDNF, neurturin, persephin, artemin, growth differentiation factor-9, hepatocyte growth factor, hepatocyte-derived growth factor, insulin, insulin-like growth factor ½, keratinocyte growth factor, migration-stimulating factor, macrophage-stimulating protein, neuregulin 1-4, neurotrophin ¾, nerve growth factor, placental growth factor, platelet-derived growth factor, renalase, T-cell growth factor, TGF-α, TGF-β, VEGF, Wnt signaling pathway, NKT Activators, α-galactosylceramide, α-glucoronosylceramide, α-galcturonsylceramide, α-galactosyldiacylgylocerol, phosphatidylinositol-manosidase, α-glucosyldiacylglycerol, cholesterol α-glucoside, β-glaactocsylceramide, isoglobotrihexosylceramide, diasialoganglioside, phosphatidylethanolamine, phosphatidylinositol, phosphatidylcholine, house dust extract, GSL-1, NKp44L, ULBP, Pathogen-derived molecular structures, PAMP, LPS, pathogen-derived RNA, pathogen-derived DNA, viral ligands, Synthetic α-galacosylceramide, KRN7000, PBS44, PBS57, Anti-inflammatory, IL-10, IL-19, IL-20, IL-22, IL-24, IL-28A, IL-28B, IL-29.

In some preferred embodiments of the methods of the disclosure, the T cell activator may be selected from the group consisting of: zoledronate, mevastatin, or a T cell-activating antibody.

In some embodiments of the disclosure, the T cell activator may not be one or more of the above recited agents.

As used herein, the term NK cell activator includes any agent or molecule triggering activation of NK cells.

Some such NK cell activators include, but are not limited to, Adipokines, Leptin, adiponectin, apelin, chemerin, MCP-1, PAI-1, RBP4, visfatin, omentin, vaspin, progranulin, CTRP-4, Cytokines, IL-1α, IL-1β, IL-1RA. IL-18, IL-33, IL-36a, IL-36β, IL-36γ. IL-36RA, IL-37, IL-38, IL-2, IL-4, IL-7, IL-9, IL-15, IL-21, IFN-α, IFN-β, IFN-δ, IFN-ε, IFN-κ, IFN-τ, IFN-ω, IFN-γ, IFN-λ1, IFN-λ2, IFN-λ3, IFN-λ4, IL-6, IL-11, IL-31, CLCF1, CNTF, leptin, LIF, OSM, iL-12, IL-17A, IL-17B, IL-17C, IL-17D, IL-17E, IL-17F, 4-1BBL, BAFF, CD40LG, CD70, CD95L/CD178, EDA-A1, LTA/TNF-β, TNF-α, TNFSF4, TNFS8, TNFSF10, TNFSF11, TNFSF12, TNFSF13, TNFSF15, TGF-β1, TGF-β2, TGF-β3, IL-13, G-CSF, GM-CSF, CSF1. Chemokines, CXCL1-CXCL17, CC, CCL1-CCL28, CX3CL1, XCL1, XCL2, Myokines, BDNF, Decorin, irisin, myostatin, myonectin, osteonectin, Prostaglandins, PGI2, PGD2, PGE2, PGF2a, Prostamides, Prostamide 12, prostamide D2, prostamide E2, prostamide F2a, Virokines, Growth Factors, Adrenomedullin, angiopoietin, autocrine motility factor, bone morphogenic proteins, ciliary neurotrophic factor, leukemia inhibitory factor, M-CSF, EGF, ephrine A1-A5, ephrine B1-B3, erythropoietin, FGF1-FGF23, fetal bovine somatotrophin, GDNF, neurturin, persephin, artemin, growth differentiation factor-9, hepatocyte growth factor, hepatocyte-derived growth factor, insulin, insulin-like growth factor ½, keratinocyte growth factor, migration-stimulating factor, macrophage-stimulating protein, neuregulin 1-4, neurotrophin ¾, nerve growth factor, placental growth factor, platelet-derived growth factor, renalase, T-cell growth factor, TGF-α, TGF-β, VEGF, Wnt signaling pathway, NKT Activators, α-galactosylceramide, α-glucoronosylceramide, α-galcturonsylceramide, α-galactosyldiacylgylocerol, phosphatidylinositol-manosidase, α-glucosyldiacylglycerol, cholesterol α-glucoside, β-glaactocsylceramide, isoglobotrihexosylceramide, diasialoganglioside, phosphatidylethanolamine, phosphatidylinositol, phosphatidylcholine, house dust extract, GSL-1, NKp44L, ULBP, Pathogen-derived molecular structures, PAMP, LPS, pathogen-derived RNA, pathogen-derived DNA, viral ligands, Synthetic α-galacosylceramide, KRN7000, PBS44, PBS57, Anti-inflammatory, IL-10, IL-19, IL-20, IL-22, IL-24, IL-28A, IL-28B, IL-29.

In some preferred embodiments of the methods of the disclosure, the NK cell activator may be selected from the group consisting of: IL-2, IL-12, IL-15, IL-18, IL-21, or an NK cell-activating antibody.

In some embodiments of the disclosure, the NK cell activator may not be one or more of the above recited agents.

In some embodiments of the methods of the disclosure, the NKT cell activator, T cell activator, and/or NK cell activator may be administered within 1, 3, 24, 48, 72, 96, 120, 144, or 168 hours of administration of a dose of glucocorticoid-receptor (GR) modulating agent or ICAM3 modulating agent. In some preferred embodiments the NKT cell activator, T cell activator, and/or NK cell activator may be administered within or around 1, 3, or 48 hours after administration of a dose of glucocorticoid-receptor (GR) modulating agent or ICAM3 modulating agent. In some particularly preferred embodiments the NKT cell activator, T cell activator, and/or NK cell activator may be administered within or around 1, 3, or 48 hours after administration of a dose of glucocorticoid.

The terms “subject” and “patient” are used interchangeably herein, and refer to a human or animal. In some embodiments of the methods of the disclosure, the subject may be mammalian. In some preferred embodiments, the subject may be human of any sex or race. In some embodiments, the human is an adult human. In some embodiments of the methods of the disclosure, the subject may be a healthy subject, such as a healthy adult human subject. In this context a healthy subject is a subject which is not afflicted with disease. Preferably, the subject is human or a mammal with a humanised immune system, such as a human immune system (HIS) mouse. Most preferably, the subject is human.

In some embodiments of the methods of the disclosure, the subject may have, be suspected of having, or have been diagnosed with a disease selected from the group consisting of: cancer, autoimmune disease, or infectious disease (also called microbial disease).

As used herein, “cancer” refers to a disease characterized by the uncontrolled growth of aberrant cells. Cancer cells can spread locally or through the bloodstream and lymphatic system to other parts of the body. Examples of various cancers are described herein and include but are not limited to, breast cancer, prostate cancer, ovarian cancer, cervical cancer, skin cancer, pancreatic cancer, colorectal cancer, renal cancer, liver cancer, brain cancer, lymphoma, leukemia, lung cancer and the like. The terms “tumor” and “cancer” are used interchangeably herein, e.g., both terms encompass solid and liquid, e.g., diffuse or circulating, tumors. As used herein, the term “cancer” or “tumor” includes premalignant, as well as malignant cancers and tumors.

In some embodiments of the disclosure, the cancer may be: Malignant neoplasm of lip, Malignant neoplasm of tonsil, Malignant neoplasm of tongue, Malignant neoplasm of gum, Malignant neoplasm of mouth, Malignant neoplasm of parotid gland, Malignant neoplasm of salivary glands, Malignant neoplasm of pharynx, Malignant neoplasm of esophagus, Malignant neoplasm of stomach, Malignant neoplasm of small intestine, Malignant neoplasm of colon, Malignant neoplasm of recto sigmoid junction, Malignant neoplasm of rectum, Malignant neoplasm of anus, Malignant neoplasm of liver, Malignant neoplasm of gallbladder, Malignant neoplasm of biliary tract, Malignant neoplasm of pancreas, Malignant neoplasm of intestinal tract, Malignant neoplasm of spleen, Malignant neoplasm of nasal cavity and middle ear, Malignant neoplasm of accessory sinuses, Malignant neoplasm of larynx, Malignant neoplasm of trachea, Malignant neoplasm of bronchus and lung, Malignant neoplasm of thymus, Malignant neoplasm of heart, mediastinum and pleura, Malignant neoplasm of sites in the respiratory system and intrathoracic organs, Malignant neoplasm of bone and articular cartilage of limbs, Malignant neoplasm of bones of skull and face, Malignant neoplasm of vertebral column, Malignant neoplasm of ribs, sternum and clavicle, Malignant neoplasm of pelvic bones, sacrum and coccyx, Malignant melanoma of skin, Malignant melanoma of lip, Malignant melanoma of eyelid, including canthus, Malignant melanoma of ear and external auricular canal, Malignant melanoma of face, Malignant melanoma of anal skin, Malignant melanoma of skin of breast, Malignant melanoma of limbs, including shoulder, Merkel cell carcinoma, Basal cell carcinoma of skin of lip, Squamous cell carcinoma of skin of lip, Other and unspecified malignant neoplasm skin/eyelid, including canthus, Malignant neoplasm skin/ear and external auric canal, Other and unspecified malignant neoplasm skin/and unspecified parts of face, Basal cell carcinoma of skin of other and unspecified parts of face, Squamous cell carcinoma of skin of and unspecified parts of face, Basal cell carcinoma of skin of scalp and neck, Squamous cell carcinoma of skin of scalp and neck, Basal cell carcinoma of skin of trunk, Basal cell carcinoma of anal skin, Basal cell carcinoma of skin of breast, Squamous cell carcinoma of skin of trunk, Squamous cell carcinoma of anal skin, Squamous cell carcinoma of skin of breast, Squamous cell carcinoma of skin of other part of trunk, Other and unspecified malignant neoplasm skin/limbs including shoulder, Basal cell carcinoma skin/limbs, including shoulder, Squamous cell carcinoma skin/limbs, including shoulder, Basal cell carcinoma of skin of limbs, including hip, Squamous cell carcinoma of skin of limbs, including hip, Mesothelioma, Kaposi's sarcoma, Malignant neoplasm of peripheral nerves and autonomic nervous sys, Malignant neoplasm of retroperitoneum and peritoneum, Malignant neoplasm of other connective and soft tissue, Malignant neoplasm of connective and soft tissue of thorax, Malignant neoplasm of connective and soft tissue of abdomen, Malignant neoplasm of connective and soft tissue of pelvis, Malignant neoplasm of conn and soft tissue of trunk, unspecified, Malignant neoplasm of overlapping sites of connective and soft tissue, Malignant neoplasm of connective and soft tissue, unspecified, Gastrointestinal stromal tumor, Malignant neoplasm of breast, Malignant neoplasm of vulva, Malignant neoplasm of vagina, Malignant neoplasm of cervix uteri, Malignant neoplasm of corpus uteri, Malignant neoplasm of uterus, part unspecified, Malignant neoplasm of ovary, Malignant neoplasm of other and unspecified female genital organs, Malignant neoplasm of placenta, Malignant neoplasm of penis, Malignant neoplasm of prostate, Malignant neoplasm of testis, Malignant neoplasm of other and unspecified male genital organs, Malignant neoplasm of kidney, Malignant neoplasm of renal pelvis, Malignant neoplasm of ureter, Malignant neoplasm of bladder, Malignant neoplasm of other and unspecified urinary organs, Malignant neoplasm of eye and adnexa, Malignant neoplasm of meninges, Malignant neoplasm of brain, Malignant neoplm of spinal cord, cranial nerves, Malignant neoplasm of optic nerve, Malignant neoplasm of other and unspecified cranial nerves, Malignant neoplasm of central nervous system, unspecified, Malignant neoplasm of thyroid gland, Malignant neoplasm of adrenal gland, Malignant neoplasm of endo glands and related structures, Malignant neuroendocrine tumors, Malignant carcinoid tumors, Secondary neuroendocrine tumors, Malignant neoplasm of head, face and neck, Malignant neoplasm of thorax, Malignant neoplasm of abdomen, Malignant neoplasm of pelvis, Malignant neoplasm of limbs, Malignant neoplasm of lower limb, Secondary and unspecified malignant neoplasm of lymph nodes, Secondary malignant neoplasm of respiratory and digestive organs, Secondary malignant neoplasm of kidney and renal pelvis, Secondary malignant neoplm of bladder and other and unspecified urinary organs, Secondary malignant neoplasm of skin, Secondary malignant neoplasm of brain and cerebral meninges, Secondary malignant neoplasm of and unspecified parts of nervous sys, Secondary malignant neoplasm of bone and bone marrow, Secondary malignant neoplasm of ovary, Secondary malignant neoplasm of adrenal gland, Hodgkin lymphoma, Follicular lymphoma, Non-follicular lymphoma, Small cell B-cell lymphoma, Mantle cell lymphoma, Diffuse large B-cell lymphoma, Lymphoblastic (diffuse) lymphoma, Burkitt lymphoma, Other non-follicular lymphoma, Non-follicular (diffuse) lymphoma, unspecified, Mature T/NK-cell lymphomas, Sezary disease, Peripheral T-cell lymphoma, not classified, Anaplastic large cell lymphoma, ALK-positive, Anaplastic large cell lymphoma, ALK-negative, Cutaneous T-cell lymphoma, unspecified, Other mature T/NK-cell lymphomas, Mature T/NK-cell lymphomas, unspecified, Other and unspecified types of non-Hodgkin lymphoma, Malignant immunoproliferative dis and certain other B-cell lymph, Multiple myeloma and malignant plasma cell neoplasms, Lymphoid leukemia, Acute lymphoblastic leukemia [ALL], Chronic lymphocytic leukemia of B-cell type, Prolymphocytic leukemia of B-cell type, Hairy cell leukemia, Adult T-cell lymphoma/leukemia (HTLV-1-associated), Prolymphocytic leukemia of T-cell type, Mature B-cell leukemia Burkitt-type, Other lymphoid leukemia, Lymphoid leukemia, unspecified, Myeloid leukemia, Acute myeloblastic leukemia, Chronic myeloid leukemia, BCR/ABL-positive, Atypical chronic myeloid leukemia, BCR/ABL-negative, Myeloid sarcoma, Acute promyelocytic leukemia, Acute myelomonocytic leukemia, Acute myeloid leukemia with 11q23-abnormality, Other myeloid leukemia, Myeloid leukemia, unspecified, Monocytic leukemia, Chronic myelomonocytic leukemia, Juvenile myelomonocytic leukemia, Other monocytic leukemia, Monocytic leukemia, unspecified, Other leukemias of specified cell type, Acute erythroid leukemia, Acute megakaryoblastic leukemia, Mast cell leukemia, Acute panmyelosis with myelofibrosis, Myelodysplastic disease, not classified, Other specified leukemias, Leukemia of unspecified cell type, Chronic leukemia of unspecified cell type, Leukemia, unspecified, Other & unspecified malignant neoplasm of lymphoid, hematopoietic tissue, Carcinoma in situ of oral cavity, esophagus and stomach, Carcinoma in situ of colon, Carcinoma in situ of recto sigmoid junction, Carcinoma in situ of rectum, Carcinoma in situ of anus and anal canal, Carcinoma in situ of other and unspecified parts of intestine, Carcinoma in situ of unspecified part of intestine, Carcinoma in situ of other parts of intestine, Carcinoma in situ of liver, gallbladder and bile ducts, Carcinoma in situ of other specified digestive organs, Carcinoma in situ of digestive organ, unspecified, Carcinoma in situ of middle ear and respiratory system, Carcinoma in situ of larynx, Carcinoma in situ of trachea, Carcinoma in situ of bronchus and lung, Carcinoma in situ of other parts of respiratory system, Melanoma in situ, Melanoma in situ of lip, Melanoma in situ of eyelid, including canthus, Melanoma in situ of ear and external auricular canal, Melanoma in situ of unspecified part of face, Melanoma in situ of scalp and neck, Melanoma in situ of trunk, Melanoma in situ of anal skin, Melanoma in situ of breast (skin) (soft tissue), Melanoma in situ of upper limb, including shoulder, Melanoma in situ of lower limb, including hip, Melanoma in situ of other sites, Carcinoma in situ of skin, Carcinoma in situ of skin of lip, Carcinoma in situ of skin of eyelid, including canthus, Carcinoma in situ skin of ear and external auricular canal, Carcinoma in situ of skin of other and unspecified parts of face, Carcinoma in situ of skin of scalp and neck, Carcinoma in situ of skin of trunk, Carcinoma in situ of skin of upper limb, including shoulder, Carcinoma in situ of skin of lower limb, including hip, Carcinoma in situ of skin of other sites, Carcinoma in situ of breast, Lobular carcinoma in situ of breast, Intraductal carcinoma in situ of breast, Other specified type of carcinoma in situ of breast, Unspecified type of carcinoma in situ of breast, Carcinoma in situ of cervix uteri, Carcinoma in situ of other parts of cervix, Carcinoma in situ of cervix, unspecified, Carcinoma in situ of other and unspecified genital organs, Carcinoma in situ of endometrium, Carcinoma in situ of vulva, Carcinoma in situ of vagina, Carcinoma in situ of other and unspecified female genital organs, Carcinoma in situ of penis, Carcinoma in situ of prostate, Carcinoma in situ of unspecified male genital organs, Carcinoma in situ of scrotum, Carcinoma in situ of other male genital organs, Carcinoma in situ of bladder, Carcinoma in situ of other and unspecified urinary organs, Carcinoma in situ of eye, Carcinoma in situ of thyroid and other endocrine glands, Benign neoplasm of mouth and pharynx, Benign neoplasm of major salivary glands, Benign neoplasm of colon, rectum, anus and anal canal, Benign neoplasm of and ill-defined parts of digestive system, Benign neoplasm of esophagus, Benign neoplasm of stomach, Benign neoplasm of duodenum, Benign neoplasm of other and unspecified parts of small intestine, Benign neoplasm of liver, Benign neoplasm of extrahepatic bile ducts, Benign neoplasm of pancreas, Benign neoplasm of endocrine pancreas, Benign neoplasm of ill-defined sites within the digestive system, Benign neoplasm of middle ear and respiratory system, Benign neoplasm of respiratory system, unspecified, Benign neoplasm of other and unspecified intrathoracic organs, Benign neoplasm of thymus, Benign neoplasm of heart, Benign neoplasm of mediastinum, Benign neoplasm of other specified intrathoracic organs, Benign neoplasm of intrathoracic organ, unspecified, Benign neoplasm of bone and articular cartilage, Benign neoplasm of short bones of upper limb, Benign neoplasm of long bones of lower limb, Benign neoplasm of short bones of lower limb, Benign neoplasm of bones of skull and face, Benign neoplasm of lower jaw bone, Benign neoplasm of vertebral column, Benign neoplasm of ribs, sternum and clavicle, Benign neoplasm of pelvic bones, sacrum and coccyx, Benign neoplasm of bone and articular cartilage, unspecified, Benign lipomatous neoplasm, Ben lipomatous neoplm of skin, subcutaneous of head, face and neck, Benign lipomatous neoplasm of intrathoracic organs, Benign lipomatous neoplasm of intra-abdominal organs, Benign lipomatous neoplasm of spermatic cord, Benign lipomatous neoplasm of other sites, Benign lipomatous neoplasm of kidney, Benign lipomatous neoplasm of other genitourinary organ, Hemangioma and lymphangioma, any site, Hemangioma, Hemangioma unspecified site, Hemangioma of skin and subcutaneous tissue, Hemangioma of intracranial structures, Hemangioma of intra-abdominal structures, Hemangioma of other sites, Lymphangioma, any site, Benign neoplasm of mesothelial tissue, Benign neoplm of soft tissue of retroperitoneum and peritoneum, Other benign neoplasms of connective and other soft tissue, Melanocytic nevi, Melanocytic nevi of lip, Melanocytic nevi of eyelid, including canthus, Melanocytic nevi of unspecified eyelid, including canthus, Melanocytic nevi of ear and external auricular canal, Melanocytic nevi of other and unspecified parts of face, Melanocytic nevi of scalp and neck, Melanocytic nevi of trunk, Melanocytic nevi of upper limb, including shoulder, Melanocytic nevi of lower limb, including hip, Melanocytic nevi, unspecified, Other benign neoplasm of skin of eyelid, including canthus, Other benign neoplasm skin/ear and external auricular canal, Other benign neoplasm skin/left ear and external auric canal, Other benign neoplasm of skin of other and unspecified parts of face, Other benign neoplasm of skin of other parts of face, Other benign neoplasm of skin of scalp and neck, Other benign neoplasm of skin of trunk, Other benign neoplasm skin/upper limb, including shoulder, Other benign neoplasm of skin of lower limb, including hip, Other benign neoplasm of skin, unspecified, Benign neoplasm of breast, Benign neoplasm of unspecified breast, Leiomyoma of uterus, Other benign neoplasms of uterus, Benign neoplasm of ovary, Benign neoplasm of other and unspecified female genital organs, Benign neoplasm of male genital organs, Benign neoplasm of urinary organs, Benign neoplasm of kidney, Benign neoplasm of renal pelvis, Benign neoplasm of ureter, Benign neoplasm of bladder, Benign neoplasm of urethra, Benign neoplasm of other specified urinary organs, Benign neoplasm of urinary organ, unspecified, Benign neoplasm of eye and adnexa, Benign neoplasm of conjunctiva, Benign neoplasm of cornea, Benign neoplasm of retina, Benign neoplasm of choroid, Benign neoplasm of ciliary body, Benign neoplasm of lacrimal gland and duct, Benign neoplasm of unspecified site of orbit, Benign neoplasm of unspecified part of eye, Benign neoplasm of meninges, Benign neoplasm of brain and central nervous system, Benign neoplasm of thyroid gland, Benign neoplasm of other and unspecified endocrine glands, Benign neoplasm of other and unspecified sites, Benign neoplasm of lymph nodes, Benign neoplasm of peripheral nerves and autonomic nervous sys, Benign neoplasm of other specified sites, Benign neuroendocrine tumors, Other benign neuroendocrine tumors, Neoplasm of uncertain behavior of oral cavity and digestive organs, Neoplasm of uncertain behavior of the major salivary glands, Neoplasm of uncertain behavior of pharynx, Neoplasm of uncertain behavior of sites of the oral cavity, Neoplasm of uncertain behavior of stomach, Neoplasm of uncertain behavior of small intestine, Neoplasm of uncertain behavior of appendix, Neoplasm of uncertain behavior of colon, Neoplasm of uncertain behavior of rectum, Neoplasm of uncertain behavior of liver, GB & bile duct, Neoplasm of uncertain behavior of other digestive organs, Neoplasm of uncertain behavior of digestive organ, Neoplm of mid ear and intrathoracic organs, Neoplasm of uncertain behavior of larynx, Neoplasm of uncertain behavior of trachea, bronchus and lung, Neoplasm of uncertain behavior of pleura, Neoplasm of uncertain behavior of mediastinum, Neoplasm of uncertain behavior of thymus, Neoplasm of uncertain behavior of other respiratory organs, Neoplasm of uncertain behavior of respiratory organ, unspecified, Neoplasm of uncertain behavior of female genital organs, Neoplasm of uncertain behavior of uterus, Neoplasm of uncertain behavior of ovary, Neoplasm of uncertain behavior of unspecified ovary, Neoplasm of uncertain behavior of placenta, Neoplasm of uncertain behavior of male genital organs, Neoplasm of uncertain behavior of urinary organs, Neoplasm of uncertain behavior of kidney, Neoplasm of uncertain behavior of unspecified kidney, Neoplasm of uncertain behavior of renal pelvis, Neoplasm of uncertain behavior of ureter, Neoplasm of uncertain behavior of bladder, Neoplasm of uncertain behavior of other urinary organs, Neoplasm of uncertain behavior of unspecified urinary organ, Neoplasm of uncertain behavior of meninges, Neoplasm of uncertain behavior of cerebral meninges, Neoplasm of uncertain behavior of spinal meninges, Neoplasm of uncertain behavior of meninges, unspecified, Neoplasm of uncertain behavior of brain, Neoplasm of uncertain behavior of brain, Neoplasm of uncertain behavior of brain, infratentorial, Neoplasm of uncertain behavior of brain, unspecified, Neoplasm of uncertain behavior of cranial nerves, Neoplasm of uncertain behavior of spinal cord, Neoplasm of uncertain behavior of central nervous system, Neoplasm of uncertain behavior of endocrine glands, Neoplasm of uncertain behavior of thyroid gland, Neoplasm of uncertain behavior of adrenal gland, Neoplasm of uncertain behavior of unspecified adrenal gland, Neoplasm of uncertain behavior of parathyroid gland, Neoplasm of uncertain behavior of pituitary gland, Neoplasm of uncertain behavior of craniopharyngeal duct, Neoplasm of uncertain behavior of pineal gland, Neoplasm of uncertain behavior of carotid body, Neoplasm of uncertain behavior of aortic body and other paraganglia, Neoplasm of uncertain behavior of unspecified endocrine gland, Polycythemia vera, Myelodysplastic syndromes, Refractory anemia without ring sideroblasts, so stated, Refractory anemia with ring sideroblasts, Refractory anemia with excess of blasts [RAEB], Myelodysplastic syndrome, unspecified, Other neoplm of uncertain behavior of lymphoid, hematopoietic tissue, Histiocytic and mast cell tumors of uncertain behavior, Chronic myeloproliferative disease, Monoclonal gammopathy, Essential (hemorrhagic) thrombocythemia, Osteomyelofibrosis, Other neoplasm of uncertain behavior of lymphoid, hematopoietic tissue, Neoplasm of uncertain behavior of lymphoid, hematopoietic & unspecified, Neoplasm of uncertain behavior of other and unspecified sites, Neoplasm of uncertain behavior of bone/artic cartilage, Neoplasm of uncertain behavior of connective/soft tissue, Neoplasm of uncertain behavior of peripheral nerves and autonomous nervous sys, Neoplasm of uncertain behavior of retroperitoneum, Neoplasm of uncertain behavior of peritoneum, Neoplasm of uncertain behavior of skin, Neoplasm of uncertain behavior of breast, Neoplasm of unspecified behavior of digestive system, Neoplasm of unspecified behavior of respiratory system, Neoplasm of unspecified behavior of bone, soft tissue, and skin, Neoplasm of unspecified behavior of breast, Neoplasm of unspecified behavior of bladder, Neoplasm of unspecified behavior of other genitourinary organs, Neoplasm of unspecified behavior of kidney, Neoplasm of unspecified behavior of other GU organ, Neoplasm of unspecified behavior of brain, Neoplasm of unspecified behavior of endo glands and other parts of nervous sys, Neoplasm of unspecified behavior of retina and choroid, or Neoplasm of unspecified behavior of unspecified site.

In some embodiments of the disclosure, the cancer may not be one of the above recited cancers.

In some preferred embodiments of the disclosure, the cancer may be selected from the group consisting of: lymphoma, squamous cell cancer (such as epithelial squamous cell cancer); lung cancer, including small-cell lung cancer, non-small cell lung cancer, adenocarcinoma of the lung and squamous carcinoma of the lung; cancer of the peritoneum; hepatocellular cancer; gastric or stomach cancer, including gastrointestinal cancer; pancreatic cancer; glioblastoma; cervical cancer; ovarian cancer; liver cancer; bladder cancer; hepatoma; breast cancer; colon cancer; rectal cancer; colorectal cancer; endometrial or uterine carcinoma; salivary gland carcinoma; kidney or renal cancer; prostate cancer; vulval cancer; thyroid cancer; hepatic carcinoma; anal carcinoma; penile carcinoma; and head and neck cancer. In some particularly preferred embodiments of the disclosure the cancer may be lymphoma. In more particularly preferred embodiments of the disclosure the cancer may be a B cell lymphoma or a T cell lymphoma. In some particularly preferred embodiments of the disclosure the cancer may be non-Hodgkin lymphoma. In some particularly preferred embodiments of the disclosure the cancer may be Burkitt's Lymphoma, T-cell Acute Lymphoblastic Leukaemia (T-ALL), B-Cell Acute Lymphoblastic Leukemia (B-ALL), or diffuse large B-cell lymphoma (DLBCL). In other preferred embodiments, the cancer may be a post-transplant lymphoproliferative disorder. In some other particularly preferred embodiments of the disclosure the cancer may be a solid tumor cancer.

In embodiments in which the methods of the disclosure are carried out on a subject having, suspected of having, or having been diagnosed with cancer, the NKT-like cells produced by these methods may treat the cancer. In this context, “treat” means to exert a beneficial therapeutic effect in the subject, which can be any overall clinical benefit derived from the methods of the disclosure. This overall clinical benefit can be any of, for example: prolonged survival, partial or complete disease remission, (for example, as assessed by % bone marrow myeloblasts and/or normal maturation of cell lines), slowing or absence of disease progression (for example, as assessed by change in % bone marrow myeloblasts), tumour shrinkage (for example, a reduction in tumour volume of 5, 10, 20, 30, 40% or more), reduction in tumour burden (for example, a reduction in tumour burden of 5, 10, 20, 30, 40% or more), slowing or absence of tumour enlargement, slowing or absence of increase in tumour burden, improved quality of life (for example, as assessed using a health-related quality of life questionnaire such as a Functional Assessment of Cancer Therapy (FACT) questionnaire), progression-free survival, overall survival, hematologic improvement (for example: increased blood haemoglobin, platelet count, and/or neutrophil count), bone marrow response (for example: bone marrow with ≤5% myeloblasts; 30%, 40%, 50% or more reduction in bone marrow myeloblasts; absence of circulating myeloblasts and myeloblasts with Auer rods; absence of extramedullary disease), hematologic recovery (for example: ≥11 g/dL haemoglobin, ≥100×109/L platelets, and/or ≥1×109/L neutrophils in peripheral blood), negative response for a genetic marker (for example, CEBPA, NPM1, or FLT3), or any other positive patient outcome.

The overall clinical benefit may be an “anti-tumor effect”. As used herein, an “anti-tumor effect” refers to a biological effect that can present as a decrease in tumor volume, a decrease in the number of tumor cells, a decrease in tumor cell proliferation, a decrease in the number of metastases, an increase in overall or progression-free survival, an increase in life expectancy, or amelioration of various physiological symptoms associated with the tumor. An anti-tumor effect can also refer to the prevention of the occurrence of a tumor, e.g., a vaccine. Suitable methods for determining tumour volume/burden are well known to the skilled person, for example, using: computed tomography (CT), or magnetic resonance imaging (MRI) imaging technologies; X-ray imaging, for example, mammography; ultrasound imaging; nuclear imaging, for example positron emission tomography (PET), PET/CT scans, bone scans, gallium scans, or metaiodobenzylguanidine (MIBG) scans; bioluminescence imaging (BLI); fluorescence imaging (FLI); BD ToF (infrared-based 3D Time-of-Flight camera) imaging.

Accordingly, in some embodiments, the NKT-like cells of the disclosure may treat the cancer via tumour infiltration. In some embodiments, the NKT-like cells of the disclosure may treat the cancer via release of immune activating cytokines. In some embodiments, the NKT-like cells of the disclosure may engulf and kill cancer cells in the subject. In some embodiments, the NKT-like cells of the disclosure promote infiltration of other immune cells into a tumor. In some embodiments, the NKT-like cells of the disclosure directly kill cancer cells via CD1d-directed apoptosis.

In some embodiments, the NKT-like cells of the disclosure directly kill cancer cells by inducing apoptosis, for example by expressing ligands which engage death receptors on target cells. In some embodiments, the NKT-like cells of the disclosure may ingest or engulf cancer cells in the subject. In some embodiments, the NKT-like cells may secrete cytotoxic molecules which kill the cancer cells. In some embodiments, the NKT-like cells may treat the cancer via bi-specific attack through both the TCR gamma/delta and the invariant TCR (iTCR).

“Autoimmune disease” as used herein refers to autoimmune disorders and other diseases arising from an abnormal immune state in which the immune system aberrantly attacks a subject's own constituents. (In healthy subjects, the immune system avoids damaging autoimmune reactions by establishing tolerance to the subject's own constituents). Examples of various autoimmune diseases are described herein and include but are not limited to, celiac disease, diabetes mellitus type 1, Graves' disease, inflammatory bowel disease, transient osteoporosis, multiple sclerosis, psoriasis, rheumatoid arthritis, and systemic lupus erythematosus.

Autoreactive immune cells express high levels of phosphoantigens, which are diphosphate-containing metabolites, as do stressed cells and microorganisms like Mycobacteria, E. coli, and Plasmodium, in particular the phosphoantigen produced by (E)-4-hydroxy-3-methyl-but-2-enyl pyrophosphate (HMB-PP). Humans do not produce HMB-PP.

but the majority of gram-negative bacteria do produce it including Mycobacterium tuberculosis, Mycobacterium bovus, Clostidrium difficile, Listeria monocytogenes, malaria parasites and Toxoplasma gondii and Schistosoma japonicum. Gamma delta T cells/receptors are very responsive to HMB-PP, zoledronate and isopentyl pyrophosphate (IPP), mycolylarabinogalactan peptidoglycan (mAGP), and iso-butylamine (IBA). Butyrophilin family members like BTN2A1, BTN3A1, BTNL3, BTNL8, BTNL1, BTNL6, Skint1, Skint2, play an important role in gamma delta T cell recognition of phosphoantigens.

Aminobisphosphonate stimulation of peripheral blood mononuclear cells (PBMC) can also activate gamma delta T cell receptors. IL-18 can enhance the response of the gamma delta T cell receptor to phosphoantigens.

In some embodiments of the disclosure the autoimmune disease may be: allergies, asthma, graft versus host disease (GvHD), steroid-resistant GvHD, Achalasia, Addison's disease, Adult Still's disease, Agammaglobulinemia, Alopecia areata, Alopecia, transient osteoporosis, Amyloidosis, Ankylosing spondylitis, Anti-GBM/Anti-TBM nephritis, Antiphospholipid syndrome, Autoimmune angioedema, Autoimmune dysautonomia, Autoimmune encephalomyelitis, Autoimmune hepatitis, Autoimmune inner ear disease (AIED), Autoimmune myocarditis, Autoimmune oophoritis, Autoimmune orchitis, Autoimmune pancreatitis, Autoimmune retinopathy, Autoimmune urticaria, Axonal & neuronal neuropathy (AMAN), Baló disease, Behcet's disease, Benign mucosal pemphigoid, Bullous pemphigoid, Castleman disease (CD), Celiac disease, Chagas disease, Chronic inflammatory demyelinating polyneuropathy (CIDP), Chronic recurrent multifocal osteomyelitis (CRMO), Churg-Strauss Syndrome (CSS) or Eosinophilic Granulomatosis (EGPA), Cicatricial pemphigoid, Cogan's syndrome, Cold agglutinin disease, Congenital heart block, Coxsackie myocarditis, CREST syndrome, Crohn's disease, Dermatitis herpetiformis, Dermatomyositis, Devic's disease (neuromyelitis optic), Discoid lupus, Dressler's syndrome, Endometriosis, Eosinophilic esophagitis (EoE), Eosinophilic fasciitis, Erythema nodosum, Essential mixed cryoglobulinemia, Evans syndrome, Fibromyalgia, Fibrosing alveolitis, Giant cell arteritis (temporal arteritis), Giant cell myocarditis, Glomerulonephritis, Goodpasture's syndrome, Granulomatosis with Polyangiitis, Graves' disease, Guillain-Barre syndrome, Hashimoto's thyroiditis, Hemolytic anemia, Henoch-Schonlein purpura (HSP), Herpes gestationis or pemphigoid gestationis (PG), Hidradenitis Suppurativa (HS) (Acne Inversa), Hypogammalglobulinemia, IgA Nephropathy, IgG4-related sclerosing disease, Immune thrombocytopenia purpura (ITP), Inclusion body myositis (IBM), Interstitial cystitis (IC), Juvenile arthritis, Juvenile diabetes (Type 1 diabetes), Juvenile myositis (JM), Kawasaki disease, Lambert-Eaton syndrome, Leukocytoclastic vasculitis, Lichen planus, Lichen sclerosus, Ligneous conjunctivitis, Linear IgA disease (LAD), Lupus, Lyme disease chronic, Meniere's disease, Microscopic polyangiitis (MPA), Mixed connective tissue disease (MCTD), Mooren's ulcer, Mucha-Habermann disease, Multifocal Motor Neuropathy (MMN) or MMNCB, Multiple sclerosis, Myasthenia gravis, Myositis, Narcolepsy, Neonatal Lupus, Neuromyelitis optica, Neutropenia, Ocular cicatricial pemphigoid, Optic neuritis, Palindromic rheumatism (PR), PANDAS, Paraneoplastic cerebellar degeneration (PCD), Paroxysmal nocturnal hemoglobinuria (PNH), Parry Romberg syndrome, Pars planitis (peripheral uveitis), Parsonage-Turner syndrome, Pemphigus, Peripheral neuropathy, Perivenous encephalomyelitis, Pernicious anemia (PA), POEMS syndrome, Polyarteritis nodosa, Polyglandular syndromes type I, II, III, Polymyalgia rheumatica, Polymyositis, Postmyocardial infarction syndrome, Postpericardiotomy syndrome, Primary biliary cirrhosis, Primary sclerosing cholangitis, Progesterone dermatitis, Psoriasis, Psoriatic arthritis, Pure red cell aplasia (PRCA), Pyoderma gangrenosum, Raynaud's phenomenon, Reactive Arthritis, Reflex sympathetic dystrophy, Relapsing polychondritis, Restless legs syndrome (RLS), Retroperitoneal fibrosis, Rheumatic fever, Rheumatoid arthritis, Sarcoidosis, Schmidt syndrome, Scleritis, Scleroderma, Sjögren's syndrome, Sperm & testicular autoimmunity, Stiff person syndrome (SPS), Subacute bacterial endocarditis (SBE), Susac's syndrome, Sympathetic ophthalmia (SO), Takayasu's arteritis, Temporal arteritis/Giant cell arteritis, Thrombocytopenia purpura (TTP), Tolosa-Hunt syndrome (THS), Transverse myelitis, Type 1 diabetes, Ulcerative colitis (UC), Undifferentiated connective tissue disease (UCTD), Uveitis, Vasculitis, Vitiligo, Vogt-Koyanagi-Harada Disease, Hemophagocytic lymphohistiocytosis, multiple myeloma, allergen specific immunotherapy, autosomal dominant haploinsufficiency, anterior interosseous nerve syndrome, Churg-Strauss syndrome, Systemic vasculitis, chronic graft versus host disease, Opsoclonus-Myoclonus Syndrome, Necrotising Autoimmune Myopathy (NAM), Pulmonary Sarcomatoid carcinomas, Waldenstrom's macroglobulinaemia (WM), fertility, Behcets Disease, Alopecia areata (AA), Acute-on-chronic Liver Failure, melanoma, ‘organizing bronchiolitis syndrome’, or encephalitis. In some embodiments the autoimmune disease may be: rheumatoid arthritis, rheumatic fever, multiple Sclerosis, experimental autoimmune encephalomyelitis, psoriasis, uveitis, diabetes mellitus, Systemic lupus erythematosus (SLE), lupus nephritis, eczema, Scleroderma, polymyositis/scleroderma, polymyositis/dermatomyositis, uncerative protitis, severe combined immunodeficiency (SCID), DiGeorge syndrome, ataxia-telangiectasia, seasonal allergies, perennial allergies, food allergies, anaphylaxis, mastocytosis, allergic rhinitis, atopic dermatitis, Parkinson's, Alzheimer's, hypersplenism, leukocyte adhesion deficiency, X-linked lymphoproliferative disease, X-linked agammaglobulinemia, selective immuno globulin A deficiency, hyper IgM syndrome, HIV, autoimmune lymphoproliferative syndrome, Wiskott-Aldrich syndrome, chronic granulomatous disease, common variable immunodeficiency (CVID), hyperimmunoglobulin E syndrome, Hashimoto's thyroiditis, acute idiopathic thrombocytopenia purpura, chronic idiopathic thrombocytopenia pur pura, dermatomyositis, Sydenham's chorea, myasthenia gravis, polyglandular syndromes, bullous pemphigoid, Henoch-Schonlein purpura, poststreptococcalnephritis, erythema nodosum, erythema multiforme, gA nephropathy, Takayasu's arteritis, Addison's disease, sarcoidosis, ulcerative colitis, polyarteritis nodosa, ankylosing spondylitis, Goodpasture's syndrome, thromboangitisubiterans, Sjogren's syndrome, primary biliary cirrhosis, Hashimoto's thyroiditis, thyrotoxicosis, chronic active hepatitis, polychondritis, pamphigus Vulgaris, Wegener's granulomatosis, membranous nephropathy, amyotrophic lateral Sclerosis, tabesdorsalis, giant cell arteritis/polymyalgia, peraiciousa nemia, rapidly progressive glomerulonephritis, psoriasis, fibrosing alveolitis, or cancer.

In some embodiments of the disclosure, the autoimmune disease may not be one of the above recited autoimmune diseases.

In some preferred embodiments of the disclosure, the autoimmune disease may be selected from the group consisting of: multiple sclerosis, systemic sclerosis, amyotrophic lateral sclerosis, type 1 diabetes mellitus (TID), scleroderma, pemphigus, and lupus. In some other preferred embodiments of the disclosure the autoimmune disease may be selected from the group consisting of: graft versus host disease (GvHD), and an allergic disorder such as asthma. In some particularly preferred embodiments of the disclosure the autoimmune disease may be type 1 diabetes mellitus (TID).

In embodiments in which the methods of the disclosure are carried out on a subject having, suspected of having, or having been diagnosed with autoimmune disease, the NKT-like cells produced by these methods may treat the autoimmune disease. In this context, “treat” means to exert a beneficial therapeutic effect in the subject, which can be any overall clinical benefit derived from the methods of the disclosure. This overall clinical benefit can be any of, for example: reduced fatigue, reduced achy muscles, reduced swelling and redness, reduced low-grade fever, reduced trouble concentrating, reduced numbness and tingling in the hands and feet and arms or legs, reduced urination, reduced hair loss, reduced skin rashes, restored normoglycemia, increased C peptide, improved wound healing, reduced diarrhea, reduced muscle spasms, improved muscle tone and control, reduced skin rash or scaly plaques on the skin or discoloration, improved weight maintenance, reduced muscle or joint pain, improved comfort of the digestive tract, normal heart rate, reduced anxiety, reduced expanded disability status scale (EDSS) score, reduced unique active lesions in the brain measured by gadolinium enhanced MRI.

In some embodiments, the NKT-like cells of the disclosure may treat the autoimmune disease via direct killing of autoreactive T and/or B lymphocytes, increasing Treg: T lymphocyte ratio, inhibiting the activity of autoreactive T and/or B lymphocytes, reducing inflammation, or reducing the trafficking of autoreactive lymphocytes.

“Infectious disease” (or “microbial disease”) as used herein refers to a disease or illness resulting from the infection of a subject's body by infectious agents (pathogens) such as viruses, bacteria, or fungi. In some embodiments of the disclosure the infectious disease may be: Acinetobacter infections (Acinetobacter baumannii), Actinomycosis (Actinomyces israelii, Actinomyces gerencseriae and Propionibacterium propionicus) African sleeping sickness or African trypanosomiasis (Trypanosoma brucei), AIDS (Acquired immunodeficiency syndrome) (Human immunodeficiency virus), Amebiasis (Entamoeba histolytica), Anaplasmosis (Anaplasma species), Angiostrongyliasis (Angiostrongylus), Anisakiasis (Anisakis), Anthrax (Bacillus anthracis), Arcanobacterium haemolyticum infection (Arcanobacterium haemolyticum), Argentine hemorrhagic fever (Junin virus), Ascariasis (Ascaris lumbricoides), Aspergillosis (Aspergillus species), Astrovirus infection (Astroviridae family), Babesiosis (Babesia species), Bacillus cereus infection (Bacillus cereus), Bacterial pneumonia (multiple bacteria), Bacterial vaginosis (List of bacterial vaginosis microbiota), Bacteroides infection (Bacteroides species), Balantidiasis (Balantidium coli), Bartonellosis (Bartonella), Baylisascaris infection (Baylisascaris species), BK virus infection (BK virus), Black piedra (Piedraia hortae), Blastocystosis (Blastocystis species), Blastomycosis (Blastomyces dermatitidis), Bolivian hemorrhagic fever (Machupo virus), Botulism (and Infant botulism) (Clostridium botulinum; Note: Botulism is not an infection by Clostridium botulinum but caused by the intake of botulinum toxin), Brazilian hemorrhagic fever (Sabiá virus), Brucellosis (Brucella species), Bubonic plague (the bacterial family Enterobacteriaceae), Burkholderia infection, usually Burkholderia cepacia and other Burkholderia species, Buruli ulcer (Mycobacterium ulcerans), Calicivirus infection (Norovirus and Sapovirus) (Caliciviridae family), Campylobacteriosis (Campylobacter species), Candidiasis (Moniliasis; Thrush) (usually Candida albicans and other Candida species), Capillariasis (Intestinal disease by Capillaria philippinensis, hepatic disease by Capillaria hepatica and pulmonary disease by Capillaria aerophila), Carrion's disease (Bartonella bacilliformis), Cat-scratch disease (Bartonella henselae), Cellulitis (usually Group A Streptococcus and Staphylococcus), Chagas Disease (American trypanosomiasis) (Trypanosoma cruzi), Chancroid (Haemophilus ducreyi), Chickenpox (Varicella zoster virus (VZV)), Chikungunya (Alphavirus), Chlamydia (Chlamydia trachomatis), Chlamydophila pneumoniae infection (Taiwan acute respiratory agent or TWAR) (Chlamydophila pneumoniae), Cholera (Vibrio cholerae), Chromoblastomycosis (usually Fonsecaea pedrosoi), Chytridiomycosis (Batrachochytrium dendrabatidis), Clonorchiasis (Clonorchis sinensis), Clostridium difficile colitis (Clostridium difficile), Coccidioidomycosis (Coccidioides immitis and Coccidioides posadasii), Colorado tick fever (CTF) (Colorado tick fever virus (CTFV)), Common cold (Acute viral rhinopharyngitis; Acute coryza) (usually rhinoviruses and coronaviruses), Coronavirus, Creutzfeldt-Jakob disease (CJD) (PRNP), Crimean-Congo hemorrhagic fever (CCHF) (Crimean-Congo hemorrhagic fever virus), Cryptococcosis (Cryptococcus neoformans), Cryptosporidiosis (Cryptosporidium species), Cutaneous larva migrans (CLM) (usually Ancylostoma braziliense; multiple other parasites), Cyclosporiasis (Cyclospora cayetanensis), Cysticercosis (Taenia solium), Cytomegalovirus infection (Cytomegalovirus), Dengue fever (Dengue viruses (DEN-1, DEN-2, DEN-3 and DEN-4)—Flaviviruses), Desmodesmus infection (Green algae Desmodesmus armatus), Dientamoebiasis (Dientamoeba fragilis), Diphtheria (Corynebacterium diphtheriae), Diphyllobothriasis (Diphyllobothrium), Dracunculiasis (Dracunculus medinensis), Ebola hemorrhagic fever (Ebolavirus (EBOV)), Echinococcosis (Echinococcus species), Ehrlichiosis (Ehrlichia species), Enterobiasis (Pinworm infection) (Enterobius vermicularis), Enterococcus infection (Enterococcus species), Enterovirus infection (Enterovirus species), Epidemic typhus (Rickettsia prowazekii), Erythema infectiosum (Fifth disease) (Parvovirus B19), Exanthem subitum (Sixth disease) (Human herpesvirus 6 (HHV-6) and Human herpesvirus 7 (HHV-7)), Fasciolasis (Fasciola hepatica and Fasciola gigantica), Fasciolopsiasis (Fasciolopsis buski), Fatal familial insomnia (FFI) (PRNP), Filariasis (Filarioidea superfamily), Food poisoning by Clostridium perfringens (Clostridium perfringens), Free-living amebic infection (multiple), Fusobacterium infection (Fusobacterium species), Gas gangrene (Clostridial myonecrosis) (usually Clostridium perfringens; other Clostridium species), Geotrichosis (Geotrichum candidum), Gerstmann-Sträussler-Scheinker syndrome (GSS) (PRNP), Giardiasis (Giardia lamblia) Glanders (Burkholderia mallei), Gnathostomiasis (Gnathostoma spinigerum and Gnathostoma hispidum), Gonorrhea (Neisseria gonorrhoeae), Granuloma inguinale (Donovanosis) (Klebsiella granulomatis), Group A streptococcal infection (Streptococcus pyogenes), Group B streptococcal infection (Streptococcus agalactiae), Haemophilus influenzae infection (Haemophilus influenzae) Hand, foot and mouth disease (HFMD) (Enteroviruses, mainly Coxsackie A virus and Enterovirus 71 (EV71)), Hantavirus Pulmonary Syndrome (HPS) (Sin Nombre virus), Heartland virus disease (Heartland virus), Helicobacter pylori infection (Helicobacter pylori), Hemolytic-uremic syndrome (HUS), Escherichia coli O157:H7, O111 and O104:H4, Hemorrhagic fever with renal syndrome (HFRS) (Bunyaviridae family), Hepatitis A (Hepatitis A virus), Hepatitis B (Hepatitis B virus), Hepatitis C (Hepatitis C virus), Hepatitis D (Hepatitis D Virus), Hepatitis E (Hepatitis E virus), Herpes simplex (Herpes simplex virus 1 and 2 (HSV-1 and HSV-2)), Histoplasmosis (Histoplasma capsulatum), Hookworm infection (Ancylostoma duodenale and Necator americanus), Human bocavirus infection (Human bocavirus (HBOV)), Human ewingii ehrlichiosis (Ehrlichia ewingii), Human granulocytic anaplasmosis (HGA) (Anaplasma phagocytophilum), Human metapneumovirus infection, Human metapneumovirus (hMPV), Human monocytic ehrlichiosis (Ehrlichia chaffeensis), Human papillomavirus (HPV) infection (Human papillomavirus (HPV)), Human parainfluenza virus infection (Human parainfluenza viruses (HPIV)), Hymenolepiasis (Hymenolepis nana and Hymenolepis diminuta), Epstein-Barr virus infectious mononucleosis (Mono) (Epstein-Barr virus (EBV)), Influenza (flu) (Orthomyxoviridae family) Isosporiasis (Isospora belli), Kawasaki disease (unknown; evidence supports that it is infectious) Keratitis (multiple), Kingella kingae infection (Kingella kingae), Kuru (PRNP), Lassa fever (Lassa virus), Legionellosis (Legionnaires' disease) (Legionella pneumophila), Legionellosis (Pontiac fever) (Legionella pneumophila), Leishmaniasis (Leishmania species), Leprosy (Mycobacterium leprae and Mycobacterium lepromatosis), Leptospirosis (Leptospira species), Listeriosis (Listeria monocytogenes), Lyme disease (Lyme borreliosis) (Borrelia burgdorferi, Borrelia garinii, and Borrelia afzelii), Lymphatic filariasis (Elephantiasis) (Wuchereria bancrofti and Brugia malayi), Lymphocytic choriomeningitis (Lymphocytic choriomeningitis virus (LCMV)), Malaria (Plasmodium species), Marburg hemorrhagic fever (MHF) (Marburg virus), Measles (Measles virus), Middle East respiratory syndrome (MERS) (Middle East respiratory syndrome coronavirus), Melioidosis (Whitmore's disease) (Burkholderia pseudomallei), Meningitis (multiple), Meningococcal disease (Neisseria meningitidis), Metagonimiasis (usually Metagonimus yokagawai), Microsporidiosis (Microsporidia phylum), Molluscum contagiosum (MC) (Molluscum contagiosum virus (MCV)), Monkeypox (Monkeypox virus), Mumps (Mumps virus), Murine typhus (Endemic typhus) (Rickettsia typhi), Mycoplasma pneumonia (Mycoplasma pneumoniae), Mycetoma (disambiguation) (numerous species of bacteria (Actinomycetoma) and fungi (Eumycetoma)), Myiasis (parasitic dipterous fly larvae), Neonatal conjunctivitis (Ophthalmia neonatorum) (most commonly Chlamydia trachomatis and Neisseria gonorrhoeae), Norovirus (children and babies) ((New) Variant Creutzfeldt-Jakob disease (vCJD, nvCJD), PRNP), Nocardiosis (usually Nocardia asteroides and other Nocardia species), Onchocerciasis (River blindness) (Onchocerca volvulus), Opisthorchiasis (Opisthorchis viverrini and Opisthorchis felineus), Paracoccidioidomycosis (South American blastomycosis) (Paracoccidioides brasiliensis), Paragonimiasis (usually Paragonimus westermani and other Paragonimus species), Pasteurellosis (Pasteurella species), Pediculosis capitis (Head lice) (Pediculus humanus capitis), Pediculosis corporis (Body lice) (Pediculus humanus corporis), Pediculosis pubis (Pubic lice, Crab lice) (Phthirus pubis), Pelvic inflammatory disease (PID) (multiple), Pertussis (Whooping cough) (Bordetella pertussis), Plague (Yersinia pestis), Pneumococcal infection (Streptococcus pneumoniae), Pneumocystis pneumonia (PCP) (Pneumocystis jirovecii), Pneumonia (multiple), Poliomyelitis (Poliovirus), Prevotella infection (Prevotella species), Primary amoebic meningoencephalitis (PAM) (usually Naegleria fowleri), Progressive multifocal leukoencephalopathy (JC virus), Psittacosis (Chlamydophila psittaci), Q fever (Coxiella burnetii), Rabies (Rabies virus), Relapsing fever (Borrelia hermsii, Borrelia recurrentis, and other Borrelia species), Respiratory syncytial virus infection (Respiratory syncytial virus (RSV)), Rhinosporidiosis (Rhinosporidium seeberi), Rhinovirus infection (Rhinovirus), Rickettsial infection (Rickettsia species), Rickettsialpox (Rickettsia akari), Rift Valley fever (RVF) (Rift Valley fever virus), Rocky Mountain spotted fever (RMSF) (Rickettsia rickettsii), Rotavirus infection (Rotavirus), Rubella (Rubella virus), Salmonellosis (Salmonella species), SARS (Severe Acute Respiratory Syndrome) (SARS coronavirus), Scabies (Sarcoptes scabiei), Schistosomiasis (Schistosoma species), Sepsis (multiple), Shigellosis (Bacillary dysentery) (Shigella species), Shingles (Herpes zoster) (Varicella zoster virus (VZV)), Smallpox (Variola) (Variola major or Variola minor), Sporotrichosis (Sporothrix schenckii), Staphylococcal food poisoning (Staphylococcus species), Staphylococcal infection (Staphylococcus species), Strongyloidiasis (Strongyloides stercoralis), Subacute sclerosing panencephalitis (Measles virus), Syphilis (Treponema pallidum), Taeniasis (Taenia species), Tetanus (Lockjaw) (Clostridium tetani), Tinea barbae (Barber's itch) (usually Trichophyton species), Tinea capitis (Ringworm of the Scalp) (usually Trichophyton tonsurans), Tinea corporis (Ringworm of the Body) (usually Trichophyton species), Tinea cruris (Jock itch) (usually Epidermophyton floccosum, Trichophyton rubrum, and Trichophyton mentagrophytes), Tinea manum (Ringworm of the Hand) (Trichophyton rubrum), Tinea nigra (usually Hortaea werneckii), Tinea pedis (Athlete's foot) (usually Trichophyton species), Tinea unguium (Onychomycosis) (usually Trichophyton species), Tinea versicolor (Pityriasis versicolor) (Malassezia species), Toxocariasis (Ocular Larva Migrans (OLM)) (Toxocara canis or Toxocara cati), Toxocariasis (Visceral Larva Migrans (VLM)) (Toxocara canis or Toxocara cati), Trachoma (Chlamydia trachomatis), Toxoplasmosis (Toxoplasma gondii), Trichinosis (Trichinella spiralis), Trichomoniasis (Trichomonas vaginalis), Trichuriasis (Whipworm infection) (Trichuris trichiura), Tuberculosis (usually Mycobacterium tuberculosis), Tularemia (Francisella tularensis), Typhoid fever (Salmonella enterica subsp. enterica, serovar typhi), Typhus fever (Rickettsia), Ureaplasma urealyticum infection (Ureaplasma urealyticum), Valley fever (Coccidioides immitis or Coccidioides posadasii), Venezuelan equine encephalitis (Venezuelan equine encephalitis virus), Venezuelan hemorrhagic fever (Guanarito virus), Vibrio vulnificus infection (Vibrio vulnificus), Vibrio parahaemolyticus enteritis (Vibrio parahaemolyticus), Viral pneumonia (multiple viruses), West Nile Fever (West Nile virus), White piedra (Tinea blanca) (Trichosporon beigelii), Yersinia pseudotuberculosis infection (Yersinia pseudotuberculosis), Yersiniosis (Yersinia enterocolitica), Yellow fever (Yellow fever virus), Zygomycosis (Mucorales order (Mucormycosis) and Entomophthorales order (Entomophthoramycosis)) Human immunodeficiency virus [HIV] disease, HIV disease with infectious and parasitic diseases, HIV disease with mycobacterial infection, HIV disease with cytomegaloviral disease, HIV disease with other viral infections, HIV disease with candidiasis, HIV disease with other mycoses, HIV disease with Pneumocystic carinii pneumonia, HIV disease with malignant neoplasms, HIV disease with Kaposi's sarcoma, HIV disease with Burkitt's lymphoma, HIV disease with other type's of non-Hodgkin's lymphoma, HIV disease with other malignant neoplasms of lymphoid, hematopoietic and related tissue, HIV disease with multiple malignant neoplasms, HIV disease with other malignant neoplasms, HIV disease with unspecified malignant neoplasm, HIV disease with encephalopathy, HIV disease with lymphoid interstitial pneumonitis, HIV disease with wasting syndrome, HIV disease with multiple diseases classified elsewhere, HIV disease with other conditions, HIV disease Acute HIV infection syndrome, HIV disease with (persistent) generalized lymphadenopathy, HIV disease with hematological and immunological abnormalities, HIV disease with other specified conditions, or Unspecified HIV disease. In some embodiments of the disclosure the infectious disease may be infection with a virus, such as a virus from one of the following families of viruses: a) Adenoviridae family, Such as Adenovirus species; b) Herpesviridae family, Such as Herpes simplex type 1, Herpes simplex type 2, Varicella Zoster virus, Epstein-barr virus, Human cytomegalovirus, Human herpesvirus type 8 species; c) Papillomaviridae family, Such as Human papillomavirus species; d) Polyomaviridae family, such as BK virus, JC virus species; e) Poxviridae family, Such as Smallpox species: f) Hepadnaviridae family, such as Hepatitis B virus species: g) Parvoviridae family, such as Human bocavirus, Parvovirus B19 species; h) Astroviridae family, such as Human astrovirus species: i) Caliciviridae family, such as Norwalk virus species; j) Flaviviridae family, such as Hepatitis C virus (HCV), yellow fever virus, dengue virus, West Nile virus species; k) Togaviridae family, such as Rubella virus species; 1) Hepeviridae family, such as Hepatitis E virus species; m) Retroviridae family, such as Human immunodeficiency virus (HIV) species; n) Orthomyxoviri daw family, such as Influenza virus species; o) Arenaviridae family, such as Guanarito virus, Junin virus, Lassa virus, Machupo virus, and/or Sabiá virus species; p) Bunyaviridae family, Such as Crimean-Congo hemorrhagic fever virus species; q) Filoviridae family, such as Ebola virus and/or Mar burg virus species; Paramyxoviridae family, Such as Measles virus, Mumps virus, Parainfluenza virus, Respiratory syncytial virus, Human metapneumovirus, Hendra virus and/or Nipah virus species; r) Rhabdoviridae genus, such as Rabies virus species; s) Reoviridae family, such as Rotavirus, Orbivirus, Coltivirus and/or Banna virus species.

In some embodiments of the disclosure, the infectious disease may not be one of the above recited infectious diseases.

In some embodiments, the infectious disease may be a disease caused by infection with an influenza A (Flu A) virus. In some embodiments the influenza virus can be an avian or swine-origin pandemic influenza virus, for example, H5N1, H7N3, H7N7, H7N9 and H9N2 (avian subtypes) or H1N1, H1N2, H2N1, H3N1, H3N2, or H2N3 (swine subtypes).

In some preferred embodiments of the disclosure, the infectious disease may be HIV, such as residual HIV disease, herpes, hepatitis or human papilloma virus. In other preferred embodiments, the infectious disease may be a disease resulting from infection with a coronavirus, for example COVID-19 (coronavirus 2019; the disease caused by severe acute respiratory syndrome coronavirus 2, SARS-CoV-2).

In embodiments in which the methods of the disclosure are carried out on a subject having, suspected of having, or having been diagnosed with infectious disease, the NKT-like cells produced by these methods may treat the infectious disease. In this context, “treat” means to exert a beneficial therapeutic effect in the subject, which can be any overall clinical benefit derived from the methods of the disclosure. This overall clinical benefit can be any of, for example: reduced fever, reduced diarrhea, reduced coughing, reduced muscle aches, reduced fatigue, reduced CRP, reduced time on ventilator, reduced need for extra oxygen, reduced organ damage after recovery.

In some embodiments, the NKT-like cells of the disclosure may treat the infectious disease via engulfing and killing the infectious organism, activating other innate and adaptive immune cells, recruiting other immune cells to the site of infection (e.g. an organ infected by a virus), depleting immune cells infected by the virus (e.g. monocytes activated by COVID-19).

In some embodiments, the NKT-like cells of the disclosure may treat the infectious disease via release of immune activating cytokines. In some embodiments, the NKT-like cells of the disclosure may treat the infectious disease via release of cytokines having anti-microbial or anti-viral effects (for example, TNF-alpha, IFN-gamma). In some embodiments, the NKT-like cells of the disclosure may treat the infectious disease by inducing apoptosis, for example by expressing ligands which engage death receptors on the target cells. In some embodiments, the NKT-like cells may secrete cytotoxic molecules which kill the infectious organism. In some embodiments, the NKT-like cells of the disclosure may ingest or engulf the infectious organism.

In embodiments in which the infectious disease is a disease resulting from infection with a coronavirus, for example COVID-19, the NKT-like cells of the disclosure may treat the disease via engulfing and killing the coronavirus, and/or by activating other innate and adaptive immune cells.

Thus, the present disclosure also provides methods of treating a disease resulting from infection with a coronavirus in a subject, the method comprising administering a glucocorticoid-receptor (GR) modulating agent to the subject at a dose equivalent to about at least 6 mg/kg human equivalent dose (HED) of dexamethasone base. In some embodiments, the glucocorticoid-receptor (GR) modulating agent may be a glucocorticoid, preferably dexamethasone or betamethasone. In some embodiments, the glucocorticoid-receptor (GR) modulating agent may be administered at a dose equivalent to about at least 15 mg/kg human equivalent dose (HED) of dexamethasone base. In some preferred embodiments, the glucocorticoid-receptor (GR) modulating agent may be administered at a dose equivalent to between about 18 mg/kg and 30 mg/kg human equivalent dose (HED) of dexamethasone base. In some preferred embodiments, the disease is COVID-19 (coronavirus 2019; the disease caused by severe acute respiratory syndrome coronavirus 2, SARS-CoV-2) or SARS-CoV or MERS. In some embodiments, the glucocorticoid-receptor (GR) modulating agent induces/mobilizes a population of NKT-like cells as disclosed elsewhere herein.

In some preferred embodiments, the present disclosure provides a method of treating COVID-19 (coronavirus 2019; the disease caused by severe acute respiratory syndrome coronavirus 2, SARS-CoV-2) in a subject, the method comprising administering dexamethasone or betamethasone to the subject at a dose equivalent to between about 15 mg/kg and 30 mg/kg human equivalent dose (HED) of dexamethasone base.

In embodiments in which the infectious disease is a disease resulting from infection with a coronavirus, for example COVID-19, the glucocorticoid receptor modulating agent may be administered in combination with a proton pump inhibitor (such as omeprazole) and/or hydrocortisone. In this context, “in combination with” may mean concurrent administration or may mean separate and/or sequential administration in any order.

In some embodiments of the methods of the disclosure, the methods of producing/mobilizing a population of NKT-like cells may further comprise a step of isolating an NKT-like cell of the disclosure, or a population of NKT-like cells of the disclosure from a subject or from a sample derived from a subject. Accordingly, the present disclosure provides isolated NKT-like cells, as well as isolated populations of NKT-like cells. The isolated cells and isolated populations of cells may be characterized by the pattern of surface proteins which they express, as outlined above.

Suitable methods for isolating cells and populations of cells from a mixed sample are well-known to the skilled person—for example, flow sorting (such as fluorescence-activated cell sorting; FACS) and magnetic particle sorting (such as magnetic-activated cell sorting; MACS), microfluidic cell sorting, density gradient centrifugation, immunodensity cell isolation, expansion in cell culture based on growth factors and other components in the media. In some preferred embodiments of the disclosure, the step of isolating is performed by fluorescence-activated cell sorting (FACS) or magnetic-activated cell sorting (MACS).

In embodiments in which the NKT-like cells are isolated from a sample derived from the subject, the sample may be selected from the group consisting of: blood, plasma, a tumor biopsy or surgically removed tumor, bone marrow, liver, spleen biopsy, and fat or adipose tissue.

In some embodiments, the step of isolating may be performed at least about 1, 3, 12, 24, 48, 72, 96, 120, 144, or 168 hours after administration of glucocorticoid-receptor (GR) modulating agent or ICAM3 modulating agent. In some embodiments, the step of isolating may be performed at least about 1, 3, 8, 9, 10, 11, 12, 13, 14, or 15 days after administration of glucocorticoid-receptor (GR) modulating agent or ICAM3 modulating agent. In some preferred embodiments, the step of isolating is performed at least about 48 hours after said administration. In some other preferred embodiments, the step of isolating is performed at about 1, 3, or 48 hours after said administration. In some embodiments, the step of isolating may be performed between about 1, 3, or 48 hours and 13 days, between about 1, 3, or 48 hours and 168 hours, between about 1, 3, or 48 hours and 120 hours, between about 1, 3, or 48 hours and 96 hours, or between about 1, 3, or 48 hours and 72 hours after administration of glucocorticoid-receptor (GR) modulating agent or ICAM3 modulating agent. In some preferred embodiments, the step of isolating is performed between about 1, 3, or 48 hours and 72 hours after said administration. In some embodiments the step of isolating may be performed within 1, 2, 3, 4, 5, 6, 7, 8, 9, or 10 hours after glucocorticoid administration. In some preferred embodiments, the step of isolating may be performed within 3 hours after glucocorticoid administration. In some particularly preferred embodiments the step of isolating may be performed within 1 hour after glucocorticoid administration. In other particularly preferred embodiments the step of isolating may be performed between 30 and 60 minutes after glucocorticoid administration. In some preferred embodiments in which the subject has cancer, an infectious disease, or autoimmune disease, the step of isolating the NKT-like cells may be performed on a blood sample from the subject, within 3 hours after glucocorticoid administration, and preferably within 1 hour after glucocorticoid administration, such as between 30 and 60 minutes after glucocorticoid administration.

In some preferred embodiments of methods involving a step of isolating, the subject may be a healthy subject, such as a healthy adult human subject. In this context a healthy subject is a subject which is not afflicted with disease.

The isolated NKT-like cells, and the isolated NKT-like cell populations of the disclosure can be expanded in culture. Suitable methods and reagents for culturing and expanding cells are well-known to the skilled person. For instance, long term culture with IL-2, soluble anti-CD28 antibody, anti-CD3 epsilon antibody, anti-TCRbeta antibody, and glycolipids such as KRN7000, PBS44, or PBS57 has been shown to produce robust expansion of NKT cells (Watarai et al 2008, which is hereby incorporated by reference in its entirety). Accordingly, in some embodiments of the methods of the disclosure, the method of producing a population of NKT-like cells may further comprise a step of expanding the NKT-like cell or NKT-like cells isolated by the step of isolating. In some embodiments of the method of the disclosure, the method may further comprise a step of activating the isolated cells (either before or after the step of expanding) with an NKT cell activator, T cell activator, and/or NK cell activator, which may be as described in detail above.

In some embodiments, following isolation of an NKT-like cell or population of NKT-like cells from the subject or from a sample derived from the subject, the methods of the disclosure may further comprise a step of introducing a nucleic acid encoding a protein into the isolated cell or cells. Suitable methods for introducing a nucleic acid into a cell are well known to the skilled person—for example, physical or chemical methods including electroporation, sonoporation, cell microinjection, microparticle delivery, calcium-phosphate mediated transfection, and liposome-based transfection; or, viral transduction. Following introduction of the nucleic acid encoding a protein, the cell or cells may be cultured under conditions that facilitate expression of the encoded protein. Suitable methods, reagents, and conditions for culturing cells are well-known to the skilled person. The cell or cells into which a nucleic acid encoding a protein has been introduced may be referred to herein as transfected or transformed cells.

In some embodiments of the disclosure, the nucleic acid encoding a protein is a nucleic acid which encodes a protein selected from the group consisting of one or more of: a T-cell receptor (TCR), a chimeric antigen receptor (CAR), a split, and universal and programmable CAR (SUPRA-CAR).

After isolation, the NKT-like cells of the disclosure may be genetically engineered for a particular target. For example, the cells can be expanded by IL-2 and activated with GalCer (galactosylceramide), pulsed autologous irradiated PBMCs, then transduced to express a CAR or recombinant TCR (rTCR). The CAR or rTCR may specifically bind a target selected from GD2 (disialoganglioside) and CD19. For instance, the CAR may be NCT03294954 (which specifically binds GD2) or NCT03774654 (which specifically binds CD19).

Moreover, the isolated cells can undergo targeted activation. For instance, the following procedures can be utilized: Nanovectors for passive and active delivery; a-GalCer-loaded APCs for targeted activation of NKT-like cells to tumors; i.v. administration of α-GalCer; and/or bulk PBMCs stimulation (two to three times) via addition of α-GalCer to the cultured cells (to produce an iNKT cell-enriched population, which is then infused back into the patient)

Moreover, the isolated cells can be directly linked to tumor targeting moieties (either on tumor cells or TME). Chemical modification of stimulatory agents for NKT cells (polarization of immune responses by α-GalCer analogues), T cells, and NK cells can also be employed.

The term “chimeric antigen receptor” (CAR) as used herein non-exclusively relates to constructs that contain an antigen-binding domain of an antibody fused to a strong T-cell activator domain. T-cells modified with the CAR construct can bind to the antigen and be stimulated to attack the bound cells. Artificial T cell receptors (also known as chimeric T cell receptors, chimeric immunoreceptors, chimeric antigen receptors (CARs)) are engineered receptors, which graft an arbitrary specificity onto an immune effector cell. The receptors are called chimeric because they are composed of parts from different sources. The receptor/ligand or antibody expressed by the chimeric antigen receptor T cells or cellular immunotherapy can be mono- or bi-specific or multi-specific.

In some embodiments, the TCR, CAR, and/or SUPRA-CAR may comprise an antigen-binding domain which binds to an antigen selected from the group of receptors/ligands/targets consisting of: Proto-oncogene tyrosine-protein kinase ABL1, Citrullinated Antigen, ErbB2/HER2, CD16, WT-1, KRAS, glypican 3, CD3, CD20, CD226, CD155, CD123, HPV-16 E6, Melan-A/MART-1, TRAIL Bound to the DR4 Receptor, LMP, MTCR, ESO, NY-ESO-1, gp100, 4SCAR-GD2/CD56, Mesothelin (CAK1 Antigen or Pre Pro Megakaryocyte Potentiating Factor or MSLN); DNA Synthesis Inhibitor; Histamine H1 Receptor (HRH1) Antagonist; Prostaglandin G/H Synthase 2 (Cyclooxygenase 2 or COX2 or Prostaglandin Endoperoxide Synthase 2 or PHS II or Prostaglandin H2 Synthase 2 or PTGS2 or EC 1.14.99.1) Inhibitor, CD19 (B Lymphocyte Surface Antigen B4 or Differentiation Antigen CD19 or T Cell Surface Antigen Leu 12 or CD19), Cell Adhesion Molecule 5 (Carcinoembryonic Antigen or CEA or Meconium Antigen 100 or CD66e or CEACAM5); Interleukin 2 Receptor (IL2R) Agonist, Epidermal Growth Factor Receptor (Proto Oncogene c ErbB 1 or Receptor Tyrosine Protein Kinase erbB 1 or HER1 or ERBB1 or EGFR or EC 2.7.10.1); DNA Ligase (EC 6.5.1.) Inhibitor; DNA Ligase (EC 6.5.1.), DNA Polymerase Alpha (POLA or EC 2.7.7.7) Inhibitor; DNA Primase (EC 2.7.7.6) Inhibitor; Ribonucleoside Diphosphate Reductase (Ribonucleotide Reductase or RRM or EC 1.17.4.1) Inhibitor; RNA Polymerase II (RNAP II or Pol II or EC 2.7.7.6) Inhibitor, DNA Polymerase (EC 2.7.7.7) Inhibitor; DNA Topoisomerase II (EC 5.99.1.3) Inhibitor; CD22, meso, DNA Primase (EC 2.7.7.6); Programmed Cell Death 1 Ligand 1 (PD L1 or B7 Homolog 1 or CD274) Inhibitor; RNA Polymerase II (RNAP II or Pol II or EC 2.7.7.6), Histone Lysine N Methyltransferase EZH2 (ENX 1 or Enhancer Of Zeste Homolog 2 or Lysine N Methyltransferase 6 or EZH2 or EC 2.1.1.43) Inhibitor; Programmed Cell Death 1 Ligand 1 (PD L1 or B7 Homolog 1 or CD274), C-X-C Chemokine Receptor Type 4 (FB22 or Fusin or HM89 or LCR1 or Leukocyte Derived Seven Transmembrane Domain Receptor or Lipopolysaccharide Associated Protein 3 or Stromal Cell Derived Factor 1 Receptor or NPYRL or CD184 or CXCR4) Antagonist; Granulocyte Colony Stimulating Factor Receptor (CD114 or GCSFR or CSF3R) Agonist, Adenosine Deaminase (Adenosine Aminohydrolase or ADA or EC 3.5.4.4) Inhibitor; Tumor Necrosis Factor Receptor Superfamily Member 17 (B Cell Maturation Antigen or CD269 or TNFRSF17), Cytocytotoxic To Cells Expressing Inactive Tyrosine Protein Kinase Transmembrane Receptor ROR1 (Neurotrophic Tyrosine Kinase Receptor Related 1 or ROR1 or EC 2.7.10.1); T Cell Surface Glycoprotein CD3 Epsilon Chain (T Cell Surface Antigen T3/Leu 4 Epsilon Chain or CD3E); Dihydrofolate Reductase (DHFR or EC 1.5.1.3) Inhibitor; Ephrin Type A Receptor 2 (Epithelial Cell Kinase or Tyrosine Protein Kinase Receptor ECK or EPHA2 or EC 2.7.10.1) Inhibitor; Glucocorticoid Receptor (GR or Nuclear Receptor Subfamily 3 Group C Member 1 or NR3C1) Agonist; Mast/Stem Cell Growth Factor Receptor Kit (Proto Oncogene c Kit or Tyrosine Protein Kinase Kit or v Kit Hardy Zuckerman 4 Feline Sarcoma Viral Oncogene Homolog or Piebald Trait Protein or p145 c Kit or CD117 or KIT or EC 2.7.10.1) Inhibitor; Platelet Derived Growth Factor Receptor Beta (Beta Type Platelet Derived Growth Factor Receptor or CD140 Antigen Like Family Member B or Platelet Derived Growth Factor Receptor 1 or CD140b or PDGFRB or EC 2.7.10.1) Inhibitor; Tubulin Inhibitor; Tyrosine Protein Kinase CSK (C Src Kinase or Protein Tyrosine Kinase CYL or CSK or EC 2.7.10.2) Inhibitor; Tyrosine Protein Kinase Fyn (Proto Oncogene Syn or Proto Oncogene c Fyn or Src Like Kinase or p59 Fyn or FYN or EC 2.7.10.2) Inhibitor; Tyrosine Protein Kinase Lck (Leukocyte C Terminal Src Kinase or Protein YT16 or Proto Oncogene Lck or T Cell Specific Protein Tyrosine Kinase or Lymphocyte Cell Specific Protein Tyrosine Kinase or p56 LCK or LCK or EC 2.7.10.2) Inhibitor; Tyrosine Protein Kinase Yes (Proto Oncogene c Yes or p61 Yes or YES1 or EC 2.7.10.2) Inhibitor, Tumor Necrosis Factor (Cachectin or TNF Alpha or Tumor Necrosis Factor Ligand Superfamily Member 2 or TNF a or TNF) Inhibitor, Signal Transducer And Activator Of Transcription 3 (Acute Phase Response Factor or DNA Binding Protein APRF or STAT3) Inhibitor, Bcr-Abl Tyrosine Kinase (EC 2.7.10.2) Inhibitor; Dihydrofolate Reductase (DHFR or EC 1.5.1.3); Ephrin Type A Receptor 2 (Epithelial Cell Kinase or Tyrosine Protein Kinase Receptor ECK or EPHA2 or EC 2.7.10.1); Mast/Stem Cell Growth Factor Receptor Kit (Proto Oncogene c Kit or Tyrosine Protein Kinase Kit or v Kit Hardy Zuckerman 4 Feline Sarcoma Viral Oncogene Homolog or Piebald Trait Protein or p145 c Kit or CD117 or KIT or EC 2.7.10.1); Platelet Derived Growth Factor Receptor Beta (Beta Type Platelet Derived Growth Factor Receptor or CD140 Antigen Like Family Member B or Platelet Derived Growth Factor Receptor 1 or CD140b or PDGFRB or EC 2.7.10.1); Tubulin; Tyrosine Protein Kinase CSK (C Src Kinase or Protein Tyrosine Kinase CYL or CSK or EC 2.7.10.2) Inhibitor; Tyrosine Protein Kinase Fyn (Proto Oncogene Syn or Proto Oncogene c Fyn or Src Like Kinase or p59 Fyn or FYN or EC 2.7.10.2) Inhibitor; Tyrosine Protein Kinase Lck (Leukocyte C Terminal Src Kinase or Protein YT16 or Proto Oncogene Lck or T Cell Specific Protein Tyrosine Kinase or Lymphocyte Cell Specific Protein Tyrosine Kinase or p56 LCK or LCK or EC 2.7.10.2) Inhibitor; Tyrosine Protein Kinase Yes (Proto Oncogene c Yes or p61 Yes or YES1 or EC 2.7.10.2) Inhibitor, Caspase 9 (Apoptotic Protease Mch 6 or Apoptotic Protease Activating Factor 3 or ICE Like Apoptotic Protease 6 or CASP9 or EC 3.4.22.62) Activator; Prostate Stem Cell Antigen (PSCA), Melanoma Antigen Preferentially Expressed In Tumors (Cancer/Testis Antigen 130 or Opa Interacting Protein 4 or OIP4 or Preferentially Expressed Antigen Of Melanoma or PRAME), Signal Transducer And Activator Of Transcription 3 (Acute Phase Response Factor or DNA Binding Protein APRF or STAT3) Inhibitor, CD44 Antigen (CDw44 or Epican or Extracellular Matrix Receptor III or GP90 Lymphocyte Homing/Adhesion Receptor or HUTCH I or Heparan Sulfate Proteoglycan or Hermes Antigen or Hyaluronate Receptor or Phagocytic Glycoprotein 1 or CD44), AXL (anexelekto) receptor tyrosine kinase, GAS6, TAM receptor tyrosine kinases, TYRO-3 (also known as Brt, Dtk, Rse, Sky and Tif), AXL (also known as Ark, Tyro7 and Ufo), and MER (also known as Eyk, Nym and Tyro12), CTLA4, Tumor Necrosis Factor Receptor Superfamily Member 8 (CD30L Receptor or Ki 1 Antigen or Lymphocyte Activation Antigen CD30 or CD30 or TNFRSF8), Caspase 9 (Apoptotic Protease Mch 6 or Apoptotic Protease Activating Factor 3 or ICE Like Apoptotic Protease 6 or CASP9 or EC 3.4.22.62) Activator; Cytocytotoxic To Cells Expressing Ganglioside GD2; Prostaglandin G/H Synthase 1 (Cyclooxygenase 1 or COX1 or Prostaglandin Endoperoxide Synthase 1 or Prostaglandin H2 Synthase 1 or PTGS1 or EC 1.14.99.1) Inhibitor; cytokines, interleukins, Claudin 6 (Skullin or CLDN6), NKG2D, MICA, MICB and ULBP 1-6, NKp30, B7H6 (NCR3LG1), Bag6, B7 family, CD40 Ligand (T Cell Antigen Gp39 or TNF Related Activation Protein or Tumor Necrosis Factor Ligand Superfamily Member 5 or CD154 or CD40LG) Activator; Interleukin 12 (IL12) Activator, Interleukin 3 Receptor Subunit Alpha (IL3RAMast/Stem Cell Growth F), actor Receptor Kit (Proto Oncogene c Kit or Tyrosine Protein Kinase Kit or v Kit Hardy Zuckerman 4 Feline Sarcoma Viral Oncogene Homolog or Piebald Trait Protein or p145 c Kit or CD117 or KIT or EC 2.7.10.1) Antagonist; Proto Oncogene Tyrosine Protein Kinase Receptor Ret (Cadherin Family Member 12 or Proto Oncogene c Ret or RET or EC 2.7.10.1) Inhibitor; Receptor Type Tyrosine Protein Kinase FLT3 (FMS Like Tyrosine Kinase 3 or FL Cytokine Receptor or Stem Cell Tyrosine Kinase 1 or Fetal Liver Kinase 2 or CD135 or FLT3 or EC 2.7.10.1) Antagonist; Vascular Endothelial Growth Factor Receptor 1 (Fms Like Tyrosine Kinase 1 or Tyrosine Protein Kinase Receptor FLT or Tyrosine Protein Kinase FRT or Vascular Permeability Factor Receptor or VEGFR1 or FLT1 or EC 2.7.10.1) Antagonist; Vascular Endothelial Growth Factor Receptor 2 (Fetal Liver Kinase 1 or Kinase Insert Domain Receptor or Protein Tyrosine Kinase Receptor flk 1 or VEGFR2 or CD309 or KDR or EC 2.7.10.1) Antagonist; Vascular Endothelial Growth Factor Receptor 3 (Fms Like Tyrosine Kinase 4 or Tyrosine Protein Kinase Receptor FLT4 or VEGFR3 or FLT4 or EC 2.7.10.1) Antagonist, Caspase 9 (Apoptotic Protease Mch 6 or Apoptotic Protease Activating Factor 3 or ICE Like Apoptotic Protease 6 or CASP9 or EC 3.4.22.62) Activator, Cytocytotoxic T Lymphocyte Protein 4 (Cytocytotoxic T Lymphocyte Associated Antigen 4 or CD152 or CTLA4) Antagonist, Myeloid Cell Surface Antigen CD33 (Sialic Acid Binding Ig Like Lectin 3 or gp67 or CD33), Hepatocyte Growth Factor Receptor (Proto Oncogene c Met or Tyrosine Protein Kinase Met or HGF/SF Receptor or Scatter Factor Receptor or MET or EC 2.7.10.1), Epithelial Cell Adhesion Molecule (Adenocarcinoma Associated Antigen or Cell Surface Glycoprotein Trop 1 or Epithelial Cell Surface Antigen or Epithelial Glycoprotein 314 or KS 1/4 Antigen or KSA or Tumor Associated Calcium Signal Transducer 1 or CD326 or EPCAM), Ganglioside GD2, Lewis Y Antigen (CD174), Latent Membrane Protein 1 (Protein p63 or LMP1), Mucin 1 (Breast Carcinoma Associated Antigen DF3 or Episialin or H23AG or Krebs Von Den Lungen 6 or PEMT or Peanut Reactive Urinary Mucin or Polymorphic Epithelial Mucin or Tumor Associated Epithelial Membrane Antigen or Tumor Associated Mucin or CD227 or MUC1), T Cell Receptor Beta 1 Chain C Region (TRBC1), Vascular Endothelial Growth Factor Receptor 2 (Fetal Liver Kinase 1 or Kinase Insert Domain Receptor or Protein Tyrosine Kinase Receptor flk 1 or VEGFR2 or CD309 or KDR or EC 2.7.10.1), BCMA, PD-1, interleukin-6 receptor, NKR2, CX-072, T Lymphocyte Protein 4 (Cytocytotoxic T Lymphocyte Associated Antigen 4 or CD152 or CTLA4) Antagonist; Serine/Threonine Protein Kinase B Raf (p94 or Proto Oncogene B Raf or v Raf Murine Sarcoma Viral Oncogene Homolog B1 or BRAF or EC 2.7.11.1) Inhibitor, Mucin 16 (Ovarian Cancer Related Tumor Marker CA125 or Ovarian Carcinoma Antigen CA125 or MUC16); Bcr-Abl Tyrosine Kinase (EC 2.7.10.2) Inhibitor; Tyrosine Protein Kinase CSK (C Src Kinase or Protein Tyrosine Kinase CYL or CSK or EC 2.7.10.2) Inhibitor; Tyrosine Protein Kinase Fyn (Proto Oncogene Syn or Proto Oncogene c Fyn or Src Like Kinase or p59 Fyn or FYN or EC 2.7.10.2) Inhibitor; Tyrosine Protein Kinase Lck (Leukocyte C Terminal Src Kinase or Protein YT16 or Proto Oncogene Lck or T Cell Specific Protein Tyrosine Kinase or

Lymphocyte Cell Specific Protein Tyrosine Kinase or p56 LCK or LCK or EC 2.7.10.2) Inhibitor; Tyrosine Protein Kinase Yes (Proto Oncogene c Yes or p61 Yes or YES1 or EC 2.7.10.2) Inhibitor, Cyclin Dependent Kinase 1 (p34 Protein Kinase or Cell Division Protein Kinase 1 or Cell Division Control Protein 2 Homolog or CDK1 or EC 2.7.11.22 or EC 2.7.11.23) Inhibitor; Cyclin Dependent Kinase 2 (p33 Protein Kinase or Cell Division Protein Kinase 2 or CDK2 or EC 2.7.11.22) Inhibitor; Granulocyte Macrophage Colony Stimulating Factor Receptor Subunit Alpha (CDw116 or CD116 or CSF2RA) Agonist, EGFRVIII, Tyrosine Protein Kinase SYK (Spleen Tyrosine Kinase or p72 Syk or SYK or EC 2.7.10.2) Inhibitor, Alpha Fetoprotein (Alpha 1 Fetoprotein or Alpha Fetoglobulin or AFP), Cancer/Testis Antigen 1 (Autoimmunogenic Cancer/Testis Antigen or Cancer/Testis Antigen 6.1 or L Antigen Family Member 2 or CTAGIA or CTAGIB); HBV antigen, EGFR Family Member, Herin, Tyrosine Protein Kinase BTK (Bruton Tyrosine Kinase or B Cell Progenitor Kinase or Agammaglobulinemia Tyrosine Kinase or BTK or EC 2.7.10.2) Inhibitor, CD4, Epithelial Cell Adhesion Molecule (Adenocarcinoma Associated Antigen or Cell Surface Glycoprotein Trop 1 or Epithelial Cell Surface Antigen or Epithelial Glycoprotein 314 or KS 1/4 Antigen or KSA or Tumor Associated Calcium Signal Transducer 1 or CD326 or EPCAM), Prolyl Endopeptidase FAP (170 kDa Melanoma Membrane Bound Gelatinase or Dipeptidyl Peptidase FAP or Integral Membrane Serine Protease or Fibroblast Activation Protein Alpha or Gelatine Degradation Protease FAP or Seprase or FAP or EC 3.4.21.26 or EC 3.4.14.5), Neural Cell Adhesion Molecule 1 (Antigen Recognized By Monoclonal Antibody 5.1H11 or CD56 or NCAM1); Epidermal Growth Factor Receptor (Proto Oncogene c ErbB 1 or Receptor Tyrosine Protein Kinase erbB 1 or HER1 or ERBB1 or EGFR or EC 2.7.10.1) Antagonist, Tyrosine Protein Kinase Transmembrane Receptor ROR1 (Neurotrophic Tyrosine Kinase Receptor Related 1 or ROR1 or EC 2.7.10.1); Wilms Tumor Protein (WT33 or WT1); Interleukin 13 Receptor Subunit Alpha 2 (Interleukin 13 Binding Protein or CD213a2 or IL13RA2), Trophoblast Glycoprotein (M6P1 or 5T4 Oncofetal Antigen or 5T4 Oncofetal Trophoblast Glycoprotein or Wnt Activated Inhibitory Factor 1 or TPBG), SLAM Family Member 7 (CD319 or Membrane Protein FOAP 12 or CD2 Like Receptor Activating Cytocytotoxic Cells or Novel Ly9 or Protein 19A or CD2 Subset 1 or CS1 or SLAMF7), B Cell Lymphoma 2 (Bcl 2) Inhibitor; DNA (Cytosine 5) Methyltransferase 1 (CXXC Type Zinc Finger Protein 9 or DNA Methyltransferase HsaI or MCMT or DNMTI or EC 2.1.1.37) Inhibitor, ROR1, CD19&CD40L, avidin (EGFRiiiv), a folate receptor, CD30, pmel CD*8 T, CD33, NKR2, Epithelial tumor antigen (ETA), Tyrosinase, Melanoma-associated antigen, abnormal products of ras, p53, Alphafetoprotein (AFP), CA-125, CA15-3, CA27-29, CA19-9, Calcitonin, Calretinin, CD34, CD99MIC 2, CD117, Chromogranin, Cytokeratin (various types: TPA, TPS, Cyfra21-1), Desmin, Epithelial membrane antigen (EMA), Factor VIII, CD31 FL1, Glial fibrillary acidic protein (GFAP), Gross cystic disease fluid protein (GCDFP-15), HMB-45, Human chorionic gonadotropin (hCG), immunoglobulin, inhibin, keratin (various types), lymphocyte marker (various types), BCR-ABL, Myo DI, muscle-specific actin (MSA), neurofilament, neuron-specific enolase (NSE), placental alkaline phosphatase (PLAP), prostate-specific antigen (PSA), PTPRC (CD45), S100 protein, smooth muscle actin (SMA), synaptophysin, thymidine kinase, thyroglobulin (Tg), thyroid transcription factor-1 (TTF-1), Tumor M2-PK, vimentin, SV40, Adenovirus E1b-58kd, IGF2B3, ubiquitous (low level), Kallikrein 4, KIF20A, Lengsin, Meloe, MUC5AC, Immature laminin receptor, TAG-72, HPV E6, HPV E7, BING-4, Calcium-activated chloride channel 2, Cyclin-B1, 9D7, Ep-CAM, EphA3, Telomerase, SAP-1, BAGE family, CAGE family, GAGE family, MAGE family, SAGE family, XAGE family, LAGE-1, PRAME, SSX-2, pmel 17, Tyrosinase, TRP-1/-2, P. polypeptide, MCIR, β-catenin, Prostate-specific antigen, BRCA1, BRCA2, CDK4, CML66, Fibronectin, MART-2, Ras, TGF-beta receptor II, T cell receptor (TCR), BLOC1S6, CD10/Neprilysin, CD24, CD248, CD5/Cluster of Differentiation 5, CD63/Tspan-30/Tetraspanin-30, CEACAM5/CD66e, CT45A3, CTAGIA, CXORF61, DSE, GPA33, HPSE, KLK3, LCP1, LRIG3, LRRC15, megakaryocyte potentiating factor, MOK, MUC4, NDNL2, OCIAD1, PMPCB, PTOV1, RCAS1/EBAG9, RNF43, ROPN1, RPLP1, SARNP, SBEM/MUCL1, TRP1/TYRP1, CA19-9, Inactive Tyrosine Protein Kinase Transmembrane Receptor ROR1 (Neurotrophic Tyrosine Kinase Receptor Related 1 or ROR1 or EC 2.7.10.1), ALK Tyrosine Kinase Receptor (Anaplastic Lymphoma Kinase or CD246 or ALK or EC 2.7.10.1), Prostate Stem Cell Antigen (PSCA), Melanoma Antigen Preferentially Expressed In Tumors (Cancer/Testis Antigen 130 or Opa Interacting Protein 4 or OIP4 or Preferentially Expressed Antigen Of Melanoma or PRAME), Signal Transducer And Activator Of Transcription 3 (Acute Phase Response Factor or DNA Binding Protein APRF or STAT3) Inhibitor, CD44 Antigen (CDw44 or Epican or Extracellular Matrix Receptor III or GP90 Lymphocyte Homing/Adhesion Receptor or HUTCH I or Heparan Sulfate Proteoglycan or Hermes Antigen or Hyaluronate Receptor or Phagocytic Glycoprotein 1 or CD44), CD40 Ligand (T Cell Antigen Gp39 or TNF Related Activation Protein or Tumor Necrosis Factor Ligand Superfamily Member 5 or CD154 or CD40LG) Activator; Tumor Necrosis Factor Receptor Superfamily Member 13B (Transmembrane Activator And CAML Interactor or CD267 or TACI or TNFRSF13B); Cytocytotoxic To Cells Expressing Tumor Necrosis Factor Receptor Superfamily Member 17 (B Cell Maturation Antigen or CD269 or TNFRSF17), CD276 Antigen (B7 Homolog 3 or 4Ig B7 H3 or Costimulatory Molecule or CD276), Myeloid Cell Surface Antigen CD33 (Sialic Acid Binding Ig Like Lectin 3 or gp67 or CD33), ADP Ribosyl Cyclase/Cyclic ADP Ribose Hydrolase 1 (Cyclic ADP Ribose Hydrolase 1 or T10 or 2′ Phospho ADP Ribosyl Cyclase/2′ Phospho Cyclic ADP Ribose Transferase or ADP Ribosyl Cyclase 1 or CD38 or EC 3.2.2.6 or EC 2.4.99.20), C Type Lectin Domain Family 14 Member A (Epidermal Growth Factor Receptor 5 or EGFR5 or CLEC14A), Hepatocyte Growth Factor Receptor (Proto Oncogene c Met or Tyrosine Protein Kinase Met or HGF/SF Receptor or Scatter Factor Receptor or MET or EC 2.7.10.1), Epithelial Cell Adhesion Molecule (Adenocarcinoma Associated Antigen or Cell Surface Glycoprotein Trop 1 or Epithelial Cell Surface Antigen or Epithelial Glycoprotein 314 or KS 1/4 Antigen or KSA or Tumor Associated Calcium Signal Transducer 1 or CD326 or EPCAM), Ganglioside GD3, Interleukin 13 Receptor Subunit Alpha 2 (Interleukin 13 Binding Protein or CD213a2 or IL13RA2); Kappa Myeloma Antigen (KMA), Lambda Myeloma Antigen (LMA), Latent Membrane Protein 1 (Protein p63 or LMP1), Melanoma Associated Antigen, Cytocytotoxic To Cells Expressing T Lymphocyte Activation Antigen CD80 (Activation B7-1 Antigen or CTLA 4 Counter Receptor B7.1 or CD80); Cytocytotoxic To Cells Expressing T Lymphocyte Activation Antigen CD86 (Activation B7-2 Antigen or CTLA 4 Counter Receptor B7.2 or CD86), Inactive Tyrosine Protein Kinase Transmembrane Receptor ROR1 (Neurotrophic Tyrosine Kinase Receptor Related 1 or ROR1 or EC 2.7.10.1), Fas Apoptotic Inhibitory Molecule 3 (IgM Fc Fragment Receptor or Regulator Of Fas Induced Apoptosis Toso or TOSO or FAIM3 or FCMR), T Cell Receptor Beta 1 Chain C Region (TRBC1), Vascular Endothelial Growth Factor Receptor 2 (Fetal Liver Kinase 1 or Kinase Insert Domain Receptor or Protein Tyrosine Kinase Receptor flk 1 or VEGFR2 or CD309 or KDR or EC 2.7.10.1), Alpha Fetoprotein (Alpha 1 Fetoprotein or Alpha Fetoglobulin or AFP), Cancer/Testis Antigen 1 (Autoimmunogenic Cancer/Testis Antigen NY ESO 1 or Cancer/Testis Antigen 6.1 or L Antigen Family Member 2 or CTAGIA or CTAGIB), T Cell Surface Glycoprotein CD5 (Lymphocyte Antigen Tl/Leu 1 or CD5), Prolyl Endopeptidase FAP (170 kDa Melanoma Membrane Bound Gelatinase or Dipeptidyl Peptidase FAP or Integral Membrane Serine Protease or Fibroblast Activation Protein Alpha or Gelatine Degradation Protease FAP or Seprase or FAP or EC 3.4.21.26 or EC 3.4.14.5), Neural Cell Adhesion Molecule 1 (Antigen Recognized By Monoclonal Antibody 5.1H11 or CD56 or NCAM1), C Type Lectin Domain Family 12 Member A (Myeloid Inhibitory C Type Lectin Like Receptor or Dendritic Cell Associated Lectin 2 or C Type Lectin Like Molecule 1 or CLEC12A), Integrin Alpha V (Vitronectin Receptor Subunit Alpha or CD51 or ITGAV); Cytocytotoxic To Cells Expressing Integrin Beta 6 (ITGB6), Interleukin 13 Receptor Subunit Alpha 2 (Interleukin 13 Binding Protein or CD213a2 or IL13RA2), Trophoblast Glycoprotein (M6P1 or 5T4 Oncofetal Antigen or 5T4 Oncofetal Trophoblast Glycoprotein or Wnt Activated Inhibitory Factor 1 or TPBG), Trophoblast Glycoprotein (M6P1 or 5T4 Oncofetal Antigen or 5T4 Oncofetal Trophoblast Glycoprotein or Wnt Activated Inhibitory Factor 1 or TPBG), C Type Lectin Domain Family 12 Member A (Myeloid Inhibitory C Type Lectin Like Receptor or Dendritic Cell Associated Lectin 2 or C Type Lectin Like Molecule 1 or CLEC12A), SLAM Family Member 7 (CD319 or Membrane Protein FOAP 12 or CD2 Like Receptor Activating Cytocytotoxic Cells or Novel Ly9 or Protein 19A or CD2 Subset 1 or CS1 or SLAMF7), SLAM Family Member 7 (CD319 or Membrane Protein FOAP 12 or CD2 Like Receptor Activating Cytocytotoxic Cells or Novel Ly9 or Protein 19A or CD2 Subset 1 or CS1 or SLAMF7), immunoglobulin, Multidrug resistance-associated protein 3 (MRP3), Proto-oncogene tyrosine-protein kinase ABL1, Prostatic acid phosphatase, OY-TES-1, ACSM2A, Alpha-actinin-4, Perilipin-2, Alphafetoprotein, Lymphoid blast crisis oncogene (Lbc) oncoproptein, aldehyde dehydrogenase 1 family member A1 (ALDH1A1), AML, ANKRD17, NY-BR-1, Annexin II, ARHGAP17, ARHGAP30, ARID1B, Endoplasmic reticulum-resident protein, 5′-aminoimidazole-4-carboxamide-1-beta-d-ribonucleotide transfolmylase/inosinicase (AICRT/I), ATR, ATXN2, ATXN2L, BAGE1, BCL11A, Bcl-xL, Breakpoint cluster region, Survivin, Livin/ML-IAP, HM1.24, BTB domain containing 2 (BTBD2), C6ORF89, Carbonic anhydrase IX, CLCA2, CRT2, CAMEL, CAN protein, Caspase-5, Caspase-8, KM-HN-1, CCDC88B, cyclin B1, Cyclin D1, CCNI, CDC2, CDC25A, CDC27, CDK12, intestinal carboxylesterase, CEP95, CHAF1A, Coactosin-like 1, CPSF, CRYBA1, TRAG-3, Macrophage colony stimulating factor, CSNK1A1, Melanoma-associated chondroitin sulfate proteoglycan (MCSP), Cathepsin H, Kita-kyushu lung cancer antigen 1, P450 1B1 or CYP1B1, DDR1, DEK oncogene, DEK-CAN, Dickkopf-1 (DKK1), DNAJC8, DSCAML1, EEF2, Elongation factor Tu GTP binding domain containing or SNRP116, EIF4EBP1, Human Mena protein, EP300, ETV5, TEL1 or ETV6, Polycomb group protein enhancer of zeste homolog 2 (EZH2), F2R, F4.2, FAM53C, Fibroblast growth factor 5 or FGF5, Formin-related protein in leukocytes 1 (FMNL1), Fibromodulin (FMOD), FNDC3B, FKHR, GDP-L-fucose, GAS7, GFI1, GIGYF2, GPNMB, O, A1, GPSM3, GRK2, GRM5, H3F3A, HAUS3, HERC1, HERV-K-MEL, HIVEP2, HMGN, HMHA1, heme oxygenase-1 (HO-1), HNRPL, Heparanase, HMSD-v-encoded mHA, HSPA1A, Hsp70, HSPB1, immediate early response gene X-1 (IEX-1), insulin-like growth factor (IGF)-II mRNA binding protein 3 (IMP-3), IP6K1, IRS2, ITGB8, JUP, RU2AS, KANSL3, KLF10, KLF2, KLK4, KMT2A, K-ras, Low density lipid receptor (LDLR), LDLR-FUT, Mac-2-binding protein, KIAA0205, LPP, LRP1, LRRC41, LSP1, LUZP1, lymphocyte antigen 6 complex locus K (LY6K), MACF1, MAP1A, MAP3K11, MAP7D1, Matrilin-2, Mcl-1, MDM2, Malic enzyme, MEF2D, MEFV, Milk fat globule membrane protein BA46 (lactadherin), Melanotransferrin, GNT-V or N-acetylglucosaminytransferase V, MIIP, MMP14, Matrix metalloproteinase-2, MORC2, Melanoma antigen p15, MUC2, MUM, MYC, MYL9, Unconventional myosin class I gene, N4BP2, NCBP3, NCOA1, NCOR2, NFATC2, NFYC, NIFK, Ninein, NPM, NPM1-ALK1, N-ras, OAS3, P polypeptide, OGT, OS-9, ErbB3-binding protein 1, PAGE-4, P21-activated serine kinase 2 (PAK2), neo-PAP, PARP12, PAX3, PAX3-FKHR, PCBP2, phosphoglycerate kinase 1 (PKG1), PLEKHM2, promyelocytic leukemia or PML, PML-RARA, POLR2A, Cyclophilin B, PPP1CA, PPP1R3B, Peroxiredoxin 5, Proteinase 3, Parathyroid hormone-related protein (PTHrP), Receptor-like protein tyrosine phosphatase kappa, MG50, NY-MEL-1 or RAB38, RAGE, RALGAPB, RAR alpha, RBM, RCSD1, Recoverin, RERE, RGS5, RHAMM/CD168, RPA1, Ribosomal protein L10a, Ribosomal protein S2, RREB1, RSRP1, RTCB, SART, SCAP, Mammaglobin A, Secernin 1, SDCBP, SETD2, SF3B1, Renal ubiquitous protein 1, SIK1, SIRT2, SKI, hairpin-binding protein, SLC35A4, Prostein, SLC46A1, SNRPD1, SOGA1, SON, SOX10, SOX11, SOX2, SOX-4, Sperm protein 17, SPEN, SRRM2, SRSF7, SRSF8, SSX1, SSX2 or HOM-MEL-40, SSX4, STAT1, STEAP, STRAP, ART-1, SVIL, HOM-TES-14/SCP1, CD138, SYNM, SYNPO, SYT, SYT15, SYT-SSX1, SYT-SSX2, SZT2, TAPBP, TBC1D10C, TBC1D9B, hTERT, THNSL2, THOC6, TLK1, TNS3, TOP2A, TOP2B, ATP-dependent interferon-responsive (ADIR), TP53, Triosephosphate isomerase or TPI1, Tropomyosin-4, TPX2, TRG, T-cell receptor gamma alternate reading frame protein (TARP), TRIM68, Prostate-specific protein transient receptor potential-p8 (trp-p8), TSC22D4, TTK protein kinase (TTK), Thymidylate synthase (TYMS), UBE2A, Ubiquitin-conjugating enzyme variant Kua, COA-1, USB1, NA88-A, VPS13D, BING4, WHSCIL1, WHSC2, WNK2, WT1, XBP1, XPO1, ZC3H14, ZNF106, ZNF219, Papillomavirus binding factor (PBF), E3 ubiquitin-protein ligase UBR4.

In some embodiments of the disclosure, the TCR, CAR, and/or SUPRA-CAR may not comprise an antigen-binding domain which binds to an antigen selected from the above recited group of receptors/ligands/targets.

In some preferred embodiments, the TCR, CAR, and/or SUPRA-CAR may comprise an antigen-binding domain which binds to an antigen selected from the group consisting of: CD19, CD20, CD22, GD2, CD133, EGFR, GPC3, CEA, MUC1, Mesothelin, IL-13R, PSMA, ROR1, CAIX, Her2.

Following introduction of the nucleic acid encoding a protein, the NKT-like cell or NKT-like cells may be expanded in culture. Suitable methods and reagents for culturing and expanding cells are well-known to the skilled person. Following expansion the methods of the disclosure may further comprise a step of activating the cells with an NKT cell activator, T cell activator, and/or NK cell activator. The NKT cell activator, T cell activator, and NK cell activator may be as described in detail above.

In some embodiments, cells or targeted cells of the disclosure as described above are used to deliver a payload such as nucleic acids, dsRNA, siRNA, micro RNA, dsDNA, SSDNA, cDNA, RNA, mRNA, tRNA, siRNA, dsRNAi, RNAi, organic compounds, cytotoxic drugs, antibodies, vedotin, ozogamicine, emtansine, deruxtecan, mertansine, mafodotin, tubulin inhibitors, Monomethyl auristatin-E (MMAE) and monomethyl auristatin-F (MMAF) are peptide analogs of dolastatin-10, Maytansinoids, vinca alkaloids, calicheamicin, Duocarmycins, pyrrolobenzodiazepine dimers, talirine, tesirine, indolinobenzodiazepine pseudodimers, soravtansine, DM1, DM4, neurotransmitters, DNA intercalators, antimetabolites, endostatins, neurotrophins, chemotherapy, or a growth factor, or an antibody, a toxin, radioactivity, antibiotics, anti-fungal agents, anti-viral agents, receptors, a virus, a cytokine, lipids, a chemokine, peptides and proteins, anti-parasitics, hormones, antigens, neuro-active agents, receptor agonists or antagonists, small molecules, or any type of biologic payload or biologically active payload.

In some embodiments of the disclosure, the cells of the disclosure may be used to deliver a payload that is not one or more of the above recited payloads.

Also provided by the present disclosure are methods of treating cancer, autoimmune disease, or infectious disease (also called microbial disease) in a subject. In some embodiments, the method of treatment is a method of producing a population of NKT-like cells in a subject as described elsewhere herein. In some embodiments, the method of treatment is a method of mobilizing a population of NKT-like cells in a subject as described elsewhere herein. In these embodiments, the NKT-like cells may treat the cancer, autoimmune disease, or infectious disease by one or mechanism described elsewhere herein. In other embodiments, the method of treatment is a method comprising administering to a subject a therapeutically effective dose of the isolated NKT-like cells of the disclosure. These may be any of the isolated NKT-like cell or population of NKT-like cells outlined above, including the expanded and non-expanded, and/or activated or non-activated and/or transfected or non-transfected cells described above. In these embodiments, the subject, cancer, autoimmune disease, infectious disease, and/or mechanism of therapeutic efficacy may be as described in detail above.

In embodiments in which the method of treatment is a method comprising administering to a subject a therapeutically effective dose of the isolated NKT-like cells of the disclosure, the subject to which the isolated cells are administered may be the same subject from which the cells were isolated. In such embodiments, the treatment may be referred to as an autologous cell treatment. The term “autologous” refers to any material derived from the same individual to which it is later re-introduced, whether the individual is a human or other animal. In other embodiments in which the method of treatment is a method comprising administering to a subject a therapeutically effective dose of the isolated NKT-like cells of the disclosure, the subject to which the isolated cells are administered may be different to the subject from which the cells were isolated. In such embodiments, the treatment may be referred to as an allogeneic cell treatment. The term “allogeneic” refers to any material derived from one individual which is then introduced to another individual of the same species, whether the individual is a human or other animal. That is, in embodiments in which the method of treatment is a method comprising administering to a subject a therapeutically effective dose of the isolated NKT-like cells of the disclosure, the cells can be from either an autologous or allogeneic source. Therapeutic efficacy of methods in which isolated NKT-like cells of the disclosure are administered to a subject is described in Example 16.

The methods of treating cancer, autoimmune disease, or infectious disease in a subject according to the present disclosure may further comprise a step of administering an NKT cell activator, T cell activator, and/or NK cell activator to the subject. These may be as described in detail above. The methods of treating cancer, autoimmune disease, or infectious disease in a subject according to the present disclosure may comprise administering the glucocorticoid or cells of the disclosure to a subject in combination with one or more additional agents, for example an NKT cell activator, T cell activator, and/or dendritic NK cell activator as outlined above, or a chemotherapeutic agent, such as an immune checkpoint inhibitor. In this context, “in combination with” may mean concurrent administration or may mean separate and/or sequential administration in any order.

As used herein, the term “administering” refers to the physical introduction of an agent to a subject, using any of the various methods and delivery systems known to those skilled in the art. Exemplary routes of administration for the agents disclosed herein include intravenous, intramuscular, subcutaneous, intraperitoneal, spinal or other parenteral routes of administration, for example by injection or infusion. The phrase “parenteral administration” as used herein means modes of administration other than enteral and topical administration, usually by injection, and includes, without limitation, intravenous, intramuscular, intraarterial, intrathecal, intralymphatic, intralesional, intracapsular, intraorbital, intracardiac, intradermal, intraperitoneal, transtracheal, subcutaneous, subcuticular, intraarticular, subcapsular, subarachnoid, intraspinal, epidural and intrasternal injection and infusion, as well as in vivo electroporation. In some embodiments, the agents disclosed herein may be administered via a non-parenteral route, e.g., orally. Other non-parenteral routes include a topical, epidermal, or mucosal route of administration, for example, intranasally, vaginally, rectally, sublingually or topically.

The phrase “systemic injection” as used herein non-exclusively relates to intravenous, intraperitoneally, subcutaneous, via nasal submucosa, lingual, via bronchoscopy, intravenous, intra-arterial, intra-muscular, intro-ocular, intra-striatal, subcutaneous, intradermal, by dermal patch, by skin patch, by patch, into the cerebrospinal fluid, into the portal vein, into the brain, into the lymphatic system, intra-pleural, retro-orbital, intra-dermal, into the spleen, intra-lymphatic, among others.

The term ‘site of injection’ as used herein non-exclusively relates to intra-tumor, or intra-organ such as the kidney or liver or pancreas or heart or lung or brain or spleen or eye, intra-muscular, intro-ocular, intra-striatal, intradermal, by dermal patch, by skin patch, by patch, into the cerebrospinal fluid, into the brain, among others.

In some preferred embodiments of the disclosure, the glucocorticoid-receptor modulating agents may be administered orally. In embodiments in which the method of treatment of the disclosure is a method comprising administering to a subject a therapeutically effective dose of the isolated NKT-like cells of the disclosure, the cells may be applied directly to an organ or tumor via collagen matrices, extracellular matrix compositions, biopolymer microthreads made of fibrin or other extracellular matrix material, patches containing extracellular matrix and biodegradable materials, fibrin patches, alginateor agarose based patches, scaffolds composed of extracellular matrix materials and biodegradable physiologically inert material that could non-exclusively relates to components such as dextrans, coating stem cells with organ specific antigens or binding molecules, remnant extracellular matrices also known as scaffolds or decellularized organs from ex vivo digested organ donors or cadaveric organs, and contact lenses among others. Preferably the cells are administered to the subject by a method selected from the group consisting of: intravenous injection, intraperitoneal injection, intra-lymphatic injection, intrathecal injection, injection into the cerebrospinal fluid (CSF), direct injection into a tumor, or as a gel placed on or near a solid tumor.

In some embodiments of the disclosure, the route of administration for the agents and cells disclosed herein may not be one or more of the above recited routes.

The present disclosure also provides glucocorticoid-receptor (GR) modulating agents and ICAM3 modulating agents for use in a method of producing/mobilizing a population of NKT-like cells as described in detail above. The present disclosure also provides glucocorticoid-receptor (GR) modulating agents and ICAM3 modulating agents, for use in a method of treating cancer, autoimmune disease, or infectious disease (also called microbial disease) in a subject, wherein the method of treatment is a method of producing/activating/mobilizing a population of NKT-like cells in a subject as described in detail above. Preferred embodiments include glucocorticoids for use in a method of producing and/or mobilizing a population of NKT-like cells as described in detail above, and glucocorticoids for use in a method of treating cancer, autoimmune disease, or infectious disease in a subject, wherein the method of treatment is a method of producing and/or mobilizing a population of NKT-like cells in a subject as described in detail above. Other preferred embodiments include glucocorticoids for use in a method of mobilizing a population of NKT cells as described in detail above. In some particularly preferred embodiments, the glucocorticoid is dexamethasone.

Also provided by the disclosure is the use of glucocorticoid-receptor (GR) modulating agents or ICAM3 modulating agents in the manufacture of a medicament for use in a method of producing/mobilizing a population of NKT-like cells as described in detail above. The present disclosure also provides use of glucocorticoid-receptor (GR) modulating agents or ICAM3 modulating agents in the manufacture of a medicament for use in a method of treating cancer, autoimmune disease, or infectious disease (also called microbial disease) in a subject, wherein the method of treatment is a method of producing and/or mobilizing a population of NKT-like cells in a subject as described in detail above.

The present disclosure also provides the use of a glucocorticoid-receptor (GR) modulating agent or ICAM3 modulating agent to induce and/or mobilize a population of NKT-like cells, wherein the population of NKT-like cells is induced by a method of producing and/or mobilizing a population of cells in a subject as described in detail above.

The present disclosure also provides a method of producing induced pluripotent stem cells (iPSCs), the method comprising reprogramming the NKT-like cells of the disclosure to produce iPSCs. The NKT-like cells of the disclosure to be used in a method of producing iPSCs, may be NKT-like cells produced and isolated by a method as described in detail above.

In some embodiments of the disclosed method of producing iPSCs, the reprogramming comprises introducing one or more expression cassettes encoding Oct3/4, Klf4, Sox2, and C-myc into the cells of the disclosure. In some embodiments, the reprogramming comprises introducing Oct3/4, KLF4, Sox2, and c-myc encoding mRNA into the cells. In some other embodiments of the disclosed method of producing iPSCs, the reprogramming may further comprise introducing one or more expression cassettes encoding one or more of: Sox1, Sox3, Sox15, Klf1, Klf2, Klf5, L-myc, N-myc, Nanog, and/or LIN28 into the cells. In other embodiments, the reprogramming may further comprise introducing one or more of: Sox1, Sox3, Sox15, Klf1, Klf2, Klf5, L-myc, N-myc, Nanog, and/or LIN28 encoding mRNA into the cells. Suitable methods for introducing expression cassettes or encoding mRNA into a cell are well known to the skilled person—for example by electroporation, cell microinjection, or liposome-based transfection methods. Use of retroviral systems, including lentiviral and adenoviral systems, to reprogram non-pluripotent cells in iPSCs have been described (Stadtfeld et al, 2008, which is hereby incorporated by reference in its entirety). Reprogramming of adult cells to iPSCs can also be accomplished via plasmid without use of virus transfection systems (Okita et al, 2008, which is hereby incorporated by reference in its entirety).

### Oct-3/4

Oct-3/4 (Pou5f1; cDNA available from Bioclone, San Diego CA) is one of the family of octamer (“Oct”) transcription factors, and plays a crucial role in maintaining pluripotency. The absence of Oct-3/4 in Oct-3/4+ cells, such as blastomeres and embryonic stem cells, leads to spontaneous trophoblast differentiation, and presence of Oct-3/4 thus gives rise to the pluripotency and differentiation potential of embryonic stem cells. Various other genes in the “Oct” family, including Oct-3/4's close relatives, Oct1 and Oct6, fail to elicit induction, thus demonstrating the exclusiveness of Oct-3/4 to the induction process.

### Klf Family:

Klf4 of the Klf family of genes is a factor for the generation of mouse iPS cells. Klf2 (cDNA available from Bioclone, Inc., San Diego, CA) and Klf4 (cDNA available from Bioclone, Inc., San Diego, CA) are factors capable of generating iPS cells, and related genes Klf1 (cDNA available from Bioclone, Inc., San Diego, CA) and Klf5 (cDNA available from Bioclone, Inc., San Diego, CA) did as well, although with reduced efficiency.

### Sox Family

The Sox family of genes is associated with maintaining pluripotency similar to Oct-3/4, although it is associated with multipotent and unipotent stem cells in contrast with Oct-3/4, which is exclusively expressed in pluripotent stem cells (Bowles et al, 2000, which is hereby incorporated by reference in its entirety). While Sox2 (cDNA available from Bioclone, San Diego, CA) was the initial gene used for induction, other genes in the Sox family have been found to work as well in the induction process. Sox1 (cDNA available from Bioclone, Inc., San Diego, CA) yields iPS cells with a similar efficiency as Sox2, and genes Sox3 (human cDNA available from Bioclone, Inc., San Diego, CA), Sox15, and Sox18 also generate iPS cells, although with decreased efficiency.

### Myc Family

The Myc family of genes are proto-oncogenes implicated in cancer. C-myc (cDNA available from Bioclone, Inc., San Diego, CA) is a factor implicated in the generation of mouse iPS cells. However, c-myc may be unnecessary for generation of human iPS cells. Usage of the “myc” family of genes in induction of iPS cells is troubling for the eventuality of iPS cells as clinical therapies, as 25% of mice transplanted with c-myc-induced iPS cells developed lethal teratomas. N-myc (cDNA available from Bioclone, Inc., San Diego, CA) and L-myc have been identified to induce instead of c-myc with similar efficiency.

### Nanog

In embryonic stem cells, Nanog (cDNA available from Bioclone, Inc., San Diego, CA), along with Oct-3/4 and Sox2, is necessary in promoting pluripotency (Chambers et al, 2003, which is hereby incorporated by reference in its entirety).

### LIN28

LIN28 (cDNA available from Bioclone, Inc., San Diego, CA) is an mRNA binding protein expressed in embryonic stem cells and embryonic carcinoma cells associated with differentiation and proliferation (Moss & Tang, 2003, which is hereby incorporated by reference in its entirety).

In some embodiments, the disclosed method of producing iPSCs further comprises a step of inducing differentiation of the iPSCs of the disclosure. In some preferred embodiments, the disclosed methods may further comprise inducing differentiation of the iPSCs of the disclosure into NKT cells. Thus, the present disclosure also provides a method of producing a population of NKT cells, the method comprising differentiating iPSCs produced by a method according to the disclosure into an NKT cell lineage. In some embodiments, the disclosed methods may further comprise inducing differentiation of the iPSCs of the disclosure into T cells. Thus, the present disclosure also provides a method of producing a population of T cells, the method comprising differentiating iPSCs produced by a method according to the disclosure into a T cell lineage. Such differentiated cells may be employed in the methods of treating cancer, autoimmune disease, or infectious disease (also called microbial disease) in a subject according to the present disclosure.

The present disclosure also provides in vitro methods of producing a population of natural killer T cell-like cells (NKT-like cells), isolated NKT-like cells, or isolated populations of NKT-like cells as disclosed elsewhere herein. The in vitro methods of the disclosure may comprise: obtaining a CD3 high CD49b-cell or population of cells; and, contacting the CD3 high CD49b-cell or cells with one or more cytokines; wherein the step of contacting induces the cell or cells to become the NKT-like cells of the disclosure. The NKT-like cells may be characterized by the pattern of surface proteins which they express, as described elsewhere herein. In some embodiments, the CD3 high CD49b-cell or population of cells may be obtained from a subject, such as a human subject. The subject may be a subject as defined elsewhere herein. In some embodiments, the step of contacting may comprise contacting the cell or cells with an NKT cell activator, T cell activator and/or NK cell activators as described elsewhere herein. In some embodiments, the one or more cytokines may comprise one or more cytokine described elsewhere herein as an NKT cell activator, T cell activator and/or NK cell activator. In some embodiments, the one or more cytokines may comprise one or more cytokine described elsewhere herein. In some embodiments, the one or more cytokines may comprise IL-2 and IFNgamma. In some embodiments, the one or more cytokines may comprise IL-2, IFNgamma. and one or more further cytokines. The in vitro methods of the disclosure may further comprise steps of isolation, activation, expansion, introduction of a nucleic acid, genetic engineering for a target, linkage to tumor targeting moieties, etc. as described elsewhere herein. The cell or cells produced by the in vitro methods of the disclosure may be used in methods of treatment in which the NKT-like cells are administered to a subject, as well as in methods of producing induced pluripotent stem cells (iPSCs) as described elsewhere herein.

Also provided by the present disclosure are isolated NKT-like cells produced or mobilized by any of the methods disclosed herein, as well as isolated populations of NKT-like cells produced or mobilized by any of the methods disclosed herein. Also provided are NKT-like cells and isolated populations of NKT-like cells characterized by the patterns of surface proteins described in detail elsewhere herein, and use of such cells in the methods of treatment of the disclosure.

### Examples

The following examples demonstrate that high dose glucocorticoid receptor agonists, in addition to causing near complete lymphodepletion of peripheral blood lymphocytes (without affecting the cell counts of neutrophils, platelets, RBCs and stem cells), can induce production and mobilization of a novel population of NKT-like cells in subjects including human immune system (HIS) mice and humans.

These examples also demonstrate that, in addition to presenting with the known properties of NKT cells, the population of NKT-like cells induced by high dose glucocorticoid receptor agonists have a novel pattern of expression of surface proteins, which allows them to directly engulf cancer cells and exhibit enhanced cytotoxic efficacy against solid cancers.

High doses of glucocorticoid agonists thus represent a promising therapy for use in the treatment of cancer and diseases mediated by immune cells such as lymphocytes.

### ABBREVIATIONS

### Terminology

ab Alpha beta
A20 mouse B lymphoma
AVM NKT CD56+gdTCR+invTCR+ human cell
BM bone marrow
CanMod Cancer Model
CBC Complete blood count
CD Cluster of differentiation
CD19 B lymphocyte marker
CD3 T lymphocyte marker
CD4 helper T lymphocyte marker
CD45 white blood cell marker
CD49b a mouse Natural Killer marker
CD56 a human Natural Killer marker
CD8 a cytotoxic T lymphocyte marker
CNS Central nervous system
CR complete response
Cy/Flu cyclophosphamide/fludarabine
DN double negative
DP Dexamethasone phosphate
DOB Date of birth
DOM Date of manufacture
DSP Dexamethasone sodium phosphate
FDA Food and Drug Administration
FSC forward scatter (cell size)
gd Gamma Delta
GMP Good Manufacturing practices
GP Grandparent
hCD45 human CD45
HED Human equivalent dose
Inv
Invariant
Lymphodepletion Study
LyDep
mCD45 mouse CD45
MCL Mantle Cell Lymphoma
MFI Mean fluorescence intensity
Neoadj
neoadjuvant
Non-Hodgkin's Lymphoma.
NHL
NK Natural Killer cells
NKT Natural Killer T cells
NKT new AVM NKT cells
NOD nonobese-diabetic mouse
PBS Phosphate buffered saline
PFA Paraformaldehyde
PR partial response
R/R relapsed/refractory
SBIR small business innovation research
SD stable disease.
SSC S ide scatter (cell complexity)
TCRα/β T cell receptor alpha beta
TCRg/d T cell receptor gamma delta
TCRinv T cell receptor invariant
UC umbilical cord
WBC white blood cells

### Materials and Methods

Acute high dose dexamethasone may also be referred to herein as Dex, AugmenStem™, PlenaStem™ or AVM0703. The novel population of NKT-like cells induced following administration of acute high dose dexamethasone (AVM0703) may also be referred to herein as NKT cells or AVM-NKT cells.

For initial lymphodepletion studies, naïve C57Bl/6 mice were treated with 18 mg/kg HED DP by oral gavage. Male C57BL/6 mice were obtained from Taconic Bioscience (Germantown, NY) and acclimated to laboratory conditions for at least one week. Mice were dosed once orally with 18 mg/kg Dexamethasone Phosphate (DP) or placebo and kept until timepoint. Each dosed timepoint group was accompanied by a placebo group of the same age and condition according to Table 3. Timepoints 24 hours, 48 hours, 72 hours, 5 days, 7 days, 11 days, 13 days were dosed using GLP grade AVM0703 and placebo. Timepoints 6 hours, 21 days, 28 days, 35 days were dosed using GMP grade AVM0703 and placebo. When mice reached study timepoint, they were euthanized as follows. Mice were anesthetized with isoflurane gas. Once anesthetized, blood was drawn via cardiac puncture and place immediately in heparin-lined microtubes. 10 mL of 5 U/mL of Heparin/PBS was used for infused by slow push for retrograde perfusion via the abdominal aorta to flush out all remaining blood from the vasculature. Subsequently, 250 μL of blood was transferred to a lavender-topped EDTA-lined microtube and transported to by Lynette Brown at Flow Contract Site Labs (Bothell, WA) for analysis by flow cytometry. The remaining blood was sent to Phoenix Labs (Mukilteo, WA) for Complete Blood Counts and Clinical Chemistries

For characterisation of the induced/mobilized population of NKT-like cells (AVM-NKT) in humans, whole blood from human subjects was drawn in K2 EDTA Vacutainer tube (367862, BD Biosciences, NJ) and shipped to AVM Biotechnology at room temperature. 100 μl of whole blood was stained with the following antibodies: CD45 AF700 (2D1), CD16 APC (3G8), INKT PECy7 (6B11), CD8 PE (SK1), CD14 FITC (M5E2), CD56 BV650 (5.1H11), γδTCR BV510 (B1), CD19 BV421 (HIB19), NKp44 APC (P44-8) (all from Biolegend, San Diego, CA) and CD3 APCVio770 (BW264/56), (Miltenyi Biotec, San Jose, CA), 7-AAD, (Biolegend, San Diego CA) was included to distinguish the live and dead cells. Antibody staining was performed for 15 minutes at room temperature. Erythrocytes present in the sample were lysed using BD FACS lyse (BD Biosciences, San Diego CA) for 10 minutes at room temperature, then washed and resuspended in 300 μl 1×DPBS CMF. Healthy patient blood from BloodWorks (Seattle, WA), unstained and fluorescence minus one (FMO) were included as controls. 250 μl sample was analyzed on MACSQuant 16 (Serial #40150, Miltenyi Biotec, San Jose, CA) flow cytometer. Data was analyzed using Kaluza 2.1 (Beckman Coulter Lifesciences, Indianapolis, IN) software for different immune population. Data obtained from flow cytometry analyzed by Kaluza were combined to determine different cell populations gated from Live CD45 Lymphocytes: CD3+ T cell, CD8+ cytotoxic T cell, CD3+CD56+ (NKT cells) CD3-CD56+, CD3-CD56bright (NK cells) and CD3-CD19+ (B cells), CD3+γδTCR+ cells, CD3+γδTCR bright cells, CD3+iTCR+ cells, Live WBC CD3-CD16bright granulocytes, Live WBC CD3-CD14+ monocytes. Numbers were reported as % WBC and as cells per μl.

Humanized mouse experiments employed a high concentration, large volume formulation of AVM0703 that contains the active pharmaceutical ingredient dexamethasone sodium phosphate. AVM0703 contains 26.23 mg/mL dexamethasone sodium phosphate (equivalent to 24 mg/mL dexamethasone phosphate, DP), 10 mg/mL sodium citrate, 0.5 mg/mL disodium edetate, and 0.035 mg/mL sodium sulfite (anhydrous). AVM0703 material used for these studies was GMP grade and manufactured by Hospira, Australia. All AVM0703 dosing information in this report is referred to in terms of Dexamethasone Phosphate. Female huNOG-EXL mice (n=6) were obtained from Taconic Bioscience (Germantown, NY). Female huCD34-NCG mice (n=8) were obtained from Charles River (Wilmington, MA). Mice from both facilities were acclimated to laboratory conditions for at least 5-6 days.

Mice were dosed three times orally with 32 mg/kg Dexamethasone Phosphate (DP) or placebo and kept until timepoint. After first (03/01/2021) and second dose 1 week later (03/08/2021), when mice reached predetermined time point, they were bled up to 70 μL of blood/mouse via cheek puncture. The blood was analyzed via flow cytometry. After third dose 28 days after the previous dose (on Apr. 5, 2021), when mice reached the study time point, they were euthanized under standard operating procedure summarized here: Mice were anesthetized with isoflurane gas. Once anesthetized, blood was drawn via cardiac puncture, at least 300 uL blood was immediately placed in EDTA-lined microtubes to analyze via flow cytometry, and 300-400 uL of blood was collected separately in standard microcentrifuge tubes to allow clotting for serum collection.

10 mL of 5 U/mL of Heparin/PBS was used for infusion by slow push for retrograde perfusion via the abdominal aorta to flush out all remaining blood from the vasculature. Spleen, Thymus, femurs, and sternum were collected for all mice. Spleen and thymus were processed into single cell suspension for analysis via flow cytometry. Bones were cleaned of muscle using gauze soaked with 70% ethanol and processed for bone marrow to analyze via flow cytometry. Pancreas and colon dissection were performed according to video and instructional JoVE files. The feces were expelled via administering ice-cold PBS with a 22-gauge gavage tube from colon. Other organs like small intestines, cecum, kidneys, lungs, and liver were also collected. All organ weights were recorded after removing excess liquid from the exterior. Soft organs were first fixed in 4% PFA and then transferred to 70% ethanol 24 hrs later and stored at 4° C. Bones were stored directly in 70% ethanol at 4° C. Blood was processed for flow cytometry analysis, and serum collection. CBCs were not done.

All flow cytometry was performed in house. Blood, spleen, thymus, and bone marrow were processed via standard staining protocol for flow cytometry. MACSQUANT 16 was used for flow cytometry. Antibody panels slightly varied between first, second and third dose. Markers in the flow panel after first dose analysis included Live/Dead, hCD45, mCD45, hCD56, hTCRgd, hCD3, hCD8, hCD16, hCD19, hCD14. Markers in the flow panel after second dose included all the above, plus hTCRab and hiTCR. In the blood analysis after second dose, 7AAD, CD14 and CD19 were included together in a dump channel. It was later discovered that that novel AVM-NKT might have a unique CD19 and CD14 expression profile as well. For the third dose panel design, when full sac was performed, all markers mentioned above were assigned unique channels to avoid overlap, but TCRab was not included in the final panel due to open channel limitations.

For cheek bleeds, 70 ul of blood was collected in EDTA coated microtainer and proceeded for immunostaining. Antibodies used in the panel were Mouse CD45 (clone: 30F-11), human CD45 (Clone: 2D1); human CD3 (clone: HIT3a); human CD8 (clone: SK1); human CD16 (clone: 3G8); human CD14 (clone: M5E2); human TCRgd (clone: B1); human CD56 (clone: 5.1H11); human CD19 (clone: HIB19). Blood was stained for live dead marker for 10 min at Room temperature and then for the surface antibodies mentioned above for 20 minutes at room temperature in dark. RBCs were lysed with BD FACS lysing buffer and washed with DPBS and resuspended in 300 ul DBPS. 250 ul of sample was run on MACSquant 16 flow cytometer.

### Example 1-Acute High-Dose of Glucocorticoid Receptor Agonists Results in Near Complete Lymphodepletion of Peripheral Blood Lymphocytes, but Induces a Unique Population of NKT Cells

In international patent application PCT/US2019/054395 the present authors have presented a series of experiments demonstrating that high dose glucocorticoid receptor agonists can cause near complete lymphodepletion of peripheral blood lymphocytes as well as reduce the number of germinal centers in lymphoid organs and deplete thymus lymphocytes. These effects are achieved without substantially affecting cell counts of neutrophils, platelets, RBCs and stem cells (both hematopoietic stem cells, HSCs, and mesenchymal stem cells, MSCs).

Here, studies performed in naïve mice show that administration of high-doses of glucocorticoid receptor agonists results in near complete lymphodepletion of peripheral blood lymphocytes without substantially affecting the cell counts of neutrophils, platelets, red blood cells (RBCs) and stem cells (both HSCs and MSCs). Intriguingly, high-dose glucocorticoid receptor agonists were also found to induce upregulation of NKT cells.

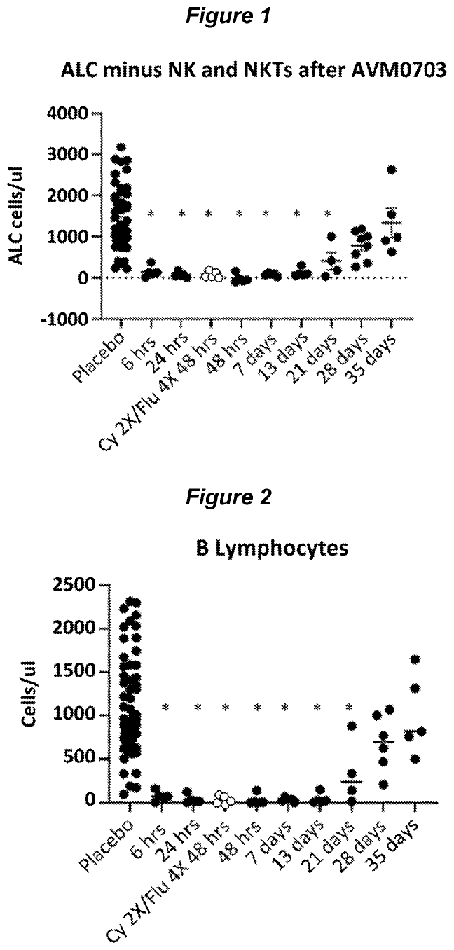

As shown in FIG. 1, in naïve mice, high-dose dexamethasone (18 mg/kg HED DP) significantly reduces absolute lymphocyte count (ALC minus NK and NKT cells) as compared to Placebo—an effect that persists for up to 21 days following administration. At 6 and 48 hours after administration almost complete lymphoablation is observed, with the effect comparable to that achieved with standard Cy/Flu chemotherapy (13 mg/kg HED cyclophosphamide and 0.8 mg/kg HED fludarabine).

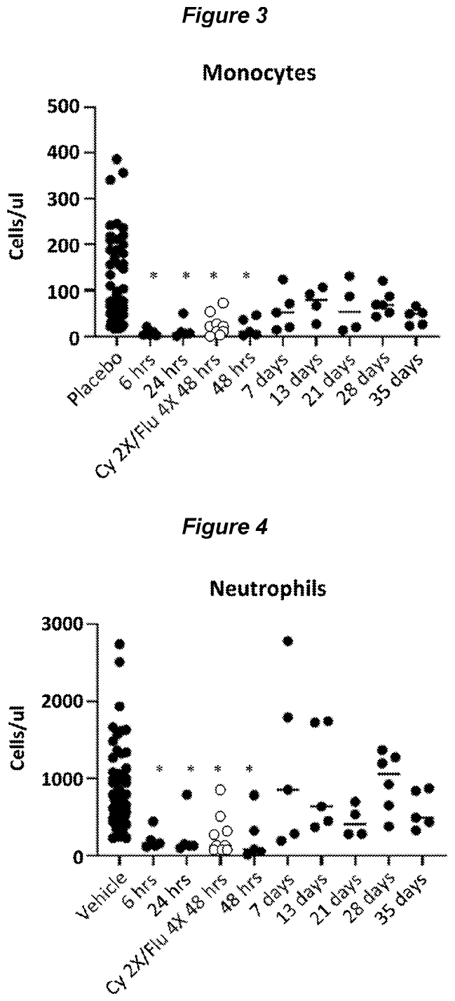

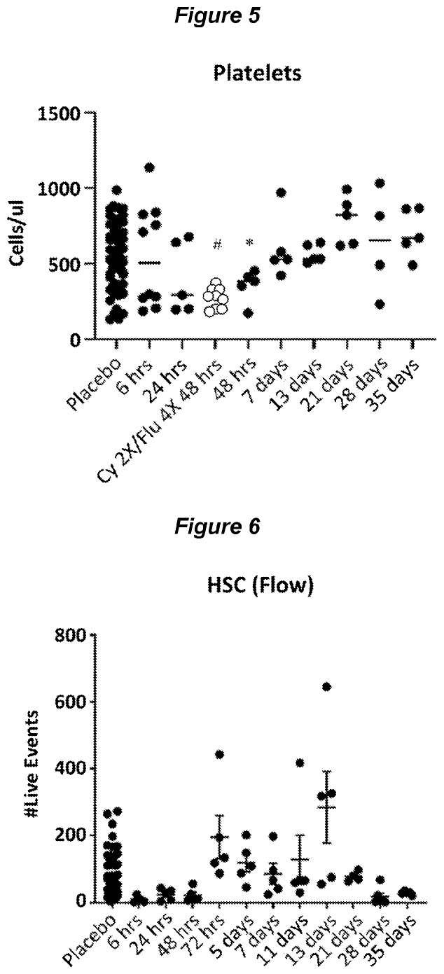

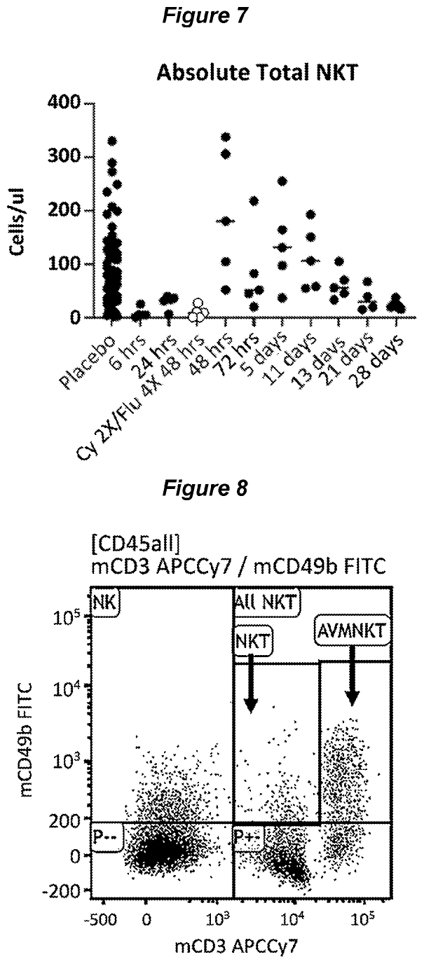

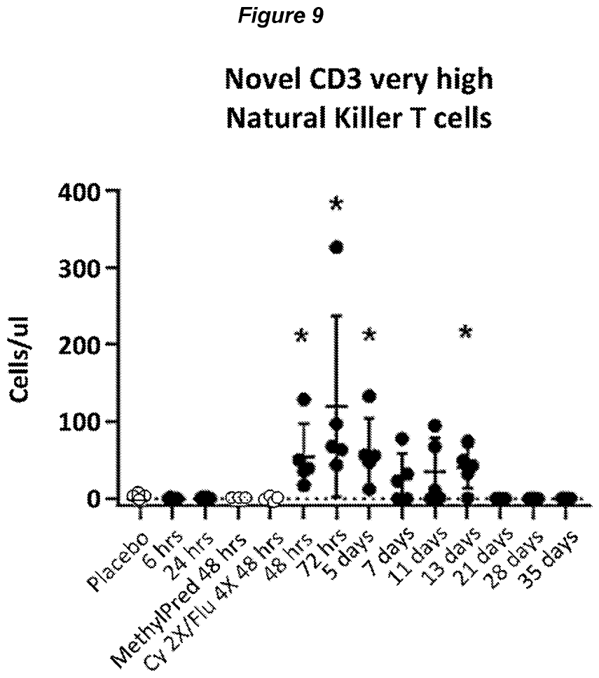

In naïve mice, high-dose dexamethasone selectively ablates T and B lymphocytes (equivalently to standard Cy/Flu chemotherapy; FIG. 2), monocytes (superior to Cy/Flu chemotherapy; FIG. 3), and lymphodepletes neutrophils at the target clinical dose (FIG. 4). Basophils (reduced only at the 6 hour time point), eosinophils (reduced only at the 24 and 48 hour time points), platelets (see FIG. 5), and RBCs are all spared, while HSCs (FIG. 6) and MSCs are spared or increased. (* p<0.05; #p<0.0001).

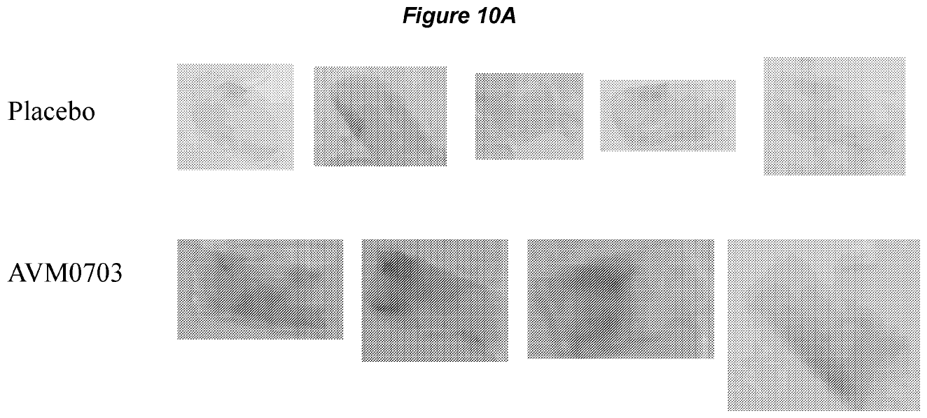

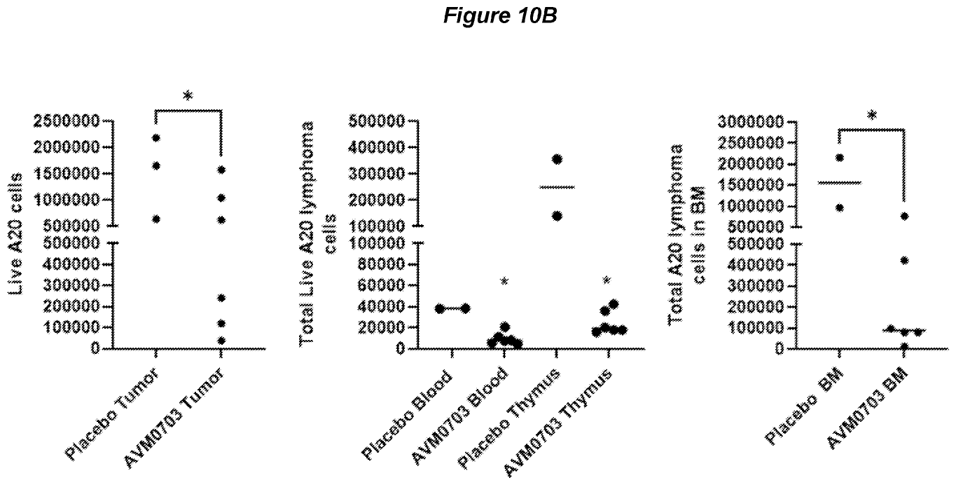

Surprisingly, high-dose dexamethasone was also shown to induce NKT upregulation (FIG. 7) and production of a novel population of NKT cells (AVM-NKT). When examined by flow cytometry these novel AVM-NKT cells are CD49b+ and CD3 very bright (CD3highCD49b+). Previously described NKT cells express CD3 with MFI one log lower than the AVM-NKT cells (CD3medCD49b+; FIG. 8). The AVM-NKT cells appear in the blood of naïve mice 48 hours after administration of high doses (HED 18.1 mg/kg) of the glucocorticoid receptor agonists dexamethasone and betamethasone, but are not induced by standard Cy/Flu chemotherapy.

Dose escalation studies show that a single dose between 6-12 mg/kg HED dexamethasone base can induce AVM-NKT cells. 15 mg/kg HED dexamethasone base induces particularly robust production of the AVM-NKT cells, as does a 6+6 mg/kg HED dosing schedule administered at 6 mg/kg at time 0 and 6 mg/kg 24 hours later.

### Example 2—the AVM-NKT Cell is Responsible for In Vivo T and B Lymphoablation

Mononuclear cells from peripheral blood of naïve male C57Bl/6 mice or single cell splenocytes were incubated with equivalent concentrations of AVM0703 as the peak blood concentrations of acute high dose AVM0703 achieve in vivo. Out to 72 hours after addition of AVM0703 to in vitro peripheral blood mononuclear cells or single cell splenocytes, no apoptosis was observed. The lack of in vitro apoptosis of peripheral blood mononuclear cells or splenocytes indicates that the in vivo lymphoablation is due largely to the induction of the AVM-NKT cells.

### Example 3-AVM-NKT Cells Home to Tumor Sites

In preliminary studies, naïve C57Bl/6 mice were treated with high dose dexamethasone with peripheral blood examined by flow cytometry at pre-determined time intervals to characterize the different immune populations. After treatment with high dose dexamethasone, two NKT populations were identified: NKT cells defined as CD3medCD49b+ and the novel population of AVM-NKT defined as CD3highCD49b+ (FIG. 8).

AVM-NKT cells were found to appear in the blood of naïve mice 48 hours after supra-pharmacologic doses (HED 18.1 mg/kg) of dexamethasone (AVM0703) or betamethasone. Conversely, these cells are not induced by standard Cy/Flu chemotherapy nor by methylprednisone to any significant extent.

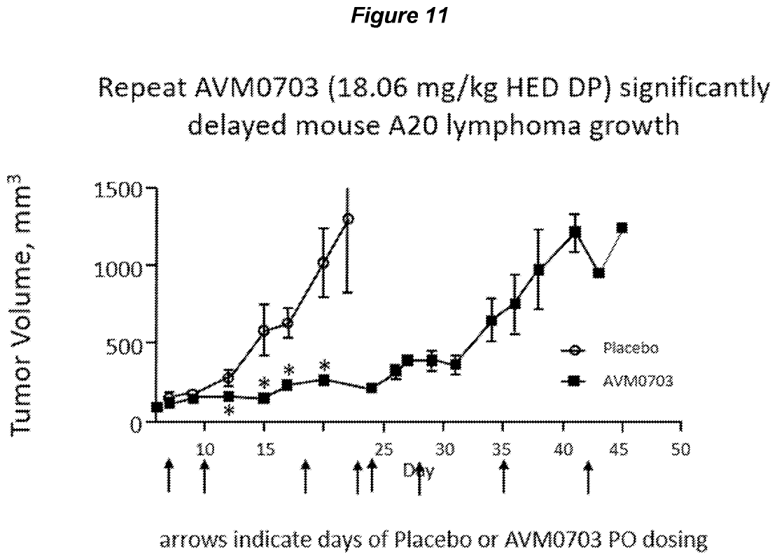

As shown in FIG. 9 and Table 2, in normal mice the AVM-NKT cells are induced in the spleen within 48 hours of dexamethasone dosing, are apparent in peripheral blood from 48 hours after dexamethasone administration, and remain evident in the blood stream until day 13 after dexamethasone administration. AVM-NKT cells are not detected in the spleens of naïve placebo treated mice. Cyclophosphamide/fludarabine dosing does not induce this novel NKT population.

TABLE 2
Presence of AVM-NKT cells in blood, spleen, and tumor in naïve
and A20 mice with and without AVM0703 treatment

| Presence of | | 3 hrs | | 48 hrs | 48 hrs | 48 hrs |
|---|---|---|---|---|---|---|
| AVM-NKT cells | 3 hrs blood | spleen | 3 hrs tumor | blood | spleen | tumor |
| Naïve placebo | ND | ND | NA | negative | negative | NA |
| Naïve AVM0703 | ND | ND | NA | +++ | positive | NA |
| A20 model | + | +++ | + | negative | ND | + |
| placebo | | | | | | |
| A20 model | ++ | −−−− | ++++ | negative | ND | +++ |
| AVM0703 | | | | | | |

NA: not applicable;
ND: not done

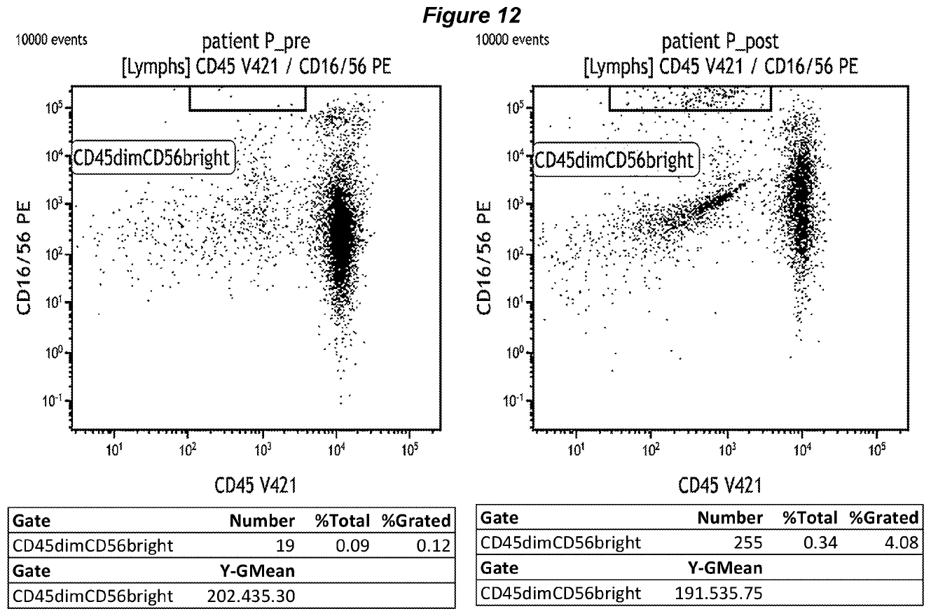

In contrast to the time course of AVM-NKT upregulation observed in normal, disease free mice, quantification of AVM-NKT cells in A20 B cell lymphoma tumor-bearing mice found that AVM-NKT cells are not present in peripheral blood. Instead, in these tumor-bearing mice the AVM-NKT cells appear to home to tumor sites—where increased necrosis is evident when examined 48 hours after dexamethasone administration (FIG. 10A). Additional time course studies demonstrated that the AVM-NKT cells maximally ablate A20 lymphoma implanted in the flanks of mice within 3 hours after 18 mg/kg HED dexamethasone phosphate, while A20 metastasis to blood and thymus is maximally eradicated 24 hours after dosing and A20 metastasis to bone marrow is maximally eradicated 48 hours after dosing (FIG. 10B).

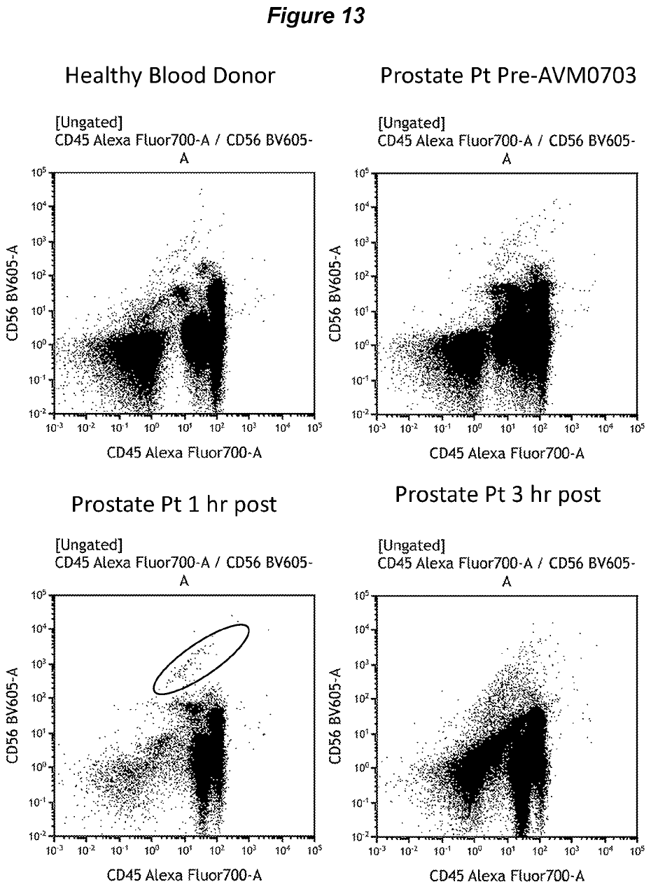

Consistent with this, high dose dexamethasone was shown to significantly delay tumor growth in the A20 model (FIG. 11; Example 6). Because A20 cells undergo only about 30% apoptosis 72 hours after high dose dexamethasone treatment in vitro, it is believed that the AVM-NKT cells play a role in controlling tumor growth.

Two million A20 B lymphoma cells at a cell density of 1.8e7 cells/mL at harvesting were mixed with an equal volume of Matrigel (100 ul each) and injected subcutaneously into the left flank (200 ul total volume) of BALB/c mice, creating a solid tumor model of B cell lymphoma. After tumors were established (approximately 7 days or around ˜100-150 mm³, which is a well-established tumor), mice were treated according to the dosing table shown below. Tumor volumes were measured with calipers three times a week and the tumor volume was calculated using the equation V=L×W2×0.5. Body weights were also taken three times a week and on days of dosing to determine the proper dosage. Mice were considered to be at study endpoint once they reached a tumor volume of 1500 mm³ or had greater than 20% body weight loss. When mice reached study endpoint, they were euthanized as follows. Mice were anesthetized with isoflurane gas. Once anesthetized, blood was drawn via cardiac puncture and then perfused with 10 mL of 5 U/mL Heparin/PBS. The tumor was removed from the right flank by skinning the right posterior side of the mouse. The skin was stretched out and pinned down, and the tumor was separated from the skin by gently scraping with a scalpel. Tumors were fixed for 48 hours before being transferred to 70% ethanol and stored in cassettes at 4° C. Tumors were shipped to HistotoxLabs (Bolder, CO) for sectioning and staining. NKT cells in the tumors were identified by NKp46 staining.

### Example 4-Blood Cancer Enhances the Concentration of AVM-NKT Cells in the Peripheral Blood

Mice are inoculated with T or B cell lymphoma by tail vein injection of 1-5M lymphoma cells in log growth phase. 6 hours to 13 days later blood is harvested from the mice and the AVM-NKT numbers in the blood are determined by flow cytometry gating on CD3 very high (at least 0.5 log higher MFI than T lymphocytes) and CD49b positive cells or by gating on NKp46. Compared to naïve or solid tumor bearing mice, such as T or B lymphoma cells encased in Matrigel and implanted sc in the flank, mice with circulating T or B lymphoma cells have significantly increased numbers of AVM-NKT in the peripheral blood.

### Example 5-AVM-NKT are Induced in Bone Marrow and Fat Tissue 48 Hours after AVM0703 Doses about 29 mg/kg and Higher (Given as DP) in Naïve Balb/c Mice

Balb/c mice have MHC haplotype “d”: H-2K is d (H-2K_{d}). H-2D is d (H-2D_{d}). H2-Lis d (H-2L_{d}). Aαβ is d, d. Eαβ is d, d. Mls1 is b. Mls 2 is a. I-A is d (I-A_{d}). I-E is d (I-E_{d}). Qa-1 is b (Qa-1_{b}). Qa-2 is a (Qa-2ₐ).

C57Bl/6 mice have MHC haplotype “b”: H-2K is b (H-2K_{b}). H-2D is b (H-2 D_{b}). H2-L is null. Aαβ is b, b. Eαβ is b, b. Mls1 is b. Mls 2 is b. I-A is b (I-A_{b}). I-E is mill. Qa-1 is b (Qa-1_{b}). Qa-2 is a (Qa-2ₐ).

The AVM NKT induced in naïve Balb/c mice are CD3 MFI high similar to the peripheral blood AVM-NKT induced in naïve C57Bl/6 mice, and the AVM-NKT in naïve Balb/c mice are TCRgamma/delta positive. Many of the cells are NKp46 negative indicating that they are not activated. This example demonstrates that MHC expression may determine the target organ.

MHC may control the trafficking of AVM_NKT cells: The AVM NKT cells are in blood in naïve AVM0703 treated male C57B16 mice. The AVM NKT cells are in fat and bone marrow in naïve AVM0703 treated male Balb/c mice. The AVM NKT cells are in tumors in AVM0703 treated male tumor bearing Balb/c mice. The new NKT in naïve Balb/c mice are also tCRgd positive, B220−, NKp46+/−, Ly6G−, CD4−, CD8−, CD3high, MFI 10492, and CD49b+.

### Example 6-Acute High-Dose Dexamethasone has Tumor Killing Effects in T Cell and B Cell Lymphoma, Prevents or Delays Hyperglycemia in Spontaneously Diabetic NOD Mice, and Reverses Diabetes in Early Onset and Established Diabetic NOD Mice

High dose dexamethasone was shown to significantly delay tumor growth in the A20 B cell lymphoma tumor model (FIG. 11). A subsequent series of experiments (described in PCT/US2021/019773, the contents of which are hereby incorporated by reference in their entirety) confirmed the tumor killing effect of acute high dose dexamethasone in the A20 B cell lymphoma tumor model and a xenograft model of T cell lymphoma (CCRF-CEM), and demonstrated the ability of high dose dexamethasone to prevent hyperglycemia and reverse diabetes in early onset and established diabetic NOD mice.

TABLE 3
Exemplary dosing schedules

| | | AVM0703 | | | | | | | | | | | | | |
|---|---|---|---|---|---|---|---|---|---|---|---|---|---|---|---|
| | | Dose(s) | | | | | | | | | | | | | |
| | | (DP) mg/kg | | | | | | | | | | | | | |
| Study | Design | HED | Dose 1 | | Dose 2 | | Dose 3 | | Dose 4 | | Dose 5 | Dose 6 | Dose 7 | Dose 8 | Dose 9 |
| A20_1 | Repeat | 18 | 7 | | 10 | | 18 | | 23 | | 24 | 29 | 36 | 43 | 50 |
| | | # End pt | | | | | | | | | | 1 | 3 | 1 | |
| | | after | | | | | | | | | | | | Resis- | |
| | | | | | | | | | | | | | | tance? | |
| | 1-10 totally necrotic was dosed as above; | | | | | | | | | | | | | | |
| | Mouse 1-5 totally necrotic was dosed as shown here | | | | | | | | | | X | X | X | X | X |
| A20-3 | DRC | 7, 18, 25 | 10 | | 17 | | 25 | | 32 | | 39 | 18&26 mg/kg 50-80% necrotic | | | |
| | Saw a necrosis DRC and CD3 labeling inversely related to DRC | | | | | | | | | | | | | | |
| A20_4 | Combo CyFlu | 18 | 1 | AVM | 1 | AVM | | | | | | | | | |
| | | | 1 | AVM | 4 | CyFlu | 67 | AVM | 74 | CyFlu | | | | | |
| | | | | CyFlu | | CyFlu | | | | | | | | | |
| A20_5 | DRC | 18, 22, 25 | 15 | | 31 | | 38 | | 45 | | | | | | |
| A20_7 | AVM, APP, Mylan | 18 | 9 | | 16 | | 23 | | | | | | | | |

A20 is a mouse B lymphoma

| | | | | | | | | | | | |
|---|---|---|---|---|---|---|---|---|---|---|---|
| CCRF- | Xeno human | 18 | 7 | 14 | 21 | 28 | 35 | 42 | 49 | 56 | 63 |
| CEM | T lymphoma | | | | | | | | | | |

| | No tumor growth whatsoever |
|---|---|
| DRC: dose response curve | Dose is how many days after tumor |
| | implantation |

### Example 7-Identification of AVM-NKT Cells in Human Subjects Treated with Acute High Doses of Dexamethasone

Following the identification of AVM-NKT cells in mice, data on file from human subjects treated with high doses of dexamethasone was re-analysed.

In osteoarthritis patients, under the guidelines of “Physician Practice of Medicine,” 3-6 mg/kg generic dexamethasone was administered (by Dr. Loniewski, Advanced Orthopedic Specialists, Brighton, MI) to 4 patients.

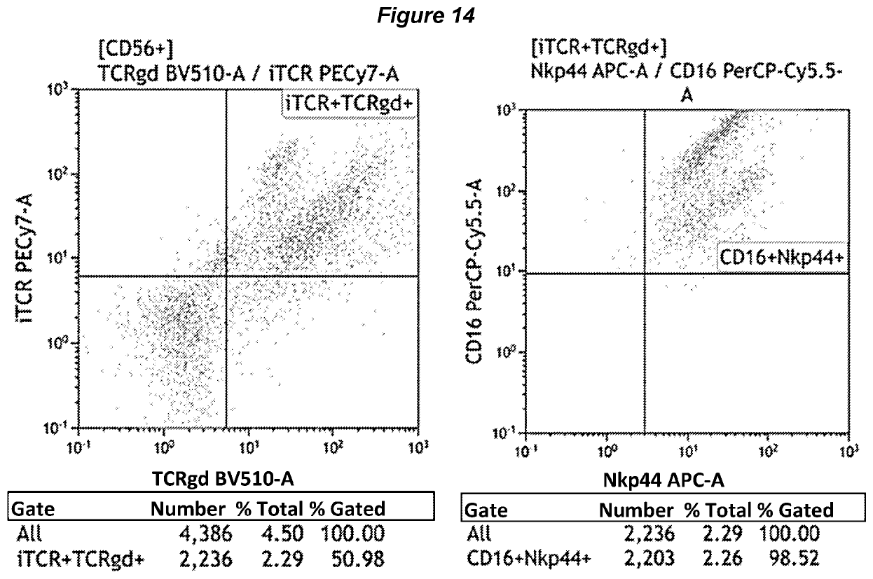

A review of flow cytometry data from the 4 patients taken 48 hours after they were treated with a dexamethasone dose that is 6-fold lower than the dose used to maximally induce AVM_NKT in mice, was performed. CD45/CD56 scattergrams from one of the four patients shows that a new population of cells corresponding to the AVM-NKT cells identified in mice emerged ˜48 hours post treatment (FIG. 12).

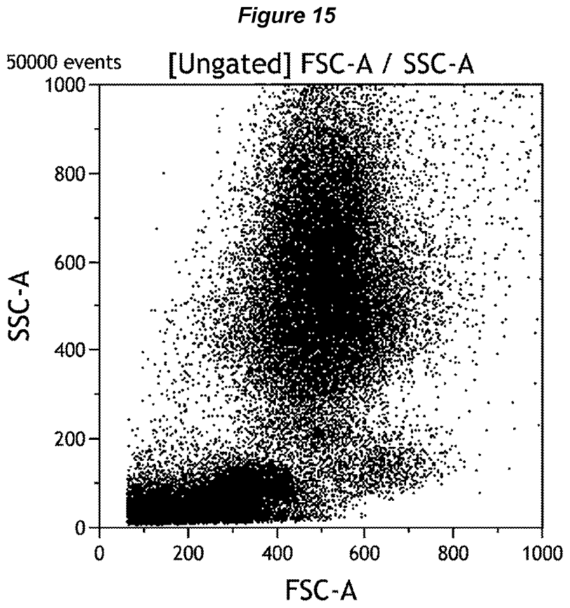

As shown in FIG. 13, a novel CD56 very bright cell population has also been observed in a prostate cancer patient one hour after his fourth AVM0703 treatment was infused at 6 mg/kg. The prostate cancer patient was a no-option patient after multi-year cancer treatment and has received a total of 4 AVM0703 infusions as least 28 days apart.

Compared to a healthy blood donor, the prostate cancer patient had evidence of a novel CD3 dim population, which was no longer evident one hour after AVM0703, however, a new CD56 very bright cell population was then evident in the blood which was no longer observed 3 hours after the infusion.

Compared to a healthy blood donor the prostate cancer patient had a CD3 dim and a NKp46dim population of cells pre-infusion, and one hour post-infusion of AVM0703 at 6 mg/kg the patient has a new CD56 very bright CD3dim population that was CD45 dim/negative and CD4/CD8 double negative.

### Example 8-Production and Mobilization of Human AVM-NKT Cells in Humanized Mice

BRGSF humanized mice on a Balb/c background from Genoway generated by transplanting human umbilical cord blood CD34+ stem cells into irradiated mice that lack mouse B and T lymphocytes and NK cells but have a functional mouse complement system are orally dosed with HED 18-45 mg/kg DSP. 24-48 hours later, a new population of cells corresponding to the AVM-NKT cells identified in non-human mice can be observed. The human CD56+ cells can be observed in the blood between about 36 hours out to 13 days later.

### HuCD34-NCG Mouse Model

The HuCD34-NCG mouse from Charles River is a study-ready mouse model with a human-like immune system, created by adoptive transfer of CD34+ stem cells. HuCD34-NCG mice are an ideal in vivo platform to evaluate the effectiveness of compounds modulating the human immune system. The lack, or late onset, of graft-versus-host disease (GvHD) in humanized mice make them ideal for long-term studies.

NCG mice are humanized by adoptive transfer using human umbilical cord blood-derived CD34+ stem cells, following myeloablation treatment. NCG mice from 4 donors (n=2 per donor) are orally dosed with HED 18-45 mg/kg DSP. 24-48 hours later cells corresponding to the AVM-NKT cells identified in non-human mice can be observed, as about 0.2-3% of total splenocytes by flow cytometry. The human CD56+ cells can be observed in the blood between about 36 hours out to 13 days later.

huNOG-EXL Mouse Model

huNOG EXL from Taconic have an average of 54% of CD45 cells positive for human CD45. Six huNOG EXL humanized immune system mice from 3 donors (n=2 per donor) are orally dosed with HED 18-45 mg/kg DSP. 24-48 hours later cells corresponding to the AVM-NKT cells identified in non-human mice can be observed to be about 0.2-3% of total splenocytes by flow cytometry. The human CD56+ cells can be observed in the blood between about 36 hours out to 13 days later.

### Example 9-Characterisation of AVM-NKT (NKT-Like Cells) Induced/Mobilized in Human Subjects

Preclinically, AVM0703 induced AVM-NKT cells have been characterised, and have demonstrated activity against mouse melanoma, mouse B lymphoma, human xenograft T lymphoma and diabetes.

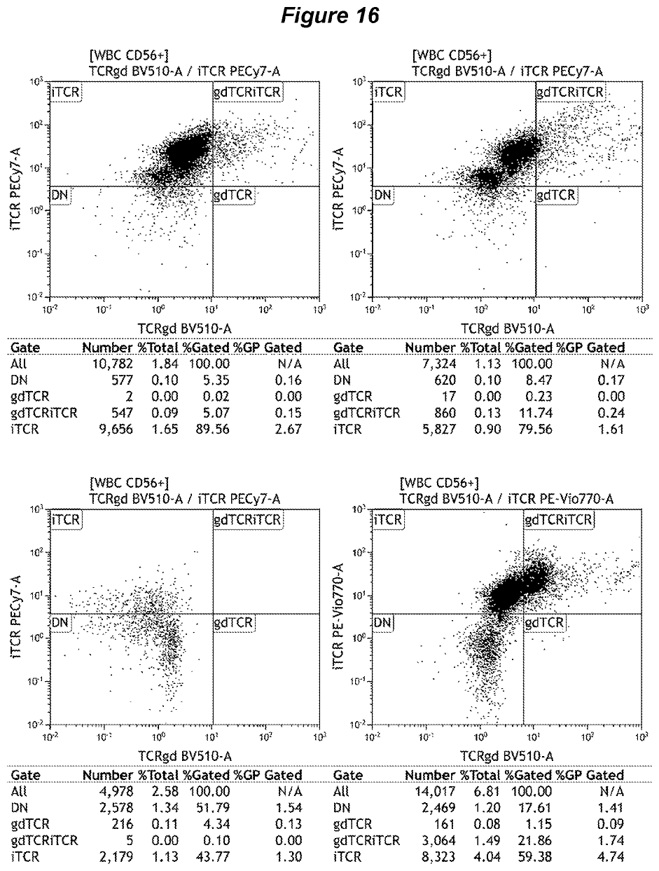

Blood samples were collected from human cancer subjects treated with AVM0703, and cells were characterized by the human whole blood surface staining protocol outlined above. AVM0703, induces the production and mobilization of a γδ Natural Killer T-like cell (CD56+γδTCR+). Intriguingly, the mobilized NKT-like cell was also found to expresses the iTCR (FIG. 14). This finding may explain why AVM0703 induced cells have activity against both cancer and type 1 diabetes, while iNKT and γδT cells are generally described as active against one disease but not the other. The mobilized cells are typically also CD16+ and NKp44+ (FIG. 14).

CD56+γδTCR+ (1.64% of WBC) cells were mobilized into whole blood after AVM0703 administration within 30 minutes post infusion. These cells were gated from all live WBC (white blood cells). In a representative subject, these cells were also positive for iNKT (˜96% of novel cells), NKp44 (˜97% of novel cells), CD8 dim/-(˜98% of novel cells), CD19+ (85% of novel cells), CD16+ (86% of novel cells), CD14+ (67% of novel cells). Numbers reported here are as % CD56+γδTCR+. CD56+γδTCR+iTCR+ cells were also found to express CD3, CD45 and in some cases to not express CD4. Some CD56+γδTCR+iTCR+ cells were also found to express αβTCR.

### Example 10-AVM-NKT Cells are Isolated and Expanded then Used to Precondition a Patient Before a Cell Therapy

Autologous or allogeneic AVM-NKT cells are administered either IV or IP to a patient between 6 to 96 hours before a cell therapy is administered. The cell therapy can be for a regenerative purpose, for treating a cancer, for treating an autoimmune disease or for treating an infection or any other medical condition that warrants cell therapy.

### Example 11-AVM-NKT Induce Tumor Lysis Syndrome

AVM-NKT target to tumors and form bands of attacking cells invading the tumor like an army from all sides. Tumor lysis syndrome occurs, and in mice, cannot be treated and can cause death. Clinical chemistry markers of tumor lysis syndrome are elevated, such as uric acid. Gross examination of tumors shows a sludge-like oil encased in the tumor membrane.

### Example 12-AVM-NKT Cells are Used to Prepare a Patient for Cancer or Other Serious Medical Treatment

Autologous or allogeneic AVM-NKT cells are administered either IV or IP to a patient with a performance status that prevents them from having a medical therapy such as chemotherapy, cell therapy, organ or bone marrow transplant. The patient's performance status improves such that they become eligible for medical treatment.

### Example 13-AVM-NKT Cells Cause Tumor Pseudoprogression

Tumors treated with AVM-NKT cells continue to appear to grow, however, the growth is pseudoprogression of the tumor because of the other immune cells that the AVM-NKT cell attracts to the tumor, either through the release of cytokines and chemokines or by direct engagement of other immune cells. Eventually, the tumor becomes completely acellular and is resorbed.

### Example 14-AVM-NKT Cells is Used to Treat any Type of Cancer, Graft Versus Host Disease, Autoimmunity, or Immune-Related Adverse Events of Immunotherapies

AVM-NKT cells home to and target both blood and solid cancers, and fibroid tumors, benign tumors, and autoreactive T and B lymphocytes.

### Example 15-AVM-NKT Cells are Detected in Human R/R NHL Subjects Treated with Acute High Doses of Dexamethasone as Well as in Humanized Mice Treated with Acute High Doses of Dexamethasone

### SUMMARY

In human R/R NHL clinical trial patients a bi-specific gamma delta TCR+ and invariant TCR+ cell is mobilized into the blood within 30-60 minutes after AVM0703 dosing between 6 mg/kg and 18 mg/kg. This novel induced immune cell is not apparent in the blood of healthy mice in a pathogen free environment or in the blood of healthy human donors to any extent (who are not in a pathogen free environment), however, in the cancer setting, at baseline, patients expressed low levels of this novel bi-specific cell that also expresses CD56 (a Natural Killer marker). Thus, the cells are gamma delta TCR+ invariant TCR+bi-specific Natural Killer T-like cells.

The authors hypothesize that a cancer environment may induce induction of these cells, but that these are not maximally mobilized into the blood until after AVM0703 dosing, similar to results seen in the mouse A20 lymphoma model (FIG. 11, Examples 3 and 6). For one compassionate use patient with CNS squamous cell carcinoma, immune infiltrate, characterized by skin turning red confined to the area of the CNS tumor on the left side of the brain, was observed within 30 minutes after starting an 18 mg/kg AVM0703 infusion (data not shown).

Intriguingly, the presence of these CD56+γδTCR+iTCR+ cells is associated with clinical response in the R/R NHL clinical trial patients. The only patient (108-004) who had no evidence of these cells at baseline or after AVM0703 dosing is the only patient who did not have any objective clinical beneficial response after AVM0703.

In mice, AVM0703 at human equivalent doses (HED) of 18 mg/kg, calculated as dexamethasone phosphate, induces the production of CD3 high cells in the spleen, bone marrow and thymus and mobilization of these cells out of the spleen and into the blood and towards A20 mouse B cell lymphoma whether the A20 cells are in a solid tumor injected in the flank, in the bone marrow, in the spleen, in the thymus or in the blood. The most rapid and primary mobilization is to the tumor where maximal effect to kill A20 is observed ˜3 hours after dosing.

Naïve mice do not typically express these cells in any organ examined. Different strains of mice have different sensitivity in response to AVM0703 to induce and mobilize these cells, and the tumor environment itself may induce the production of these cells in the spleen but require AVM0703 for optimal mobilization and tumor targeting of the cells.

Humanized mice purchased from both Charles River and Taconic bearing a human lymphoid compartment also mobilize a hCD45+CD56+γδTCR+invTCR+ cell after AVM0703 dosing.

Summary of Human Data from AVM0703 R/R NHL Clinical Trial Patients

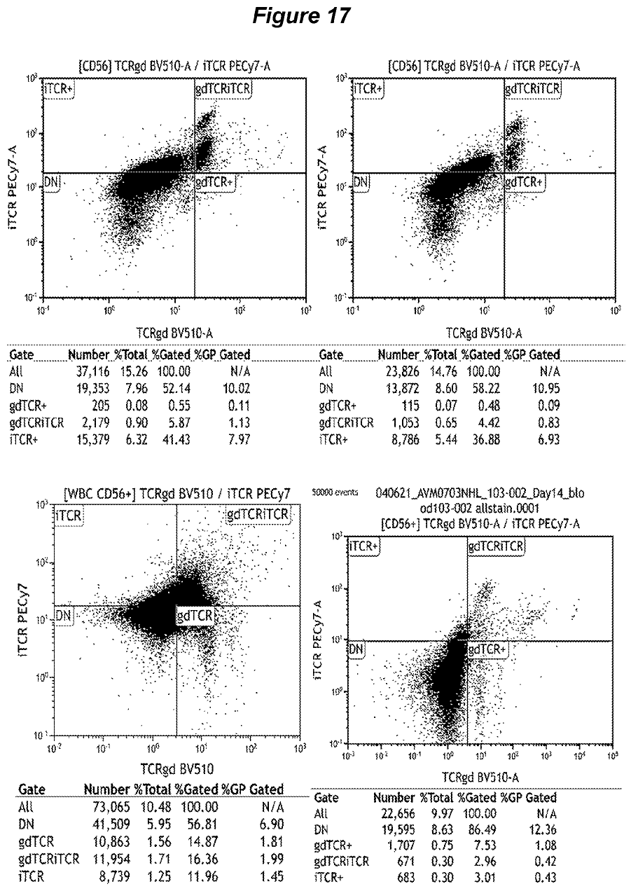

101-001 (6 mg/kg): CD56+γδTCR+ are present at low levels in the blood pre-infusion and increase from 1.6% (76 cells/uL blood) to 3.48% (165 cells/uL blood) of all CD45+ cells one hour after 6 mg/kg AVM0703 infusion. The cells are also invTCR+ and in this patient also αβTCR+ when CD56+γδTCR+ cells are gated into histograms for invTCR and αβTCR expression. By size and complexity they are predominantly large granular lymphocyte-like cells (appearing as red dots on FSC vs SSC plots; FIG. 15).

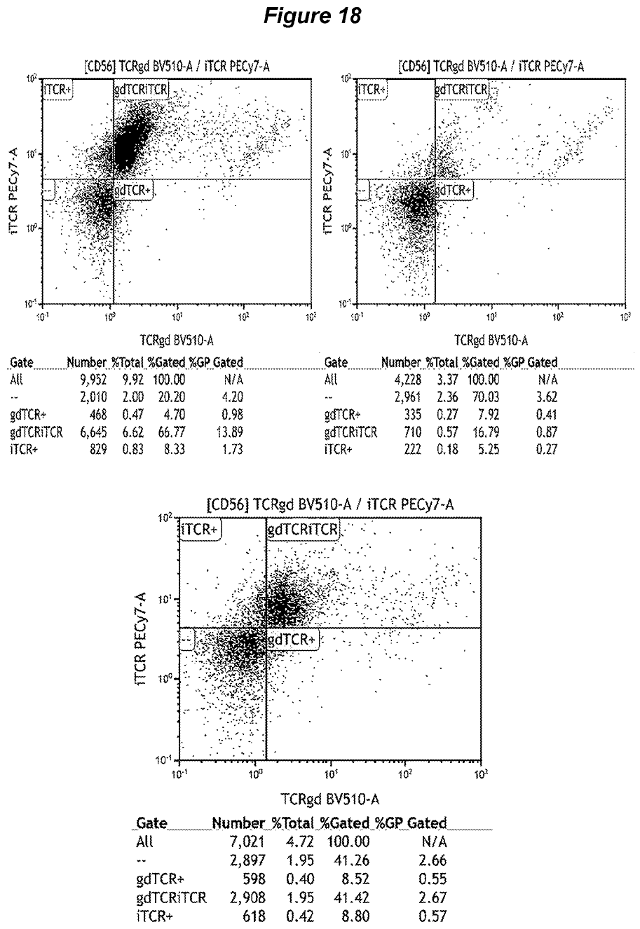

Using a different gating approach gating CD56+ WBCs into a scattergram with γδTCR on X-axis and invTCR on Y-axis, CD56+γδTCR+iTCR+ triple positive cells are present among WBCs at 0.15% pre-infusion (7 cells/uL) and 0.24% 1 hour post-infusion (11.4 cells/uL). These cells are not evident on day 3 post-infusion suggesting homing of the cells to the tumor sites, and are 1.74% of all WBCs on day 14 (78.7 cells/uL). This patient had evidence of tumor flare and stable disease (SD) by day 28 PET/CT. These data are shown in FIG. 16 and, together with % GP gated (GP is WBCs), are summarised in table 4 below.

TABLE 4
101-001 CD56+ γδTCR+ invTCR+ characteristics

| CD56+ γδTCR+ | CD56+ | | | | | | |
|---|---|---|---|---|---|---|---|
| ITCR+ % of | γδTCR+ | Below as % of CD56+ γδTCR+ iTCR+ cells | | | | | |
| total WBCs | ITCR+ cells/uL | CD19+ | TCRαβ | CD8med | CD8hi | CD3 | CD45 |

| | | | | | | | |
|---|---|---|---|---|---|---|---|
| Pre-inf | 0.15 | 7 | 55 | 99 | 84 | 56 | 99 |
| 1 hour | 0.24 | 11.4 | 54 | 100 | 86 | 39 | 99 |
| Day 3 | 0.00 | | | | | | |
| Day 14 | 1.74 | 78.7 | 22 | 99 | 94 | 99 | 99.5 |

103-002 (6 mg/kg): CD56+γδTCR+ cells were high pre-infusion (9.3% of all CD45+ cells, 735 cells/uL blood) and reduced in the blood 1 hour post-infusion (6.54% of all CD45+ cells, 517 cells/uL blood). Since this patient had evidence of tumor flare and a PR by day 28 PET/CT this suggests that AVM-NKT were targeted to the tumor after AVM0703 infusion. This patient's cells are not all bi-specific, with only about 10% of CD56+γδTCR+ cells co-expressing invTCR. This patient also expressed higher levels of CD8, including CD8 MFI high cells.

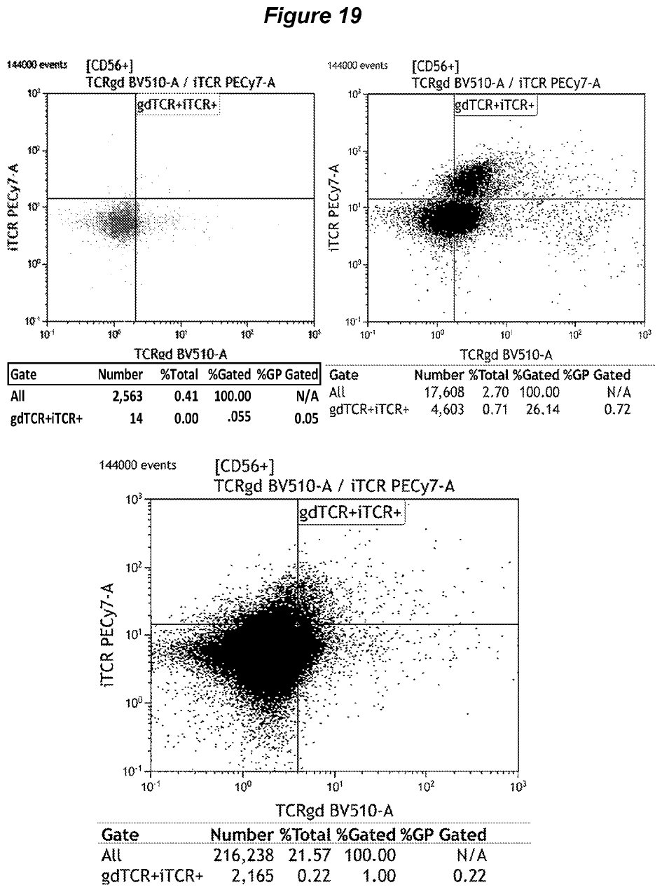

Using a different gating approach gating CD56+ WBCs into a scattergram with γδTCR on X-axis and invTCR on Y-axis, CD56+γδTCR+iTCR+ triple positive cells were measured and expressed markers characterized. On day 3 there was no CD19 staining of any cell except 4% of the CD56+γδTCR+iTCR+ cells, although a lymphocyte population was obvious by FCS vs SSC and only 60% were CD3 positive by flow. These data are shown in FIG. 17 and summarised in table 5 below.

TABLE 5
103-002 CD56+ γδTCR+ invTCR+ characteristics

| | CD56+ γδTCR+ | CD56+ | | | | | | |
|---|---|---|---|---|---|---|---|---|
| | iTCR+ % of | γδTCR+ | Below as % of CD56+ γδTCR+ iTCR+ cells | | | | | |
| | total WBCs | iTCR+ cells/uL | CD19+ | TCRαβ | CD8med | CD8hi | CD3 | CD45 |
| Pre-inf | 1.13 | 89.3 | 0.91 | 72 | 4.6 | 3.9 | 5.6 | 81 |
| 1 hour | 0.83 | 65.6 | 0.78 | 79 | 2.9 | 2.6 | 4.2 | 92 |
| Day 3 | 1.99 | 366.2 | 4.2 | 54 | 30 | 2 | 67 | 17 |
| Day 14 | 0.42 | 27 | 1.76 | 78 | 70 | 2.4 | 31.5 | 98 |

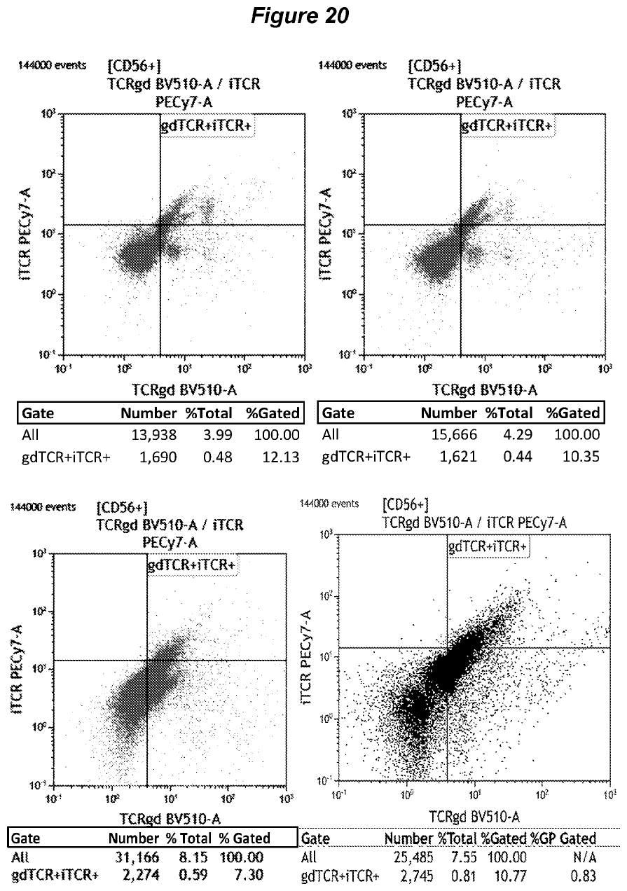

103-005 (9 mg/kg): Baseline CD56+γδTCR+ cells were 2.7% of CD45+ cells and all were bi-specific for invTCR (FIG. 18 upper left). αβTCR was not included in this flow panel. They were largely CD8 negative but 95% were CD14 positive and 60% were CD16 positive indicating an activated state. 13% were CD19 positive.

One hour after 9 mg/kg AVM0703 (FIG. 18 upper right) CD56+γδTCR+ cells decreased to 0.21% of CD45+ cells and since this patient had evidence of tumor flare and a PR by day 28 PET/CT, this suggests that AVM-NKT cells were targeted to the tumor sites.

Using a different gating approach gating CD56+ WBCs into a scattergram with γδTCR on X-axis and invTCR on Y-axis, CD56+γδTCR+iTCR+ triple positive cells were measured and expressed markers characterized. This patient, who had a clinical response noted by the PI within 1 week and had a PR by Day 28 PET/CT, had high levels of the cells that went down in blood dramatically one hour after 9 mg/kg AVM0703 infusion, suggesting tumor homing. These data are shown in FIG. 18 and summarised in table 7 below.

TABLE 7
103-005 CD56+ γδTCR+ invTCR+ characteristics

| | CD56+ | CD56+ | | | | | | | |
|---|---|---|---|---|---|---|---|---|---|
| | γδTCR+ | γδTCR+ | | | | | | | |
| | iTCR+ % of | iTCR+ | Below as % of CD56+ γδTCR+ iTCR+ cells | | | | | | |
| | total WBCs | cells/uL | CD19+ | CD14+ | CD8med | CD8hi | CD16 | CD3 | CD45 |
| Pre- | 13.89 | 347.3 | 8 | 76 | 4 | 2 | 67 | 30 | 98 |
| inf | | | | | | | | | |
| 1 | 0.87 | 21.8 | 33.9 | 76 | 17 | | 66 | 6 | 99 |
| hour | | | | | | | | | |
| Day 3 | 2.67 | 82.8 | 28 | 8 | 15 | | 84 | 10 | 63 |
| Day | 0.66 | 22.4 | 99 | 81 | 16 | | 37 | 66 | 84 |
| 14 | | | | | | | | | |

103-006 (9 mg/kg): Baseline CD56+γδTCR+ cells were 3.84% of CD45+. αβTCR and invTCR were not included in this flow panel. The CD56+γδTCR+ cells were also positive for CD16, CD34 and ICAM3 (ICAM3 MFI was 294 compared to healthy control MFI of 760), and 42% were positive for NKp44. They were largely CD8 negative but 95% were CD14 positive and 20% were CD16 positive indicating an activated state. 13% were CD19 positive.

One hour after 9 mg/kg AVM0703 CD56+γδTCR+ the cells had not changed by number or expression in the blood, except only 25% were then NKp44+. There was no change in ICAM3 MFI. On day 3 the CD56+γδTCR+ cells were 2.2% of all WBCs and 92% expressed CD16, 19% expressed NKp44, and ICAM3 MFI was 240. On day 14 the CD56+γδTCR+ cells were 2.2% of all WBCs and 92% expressed CD16, 19% expressed NKp44, and ICAM3 MFI was 240. On day 14 the CD56+γδTCR+ cells were only 0.11% of all WBCs.

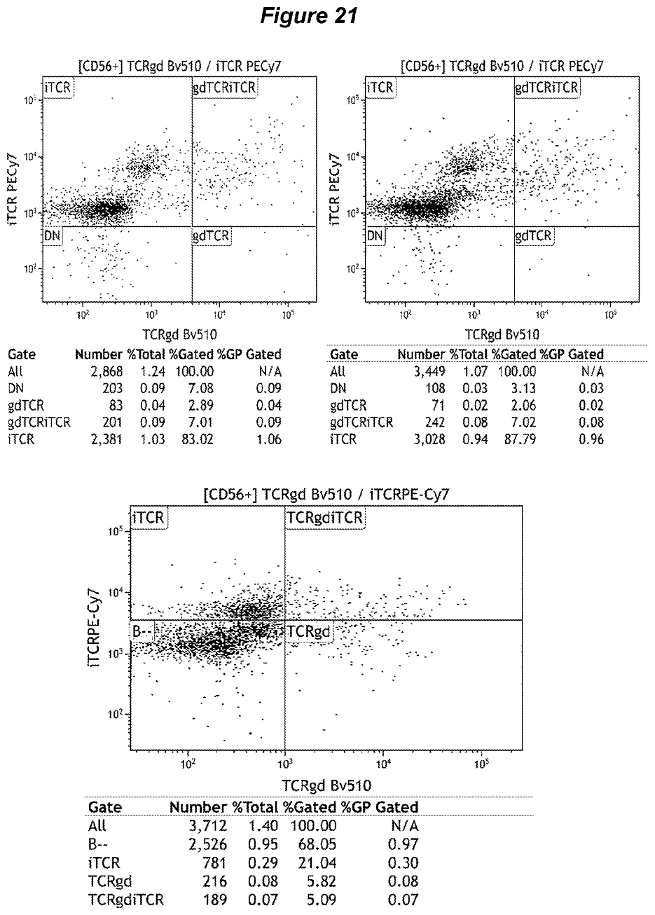

108-001 (9 mg/kg): One hour after AVM0703 infusion CD56+γδTCR+iTCR+ cells in blood increased ˜20 fold from baseline (5.8 to 112 cells/uL), and remained elevated on day 3 (36 cells/uL). This patient had a significant clinical response with restoration of vision on day 3 after AVM0703 infusion. Interestingly there were very few CD19+ lymphocytes at baseline or any time point for this patient. The CD56+γδTCR+iTCR+ flow cytometry characterization after the first infusion shown in FIG. 19 and summarized in table 8 below. αβTCR was not included in this flow panel.

TABLE 8
108-001 1^{st} infusion CD56+ γδTCR+ invTCR+ characteristics

| | CD56+ | CD56+ | | | | | | | |
|---|---|---|---|---|---|---|---|---|---|
| | γδTCR+ | γδTCR+ | | | | | | | |
| | iTCR+ % of | iTCR+ | Below as % of CD56+ γδTCR+ iTCR+ cells | | | | | | |
| | total WBCs | cells/uL | CD19+ | CD14+ | CD8med | CD8hi | CD16 | CD3 | CD45 |
| Pre- | 0.05 | 5.8 | | | | | | | |
| inf | | | | | | | | | |
| 1 | 0.72 | 112 | 11.3 | 35 | 11 | 1 | 90 | 4 | 96 |
| hour | | | | | | | | | |
| Day 3 | 0.22 | 36.0 | 24 | 50 | 16 | 2 | 80 | 24 | 98 |
| Day | 0.06 | 3 | | | | | | | |
| 14 | | | | | | | | | |

The AVM_NKT CD56+γδTCR+iTCR+ cells at baseline or 1 hour after a second 9 mg/kg AVM0703 infusion, but had increased 10 fold on day 3. This patient had an ongoing response with CNS solid tumor not evident and CSF blasts reduced by 40% after the second 9 mg/kg infusion. Data are summarised in table 9 below.

TABLE 9
108-001 2^{nd} infusion CD56+ γδTCR+ invTCR+ characteristics

| | CD56+ | CD56+ | | | | | | | |
|---|---|---|---|---|---|---|---|---|---|
| | γδTCR+ | γδTCR+ | | | | | | | |
| | iTCR+ % of | iTCR+ | Below as % of CD56+ γδTCR+ iTCR+ cells | | | | | | |
| | total WBCs | cells/uL | CD19+ | CD14+ | CD8med | CD8hi | CD16 | CD3 | CD45 |
| Pre- | 0.98 | 31 | 29 | 85 | 6 | 2 | 80 | 17 | 13 |
| inf | | | | | | | | | |
| 1 | 0.84 | 26 | 26 | 81 | 4.4 | 2.2 | 89 | 23 | 13 |
| hour | | | | | | | | | |
| Day 3 | 0.19 | 10.2 | 17 | 35 | 55 | 1 | 58 | 49 | 11 |
| Day | 0.43 | 20 | 28 | 76 | 20 | 4 | 82 | 22 | 84 |
| 14 | | | | | | | | | |

The AVM_NKT CD56+γδTCR+iTCR+ cells were not evident in the blood after the third 9 mg/kg AVM0703 infusion, consistent with the patient's loss of response to the third infusion.

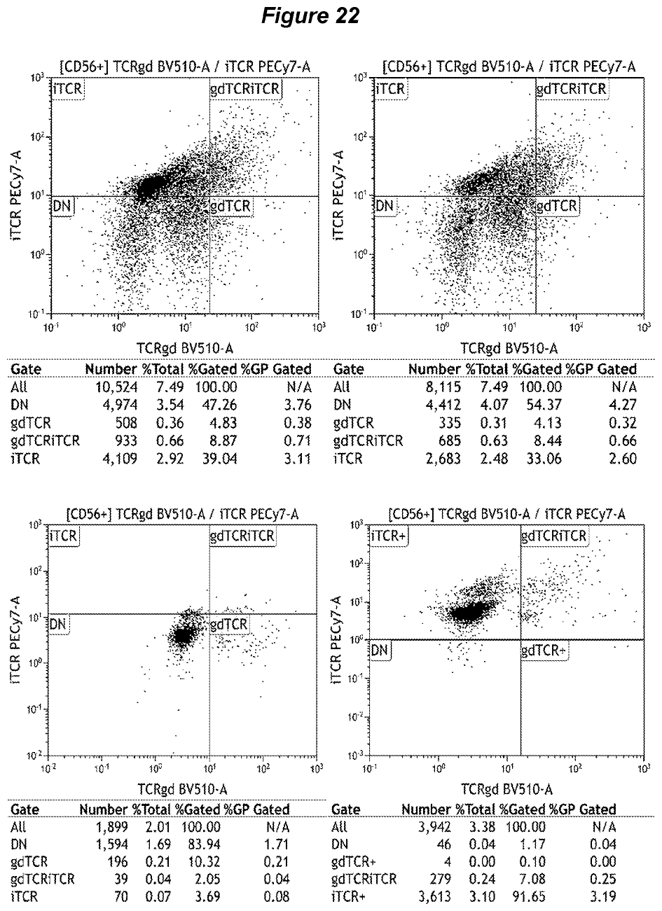

108-003 (12 mg/kg): Data for CD56+γδTCR+iTCR+ cells in patient 108-003 are shown in FIG. 20 and summarised in Table 10 below.

TABLE 10
108-003 CD56+ γδTCR+ invTCR+ characteristics

| | CD56+ | CD56+ | | | | | | | |
|---|---|---|---|---|---|---|---|---|---|
| | γδTCR+ | γδTCR+ | | | | | | | |
| | iTCR+ % of | iTCR+ | Below as % of CD56+ γδTCR+ iTCR+ cells | | | | | | |
| | total WBCs | cells/uL | CD19+ | CD14+ | CD8med | CD8hi | CD16 | CD3 | CD45 |
| Pre- | 0.3 | 23.9 | 40 | 54 | 21 | 4 | 70 | 20 | 96 |
| inf | | | | | | | | | |
| 1 | 0.4 | 31.8 | 40 | 43 | 9 | 1.7 | 85 | 14 | 95 |
| hour | | | | | | | | | |
| Day 3 | 0.6 | 58.4 | 11.5 | 68 | 45 | 12.6 | 75 | 80 | 67 |
| Day | 0.83 | 94 | 12.4 | 87 | 84 | 8 | 89 | 90 | 94 |
| 14 | | | | | | | | | |

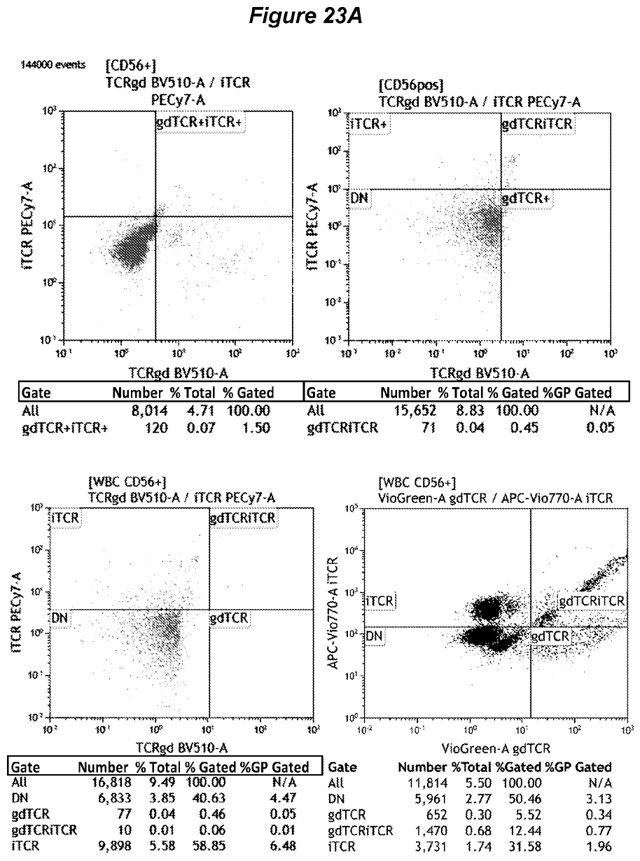

108-004 (12 mg/kg): Pre-infusion CD56+γδTCR+invTCR+ cells are 0.09% of total cells (4.6 cells/uL). There was no increase one hour, 3 days or 14 days after AVM0703 infusion. Interestingly, Patient 108-004 is the only patient who did not have an objective beneficial response measured by either PET/CT, clinical chemistries, CBCs or clinical symptoms. As shown in FIG. 21, 108-004 did not mobilize CD56+γδTCR+invTCR+ cells.

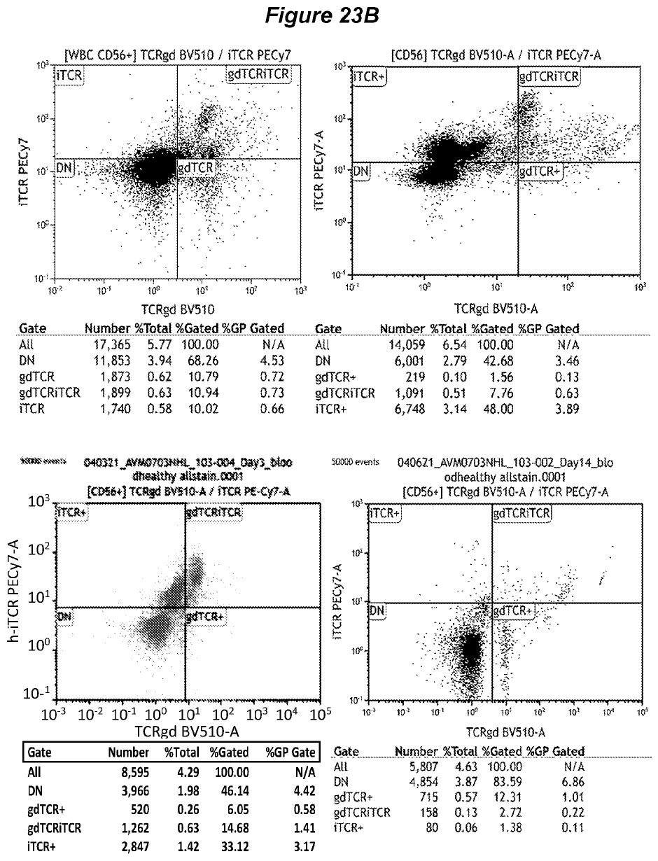

108-002 (18 mg/kg): 2.8% of all WBCs were CD56+γδTCR+iTCR+ (163.2 cells/uL) at baseline which was not changed after AVM0703 18 mg/kg infusion at the one hour time point. On day 3 these novel cells were reduced to 0.04% in the blood, suggestive of tumor homing as has been observed in mouse models, and on day 14 they had returned to 2.12% in the blood. Patient 108-002 has had an ongoing PR with SD by PET/CT and has survived since 30 Aug. 2021 dosing date. Data are shown in FIG. 22 and summarised in table 11 below.

TABLE 11
108-002 CD56+ γδTCR+ invTCR+ characteristics

| | CD56+ | CD56+ | | | | | | | |
|---|---|---|---|---|---|---|---|---|---|
| | γδTCR+ | γδTCR+ | | | | | | | |
| | iTCR+ % of | iTCR+ | Below as % of CD56+ γδTCR+ iTCR+ cells | | | | | | |
| | total WBCs | cells/uL | CD19+ | CD14+ | CD8med | CD8hi | CD16 | CD3 | CD45 |
| Pre- | 0.71 | 53.3 | 97 | 86 | 18 | 82 | 57 | 80 | 100 |
| inf | | | | | | | | | |
| 1 | 0.66 | 49.5 | 98 | 79 | 12.3 | 88 | 87 | 83.2 | 100 |
| hour | | | | | | | | | |
| Day 3 | 0.04 | 2.3 | | | | | | | |
| Day | 0.25 | 9.7 | 80 | 80 | 16.5 | 73 | 41.2 | 72 | 84 |
| 14 | | | | | | | | | |

Summary of Dose-Escalation AVM-NKT and Association with Clinical and Pet/Ct Responses

Summary of AVM0703 for R/R NHL, dose-escalation phase is shown in Table 12 below. As of 15 Jul. 2022; 12 patients dosed; Average 5.6 prior lines with 7 of 12 having failed HSCT or CarT. The only patient with flow cytometry measured who did not have evidence of the novel AVM-NKT also had no response to treatment (108-004). All patients with evidence of the novel AVM-NKT cells had clinical and/or PET/CT response of SD/PR/CR.

| AVM0703 | 6 mg/kg | | | 9 mg/kg | | |
|---|---|---|---|---|---|---|
| monotherapy | 101-001 | 103-002 | 103-004 | 108-001 | 103-005 | 103-006 |
| (X# infusions) | (X1) | (X1) | (X1) | (X3) | (X1) | (X1) |
| Diagnosis | B-ALL | PTCL | T-ALL | CNS B- | MCL | DLBCL |
| | | | | ALL | | |
| Heavily | 5 + 2 | 4 + | 5 | 5 + 1 | 9 + | 2 + 1 |
| Pretreated - | alloHSCT + 6 | auto | | XRT | CarT | XRT |
| prior lines | XRT | HSCT | | | | |
| Survival from | 4.8 | 7 | 2.7 | >11.8 | 8.3 | >12.6 |
| 1^{st} AVM0703 | | | | ongoing | | ongoing |
| (months) | | | | | | |
| central Lugano | SD | PR | PR | ND | PR | ND |
| 28 Day | | | | | | |
| Objective | | | | A | | |
| Clinical | | | | | | |
| Response | | | | | | |
| CD56+gdTCR+ | YES | YES | YES | YES | YES | NE |
| inv TCR+ cells | | | | | | |
| induced | | | | | | |
| +additional | +2 | +3 | none | +2 + CarT | +1 + CarT | CarT + 1 |
| anti-cancer | | | | | SD | CR |
| therapy | Compassionate | Venetoclax + | | HD MTX | IGM | Tisagenlecleucel + |
| (number lines, | use | romidepsin | | | clinical | IL-7 |
| response and | program | Cisplatin − | | IT AraC | trial | |
| therapy | AVM0703 | gemcitabine | | | | |
| details) | 18 mg/kg | | | | | |
| | Vincristine | bendamustine | | Brexacabtagene | CarT | Polatuzumab + |
| | | | | Autoleucel | | Mosunetuzumab |
| Survival | 4.8 | 7 | 2.7 | >11.8 | 8.3 | >12.6 |
| (months) | | | | | | |

| AVM0703 | 12 mg/kg | | | 18 mg/kg | | |
|---|---|---|---|---|---|---|
| monotherapy | 108-003 | 108-004 | 103-007 | 108-002 | 103-008 | 103-009 |
| (X# infusions) | (X2) | (X1) | (X1) | (X1) | (X1) | (X1) |
| Diagnosis | AITCL | DLBCL | MCL | PTCL | DLBCL | DLBCL |
| Heavily | 1 + | 2 + | 1 + | 4 | 8 + | 3 + |
| Pretreated - | auto | CarT | Splx + | | CarT | HSCT + |
| prior lines | HSCT | | 1 XRT | | | CarT |
| Survival from | >10.2 | >8 | >7.6 | >10.3 | >2.1 | NE |
| 1^{st} AVM0703 | ongoing | ongoing | ongoing | ongoing | ongoing | |
| (months) | | | | | | |
| central Lugano | PD | PD | ND | PR/SD | ND/NE | |
| 28 Day | | | | | | |
| Objective | B | | C | | D | |
| Clinical | | | | | | |
| Response | | | | | | |
| CD56+gdTCR+ | YES | NO | NE | YES | NE | NE |
| inv TCR+ cells | | | | | | |
| induced | | | | | | |
| +additional | +2 + CarT | + 2 | +2 | none | none | none |
| anti-cancer | SD | SD | CR | | | |
| therapy | ICE | PBR | R- | | | |
| (number lines, | | | GEMOX + | | | |
| response and | Romidepsin | | Acalabrutinib | | | |
| therapy | | | | | | |
| details) | | | | | | |
| | CarT | Loncastuximab | Rituximab | | | |
| | | Tesirine | | | | |
| Survival | >10.2 | >8 | >7.6 | >10.3 | >2.1 | |
| (months) | | | | | | |

ND not done
NE not evaluable/not evaluated yet
CR complete remission
PR partial response
SD stable disease
PD progressive disease
XRT radiation monotherapy
Splx splenectomy
Objective Clinical Responses: A 3 month vision restoration B pharyngitis & local Lugano 28 day SD C Lymphocytosis reversed & hemoglobin increased D increased edema/inflammation brain stem tumor by MRI
CR: after additional anti-cancer therapy 2/10 evaluable
PR: to monotherapy 4/10 evaluable
SD: to monotherapy 2/10 evaluable
Clinical Response Only: 2/11 evaluable
CR + PR + SD + Clinical Response = 10 of 11 evaluable patients

Summary of Data from Human Healthy Control Blood Donors

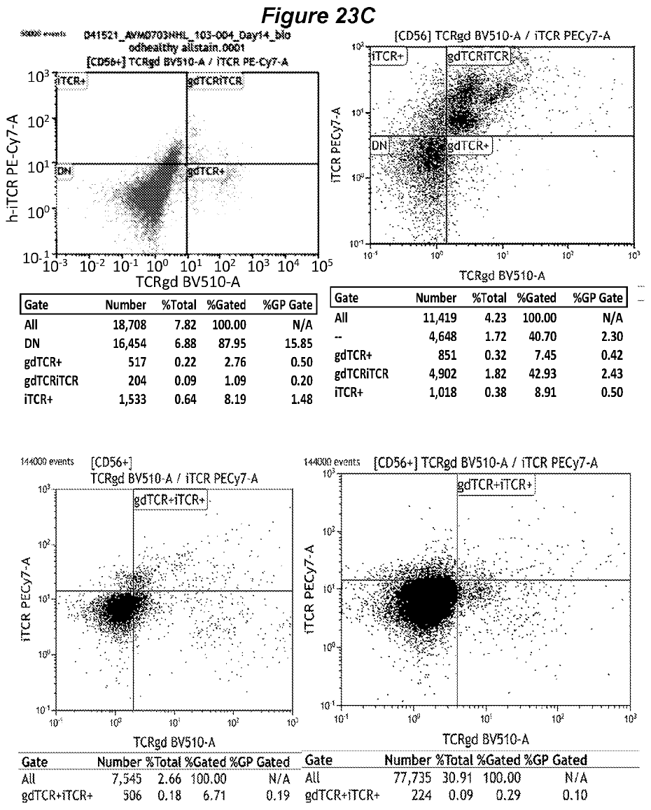

In healthy control blood donors: CD56+γδTCR+ cells are typically not present. The few cells that are present are typically invTCR co-expressers, and also positive for CD14 and CD16. Scatter plots of γδTCR+iTCR+ cells from total CD56+ WBCs are shown for 12 healthy blood donors in FIG. 23.

CD56+γδTCR+iTCR+ marker expression for healthy donors who have some low levels apparent in their blood is shown in table 13 below. The markers expressed are consistent with the markers expressed by these cells in the blood of our R/R NHL patients and we hypothesize that these ‘healthy’ blood donors may actually have an infection or other asymptomatic issue that has induced the production of these cells that are never seen in placebo mice who are kept in a pathogen free environment. The characteristics of these cells when they are present in our healthy blood donors is not dissimilar to the characteristics of these cells in AVM0703-001 trial patients. The % GP gated is the % listed in the tables (GP is WBCs).

TABLE 13
CD56+ γαTCR+ invTCR+ cell characteristics in apparently healthy blood donors

| | CD56+ | | | | | | | |
|---|---|---|---|---|---|---|---|---|
| Healthy | γαTCR+ | | | | | | | |
| donor | ITCR+ % of | Below as % of CD56+ γαTCR+ iTCR+ cells | | | | | | |
| for: | total WBCs | CD19+ | TCRαβ | CD8med | CD8hi | CD16 | CD3 | CD45 |
| 103-004 | 0.77 | 100 | 100 | 82 | 17 | | 99 | 100 |
| D0 | | | | | | | | |
| 103-002 | 0.73 | 50 | 46 | 40 | 9 | | 54 | 74 |
| D3 | | | | | | | | |
| 103-005 | 2.43 | 29 | 11 | 22 | 0 | 85 | 14 | 57 |
| D0 | | | | | | | | |

Summary of Data from Humanized Mice Experiments LYDEP 43 and 45

In mice with partially human blood cells, created by irradiating newborn mice and engrafting umbilical cord (UC) CD34+ cells, novel human immune cells, similar to cells observed in naïve mice and human patients treated with AVM0703, are increased in the blood after AVM0703 treatment, when treated mice are compared to placebo mice derived from the same human UC blood CD34+ donor. When mice were redosed 1 week later, a larger number of mice increased hCD45+CD56+ TCRγδ+ human immune cells compared to placebo treated mice. These humanized mice lack myeloid cells, both mouse and human, and intriguingly, after AVM0703 they began to make both human and mouse myeloid cells. Additionally, after the third AVM0703 dose the humanized mice had hCD45+mCD45+ double positive cells.

Female huNOG-EXL mice (n=6) were obtained from Taconic Bioscience (Germantown, NY). Female huCD34-NCG mice (n=8) were obtained from Charles River (Wilmington, MA). Mice from both facilities were acclimated to laboratory conditions for at least 5-6 days.

Mice were dosed three times orally with 32 mg/kg Dexamethasone Phosphate (DP) or placebo and kept until timepoint. After first (03/01/2021) and second dose 1 week later (03/08/2021), when mice reached predetermined time point, they were bled up to 70 μL of blood/mouse via cheek puncture. The blood was analyzed via flow cytometry.

After third dose 28 days after the previous dose (on Apr. 5, 2021), when mice reached the study time point of 48 or 60 hours, they were euthanized under standard operating procedure.

The female humanized mice studied were from a total of 6 different umbilical cord blood donors and two different vendors. Response to AVM0703 was consistent across both vendors and all 6 donors and is summarized below. AVM0703 induced the expression and mobilization of human CD56+γδTCR+invTCR+ immune cells and also induced myeloid cell production in mice that largely lack myeloid compartments.

TABLE 14
Humanized mice vendor and umbilical cord blood donor

| Vendor | Donor | Placebo treated | AVM0703 treated |
|---|---|---|---|
| CRL-NCG | 1 | | M1 M2 |
| | 2 | | M3 M4 |
| | 3 | | M5 M6 |
| | 4 | | M7 M8 |
| Taconic- | 1 | M12 | M10 M11 |
| NOG-EXL | 2 | M90 | M88 M89 |

The humanized mice purchased from Taconic mobilized higher numbers of hCD45+CD56+γδTCR+ cells than mice purchased from Charles River after the first dose of AVM0703. A marker for invTCR was included in the flow panels done for the second dose but not the first dose. However, upon redose, mice from Charles River mobilized higher numbers of hCD45+CD56+γδTCR+invTCR+ cells than they did after the first dose, while mice from Taconic had the same mobilization compared to placebo upon redose. Placebo treated mice cannot be considered naïve mice because all mice are lethally irradiated and then transplanted with human umbilical cord blood CD34+ cells, so it is not surprising that Placebo treated mice might have baseline levels of these novel immune cells since we have shown that the cells are present but not optimally mobilized until after AVM0703 treatment when mice have cancer or diabetes.

TABLE 15
Humanized mice vendor, treatment, AVM_NKT cells

| | | | | hCD45+(CD56+gdTCR+ |
|---|---|---|---|---|
| Vendor | Mouse | Treatment | hCD45+(CD56+gdTCR+) | inTCR+) |
| CR | 1 | AVM0703 72 hrs | 1.1% | 2.3% |
| CR | 2 | AVM0703 96 hrs | 4.4% | 30.7% |
| CR | 3 | AVM0703 72 hrs | 1.6% | 3.5% |
| CR | 4 | AVM0703 96 hrs | 1.4% | 47.5% |
| CR | 5 | AVM0703 72 hrs | 4.7% | 4.4% |
| CR | 6 | AVM0703 96 hrs | 1.1% | 24.1% |
| CR | 7 | AVM0703 72 hrs | 6.3% | 1.8% |
| CR | 8 | AVM0703 96 hrs | 0.4% | 50% |
| Tac | 10 | AVM0703 72 hrs | 10.5% | 0.7% |
| Tac | 11 | AVM0703 96 hrs | 11% | 22.6% |
| Tac | 12 | Placebo 72 hrs | 7.1% | 0.8% |
| Tac | 88 | AVM0703 72 hrs | 3.4% | 0.7% |
| Tac | 89 | AVM0703 96 hrs | 0.6% | 41.9% |
| Tac | 90 | Placebo 96 hrs | 4.0% | 20% |

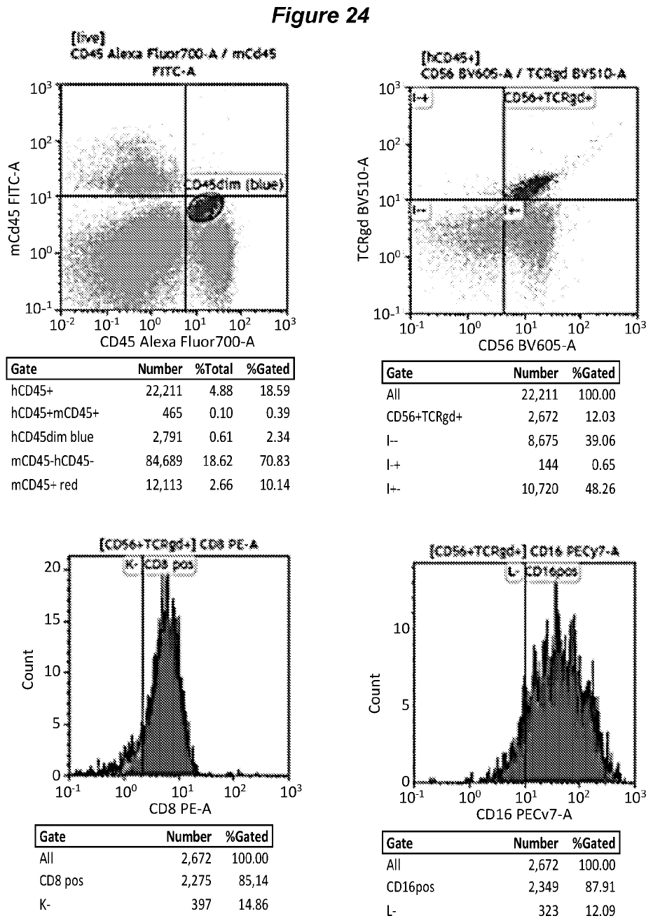

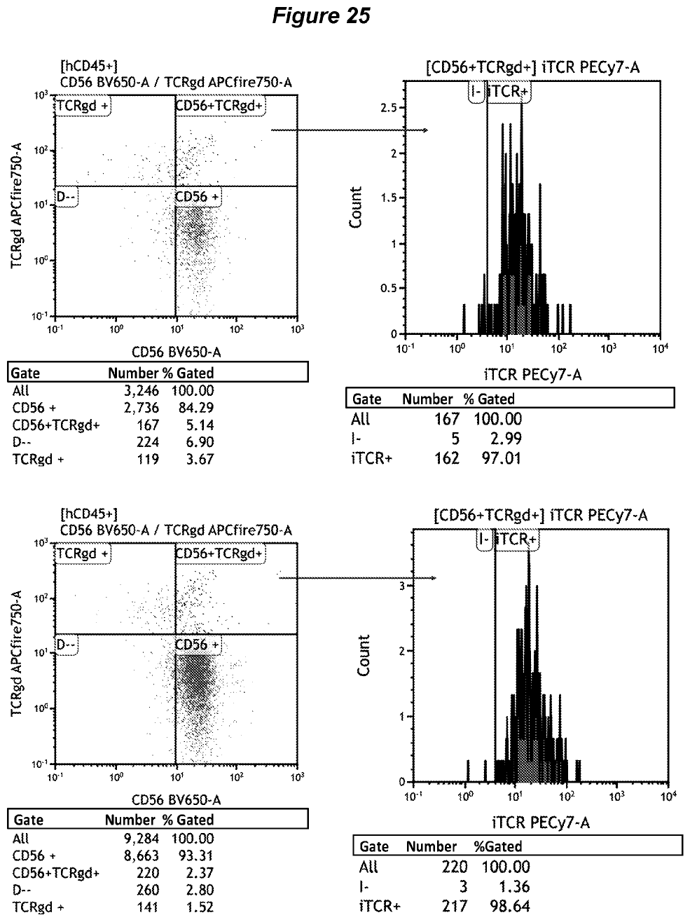

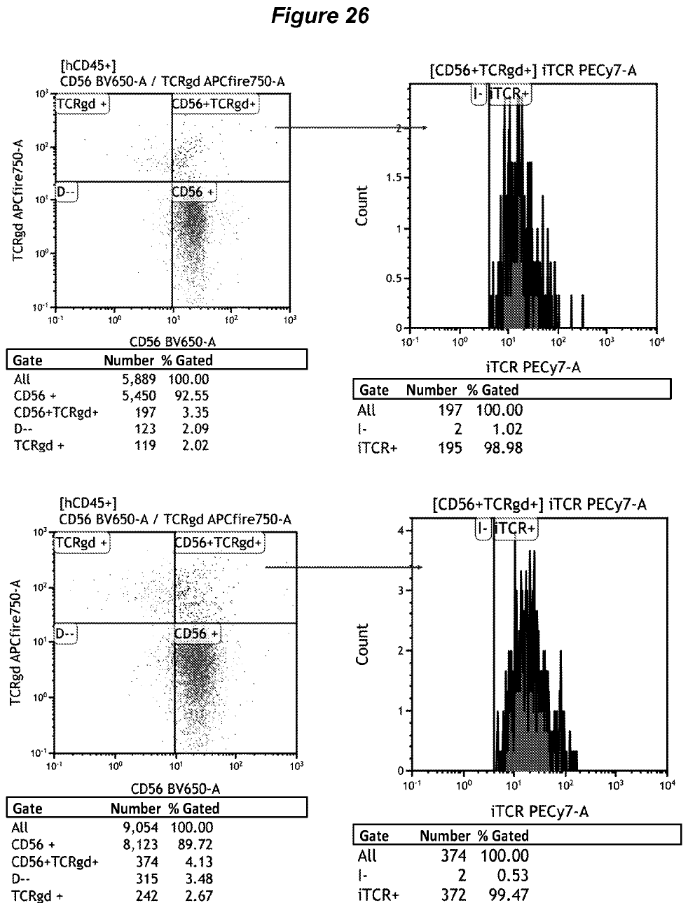

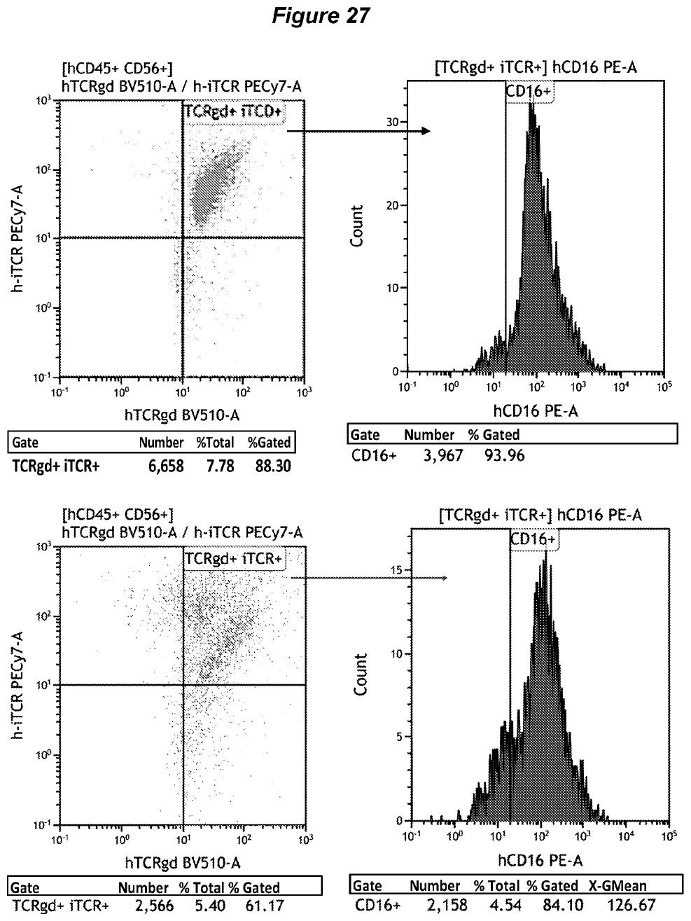

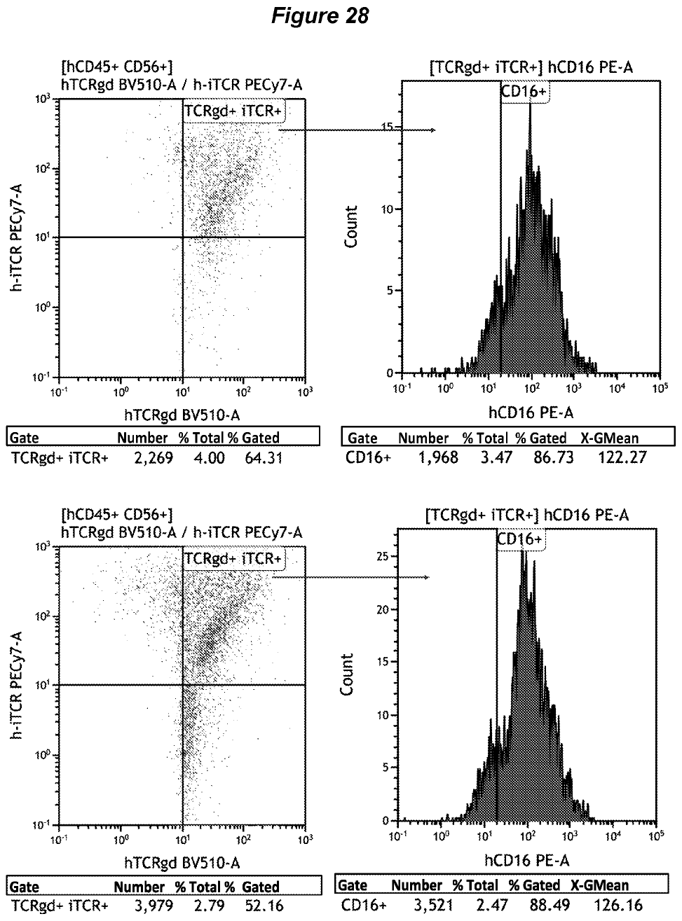

Similar to human patients, AVM0703 induces CD56+ TCRγδ+invTCR+bi-specific immune cell mobilization in humanized mice. As shown in FIG. 24, AVM0703 induced CD56+ TCRγδ+ cells (12% of hCD45+ cells) that are CD16+, suggesting an activated state (mouse 10 Taconic NOG-EXL). As shown in FIGS. 25 and 26, >18 mg/kg AVM0703 HED induces bi-specific immune cell mobilization between 2-12% of hCD45+ cells. As shown in FIGS. 27 and 28, AVM0703 induces γδTCR+invTCR+ bispecific activated CD56+ bone marrow cells in humanized mice, which correlates with data from human patients. More than 60% of human CD45+CD56+ cells were bispecific for TCRγδ and invariant TCR, and were CD16 positive, indicating an activated state. Bone marrow was analyzed 48-60 hours after a third repeat dose of AVM0703 32 mg/kg HED.

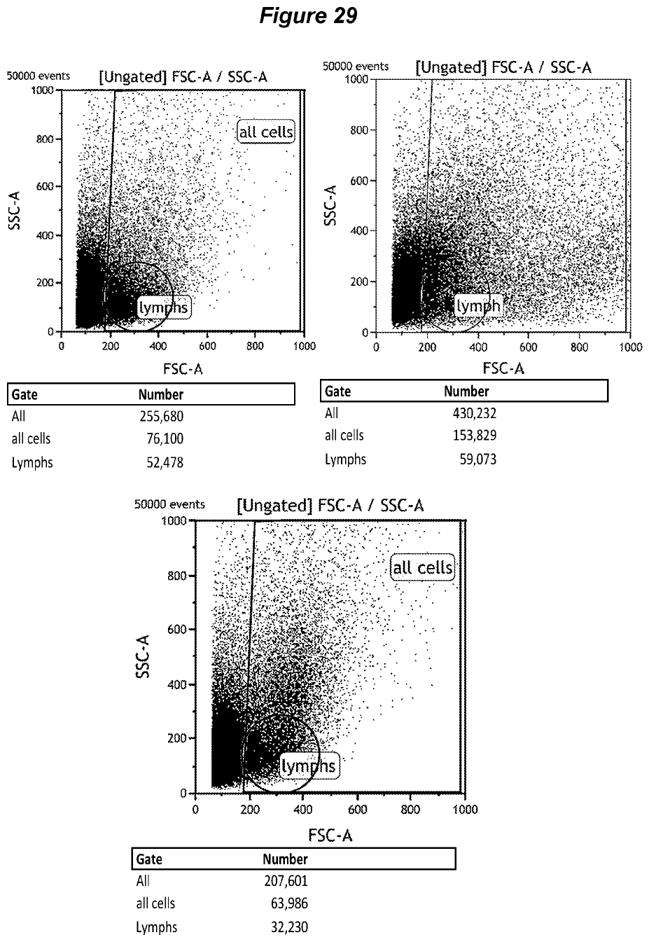

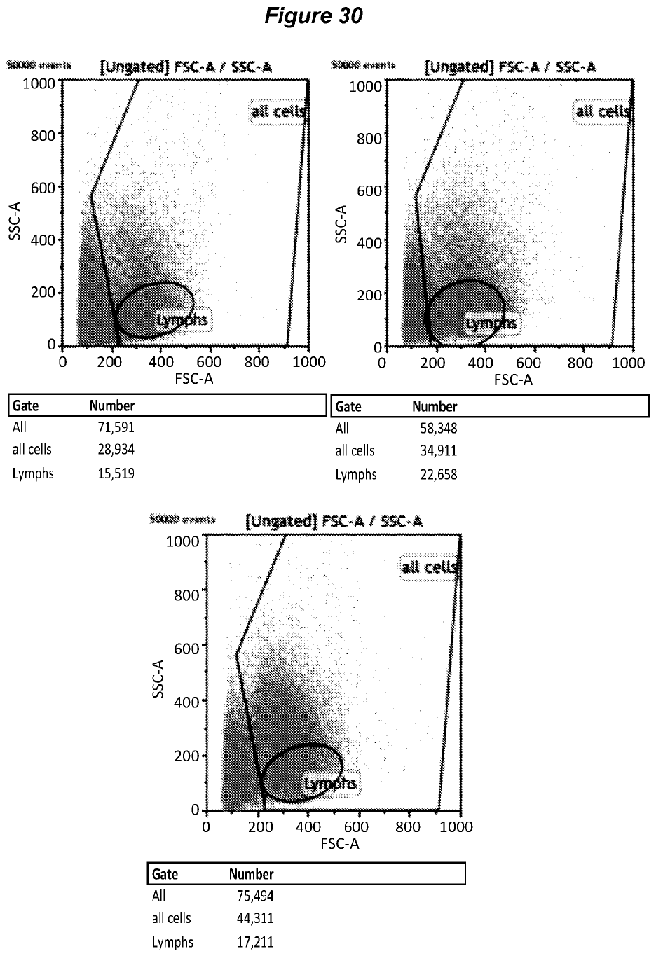

FIGS. 29 and 30 show FSC vs SSC for humanized mice after first (FIG. 29) and second (FIG. 30) doses of AVM0703. The mice are reported by Charles River and Taconic to not have myeloid compartments, however, after dosing with AVM0703 the mice have begun making both human and mouse myeloid cells. Scatter plots for two Placebo mice (FIGS. 29 and 30 upper plots; placebo mouse M12 upper left, placebo mouse M90 upper right) and for an AVM0703 treated mouse (FIGS. 29 and 30 lower plot; mouse M88) are shown. Mouse 88 had the earliest increase in myeloid cells of all 12 AVM0703 treated humanized mice. On average, Placebo treated mice had 12.7% of total mouse WBCs that were lymphoid cells while AVM0703 treated mice had 10.62% of total mouse WBCs that were lymphoid cells (ranged from 2%-20.4%).

This observation that the mice begin making myeloid cells, which include neutrophils, is consistent with reports from a compassionate use patient in Germany who began making healthy active neutrophils after AVM0703. This 18 year old male had not made neutrophils since after his first chemotherapy cycle 6 years prior to being treated with AVM0703. Similarly, human patients in our AVM0703-001 trial in R/R NHL all demonstrate some evidence of increased neutrophils.

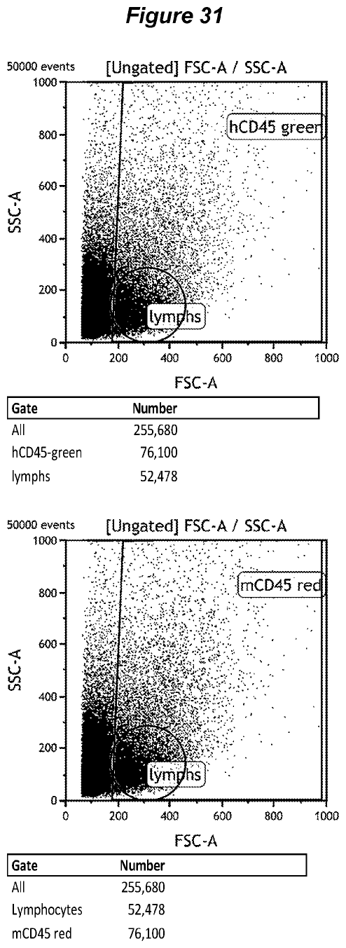

FIGS. 31-33 show that humanized mice have largely human lymphoid cells. These figures represent another way at looking at the origins of lymphoid versus myeloid cells in placebo treated mice and again demonstrate that the few myeloid cells are largely of mouse origin while the majority of lymphoid cells are of human origin. There was significant debris in these flow cytometry samples, which is why so many points in the ungated hCD45 versus mCD45 scatter plot are negative for both human and mouse CD45.

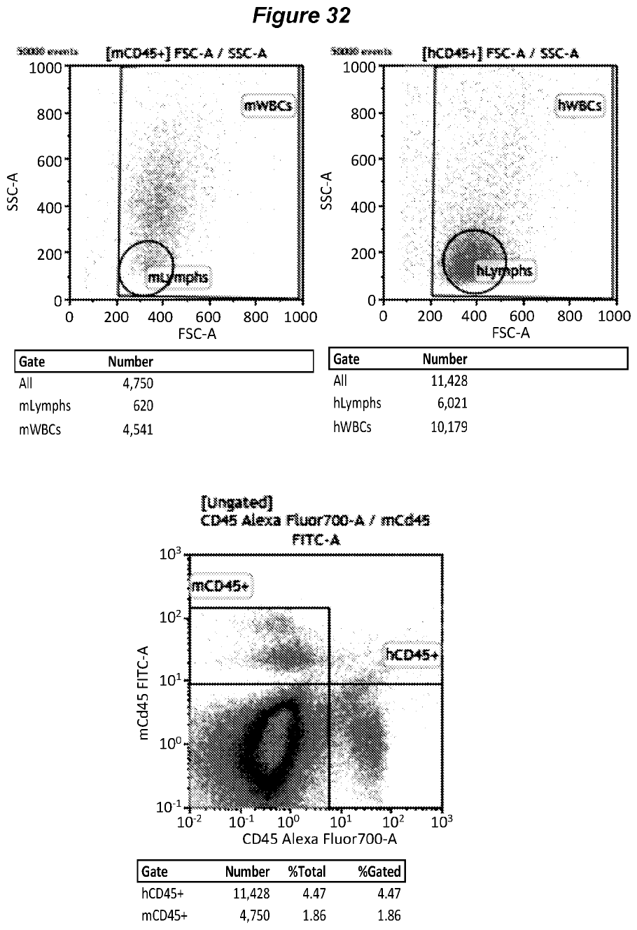

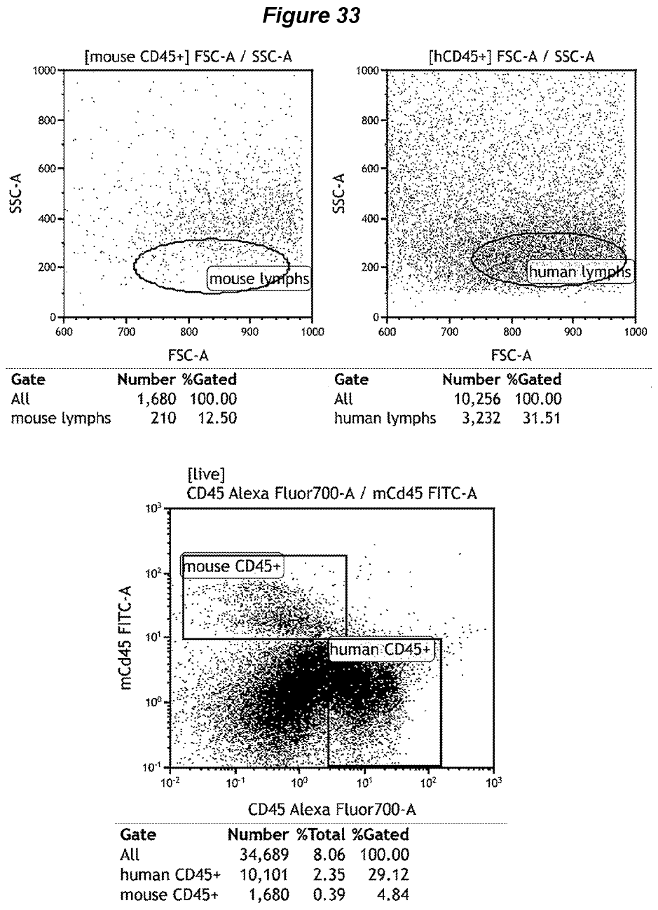

FIG. 31 shows that in placebo treated mice, lymphocytes are mostly human CD45+ (FIG. 31 upper) and the few myeloid cells are mostly mCD45+ (FIG. 31 lower). FIGS. 32-33 show that AVM0703 dosing induces myeloid cell production in humanized mice. Shown are FSC vs SSC gated on mCD45+ cells (upper left) and hCD45+ cells (upper right) and a scatter plot of hCD45+vs mCD45+ cells (lower). FIG. 32 shows data from a placebo mouse M12, in which mouse lymphocytes are 13% of total mouse WBCs (FIG. 32 upper left); human lymphocytes are 60% of total human WBCs (FIG. 32 upper right); and total lymphocytes are 45% of total WBCs. FIG. 33 shows data from a placebo mouse M90, in which mouse lymphocytes are 12.5% of total mouse WBCs (FIG. 33 upper left); human lymphocytes are 31.5% of total human WBCs (FIG. 33 upper right); and total lymphocytes are 30% of total WBCs.

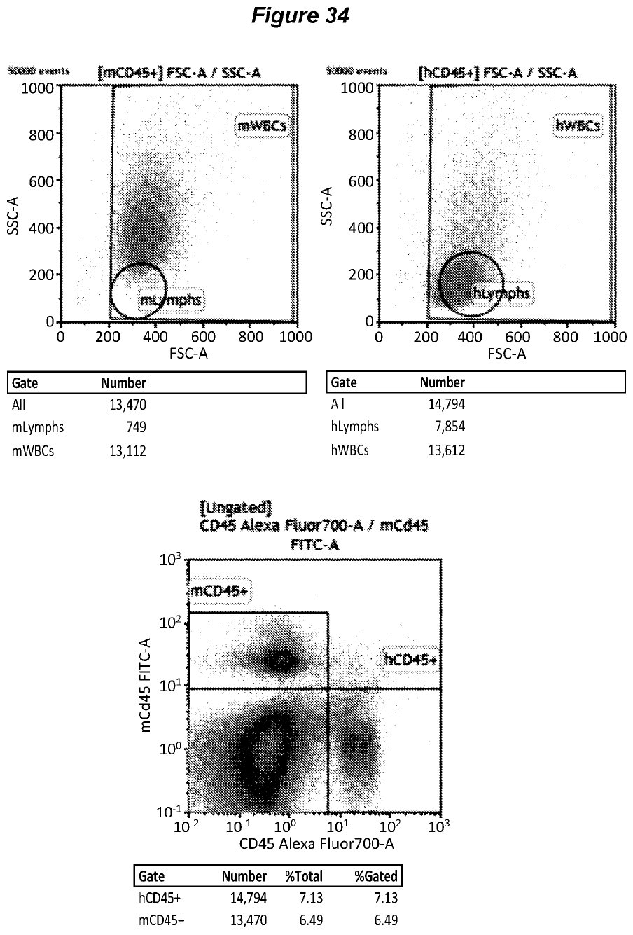

FIGS. 34-39 show that AVM0703 dosing induces myeloid cell production in humanized mice. These show flow cytometry scatter plots for AVM0703 treated mice after a first dose of AVM0703. In the scatter plots lymphocytes are circled, while myeloid cells have higher SSC and plot above the lymphocytes. These FSC versus SSC scatter plots show significantly higher numbers of myeloid cells of mouse origin (upper left) compared to placebo, suggesting that AVM0703 dosing induces myeloid production as has been observed in human clinical trial patients and compassionate use patients. The lymphoid population remains largely hCD45+ origin (upper right). In comparison to Placebo treated humanized mice where human CD45+ cells are about double the number of mCD45+ cells, AVM0703 treated humanized mice have about equal numbers of mouse CD45+ cells and human CD45+ cells (lower).

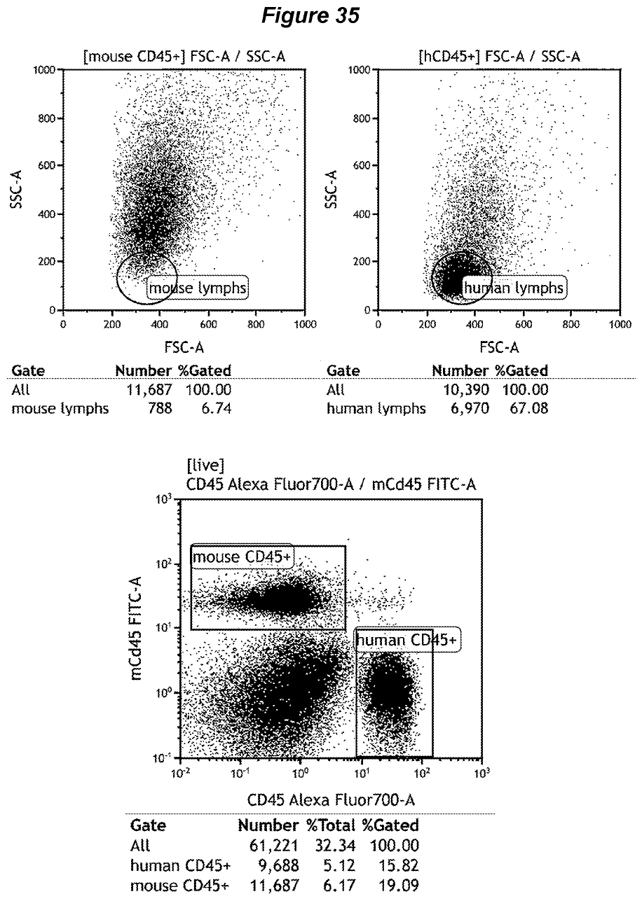

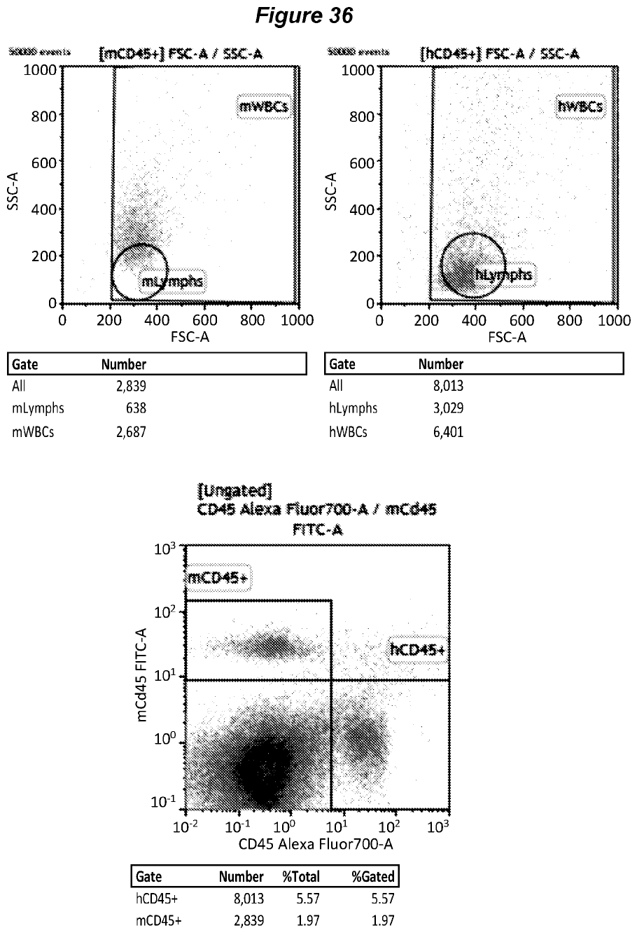

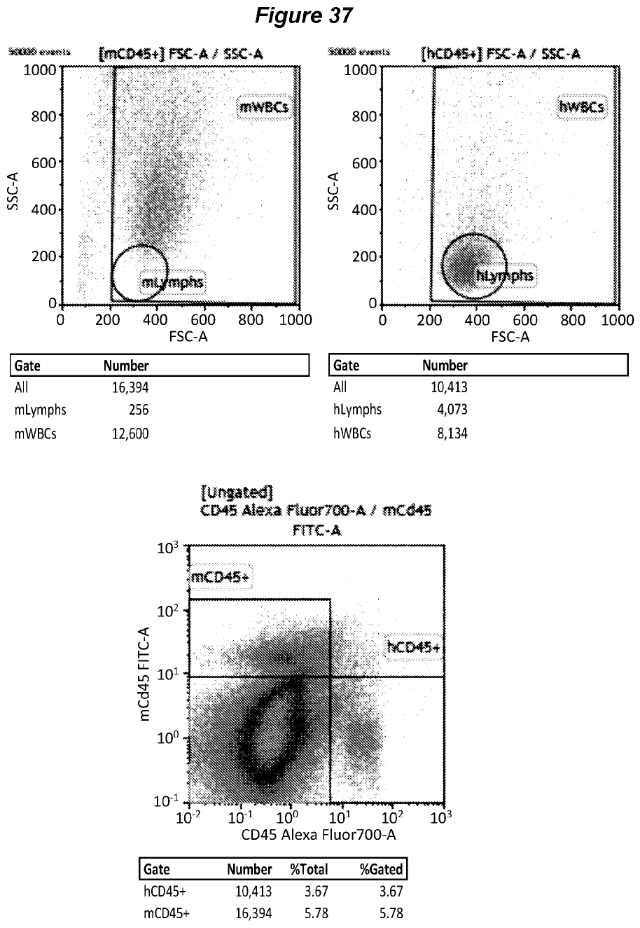

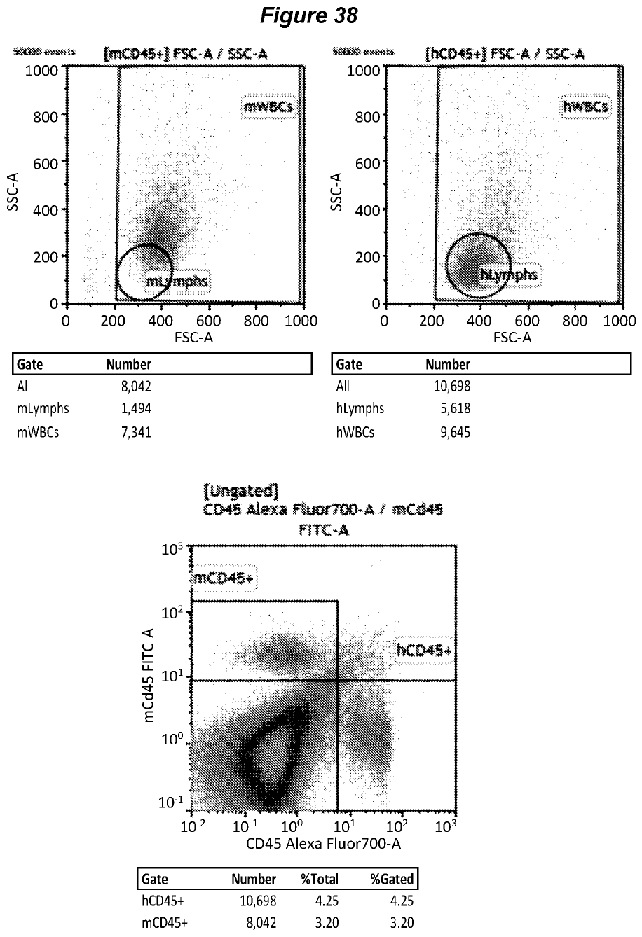

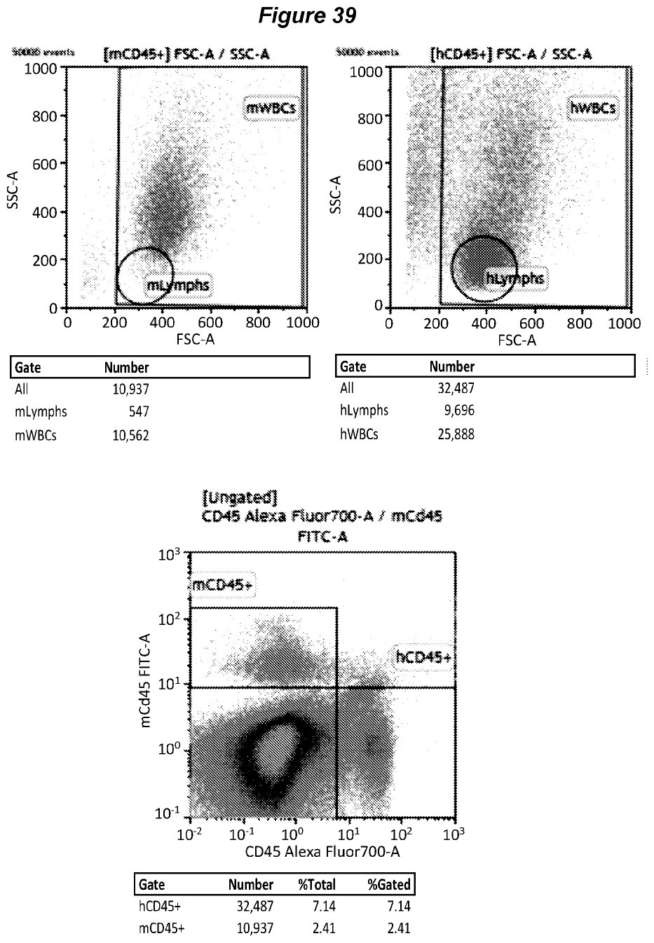

FIG. 34 shows data from AVM0703 treated mouse M88, in which mouse lymphocytes are only 5.7% of total WBCs (FIG. 34 upper left); human lymphocytes are 58% of total human WBCs (FIG. 34 upper right); and total lymphocytes are 32% of total WBCs. FIG. 35 shows data from AVM0703 treated mouse M01, in which mouse lymphocytes are only 6.7% of total WBCs (FIG. 35 upper left); human lymphocytes are 67% of total human WBCs (FIG. 35 upper right); and total lymphocytes are 35% of total WBCs. FIG. 36 shows data from AVM0703 treated mouse M03, in which mouse lymphocytes are only 23.7% of total WBCs (FIG. 36 upper left); human lymphocytes are 47% of total human WBCs (FIG. 36 upper right); and total lymphocytes are 40% of total WBCs. FIG. 37 shows data from AVM0703 treated mouse M05, in which mouse lymphocytes are only 2.0% of total WBCs (FIG. 37 upper left); human lymphocytes are 50.1% of total human WBCs (FIG. 37 upper right); and total lymphocytes are 20.9% of total WBCs. FIG. 38 shows data from AVM0703 treated mouse M07, in which mouse lymphocytes are only 20.4% of total WBCs (FIG. 38 upper left); human lymphocytes are 58.2% of total human WBCs (FIG. 38 upper right); and total lymphocytes are 41.9% of total WBCs. FIG. 39 shows data from AVM0703 treated mouse M10, in which mouse lymphocytes are only 5.2% of total WBCs (FIG. 39 upper left); human lymphocytes are 37.5% of total human WBCs (FIG. 39 upper right); and total lymphocytes are 28.1% of total WBCs.

### Example 16-In Vivo Anti-Cancer Activity of AVM0703 Induced and then Adoptively Transferred Immune Cells

In vivo activated immune cells were adoptively transferred (ACT) to mice with MOPC315 Multiple Myeloma cells injected into their flanks which also metastasized to spleen, blood and bone marrow. AVM0703 was used not only to induce bi-specific γδTCR+invTCR+ NKT-like cells that were isolated and then adoptively transferred, but also as a preconditioning agent to determine whether AVM0703 could replace cytotoxic preconditioning regimens such as cyclophosphamide/fludarabine (Cy/Flu). As expected, preconditioning was required for statistically significant effects of ACT cells from AVM0703 treated mice. Mice preconditioned with AVM0703 will also mobilize their own endogenous novel immune cells, in addition to the ACT cells that they receive.

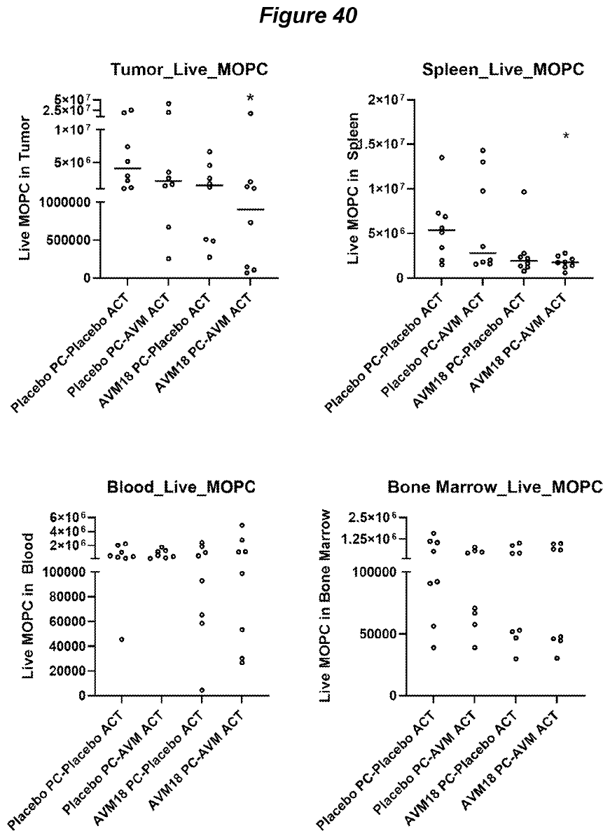

ACT cells from AVM0703 treated mice significantly reduced the total number of live MOPC315 cells in tumors (FIG. 40 upper left) and spleens (FIG. 40 upper right) of mice preconditioned with AVM0703. Additionally, while the reductions were not statistically significant, preconditioning with AVM0703 followed by ACT of cells from placebo treated mice showed trends towards reduced live MOPC315 cells. This was expected based on the ability of AVM0703 preconditioning to induce/mobilize endogenous bi-specific NKT-like cells in MOPC315 inoculated mice. While results were not statistically significant, AVM0703 preconditioning followed by ACT showed trends of reduced live MOPC315 in blood (FIG. 40 lower left) and bone marrow (FIG. 40 lower right) also. This MOPC315 research was supported by NCI SBIR grant 1R43CA246896-01A1.

### Example 17-High Dose Glucocorticoids Such as Dexamethasone Bind to ICAM3 Via Low Affinity Hydrogen Bonding, which May Mediate Induction and/or Mobilization of the Novel NKT-Like Cells of the Invention

The present authors have also discovered that, following high dose administration, glucocorticoid molecules can bind and block intercellular adhesion molecules such as ICAM3-described, for example, in WO 2021 247473. Molecular modelling of the interaction between dexamethasone and ICAM3 predicts that the interaction between these is via low affinity hydrogen bonding, including interactions between a hydrogen molecule in dexamethasone and the SER31 residue in ICAM3, and an oxygen molecule in dexamethasone and the MET49 residue in ICAM3. Molecular modelling of the interaction between and ICAM3 and a number of other ligands predicts: that agonistic antibodies bind to ICAM3 at a hydrophobic pocket and interact with the residues THR38, LEU40, LEU56, VAL59, and ILE65; that the anti-ICAM3 antibody ICR 8.1 interact with the residues PHE21, VAL22, GLU32, LYS33, TRP51, and ALA52; and, that the integrin lymphocyte function-associated antigen 1 (LFA-1) interacts with the residues at SER25, ASN23, GLU37, PHE54, and GLN75.

The authors believe that the induction and/or mobilization of the novel NKT-like cells of the invention may be mediated by this interaction between ICAM3 and glucocorticoids, in a glucocorticoid receptor-independent mechanism of action.

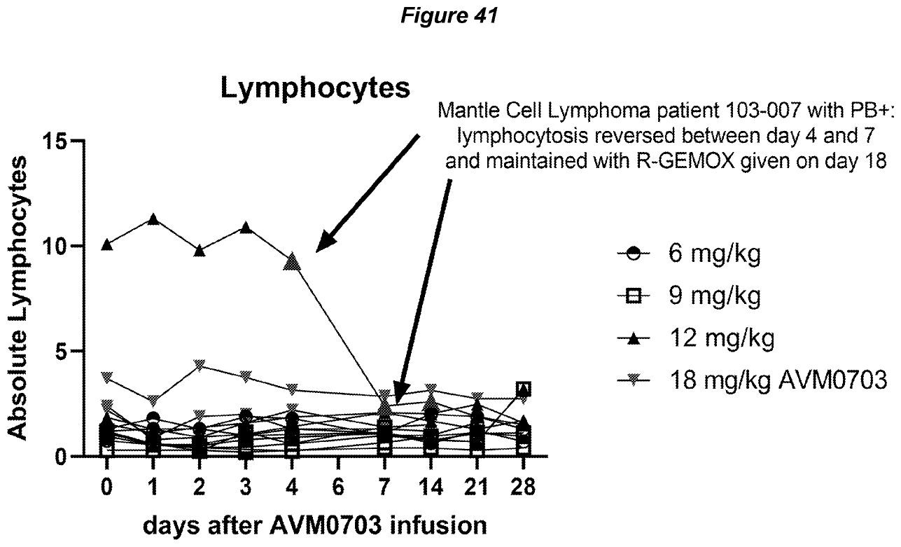

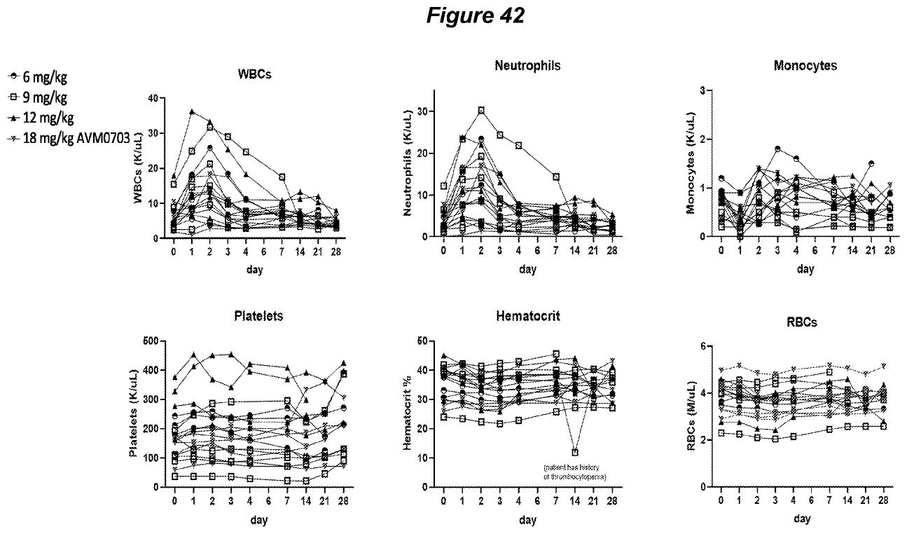

As shown in FIG. 41, in a patient with Mantle Cell Lymphoma (Patient 103-007) following a 12 mg/kg AVM0703 infusion, lymphocytosis was reduced to normal lymphocyte levels between day 4 and 7 after AVM0703, suggesting that AVM0703 immune activation preferentially recognizes cancerous cells and spares normal blood cells, including lymphocytes, monocytes, platelets and RBCs (FIG. 42). Hematocrit and hemoglobin were also spared, and hemoglobin was actually clinically significantly increased after 9 mg/kg AVM0703 infusion in a R/R MCL patient. The absence of lymphocytopenia in response to AVM0703 supports a glucocorticoid receptor (GCR) independent mechanism of action. Similarly, absence of effect on platelets and increased neutrophils also support a GCR-independent mechanism of action.

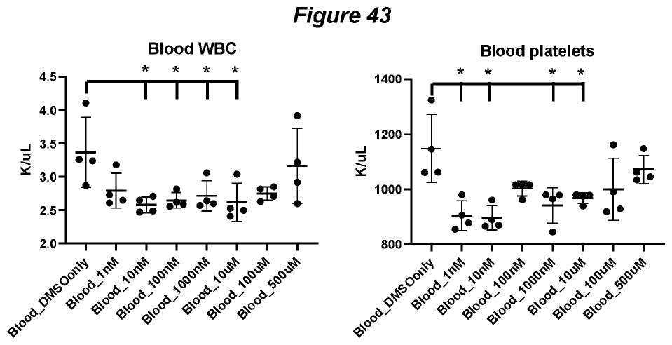

Concentration-response curves show the expected apoptotic effect of dexamethasone base on isolated mouse splenocytes and whole blood at concentrations known to bind the transmembrane GCR (10 nM to 100 uM), however, a biphasic curve is observed with apoptosis decreasing as concentration is increased above 100 uM (which is an in vivo equivalent blood concentration peak from about a 2.8 mg/kg human equivalent dose (HED), as shown in FIG. 43. Biphasic response curves have been well described for chemokines (Olsen I, J Immunol Methods. 2013 Apr. 30; 390 (1-2): 106-12; Florini J R, Am J Physiol. 1986 May; 250 (5 Pt 1): C771-8) and growth factors (Parris, Dose Response. 2015 May 20; 13 (2): dose-response. 14-020; Kanodia J, Cell Commun Signal. 2014 May 15; 12:34), and have been shown to be a result of receptor desensitization/internalization or a new low affinity (Olsen, 2013; Florini, 1986) but very dense receptor soaking up the factor so that it is not available to bind the higher affinity but less accessible receptors (Kanodia, 2014; Koledova Z, Front Cell Dev Biol. 2019 Dec. 12; 7:331).

Similarly, from the AVM0703-001 dose-escalation phase in R/R NHL patients, WBCs, lymphocytes, platelets and RBCs were not depleted at doses between 6 mg/kg and 18 mg/kg. WBC, platelets, monocytes, lymphocytes and splenocytes are known to express the GCRalpha, consistent with the effects seen on these cell populations at dexamethasone base concentrations between 1 nM and 10 uM. The bi-phasic CRC suggests that a low affinity but very dense non-GCR receptor soaks up the high concentrations, preventing binding and activation of the GCRs (Kanodia, 2014). RBCs, which do not express the GCRs, had no response to ex vivo dexamethasone base at any concentration (data not shown). ICAM3 was identified as a potential low affinity receptor for dexamethasone suprapharmacologic concentrations through literature and Human Proteome database searching and confirmed by molecular docking studies conducted by two independent consultants.

ICAM3 has been reported to be shed after dexamethasone binding (Juan M, 1999), and the authors hypothesize that this binding is covalent at suprapharmacologic doses, preventing AVM0703 from binding to GCRs and explaining why GCR activation is not observed as AVM0703 is seemingly cleared from the blood from the PK analysis. AVM0703 covalently bound to shed ICAM3 would be found in the plasma/serum fraction of blood and bound AVM0703 would be released from the ICAM3 during LC-MS/MS analysis, but prevented from binding to and activating GCRs. Alternatively, AVM0703's structure could be modified after ICAM3 binding such that it can no longer bind GCRs even if free in the blood.

### REFERENCES

A number of publications are cited above in order to more fully describe and disclose the invention and the state of the art to which the invention pertains. Each reference is hereby encorporated by reference in its entirety. Full citations for these references are provided below:
Tarazona, Raquel & Peralbo, E. & Casado, Javier & Pena, Jose & Solana, Rafael. Human NKT cells in health and disease. Inmunologia. 2003; 22 Watarai H, Nakagawa R., Omori-Miyake M, Dashtsoodol N, & Taniguchi M. Methods for detection, isolation and culture of mouse and human invariant NKT cells. Nat Protoc. 2008; 3, 70-78.
Wolf B J, Choi J E, & Exley M A. Novel Approaches to Exploiting Invariant NKT Cells in Cancer Immunotherapy. Frontiers in immunology. 2018; 9, 384 Nair S, & Dhodapkar M V. Natural Killer T Cells in Cancer Immunotherapy. Frontiers in immunology. 2017:8, 1178 Lodisch M. B. Clinical review: kinase inhibitors: adverse effects related to the endocrine system. The Journal of clinical endocrinology and metabolism. 2013; 98 (4), 1333-1342
Bhullar, K S, Lagarón NO, McGowan E M. et al. Kinase-targeted cancer therapies: progress, challenges and future directions. Mol Cancer. 2018; 17, 48
Mato A R, Thompson M, Allan J N, Brander D M, Pagel J M, Ujjani C S, Nabhan C. Real-world outcomes and management strategies for venetoclax-treated chronic lymphocytic leukemia patients in the United States. Haematologica. 2018; 103 (9), 1511-1517
Kadri S, Lee J, Fitzpatrick C, Galanina, N, Sukhanova, M, Venkataraman, G, Wang Y L. Clonal evolution underlying leukemia progression and Richter transformation in patients with ibrutinib-relapsed CLL. Blood advances. 2017; 1 (12), 715-727
Mato A R, Nabhan C, Barr P M, Ujjani C S, Hill B T, Lamanna N, Skarbnik A P, Howlett C, Pu J J, Sehgal A R, Strelec L E, Vandegrift A, Fitzpatrick D M, Zent C S, Feldman T, Goy A, Claxton D F, Bachow S H, Kaur G O, Svoboda J, Nasta S D, Porter D, Landsburg D J, Schuster S J, Cheson B D, Kiselev P, Evens A M. Outcomes of CLL patients treated with sequential kinase inhibitor therapy: a real world experience. Blood. 2016; 128 (18): 2199-2205
Barrett A J, & Battiwalla M. Relapse after allogeneic stem cell transplantation. Expert review of hematology. 2010; 3 (4), 429-441
Nie Y, Lu W, Chen D, Tu H, Guo Z, Zhou X, Li M, Tu S, Li Y. Mechanisms underlying CD19-positive ALL relapse after anti-CD19 CAR T cell therapy and associated strategies. Biomark Res. 2020 May 27; 8 (1)./pmc/articles/PMC725465
Gill S, Brudno J.CAR T-Cell Therapy in Hematologic Malignancies: Clinical Role, Toxicity, and Unanswered Questions. Am Soc Clin Oncol Educ book Am Soc Clin Oncol Annu Meet. 2021 June: 41 (41): e246-65. https://pubmed.ncbi.nlm.nih.gov/33989023/Stadtfeld et al, Science. 2008 Nov. 7; 322 (5903); 945-9
Okita et al, Science. 2008 Nov. 7; 322 (5903); 949-53
Bowles et al, Dev Biol. 2000 Nov. 15; 227 (2); 239-55.
Chambers et al, Cell. 2003 May 30; 113 (5); 643-55.
Moss & Tang, 2003, Dev Biol. 2003 Jun. 15; 258 (2); 432-42.
Shiina et al, Immunology 2016; 150:127-138
Nakamura et al 2019 Int. J. Mol. Sci 20; 4544
Kaczmarek et al 2017 Arch. Immunol. Ther. Exp. 65:201-214
WO 2018/183927
Zheng et al; Cell Mol Immunol. 2013 January; 10 (1): 50-7.
Gogoi et al; Indian J Med Res. 2013 November; 138 (5): 755-761.
Dong et al; Front Immunol. 2018 Dec. 4; 9:2812.
Zhao et al, 2018; J Immunol Res. 2018 Jul. 10; 2018:5
Pauza et al, 2018; Front Immunol. 2018; 9:1305.
Himoudi et al, 2012; J Immunol. 2012 Feb. 15; 188 (4): 1708-16

### Statements of Disclosure

The following numbered statements, outlining aspects of the present disclosure, are part of the description.

### Methods of Producing Human Immune System NKT-Like Cells (AVM-NKT Cells)

101. A method of producing and/or mobilizing a population of natural killer T cell-like cells (NKT-like cells), the method comprising administering to a subject a glucocorticoid-receptor (GR) modulating agent or ICAM3 modulating agent at a dose equivalent to about at least 6 mg/kg human equivalent dose (HED) of dexamethasone base,
wherein the glucocorticoid receptor (GR) modulating agent or ICAM3 modulating agent induces and/or mobilizes the population of NKT-like cells in the subject.

### Cell Marker Expression

102. The method of statement 101, wherein the population of cells are characterized in that at least 60, 70, 80, 90, 95, 96, 97, 98, or 99% of the cells:
i) express CD56, TCR gamma/delta, iTCR, CD16, NKp44, CD3, CD8, CD14, CD19, CD45, TCR alpha/beta, CD34 and/or ICAM3;
ii) express CD56, TCR gamma/delta, iTCR, CD16, NKp44, CD3, CD8, CD14, CD19, CD45, and/or TCR alpha/beta; and/or
iii) do not express: CD4.
   103. The method of statement 102, wherein the cells express:
(i) TCR gamma/delta, and iTCR;
(ii) CD56, TCR gamma/delta, and iTCR;
(iii) CD45, TCR gamma/delta, and iTCR;
(iv) CD45, CD56, TCR gamma/delta, and iTCR;
(vi) TCR gamma/delta, iTCR, and TCR alpha/beta;
(vii) CD56, TCR gamma/delta, iTCR, and TCR alpha/beta;
(viii) CD45, TCR gamma/delta, iTCR, and TCR alpha/beta;
(ix) CD45, CD56, TCR gamma/delta, iTCR, and TCR alpha/beta;
(x) CD56, TCR gamma/delta, iTCR, and CD16;
(xi) CD45, TCR gamma/delta, iTCR, and CD16;
(xii) CD16 and NKp44;
(xiii) CD56, TCR gamma/delta, iTCR, CD16, and NKp44;
(xiv) CD56, TCR gamma/delta, iTCR, and TCR alpha/beta;
(xv) CD56, TCR gamma/delta, iTCR, CD16, NKp44, and TCR alpha/beta;
(xvi) CD56, TCR gamma/delta, iTCR, CD16, NKp44, CD8, CD14, and CD19;
(xvii) CD56, TCR gamma/delta, iTCR, CD16, NKp44, CD3, CD8, CD14, CD19, and CD45; or
(viii) CD56, TCR gamma/delta, iTCR, CD16, NKp44, CD3, CD8, CD14, CD19, CD45, and TCR alpha/beta.
   104. The method of any one of statements 102-103, wherein the cells do not express CD4.
   105. The method according to any one of statements 102-104, wherein the cells are:
i) CD3+/dim;
ii) CD8+/dim;
iii) CD3+/dim and CD8+/dim:
   optionally, wherein the expression levels are determined relative to the average expression level in a population of reference cells derived from a common source, which have not been contacted with the glucocorticoid-receptor (GR) modulating agent or ICAM3 modulating agent.
      106. The method according to any one of statements 102-105, wherein expression is measured by flow cytometry, optionally wherein the flow cytometry is performed using the equipment, reagents, and/or conditions described herein (taken in isolation or in combination).

### Glucocorticoid

107. The method according to any one of statements 101-106, wherein the glucocorticoid-receptor (GR) modulating agent or the ICAM3 modulating agent is a glucocorticoid, optionally wherein the glucocorticoid is selected from the group consisting of: dexamethasone, hydrocortisone, methylprednisolone, prednisone, prednisolone, prednylidene, cortisone, budesonide, betamethasone, flumethasone and beclomethasone.
108. The method according to statement 107, wherein the glucocorticoid is selected from the group consisting of: dexamethasone, betamethasone, and methylprednisone, preferably wherein the glucocorticoid is dexamethasone or betamethasone.
109. The method according to any one of statements 107-108, wherein the glucocorticoid is selected from the group consisting of dexamethasone base, dexamethasone sodium phosphate, dexamethasone hemisuccinate, dexamethasone sodium succinate, dexamethasone succinate, dexamethasone isonicotinate, dexamethasone-21-acetate, dexamethasone phosphate, dexamethasone-21-phosphate, dexamethasone tebutate, dexamethasone-17-valerate, dexamethasone acetate monohydrate, dexamethasone pivalate, dexamethasone palmitate, dexamethasone-21-palmitate, dexamethasone dipropionate, dexamethasone propionate, dexamethasone acetate anhydrous, dexamethasone-21-phenylpropionate, dexamethasone-21-sulfobenzoate, dexamethasone hemo-sulfate, dexamethasone sulfate, dexamethasone beloxil, dexamethasone acid, dexamethasone acefurate, dexamethasone carboximide, dexamethasone cipecilate, dexamethasone 21-phosphate disodium salt, dexamethasone mesylate, dexamethasone linoleate, dexamethasone glucoside, dexamethasone glucuronide, dexamethasone iodoacetate, dexamethasone oxetanone, carboxymethylthio-dexamethasone, dexamethasonebisethoximes, dexamethasone epoxide, dexamethasonelinolelaidate, dexamethasone methylorthovalerate, dexamethasone spermine, 6-hydroxy dexamethasone, dexamethasone tributylacetate, dexamethasone aspartic acid, dexamethasone galactopyranose, dexamethasone hydrochloride, hydroxy dexamethasone, carboxy dexamethasone, desoxy dexamethasone, dexamethasone butazone, dexamethasone cyclodextrin, dihydro dexamethasone, oxo dexamethasone, propionyloxy dexamethasone, dexamethasone galactodie, dexamethasone isonicotinate, dexamethasone sodium hydrogen phosphate, dexamethasone aldehyde, dexamethasone pivlate, dexamethasone tridecylate, dexamethasone crotonate, dexamethasone methanesulfonate, dexamethasone butylacetate, dehydro dexamethasone, dexamethasone Isothiocyanatoethyl)Thioether, dexamethasone bromoacetate, dexamethasone hemiglutarate, deoxy dexamethasone, dexamethasone chlorambucilate, dexamethasone melphalanate, formyloxy dexamethasone, dexamethasone butyrate, dexamethasone laurate, dexamethasone acetate, and any combination treatment that contains a form of dexamethasone.
110. The method according to statement 109, wherein the dexamethasone is dexamethasone sodium phosphate.

### Glucocorticoid Dose

111. The method according to any one of statements 101-110, wherein the glucocorticoid is administered at a dose equivalent to about:
i) at least 6-12 mg/kg human equivalent dose (HED) of dexamethasone base;
ii) at least 6 mg/kg human equivalent dose (HED) of dexamethasone base;
iii) at least 12 mg/kg human equivalent dose (HED) of dexamethasone base;
iv) at least 15 mg/kg human equivalent dose (HED) of dexamethasone base;
v) at least 18 mg/kg human equivalent dose (HED) of dexamethasone base;
vi) at least 24 mg/kg human equivalent dose (HED) of dexamethasone base;
vii) 15 mg/kg human equivalent dose (HED) of dexamethasone base;
viii 24 mg/kg human equivalent dose (HED) of dexamethasone base;
ix) 30 mg/kg human equivalent dose (HED) of dexamethasone base;
x) 45 mg/kg human equivalent dose (HED) of dexamethasone base; or.
xi) a human equivalent dose (HED) of dexamethasone base taking a value in mg/kg from a range of mg/kg values, wherein said range is bound by two of the mg/kg values set forth in parts i) to x) above.
   112. The method according to any one of statements 101-111, wherein the glucocorticoid is administered as a single acute dose, or as a total dose given over about a 72 hour period.
   113. The method according to any one of statements 101-112, wherein the method comprises administering one or more further doses of a glucocorticoid.
   114. The method according to statement 113, wherein the one or more further doses are administered:
i) between 24 hours and 120 hours after a preceding glucocorticoid administration;
ii) between 24 hours and 48 hours after a preceding glucocorticoid administration;
iii) between 72 hours and 120 hours after a preceding glucocorticoid administration;
iv) every 24, 48, 72, 96, 120, 144, or 168 hours after a first glucocorticoid administration;
v) once every two weeks after a first glucocorticoid administration;
vi) once monthly after a first glucocorticoid administration; or
vii) twice weekly after a first glucocorticoid administration.

### Cell Activation

115. The method according to any one of statements 101-114, further comprising a step of administering an NKT cell activator, T cell activator, and/or NK cell activator to the subject.
116. The method according to statement 115, wherein the NKT cell activator is selected from the group consisting of: alpha GalCer, Sulfatide, or an NKT-activating antibody.
117. The method according to statement 116, wherein the NKT cell activator is alpha GalCer loaded dendritic cells or monocytes.
118. The method according to statement 115, wherein the T cell activator is selected from the group consisting of: zoledronate, mevastatin, or a T cell-activating antibody.
119. The method according to statement 115, wherein the NK cell activator is selected from the group consisting of: IL-2, IL-12, IL-15, IL-18, IL-21, or an NK cell-activating antibody.
120. The method according to any one of statements 115-119, wherein the NKT cell activator, T cell activator, and/or NK cell activator is administered within or around 48 hours after administration of glucocorticoid.

### Subject

121. The method according to any one of statements 101-120, wherein the subject is human or a mammal with a humanised immune system, such as a human immune system (HIS) mouse.
122. The method according to any one of statements 101-121, wherein the subject has, is suspected of having, or has been diagnosed with a disease selected from the group consisting of: cancer, autoimmune disease, or infectious disease.
123. The method according to statement 122, wherein the cancer is a solid tumour cancer.
124. The method according to statement 122, wherein the cancer is selected from the group consisting of: squamous cell cancer (such as epithelial squamous cell cancer); lung cancer, including small-cell lung cancer, non-small cell lung cancer, adenocarcinoma of the lung and squamous carcinoma of the lung: cancer of the peritoneum: hepatocellular cancer: gastric or stomach cancer, including gastrointestinal cancer; pancreatic cancer; glioblastoma: cervical cancer; ovarian cancer; liver cancer; bladder cancer; hepatoma; breast cancer; colon cancer; rectal cancer; colorectal cancer; endometrial or uterine carcinoma: salivary gland carcinoma: kidney or renal cancer: prostate cancer; vulval cancer: thyroid cancer: hepatic carcinoma: anal carcinoma: penile carcinoma; and head and neck cancer.
125. The method according to statement 122, wherein the cancer is lymphoma, preferably a B cell lymphoma, a T cell lymphoma, or non Hodgkin lymphoma.
126. The method according to any one of statements 122-125, wherein the induced/mobilized cells treat the cancer via tumour infiltration.
127. The method according to statement 126, wherein the induced/mobilized cells treat the cancer via release of immune activating cytokines.
128. The method according to statement 126 or 127, wherein the induced/mobilized cells engulf and kill cancer cells.
129. The method according to any one of statements 126 to 128, wherein the induced/mobilized cells promote infiltration of other immune cells into the tumour.
130. The method according to any one of statements 126 to 129, wherein the induced/mobilized cells directly kill cancer cells via CD1d-directed apoptosis.
131. The method according to any one of statements 126 to 130, wherein the induced/mobilized cells cause tumour necrosis.
132. The method according to statement 122, wherein the autoimmune disease is selected from the group consisting of: multiple sclerosis, systemic sclerosis, amyotrophic lateral sclerosis, type 1 diabetes mellitus (TID), scleroderma, pemphigus, and lupus.
133. The method according to statement 122, wherein the autoimmune disease is type 1 diabetes mellitus (TID).
134. The method according to statement 122, wherein the infectious disease is selected from the group consisting of: HIV and herpes, hepatitis, human papilloma virus, or a disease resulting from infection with a coronavirus, such as COVID-19.
135. The method according to statement 122, wherein the infectious disease is:
i) HIV: or
ii) COVID-19.

### Isolation Expansion Steps

136. The method according to any one of statements 101-135, further comprising a step of isolating a population of the induced/mobilized cells from the subject or from a sample derived from the subject,
optionally wherein the step of isolating is performed:
   i) between 30 and 60 minutes after glucocorticoid administration;
   ii) at least 48 hours after glucocorticoid administration;
   iii) between 48 hours and 13 days after glucocorticoid administration; or
   iv) between 6 and 48 hours after glucocorticoid administration.
      137. The method of statement 136, wherein the sample is selected from the group consisting of: blood, plasma, a tumor biopsy or surgically removed tumor, bone marrow, liver, and fat or adipose tissue.
      138. The method according to statement 136 or 137, further comprising a step of expanding the isolated cells.
      139. The method according to any one of statements 136-138, further comprising a step of activating the isolated cells with an NKT cell activator, T cell activator, and/or NK cell activator:
optionally wherein the NKT cell activator is selected from: alpha GalCer (alpha-Galactosylceramide; α-GalCer) and sulfatide (3-O-sulfogalactosylceramide: SM4; sulfated galactocerebroside);
optionally wherein the T cell activator is selected from: zoledronate and mevastatin; and
optionally wherein the NK cell activator is selected from the group consisting of: IL-2, IL-12, IL-15, IL-18, IL-21.

### Transfection of Isolated Cells

140. The method according to any one of statements 136 to 139, further comprising a step of introducing a nucleic acid encoding a protein into the isolated cells, and culturing the cells under conditions that facilitate expression of said protein.
141. The method according to statement 140, wherein the protein is selected from the group consisting of one or more of: a T-cell receptor (TCR), a chimeric antigen receptor (CAR), a split, universal and programmable CAR (SUPRA-CAR).
142. The method according to statement 141, wherein the CAR and/or TCR comprises an antigen-binding domain which binds to an antigen selected from the group consisting of: CD19, CD20, CD22, GD2, CD133, EGFR, GPC3, CEA, MUC1, Mesothelin, IL-13R, PSMA, ROR1, CAIX, Her2.
143. The method according to any one of statement 140-142, further comprising a step of expanding the cells.
144. The method according to any one of statement 140-143, further comprising a step of activating the cells with an NKT cell activator, T cell activator, and/or NK cell activator.

### Administration of Isolated Cells

145. A method of treating cancer, autoimmune disease, or infectious disease in a subject, the method comprising administering a therapeutically effective dose of cells isolated according to any one of statements 136 to 144, or a therapeutically effective dose of the isolated cells or population of cells of any one of statements 201-211, to the subject.
146. The method according to statement 145, wherein the subject to which the isolated cells are administered is the same subject from which the cells were isolated.
147. The method according to statement 145, wherein the subject to which the isolated cells are administered is different to the subject from which the cells were isolated.
148. The method according to any one of statements 145 to 147, wherein the isolated cells are administered to the subject by a method selected from the group consisting of: intravenous injection, intraperitoneal injection, intra-lymphatic injection, intrathecal injection, injection into the cerebrospinal fluid (CSF), direct injection into a tumour, and as a gel placed on or near a solid tumour.

### Medical Uses

149. A glucocorticoid for use in a method according to any one of statements 101-148.
150. Use of a glucocorticoid for the manufacture of a medicament for use in a method according to any one of statements 101-148.
151. Use of dexamethasone to induce and/or mobilize a population of NKT-like cells, wherein the population of cells is induced and/or mobilized by a method according to any one of statements 101-148.
AVM-NKT Derived iPSCs
152. A method of producing induced pluripotent stem cells (iPSCs), the method comprising reprogramming cells isolated by a method according to any one of statements 136-138 to produce iPSCs.
153. The method of statement 152, wherein the reprogramming comprises introducing one or more expression cassettes encoding Oct3/4, Klf4, Sox2, and C-myc into the cells.
154. The method of statement 152, wherein the reprogramming comprises introducing Oct3/4, KLF4, Sox2, and c-myc encoding mRNA into the cells.
155. The method of statement 153 or 154, wherein the reprogramming further comprises introducing one or more expression cassettes encoding one or more of: Sox1, Sox3, Sox15, Klf1, Klf2, Klf5, L-myc, N-myc, Nanog, and/or LIN28 into the cells.
156. The method of statement 153 or 154, wherein the reprogramming further comprises introducing one or more of: Sox1, Sox3, Sox15, Klf1, Klf2, Klf5, L-myc, N-myc, Nanog, and/or LIN28 encoding mRNA into the cells.
157. The method according to any one of statement 152-156, further comprising inducing differentiation of the iPSCs.
158. The method according to statement 157, wherein the iPSCs are differentiated into NKT cells.
159. A method of producing a population of NKT-like cells, the method comprising differentiating iPSCs produced by a method according to any one of statement 152-156 into an NKT cell lineage.

### Isolated Human Immune System NKT-Like Cells

201. An isolated natural killer T cell-like cell (NKT-like cell) or population of NKT-like cells produced by a method according to any one of statements 101-159.
202. An isolated NKT-like cell, characterized in that the cell expresses CD56, TCR gamma/delta, and iTCR.
203. The isolated cell according to statement 202, wherein the cell is characterized in that:
i) the cell expresses CD56, TCR gamma/delta, iTCR, CD16, NKp44, CD3, CD8, CD14, CD19, CD45, TCR alpha/beta, CD34, and/or ICAM3;
ii) the cell expresses CD56, TCR gamma/delta, iTCR, CD16, NKp44, CD3, CD8, CD14, CD19, CD45, and/or TCR alpha/beta;
iii) the cell does not express CD4;
iv) the cell expresses CD56, TCR gamma/delta, iTCR, CD16, and NKp44;
v) the cell expresses CD56, TCR gamma/delta, iTCR, and TCR alpha/beta;
vi) the cell expresses CD56, TCR gamma/delta, iTCR, CD16, NKp44, and TCR alpha/beta;
vii) the cell expresses CD56, TCR gamma/delta, iTCR, CD16, NKp44, CD8, CD14, and CD19
viii) the cell expresses CD56, TCR gamma/delta, iTCR, CD16, NKp44, CD3, CD8, CD14, CD19, and CD45;
ix) the cell expresses CD56, TCR gamma/delta, iTCR, CD16, NKp44, CD3, CD8, CD14, CD19, CD45, and TCR alpha/beta; and/or.
x) the cell expresses a marker combination defined in statement 103.
   204. An isolated population of NKT-like cells, characterized in that at least 60, 70, 80, 90, 95, 96, 97, 98, or 99% of the cells express CD56, TCR gamma/delta, and iTCR.
   205. The isolated population of cells according to statement 204, wherein the population of cells are characterized in that:
i) at least 60, 70, 80, 90, 95, 96, 97, 98, or 99% of the cells express CD56, TCR gamma/delta, iTCR, CD16, NKp44, CD3, CD8, CD14, CD19, CD45, TCR alpha/beta, CD34, and/or ICAM3;
ii) at least 60, 70, 80, 90, 95, 96, 97, 98, or 99% of the cells express CD56, TCR gamma/delta, iTCR, CD16, NKp44, CD3, CD8, CD14, CD19, CD45, and/or TCR alpha/beta;
iii) at least 60, 70, 80, 90, 95, 96, 97, 98, or 99% of the cells do not express CD4;
iv) at least 60, 70, 80, 90, 95, 96, 97, 98, or 99% of the cells express CD56, TCR gamma/delta, iTCR, CD16, and NKp44;
v) at least 60, 70, 80, 90, 95, 96, 97, 98, or 99% of the cells express CD56, TCR gamma/delta, iTCR, and TCR alpha/beta;
vi) at least 60, 70, 80, 90, 95, 96, 97, 98, or 99% of the cells express CD56, TCR gamma/delta, iTCR, CD16, NKp44, and TCR alpha/beta;
vii) at least 60, 70, 80, 90, 95, 96, 97, 98, or 99% of the cells express CD56, TCR gamma/delta, iTCR, CD16, NKp44, CD8, CD14, and CD19;
viii) at least 60, 70, 80, 90, 95, 96, 97, 98, or 99% of the cells express CD56, TCR gamma/delta, iTCR, CD16, NKp44, CD3, CD8, CD14, CD19, and CD45;
ix) at least 60, 70, 80, 90, 95, 96, 97, 98, or 99% of the cells express CD56, TCR gamma/delta, iTCR, CD16, NKp44, CD3, CD8, CD14, CD19, CD45, and TCR alpha/beta; And/or.
x) at least 60, 70, 80, 90, 95, 96, 97, 98, or 99% of the cells express a marker combination defined in statement 103.
   206. The isolated cell or isolated population of cells according to any one of statements 202-205, wherein the cell or cells are produced by a method according to any one of statements 101-159.
   207. The isolated cell or isolated population of cells according to any one of statements 202-205, wherein the cell or cells are naturally occurring cells.
   208. The isolated cell or isolated population of cells according to any one of statements 202-205 and 207, wherein the cell or cells have not been transfected, transduced, or otherwise modified to express TCR gamma/delta.
   209. The isolated cell or isolated population of cells according to any one of statements 202-205 and 207-208, wherein the cell or cells have not been transfected, transduced, or otherwise modified to express iTCR.
   210. The isolated cell or isolated population of cells according to any one of statements 202-205 and 207-209, wherein the cell or cells have not been transfected, transduced, or otherwise modified to express TCR alpha/beta.
   211. The isolated cell or isolated population of cells according to any one of statements 202-205 and 207-210, wherein the cell or cells have not been transfected, transduced, or otherwise modified to express CD56, TCR gamma/delta, iTCR, CD16, NKp44, CD3, CD8, CD14, CD19, CD45, TCR alpha/beta, CD34 and/or ICAM3.

### Methods of Treatment

301. A glucocorticoid for use in a method of treatment of cancer, autoimmune disease, or infectious disease in a subject, the method comprising administering a glucocorticoid to the subject at a dose equivalent to about 6-45 mg/kg human equivalent dose (HED) of dexamethasone base,
wherein the glucocorticoid induces a population of NKT-like cells characterized in that at least 60, 70, 80, 90, 95, 96, 97, 98, or 99% of the cells;
i) express CD56, TCR gamma/delta, iTCR, CD16, NKp44, CD3, CD8, CD14, CD19, CD45, TCR alpha/beta, CD34 and/or ICAM3;
ii) expresses CD56, TCR gamma/delta, iTCR, CD16, NKp44, CD3, CD8, CD14, CD19, CD45, and/or TCR alpha/beta; and/or
iii) do not express: CD4.
   302. A glucocorticoid for use in a method of treatment of cancer, autoimmune disease, or infectious disease in a subject, the method comprising administering a glucocorticoid to the subject at a dose equivalent to about 6-45 mg/kg human equivalent dose (HED) of dexamethasone base,
wherein the glucocorticoid induces a population of NKT-like cells as defined in any one of statements 101-159.
   303. A method of treating cancer, autoimmune disease, or infectious disease in a subject, the method comprising administering a glucocorticoid to the subject at a dose equivalent to about 6-45 mg/kg human equivalent dose (HED) of dexamethasone base,
wherein the glucocorticoid induces a population of NKT-like cells characterized in that at least 60, 70, 80, 90, 95, 96, 97, 98, or 99% of the cells;
i) express CD56, TCR gamma/delta, iTCR, CD16, NKp44, CD3, CD8, CD14, CD19, CD45, TCR alpha/beta, CD34 and/or ICAM3;
ii) expresses CD56, TCR gamma/delta, iTCR, CD16, NKp44, CD3, CD8, CD14, CD19, CD45, and/or TCR alpha/beta; and/or
iii) do not express: CD4.
   304. A method of treating cancer, autoimmune disease, or infectious disease in a subject, the method comprising administering a glucocorticoid to the subject at a dose equivalent to about 6-45 mg/kg human equivalent dose (HED) of dexamethasone base,
wherein the glucocorticoid induces a population of NKT-like cells as defined in any one of statements 101-159.
   305. An isolated NKT-like cell or population of NKT-like cells for use in a method of treating cancer, autoimmune disease, or infectious disease in a subject, the method comprising administering to a subject in need thereof a therapeutically effective dose of:
i) cells isolated according to any one of statements 136 to 144;
ii) cells according to any one of statements 201-211.
   306. A method of treating cancer, autoimmune disease, or infectious disease in a subject, the method comprising administering to a subject in need thereof a therapeutically effective dose of:
i) cells isolated according to any one of statements 136 to 144;
ii) cells according to any one of statements 201-211.

1. A method of producing a population of natural killer T-cell like cells (NKT-like cells), the method comprising administering to a human subject a glucocorticoid-receptor (GR) modulating agent at a dose equivalent to about at least 6 mg/kg human equivalent dose (HED) of dexamethasone base:
wherein the population of NKT cells are characterized in that at least 60, 70, 80, 90, 95, 96, 97, 98, or 99% of the cells express CD56, TCR gamma/delta, and iTCR.

2. The method according to claim 1, wherein the population of NKT-like cells are characterized in that at least 60, 70, 80, 90, 95, 96, 97, 98, or 99% of the cells:
i) express CD56, TCR gamma/delta, iTCR, CD16, NKp44, CD3, CD8, CD14, CD19, CD45, TCR alpha/beta, CD34, and/or ICAM3;
ii) express CD56, TCR gamma/delta, iTCR, CD16, NKp44, CD3, CD8, CD14, CD19, CD45, and/or TCR alpha/beta; and/or
iii) do not express: CD4.

3. The method according to claim 2, wherein the NKT-like cells express CD56, TCR gamma/delta, and iTCR, and:
i) CD16 and NKp44;
(ii) TCR alpha/beta;
(iii) CD16, NKp44, and TCR alpha/beta;
(iv) CD16, NKp44, CD8, CD14, and CD19;
(v) CD16, NKp44, CD3, CD8, CD14, CD19, and CD45; or
(vi) CD16, NKp44, CD3, CD8, CD14, CD19, CD45, and TCR alpha/beta.

4. The method according to claim 2 or 3, wherein the NKT-like cells are:
i) CD3+/dim;
ii) CD8+/dim;
iii) CD3+/dim and CD8+/dim;
optionally, wherein the expression levels are determined relative to the average expression level in a population of reference cells derived from a common source, which have not been contacted with the glucocorticoid-receptor (GR) modulating agent or ICAM3 modulating agent.

5. The method according to any one of claims 1-4, wherein the glucocorticoid-receptor (GR) modulating agent is a glucocorticoid, optionally wherein the glucocorticoid is selected from the group consisting of: dexamethasone, hydrocortisone, methylprednisolone, prednisone, prednisolone, prednylidene, cortisone, budesonide, betamethasone, flumethasone and beclomethasone.

6. The method according to claim 5, wherein the glucocorticoid is selected from the group consisting of: dexamethasone, betamethasone, and methylprednisone.

7. The method according to claim 6, wherein the glucocorticoid is dexamethasone or betamethasone.

8. The method according to any one of claims 5-7, wherein the dexamethasone is dexamethasone sodium phosphate.

9. The method according to any one of claims 1-8, wherein the glucocorticoid is administered at a dose equivalent to about:
i) at least 6-12 mg/kg human equivalent dose (HED) of dexamethasone base;
ii) at least 6 mg/kg human equivalent dose (HED) of dexamethasone base;
iii) at least 12 mg/kg human equivalent dose (HED) of dexamethasone base;
iv) at least 15 mg/kg human equivalent dose (HED) of dexamethasone base;
v) at least 21 mg/kg human equivalent dose (HED) of dexamethasone base;
vi) at least 24 mg/kg human equivalent dose (HED) of dexamethasone base;
vii) 15 mg/kg human equivalent dose (HED) of dexamethasone base;
vii) 24 mg/kg human equivalent dose (HED) of dexamethasone base: or
ix) 45 mg/kg human equivalent dose (HED) of dexamethasone base.

10. The method according to any one of claims 1-9, wherein the glucocorticoid is administered as a single acute dose, or as a total dose given over about a 72 hour period.

11. The method according to any one of claims 1-10, wherein the method comprises administering one or more further doses of a glucocorticoid.

12. The method according to any one of claims 1-11, further comprising a step of administering an NKT cell activator, T cell activator, and/or NK cell activator to the subject.

13. The method according to claim 12, wherein the NKT cell activator, T cell activator, and/or NK cell activator is administered within or around 1 hour after administration of glucocorticoid.

14. The method according to any one of claims 1-13, wherein the subject has, is suspected of having, or has been diagnosed with a disease selected from the group consisting of: cancer, autoimmune disease, or infectious disease; and/or the NKT-like cells treat a disease selected from the group consisting of: cancer, autoimmune disease, or infectious disease in the subject.

15. The method according to claim 14, wherein the cancer is a solid tumour cancer.

16. The method according to claim 15, wherein the cancer is lymphoma, preferably a B cell lymphoma, a T cell lymphoma, or non Hodgkin lymphoma, or a leukaemia, preferably T-ALL or B-ALL.

17. The method according to any one of claims 14-16, wherein the NKT-like cells treat the cancer via tumour infiltration.

18. The method according to any one of claims 14-17, wherein the NKT-like cells promote infiltration of other immune cells into the tumour.

19. The method according to any one of claims 14-18, wherein the NKT-like cells directly kill cancer cells via CD1d-directed apoptosis.

20. The method according to any one of claims 14-19, wherein the NKT-like cells treat the cancer by causing tumor necrosis.

21. The method according to claim 14, wherein the autoimmune disease is selected from the group consisting of: multiple sclerosis, systemic sclerosis, amyotrophic lateral sclerosis, type 1 diabetes mellitus (TID), scleroderma, pemphigus, and lupus.

22. The method according to claim 14, wherein the infectious disease is HIV or a disease resulting from infection with a coronavirus, such as COVID-19.

23. The method according to any one of claims 1-22, further comprising a step of isolating a population of NKT-like cells from the subject or from a sample derived from the subject,
optionally wherein the step of isolating is performed:
i) at least 48 hours after glucocorticoid administration; or
ii) between 48 hours and 13 days after glucocorticoid administration.

24. The method of claim 23, wherein the sample is selected from the group consisting of: blood, plasma, a tumor biopsy or surgically removed tumor, bone marrow, liver, and fat or adipose tissue.

25. The method according to claim 23 or 24, further comprising a step of expanding the isolated NKT-like cells.

26. The method according to any one of claims 23-25, further comprising a step of activating the isolated NKT-like cells with an NKT cell activator, T cell activator, and/or NK cell activator;
optionally wherein the NKT cell activator is selected from alpha GalCer and sulfatide;
optionally wherein the T cell activator is selected from zoledronate and mevastatin; and
optionally wherein the NK cell activator is selected from the group consisting of: IL-2, IL-12, IL-15, IL-18, IL-21.

27. The method according to any one of claims 23-25, further comprising a step of introducing a nucleic acid encoding a protein into the isolated NKT-like cells, and culturing the cells under conditions that facilitate expression of said protein.

28. The method according to claim 27, wherein the protein is selected from the group consisting of one or more of: a T-cell receptor (TCR), a chimeric antigen receptor (CAR), and a split, universal and programmable CAR (SUPRA-CAR).

29. The method according to any one of claims 23-28, further comprising a step of expanding the NKT-like cells.

30. The method according to any one of claims 23-29, further comprising a step of activating the NKT-like cells with an NKT cell activator, T cell activator, and/or NK cell activator.

31. A method of treating cancer, autoimmune disease, or infectious disease in a subject, the method comprising administering a therapeutically effective dose of the isolated NKT-like cells of any one of claims 1-30 to the subject.

32. A glucocorticoid for use in a method according to any one of claims 1-31.

33. Use of a glucocorticoid for the manufacture of a medicament for use in a method according to any one of claims 1-31.

34. An isolated NKT-like cell or population of NKT-like cells produced by a method according to any one of claims 1-33.

35. An isolated NKT-like cell, characterized in that the cell expresses CD56, TCR gamma/delta, and iTCR, and optionally:
i) express CD56, TCR gamma/delta, iTCR, CD16, NKp44, CD3, CD8, CD14, CD19, CD45, TCR alpha/beta, CD34 and/or ICAM3;
ii) expresses CD16, NKp44, CD3, CD8, CD14, CD19, CD45, and/or TCR alpha/beta; and/or
iii) does not express: CD4

36. The isolated NKT-like cell according to claim 35, wherein the NKT-like cell or its precursor has not been transfected, transduced, or otherwise modified to express TCR gamma/delta.

37. The isolated NKT-like cell according to claim 35 or 36, wherein the NKT-like cell or its precursor has not been transfected, transduced, or otherwise modified to express iTCR.

38. The isolated NKT-like cell according to claim 35-37, wherein the NKT-like cell or its precursor has not been transfected, transduced, or otherwise modified to express CD56, TCR gamma/delta, iTCR, CD16, NKp44, CD3, CD8, CD14, CD19, CD45, TCR alpha/beta, CD34, and/or ICAM3.

39. An isolated population of NKT-like cells, characterized in that at least 60, 70, 80, 90, 95, 96, 97, 98, or 99% of the cells express CD56, TCR gamma/delta, and iTCR, and optionally:
i) express CD56, TCR gamma/delta, iTCR, CD16, NKp44, CD3, CD8, CD14, CD19, CD45, TCR alpha/beta, CD34 and/or ICAM3;
ii) express CD16, NKp44, CD3, CD8, CD14, CD19, CD45, and/or TCR alpha/beta; and/or
iii) do not express: CD4.

40. A glucocorticoid for use in a method of treatment of cancer, autoimmune disease, or infectious disease in a subject, the method comprising administering a glucocorticoid to the subject at a dose equivalent to about 6-45 mg/kg human equivalent dose (HED) of dexamethasone base,
wherein the glucocorticoid induces a population of NKT-like cells characterized in that at least 60, 70, 80, 90, 95, 96, 97, 98, or 99% of the cells express CD56, TCR gamma/delta, and iTCR, and optionally;
i) express CD56, TCR gamma/delta, iTCR, CD16, NKp44, CD3, CD8, CD14, CD19, CD45, TCR alpha/beta, CD34 and/or ICAM3;
ii) express CD16, NKp44, CD3, CD8, CD14, CD19, CD45, and/or TCR alpha/beta; and/or
iii) do not express: CD4.
